(19) United States
(12) Patent Application Publication
(10) Pub. No.: US 2026/0273092 A1
(43) Pub. Date: Sep. 17, 2026

(54) COMPOSITIONS COMPRISING OLIGONUCLEOTIDE AND USES THEREOF

(71) Applicant: siRNAgen Therapeutics Incorporated, Boston, MA (US)

(72) Inventors: Han Oh PARK, Daejeon (KR); June Young PARK, Boston, MA (US); Sam Yong LEE, Daejeon (KR); Ji Young KIM, Daejeon (KR); Young Ho KO, Daejeon (KR); Jun Hong PARK, Daejeon (KR); Jang seon KIM, Daejeon (KR)

(21) Appl. No.: 18/868,326

(22) PCT Filed: May 23, 2023
(86) PCT No.: PCT/US2023/023286

§ 371 Date: Nov. 22, 2024

Related U.S. Application Data
(60) Provisional application No. 63/432,531, filed on Dec. 14, 2022.

(30) Foreign Application Priority Data
May 23, 2022 (KR) 10-2022-0062663

(51) Int. Cl. A61K 47/69; A61K 31/713; A61K 47/54; A61K 47/60; A61P 25/28; C12N 15/113

(52) U.S. Cl. CPC A61K 47/6929; A61K 31/713; A61K 47/549; A61K 47/60; A61P 25/28; C12N 15/1137; C12N 2310/14; C12Y 304/23046

(57) ABSTRACT

Provided herein are compounds comprising an oligonucleotide molecule conjugated to a ligand for a glucose transporter (GLUT) for modulation of a target molecule, compositions comprising the same, and methods of using the compositions.

### CROSS-REFERENCE TO RELATED APPLICATIONS

This application claims the benefit of U.S. Provisional Application No. 63/432,531 filed on Dec. 14, 2022 and Korean Patent Application No. 10-2022-0062663 filed on May 23, 2022, each of which are hereby incorporated by reference in their entireties.

### SEQUENCE LISTING

The instant application contains a Sequence Listing with 245 sequences.

### BACKGROUND

The development of innovative therapeutic strategies for the treatment of brain disorders and cancer has always been a subject of intense research and exploration. In recent years, RNA interference (RNAi) has emerged as a promising therapeutic approach, particularly with the use of small interfering RNA (siRNA) and SAMiRNA™ technology.

However, the successful clinical application of the therapeutics has been hindered by the challenges associated with efficient and targeted delivery of the RNAi molecules to specific tissues and cells, particularly to the brain. The blood-brain barrier (BBB), a highly selective barrier that separates the central nervous system (CNS) from the systemic circulation, poses a major hurdle for the delivery of therapeutic agents to the brain. Additionally, in the case of cancer, effective delivery of RNAi molecules to tumor cells while minimizing off-target effects is a significant challenge.

Therefore, the development of an efficient delivery system that can overcome these barriers and provide targeted delivery to the brain and tumor cells holds immense therapeutic potential. Such a system would enhance the efficacy and specificity of RNAi-based therapies, leading to improved treatment outcomes for patients with brain disorders and cancer.

### TECHNICAL FIELD

The present disclosure relates to a compound comprising an oligonucleotide molecule, and a composition comprising the same, a method of preventing or treating a disorder, a method of alleviating or delaying one or more signs or symptoms of a disorder, a method of targeted delivery of a compound, a method of reducing or inhibiting a target molecule expression and/or activity in a specific tissue, organ, or cell, and a method of increasing blood brain barrier penetration of a compound.

### SUMMARY

In an aspect, provided herein is a compound comprising the structure of Formula 16:

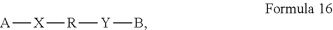

wherein:
R is an oligonucleotide molecule;
A is a hydrophilic moiety;
B is a hydrophobic moiety; and
X and Y are independently a covalent bond or a first linker;
wherein a ligand molecule for a glucose transporter (GLUT), or functional analog thereof is covalently linked to a terminus of A, a terminus of B, or a combination thereof; and
wherein R modulates the expression level and/or activity level of a target molecule.

In some embodiments, the ligand molecule for a glucose transporter (GLUT), or functional analog thereof is covalently linked to a terminus of A, a terminus of B, or a combination thereof via a second linker.

In some embodiments, the second linker is —NH(CH₂)ₙCONH—CH₂—CH(OH)—CH₂—.

In some embodiments, the ligand molecule for a glucose transporter (GLUT), or functional analog thereof is covalently linked to a terminus of A. In some embodiments, the ligand molecule for a glucose transporter (GLUT), or functional analog thereof is covalently linked to a terminus of B. In some embodiments, the ligand molecule for a glucose transporter (GLUT), or functional analog thereof is covalently linked to the terminus of both A and B.

In some embodiments, is the present disclosure provides a compound comprising the structure of Formula 16:

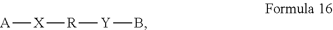

wherein:
R is an oligonucleotide molecule;
A is a hydrophilic moiety;
B is a hydrophobic moiety; and
X and Y are independently a covalent bond or a first linker;
wherein a ligand molecule for a glucose transporter (GLUT), or functional analog thereof is covalently linked to a terminus of A, a terminus of B, or a combination thereof via a second linker;
wherein the second linker is —NH(CH₂)ₙCONH—CH₂—CH(OH)—CH₂—; and
wherein R modulates the expression level and/or activity level of a target molecule.

In some embodiments, the compound comprises the structure of Formula 14 or Formula 15:

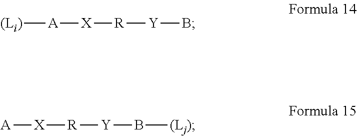

and
wherein L is the ligand molecule for a GLUT, or functional analog thereof;
wherein i and j are independently an integer of 0 through 10; and
wherein when i is 0, j is an integer of 1 through 10, and when j is 0, i is an integer of 1 through 10.

In some embodiments, the ligand molecule for a GLUT is a sugar.

In some embodiments, the sugar is selected from the group consisting of fructose, galactose, glucosamine, glucose, mannose, xylose, dehydroascorbic acid, and trehalose.

In some embodiments, the ligand molecule for a GLUT is selected from the group consisting of fructose, galactose, glucosamine, glucose, mannose, xylose, dehydroascorbic acid, trehalose, and urate.

In some embodiments, the GLUT is selected from the group consisting of GLUT1-GLUT14.

In some embodiments, the ligand molecule for a GLUT is fructose and the GLUT is selected from the group consisting of GLUT2, GLUT5, GLUT7, GLUT8, GLUT9, GLUT11, and GLUT12.

In some embodiments, the ligand molecule for a GLUT is galactose and the GLUT is selected from the group consisting of GLUT1, GLUT2, GLUT3, GLUT4, and GLUT14.

In some embodiments, the ligand molecule for a GLUT is glucose and the GLUT is selected from the group consisting of GLUT1, GLUT2, GLUT3, GLUT4, GLUT6, GLUT7, GLUT8, GLUT9, GLUT10, GLUT11, and GLUT12.

In some embodiments, the ligand molecule for a GLUT is mannose and the GLUT is selected from the group consisting of GLUT1, GLUT2, GLUT3, and GLUT4.

In some embodiments, the ligand molecule for a GLUT is xylose and the GLUT is GLUT3.

In some embodiments, the ligand molecule for a GLUT is dehydroascorbic acid and the GLUT is GLUT4.

In some embodiments, the ligand molecule for a GLUT is trehalose and the GLUT is GLUT8.

In some embodiments, the ligand molecule for a GLUT is urate and the GLUT is GLUT9.

In some embodiments, R is a double stranded oligonucleotide molecule or a single stranded oligonucleotide molecule.

In some embodiments, R is an inhibitory oligonucleotide molecule.

In some embodiments, the inhibitory oligonucleotide molecule reduces or inhibits expression and/or activity of the target molecule.

In some embodiments, the target molecule is any one selected from the group consisting of BACE1-AS, BACE1, presenilin1 (PS1), ROCK-II, 12 PP-2A, ACAT-1, Nogo receptor, mutant presenilin1 (L392V PS-1), APP, Tau, VDAC1, α-synuclein, Huntingtin (Htt), Notch1, LINGO-1, NR4A2, TRIF, caspase-2, CaMKII, glial fibrillary acidic protein (GFAP), Vimentin, EphB3, iNOS, Nischarin, and RhoA. Specifically, the target molecule can be an mRNA transcript of BACE1-AS, BACE1, presenilin1 (PS1), ROCK-II, 12 PP-2A, ACAT-1, Nogo receptor, mutant presenilin1 (L392V PS-1), APP, Tau, VDAC1, α-synuclein, Huntingtin (Htt), Notch1, LINGO-1, NR4A2, TRIF, caspase-2, CaMKII, glial fibrillary acidic protein (GFAP), Vimentin, EphB3, iNOS, Nischarin, or RhoA.

In some embodiments, the inhibitory oligonucleotide molecule is an siRNA, an shRNA, a miRNA, an amiRNA, an antisense oligonucleotide molecule, an RNAi agent, or any combination thereof.

In some embodiments, R is a double strand oligonucleotide molecule comprising a sense strand and an antisense strand comprising a sequence complementary to the sense strand.

In some embodiments, the sense strand or anti-sense strand is 19 to 31 nucleotides in length.

In some embodiments, at least one phosphate group is linked to the 5′ terminus of the anti-sense strand.

In some embodiments, the compound comprises the structure of Formula 2:

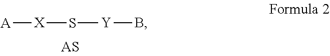

where S is the sense strand, and AS is the anti-sense strand.

In some embodiments, R is the antisense oligonucleotide molecule consisting of from 5 to 50 oligonucleotides, 10 to 40 oligonucleotides, or 15 to 30 oligonucleotides.

In some embodiments, R is DNA, RNA, or a combination thereof. In some embodiments, R is a double-stranded oligonucleotide comprising a sequence in the form of RNA/RNA, DNA/DNA or DNA/RNA hybrid.

In some embodiments, R comprises a backbone modification comprising a phosphorothioate linkage, a phosphorodiamidate linkage, a boranophosphate linkage, a methyl phosphonate linkage, or any combination thereof.

In some embodiments, R comprises a locked nucleic acid, a peptide nucleic acid, a unlocked nucleic acid, or any combination thereof.

In some embodiments, R comprises a 2′-O-methyl moiety, a 2′-fluoro moiety, a 2′-O-methoxyethyl moiety, a 2′-methyl moiety, a 2′-methoxyl moiety, a 2′-amine moiety, a 2′-O-propyl moiety, a 2′-O-2-methylthioethyl moiety, a 2′-O-3-aminopropyl moiety, a 2′-O-3-dimethylaminopropyl moiety, a 2′-O—N-methylacetamino moiety, or a 2′-O-dimethylamidooxyethyl moiety.

In some embodiments, R comprises a modified sugar moiety.

In some embodiments, the hydrophilic moiety is any one selected from the group consisting of polyethylene glycol (PEG), HEG (hexaethylene glycol), polyvinyl pyrrolidone and polyoxazoline. In some embodiments, the hydrophilic moiety is HEG (hexaethylene glycol). In some embodiments, the hydrophilic moiety comprises or consist of hexaethyleneglycol-(—PO₃⁻ hexaethyleneglycol)₃.

In some embodiments, the compound comprises the structure of Formula 7 or Formula 8:

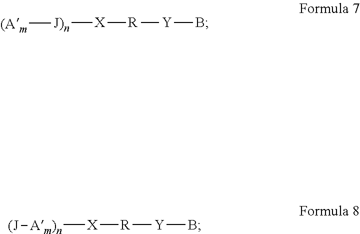

wherein:
A′ is a monomer hydrophilic moiety,
J is selected from the group comprised of PO₃⁻, SO₃⁻ and CO₂⁻, and links A′ to each other or links A′ to X,
m is an integer of 1 to 15, and
n is an integer of 1 to 10; and
wherein A′ is any one selected from the group consisting of Compound 1, Compound 2, and Compound 3:

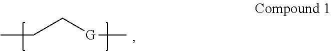

wherein G is selected from the group comprised of O, S and NH;

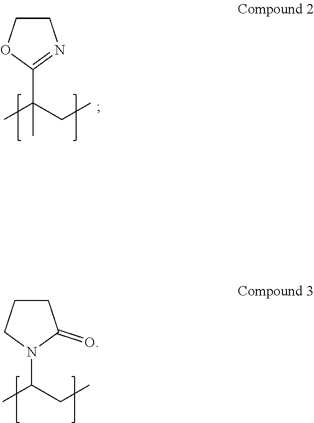

In some embodiments, the molecular weight of the hydrophilic moiety is 200 to 10,000.

In some embodiments, the hydrophobic moiety is any one selected from the group consisting of a steroid derivative, a glyceride derivative, glycerol ether, polypropylene glycol, unsaturated or saturated C₁₂-C₅₀, diacylphosphatidylcholine, a fatty acid, a phospholipid, a lipopolyamine, a lipid, tocopherol, and tocotrienol.

In some embodiments, the steroid derivative is any one selected from the group consisting of cholesterol, cholestanol, cholic acid, cholesteryl formate, cotestanyl formate, and colistanylamine.

In some embodiments, the glyceride derivative is any one selected from the group consisting of monoglyceride, diglyceride and triglyceride.

In some embodiments, the molecular weight of the hydrophobic moiety is 250 to 1,000.

In some embodiments, X and Y are independently a non-degradable bond or a degradable bond.

In some embodiments, the non-degradable bond is an amide bond or a phosphorylated bond.

In some embodiments, the degradable bond is any one selected from the group consisting of a disulfide bond, an acid-degradable bond, an ester bond, an anhydride bond, a biodegradable bond, and an enzyme degradable bond.

In another aspect, provided herein is a nanoparticle comprising the compound as provided herein.

In another aspect, provided herein is a composition comprising the compound as provided herein or the nanoparticle as provided herein.

In another aspect, provided herein is a pharmaceutical composition comprising the compound as provided herein, the nanoparticle as provided herein, or the composition as provided herein, and a pharmaceutically acceptable excipient, carrier, or diluent.

In another aspect, provided herein is a method of preventing or treating a disorder in a subject in need thereof comprising administering to the subject a therapeutically effective amount of the compound as provided herein, the nanoparticle as provided herein, the composition as provided herein, or a pharmaceutical composition as provided herein, wherein the disorder is associated with expression and/or activity of one or more pathological genes,
wherein the target molecule is the one or more pathological genes, and
wherein the administration reduces or inhibits expression and/or activity of the target molecule in the subject compared with a target molecule expression and/or activity in a subject prior to or in the absence of administration of the compound as provided herein, the nanoparticle as provided herein, the composition as provided herein, or a pharmaceutical composition as provided herein, thereby, alleviating or delaying one or more signs or symptoms of the disorder in the subject relative to a subject prior to or in the absence of the administration of the compound as provided herein, the nanoparticle as provided herein, the composition as provided herein, or a pharmaceutical composition as provided herein.

In another aspect, provided herein is a method of alleviating or delaying one or more signs or symptoms of a disorder in a subject in need thereof comprising administering a therapeutically effective amount of the compound as provided herein, the nanoparticle as provided herein, the composition as provided herein, or a pharmaceutical composition as provided herein to the subject,
wherein the disorder is associated with expression and/or activity of one or more pathological genes,
wherein the target molecule is the one or more pathological genes, and
wherein the administration reduces or inhibits expression and/or activity of the target molecule in the subject compared with a target molecule expression and/or activity in a subject prior to or in the absence of the administration of the compound of as provided herein, the nanoparticle as provided herein, the composition as provided herein, or a pharmaceutical composition as provided herein.

In another aspect, provided herein is a method of treating a disorder in a subject in need thereof comprising administering to the subject a therapeutically effective amount of a compound comprising the structure of Formula 17:

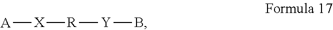

wherein:
R is an inhibitory oligonucleotide molecule;
A is a hydrophilic moiety;
B is a hydrophobic moiety; and
X and Y are independently a covalent bond or a first linker; and
wherein a ligand molecule for a glucose transporter (GLUT), or functional analog thereof is covalently linked to a terminus of A, a terminus of B, or a combination thereof via a second linker or a bond,
wherein the disorder is associated with expression and/or activity of one or more pathological genes,
wherein the target molecule is the one or more pathological genes, and
wherein the administration reduces or inhibits expression and/or activity of the target molecule in the subject compared with a target molecule expression and/or activity in a subject prior to or in the absence of the administration of the compound, thereby, alleviating or delaying one or more signs or symptoms of the disorder in the subject relative to a subject prior to or in the absence of the administration of the compound.

A method of alleviating or delaying one or more signs or symptoms of a disorder in a subject in need thereof comprising administering to the subject a therapeutically effective amount of a compound comprising the structure of Formula 17:

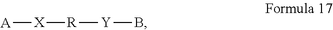

wherein:
R is an inhibitory oligonucleotide molecule;
A is a hydrophilic moiety;
B is a hydrophobic moiety; and
X and Y are independently a covalent bond or a first linker; and
wherein a ligand molecule for a glucose transporter (GLUT), or functional analog thereof is covalently linked to a terminus of A, a terminus of B, or a combination thereof via a second linker or a bond, and
wherein the administration reduces or inhibits expression and/or activity of the target molecule in the subject compared with a target molecule expression and/or activity in a subject prior to or in the absence of the administration of the compound, thereby, alleviating or delaying one or more signs or symptoms of the disorder in the subject relative to a subject prior to or in the absence of the administration of the compound.

In some embodiments, the second linker is —NH(CH₂)ₙCONH—CH₂—CH(OH)—CH₂—, an ether linkage, or Compound 4:

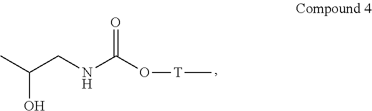

wherein T is 1-15 repetitive Compound 1:

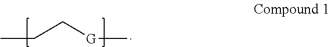

wherein G is selected from the group consisting of CH₂, O, S and NH.

In some embodiments, the compound comprises the structure of Formula 18 or Formula 19:

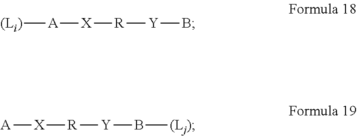

and
wherein L is the ligand molecule for a GLUT, or functional analog thereof;
wherein i and j are independently an integer of 0 through 10; and
wherein when i is 0, j is an integer of 1 through 10, and when j is 0, i is an integer of 1 through 10.

In some embodiments, the ligand molecule for a GLUT is a sugar.

In some embodiments, the sugar is selected from the group consisting of fructose, galactose, glucosamine, glucose, mannose, xylose, dehydroascorbic acid, and trehalose.

In some embodiments, the ligand molecule for a GLUT is selected from the group consisting of fructose, galactose, glucosamine, glucose, mannose, xylose, dehydroascorbic acid, trehalose, and urate.

In some embodiments, the GLUT is selected from the group consisting of GLUT1-GLUT14.

In some embodiments, the ligand molecule for a GLUT is fructose and the GLUT is selected from the group consisting of GLUT2, GLUT5, GLUT7, GLUT8, GLUT9, GLUT11, and GLUT12.

In some embodiments, the ligand molecule for a GLUT is galactose and the GLUT is selected from the group consisting of GLUT1, GLUT2, GLUT3, GLUT4, and GLUT14.

In some embodiments, the ligand molecule for a GLUT is glucose and the GLUT is selected from the group consisting of GLUT1, GLUT2, GLUT3, GLUT4, GLUT6, GLUT7, GLUT8, GLUT9, GLUT10, GLUT11, and GLUT12.

In some embodiments, the ligand molecule for a GLUT is mannose and the GLUT is selected from the group consisting of GLUT1, GLUT2, GLUT3, and GLUT4.

In some embodiments, the ligand molecule for a GLUT is xylose and the GLUT is GLUT3.

In some embodiments, the ligand molecule for a GLUT is dehydroascorbic acid and the GLUT is GLUT4.

In some embodiments, the ligand molecule for a GLUT is trehalose and the GLUT is GLUT8.

In some embodiments, the ligand molecule for a GLUT is urate and the GLUT is GLUT9.

In some embodiments, R is a double stranded oligonucleotide molecule or a single stranded oligonucleotide molecule.

In some embodiments, R reduces or inhibits expression and/or activity of a target molecule.

In some embodiments, the target molecule is any one selected from the group consisting of BACE1-AS, BACE1, presenilin1 (PS1), ROCK-II, 12 PP-2A, ACAT-1, Nogo receptor, mutant presenilin1 (L392V PS-1), APP, Tau, VDAC1, α-synuclein, Huntingtin (Htt), Notch1, LINGO-1, NR4A2, TRIF, caspase-2, CaMKII, glial fibrillary acidic protein (GFAP), Vimentin, EphB3, iNOS, Nischarin, and RhoA.

In some embodiments, the inhibitory oligonucleotide molecule is an siRNA, an shRNA, a miRNA, an amiRNA, an antisense oligonucleotide molecule, an RNAi agent, or any combination thereof.

In some embodiments, R is a double strand oligonucleotide molecule comprising a sense strand and an antisense strand comprising a sequence complementary to the sense strand.

In some embodiments, the sense strand or anti-sense strand is 19 to 31 nucleotides in length.

In some embodiments, at least one phosphate group is linked to the 5′ terminus of the anti-sense strand.

In some embodiments, the compound comprises the structure of Formula 2:

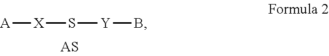

where S is the sense strand, and AS is the anti-sense strand.

In some embodiments, R is the antisense oligonucleotide molecule consisting of from 5 to 50 oligonucleotides, 10 to 40 oligonucleotides, or 15 to 30 oligonucleotides.

In some embodiments, R is DNA, RNA, or a combination thereof.

In some embodiments, R comprises a backbone modification comprising a phosphorothioate linkage, a phosphorodiamidate linkage, a boranophosphate linkage, a methyl phosphonate linkage, or any combination thereof.

In some embodiments, R comprises a locked nucleic acid, a peptide nucleic acid, a unlocked nucleic acid, or any combination thereof.

In some embodiments, R comprises a 2′-O-methyl moiety, a 2′-fluoro moiety, a 2′-O-methoxyethyl moiety, a 2′-methyl moiety, a 2′-methoxyl moiety, a 2′-amine moiety, a 2′-O-propyl moiety, a 2′-O-2-methylthioethyl moiety, a 2′-O-3-aminopropyl moiety, a 2′-O-3-dimethylaminopropyl moiety, a 2′-O—N-methylacetamino moiety, or a 2′-O-dimethylamidooxyethyl moiety.

In some embodiments, R comprises a modified sugar moiety.

In some embodiments, the hydrophilic moiety is any one selected from the group consisting of polyethylene glycol (PEG), polyvinyl pyrrolidone and polyoxazoline.

In some embodiments, the compound comprises the structure of Formula 7 or Formula 8:

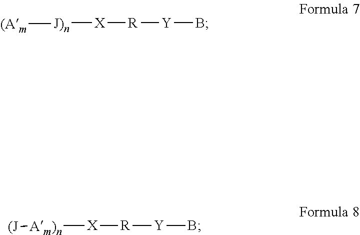

wherein:
A′ is a monomer hydrophilic moiety,
J is selected from the group comprised of PO₃⁻, SO₃⁻ and CO₂⁻, and links A′ to each other or links A′ to X,
m is an integer of 1 to 15, and
n is an integer of 1 to 10; and
   wherein A′ is any one selected from the group consisting of Compound 1, Compound 2, and Compound 3:

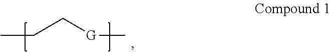

wherein G is selected from the group comprised of O, S and NH;

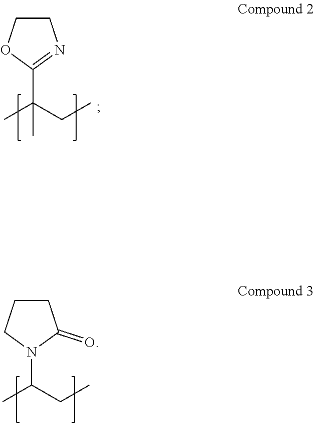

In some embodiments, the molecular weight of the hydrophilic moiety is 200 to 10,000.

In some embodiments, the hydrophobic moiety is any one selected from the group consisting of a steroid derivative, a glyceride derivative, glycerol ether, polypropylene glycol, unsaturated or saturated C₁₂-C₅₀, diacylphosphatidylcholine, a fatty acid, a phospholipid, a lipopolyamine, a lipid, tocopherol, and tocotrienol.

In some embodiments, the steroid derivative is any one selected from the group consisting of cholesterol, cholestanol, cholic acid, cholesteryl formate, cotestanyl formate, and colistanylamine.

In some embodiments, the glyceride derivative is any one selected from the group consisting of monoglyceride, diglyceride and triglyceride.

In some embodiments, the molecular weight of the hydrophobic moiety is 250 to 1,000.

In some embodiments, X and Y are independently a non-degradable bond or a degradable bond.

In some embodiments, the non-degradable bond is an amide bond or a phosphorylated bond.

In some embodiments, the degradable bond is any one selected from the group consisting of a disulfide bond, an acid-degradable bond, an ester bond, an anhydride bond, a biodegradable bond, and an enzyme degradable bond.

In some embodiments, the compound is formulated as a nanoparticle.

In some embodiments, the compound is formulated as a composition.

In some embodiments, the composition is formulated as a pharmaceutical composition, wherein the pharmaceutical composition further comprises a pharmaceutically acceptable excipient, carrier, or diluent.

In some embodiments, the disorder is a neurological disorder.

In some embodiments, the neurological disorder is Alzheimer's Disease (AD), stroke, dementia, muscular dystrophy (MD), multiple sclerosis (MS), amyotrophic lateral sclerosis (ALS), cystic fibrosis, Angel-man syndrome, Liddle syndrome, Parkinson's Disease, Pick's Disease, Pagets Disease, cancer, or traumatic brain damage.

In some embodiments, the disorder is Alzheimer's disease, and the target molecule is BACE1-AS, BACE1, presenilin1 (PS1), ROCK-II, 12 PP-2A, ACAT-1, Nogo receptor, mutant presenilin1 (L392V PS-1), APP, Tau, VDAC1, or any combination thereof.

In some embodiments, the disorder is Parkinson's disease, and the target molecule is α-synuclein.

In some embodiments, the disorder is Huntington's disease, and the target molecule is Huntingtin (Htt).

In some embodiments, the disorder is multiple sclerosis, and the target molecule is Notch1, LINGO-1, NR4A2, TRIF, caspase-2, CaMKII, or any combination thereof.

In some embodiments, the disorder is spinal cord injury, and the target molecule is glial fibrillary acidic protein (GFAP), Vimentin, EphB3, iNOS, Nischarin, RhoA, or any combination thereof.

In some embodiments, the disorder is cancer.

In some embodiments, the cancer is brain cancer, breast cancer, cervix cancer, colon cancer, kidney cancer, lung cancer, ovary cancer, prostate cancer, skin cancer, thyroid cancer, glioblastoma, pancreas cancer, leukemia, gastric cancer, colorectal cancer, or any combination thereof.

In some embodiments, the cancer is glioblastoma, non-small cell lung carcinoma, prostate carcinoma, pancreatic ductal adenocarcinoma, metastatic gastric adenocarcinoma, invasive ductal carcinoma, or colorectal carcinoma.

In some embodiments, the one or more signs or symptoms comprises Aβ peptide accumulation in the brain, tau peptide accumulation in the brain, phosphorylated tau accumulation in the brain, tau protein phosphorylation, a hippocampal damage, a cognitive deficit, or any combination thereof.

In some embodiments, the AB peptide accumulation occurs in the CA1 and subiculum of the brain of the subject.

In some embodiments, the tau peptide accumulation occurs in the CA1 and subiculum of the brain of the subject.

In some embodiments, the phosphorylated tau accumulation occurs in the CA1 and subiculum of the brain of the subject.

In some embodiments, the subject shows no or minimum systemic toxicity comparable to a control subject prior to or in the absence of the administration.

In some embodiments, the subject shows no or minimum hepatotoxicity comparable to a control subject prior to or in the absence of the administration.

In some embodiments, the hepatotoxicity is assessed by the alkaline phosphatase (ALP) level, by the alanine aminotransferase (ALT) level, the aspartate aminotransferase (AST) level, the total bilirubin (T-bil) level, the ammonia (NH3) level, or any combination thereof in the subject.

In some embodiments, the subject shows no or minimum kidney toxicity comparable to a control subject prior to or in the absence of the administration.

In some embodiments, the kidney toxicity is assessed by the lactic acid (Lac) level, the blood urea nitrogen (BUN) level, the creatinine (Crea) level, or any combination thereof in the subject.

In some embodiments, the subject shows no or minimum altered lipid metabolism comparable to a control subject prior to or in the absence of the administration.

In some embodiments, the lipid metabolism is assessed by the lipase (Lip) level, the high density lipoprotein (HDL) level, the low density lipoprotein (LDL) level, the total cholesterol (T-Chol) level, the triglycerides (TG) level, or any combination thereof in the subject.

In some embodiments, the subject shows no or minimum altered calcium homeostasis comparable to a control subject prior to or in the absence of the administration.

In some embodiments, the calcium homeostasis is assessed by the calcium (Ca) level, the phosphorus (P) level, or a combination thereof in the subject.

In some embodiments, the blood brain barrier penetration of the compound in the brain of the subject is increased compared with the blood brain barrier penetration of a compound having the same structure without the ligand molecule for a GLUT, or functional analog thereof linked.

In some embodiments, the retention time and/or the distribution of the compound in the brain of the subject is increased compared with the retention time and/or the distribution of a compound having the same structure without the ligand molecule for a GLUT, or functional analog thereof linked.

In some embodiments, the compound is more specifically delivered to one or more tissues, organs, or cells that express the GLUT compared with tissues, organs, or cells that do not express the GLUT.

In some embodiments, the target molecule expression and/or activity is reduced or inhibited in one or more tissues, organs, or cells that express the GLUT to a greater extent compared with tissues, organs, or cells that do not express the GLUT.

In some embodiments, the one or more tissues, organs, or cells that express the GLUT comprises brain, blood, brain, intestine, liver, kidney, pancreas, embryos, testis, placenta, muscle, heart, fat, adipose tissue, spleen, colon, prostate, testis, blastocysts, skeletal muscle, white adipose tissue, brown adipose tissue, or any combination thereof.

In some embodiments, (i) the GLUT is GLUT1, and the one or more tissues, organs, or cells that express the GLUT comprises brain, blood, or a combination thereof;
(ii) the GLUT is GLUT2, and the one or more tissues, organs, or cells that express the GLUT comprises brain, intestine, liver, kidney, pancreas, or any combination thereof;
(iii) the GLUT is GLUT3, and the one or more tissues, organs, or cells that express the GLUT comprises brain, embryos, testis, placenta, or any combination thereof;
(iv) the GLUT is GLUT4, and the one or more tissues, organs, or cells that express the GLUT comprises brain, muscle, heart, fat, adipose tissue, or any combination thereof;
(v) the GLUT is GLUT5, and the one or more tissues, organs, or cells that express the GLUT comprises brain, intestine, testis, muscle, kidney, fat, or any combination thereof;
(vi) the GLUT is GLUT6, and the one or more tissues, organs, or cells that express the GLUT comprises brain, spleen, or a combination thereof;
(vii) the GLUT is GLUT7, and the one or more tissues, organs, or cells that express the GLUT comprises intestine, colon, prostate, testis, prostate gland, or any combination thereof;
(viii) the GLUT is GLUT8, and the one or more tissues, organs, or cells that express the GLUT comprises brain, testis, liver, spleen, fat, blastocysts, or any combination thereof;
(ix) the GLUT is GLUT9, and the one or more tissues, organs, or cells that express the GLUT comprises liver, kidney, intestine, colon, or any combination thereof;
(x) the GLUT is GLUT10, and the one or more tissues, organs, or cells that express the GLUT comprises liver, pancreas, fat, skeletal muscle, heart, adipose tissue, placenta, kidney, or any combination thereof;
(xi) the GLUT is GLUT11, and the one or more tissues, organs, or cells that express the GLUT comprises heart, muscle, kidney, pancreas, placenta, or any combination thereof;
(xii) the GLUT is GLUT12, and the one or more tissues, organs, or cells that express the GLUT comprises brain, muscle, heart, fat, pancreas, prostate, adipose tissue, placenta, kidney, or any combination thereof;
(xiii) the GLUT is GLUT13, and the one or more tissues, organs, or cells that express the GLUT comprises brain, white adipose tissue, brown adipose tissue, kidney, or any combination thereof; or
(xiv) the GLUT is GLUT14, and the one or more tissues, organs, or cells that express the GLUT

In some embodiments, (i) the GLUT is GLUT1, and the one or more tissues, organs, or cells that express the GLUT comprises blood-brain barrier, astrocyte, erythrocyte, or any combination thereof;
(ii) the GLUT is GLUT2, and the one or more tissues, organs, or cells that express the GLUT comprises astrocyte, gastrointestinal tract, small intestine, intestinal absorptive epithelial cells, hepatocytes, pancreatic B-cells, brain nuclei, nucleus tractus solitatius, motor nucleus of the vagus, paraventricular hypothalamic nucleus, lateral hypothalamic area, arcuate nucleus, and olfactory bulb, or any combination thereof;
(iii) the GLUT is GLUT3, and the one or more tissues, organs, or cells that express the GLUT comprises neuron, blood-brain barrier, astrocyte, sperm, spermatozoa, fibroblast, platelet, retinal endothelial cell, white blood cell, or any combination thereof;
(iv) the GLUT is GLUT4, and the one or more tissues, organs, or cells that express the GLUT comprises neuron, adipocyte, skeletal muscle cell, cardiomyocyte, hypothalamus, cerebellum, cortex, and hippocampus, or a combination thereof;
(v) the GLUT is GLUT5, and the one or more tissues, organs, or cells that express the GLUT comprises microglia, blood-brain barrier, small intestine, apical membrane of the enterocytes, plasma membrane of mature spermatozoa, skeletal muscle, or any combination thereof;
(vi) the GLUT is GLUT6, and the one or more tissues, organs, or cells that express the GLUT comprises leukocyte, peripheral leukocyte, germinal cell of the testis, or any combination thereof;
(vii) the GLUT is GLUT7, and the one or more tissues, organs, or cells that express the GLUT comprises small intestine;
(viii) the GLUT is GLUT8, and the one or more tissues, organs, or cells that express the GLUT comprises neuron, brown adipose tissue, cerebellum, adrenal gland, spermatocyte, mature spermatozoa, or any combination thereof;
(ix) the GLUT is GLUT9, and the one or more tissues, organs, or cells that express the GLUT comprises small intestine, leukocyte, chondrocyte, or any combination thereof;
(x) the GLUT is GLUT10, and the one or more tissues, organs, or cells that express the GLUT comprises white adipose;
(xi) the GLUT is GLUT12, and the one or more tissues, organs, or cells that express the GLUT comprises astrocyte, prostate gland, small intestine, chondrocyte, or any combination thereof; or
(xii) the GLUT is GLUT13, and the one or more tissues, organs, or cells that express the GLUT comprises neuron.

In some embodiments, the one or more tissues, organs, or cells that express the GLUT

In some embodiments, the cancer is selected from the group consisting of brain cancer, breast cancer, cervix cancer, colon cancer, kidney cancer, lung cancer, ovary cancer, prostate cancer, skin cancer, thyroid cancer, glioblastoma, pancreas cancer, leukemia, gastric cancer, colorectal cancer, and any combination thereof.

In some embodiments, (i) the GLUT is GLUT1, and the cancer is selected from the group consisting of brain cancer, breast cancer, cervix cancer, colon cancer, kidney cancer, lung cancer, ovary cancer, prostate cancer, skin cancer, thyroid cancer, and any combination thereof;
(ii) the GLUT is GLUT3, and the cancer is selected from the group consisting of brain cancer, breast cancer, colon cancer, cervix cancer, kidney cancer, lung cancer, glioblastoma, and any combination thereof;
(iii) the GLUT is GLUT5, and the cancer is selected from the group consisting of breast cancer, colon cancer, pancreas cancer, ovary cancer, lung cancer, leukemia, and any combination thereof;
(iv) the GLUT is GLUT10, and the cancer is leukemia;
(v) the GLUT is GLUT12, and the cancer is breast cancer; or
(vi) the GLUT is GLUT14, and the cancer is brain cancer.

In some embodiments, (i) the target molecule is BACE1-AS, and the administration results in improvement of memory and learning behaviors;
(ii) the target molecule is BACE1, and the administration results in decrease of amyloid plaque rate, improvement in neuropathological and behavioral signs, suppression of BACE1 expression and/or activity, reduction of plaque load in the cerebral cortex and hippocampus, increase in the level of synaptophysin, rescue of memory loss, or any combination thereof;
(iii) the target molecule is presenilin1 (PS1), and the administration results in reduction of the level of Aβ 42;
(iv) the target molecule is ROCK-II, and the administration results in promotion of axonal regeneration;
(v) the target molecule is I2 PP-2A, and the administration results in decrease of the level of Aβ and APP and phosphorylated tau, improvement of memory and learning ability, or a combination thereof;
(vi) the target molecule is ACAT-1, and the administration results in reductio of the enzymatic process of APP, enhancement of the level of free cholesterol, or a combination thereof;
(vii) the target molecule is Nogo receptor, and the administration results in promotion of the regeneration and repair of cholinergic neurons;
(viii) the target molecule is mutant presenilin1 (L392V PS-1), BACE1, or a combination thereof, and the administration results in decrease of the level of amyloid plaque;
(ix) the target molecule is APP, Tau, VDAC1, or a combination thereof, and the administration results in improvement in synaptic activity and mitochondrial function;
(x) the target molecule is α-synuclein (SNCA), and the administration results in reduction of the level of SNCA, reduction of hSNCA-mediated behavioral deficits, improvement in motor dysfunction, protection of cells from death via apoptosis, reduction of α-syn accumulation, amelioration of inflammatory pathology, or any combination thereof;
(xi) the target molecule is Htt, and the administration results in inhibition of the Htt expression and/or activity, reduction of size and number of neuronal intranuclear inclusions (NIIs), improvement of movement deficits, survival of neurons, decreasing the level of mutant htt protein, or any combination thereof;
(xii) the target molecule is T-bet, and the administration results in regulation of interferon (IFN), prevention of the onset of disease, or a combination thereof;
(xiii) the target molecule is Notch1, and the administration results in improved motor function, repair in histopathological sections, or a combination thereof;
(xiv) the target molecule is LINGO-1, and the administration results in improvement of memory and learning behaviors;
(xv) the target molecule is NR4A2, and the administration results in inhibition of the pathogenic potentials of IFN and IL-17;
(xvi) the target molecule is TRIF, and the administration results in alleviation of the severity of the disease via the inhibition of interleukin and cytokine release;
(xvii) the target molecule is caspase-2, and the administration results in inhibition of nerve cell loss, decrease in retinal nerve fiber layer (RNFL) thickness, increased survival of retinal ganglion cells (RGC), or any combination thereof;
(xviii) the target molecule is CaMKII, and the administration results in reduction mechanical and thermal hypersensitivity, reduction of the evoked and non-evoked pain, or a combination thereof;
(xix) the target molecule is GFAP, Vimentin, or a combination thereof, and the administration results in improvement of urinary function;
(xx) the target molecule is EphB3, and the administration results in improvement of axonal regeneration, improvement of the motor function, or a combination thereof;
(xxi) the target molecule is iNOS, and the administration results in improvement of the secondary damage following spinal cord injury;
(xxii) the target molecule is Nischarin, and the administration results in improvement of motor function; or
(xxiii) the target molecule is RhoA, and the administration results in improvement in walking, declining of allodynia, or a combination thereof.

In another aspect, provided herein is a method of targeted delivery of a compound comprising the structure of Formula 20 to a specific tissue, organ, or cell in a subject in need thereof comprising:

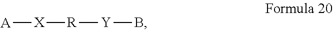

wherein:
R is an oligonucleotide molecule;
A is a hydrophilic moiety;
B is a hydrophobic moiety; and
X and Y are independently a covalent bond or a first linker,
(i) covalently linking a ligand molecule for a glucose transporter (GLUT), or functional analog thereof via a second linker or a bond to a terminus of A, a terminus of B, or a combination thereof of the compound comprising the structure of Formula 20; and
(ii) administering to the subject;
wherein the specific tissue, organ, or cell expresses the GLUT; and
wherein the compound is delivered to a greater extent to the specific tissue, organ, or cell compared with tissues, organs, or cells that do not express the GLUT.

In another aspect, provided herein is a compound for targeted delivery to a specific tissue, organ, or cell in a subject in need thereof, comprising the structure of Formula 20:

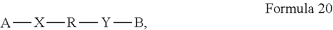

wherein:
R is an oligonucleotide molecule;
A is a hydrophilic moiety;
B is a hydrophobic moiety; and
X and Y are independently a covalent bond or a first linker,
wherein a ligand molecule for a glucose transporter (GLUT), or functional analog thereof is covalently linked to a terminus of A, a terminus of B, or a combination thereof via a second linker or a bond; and
wherein the specific tissue, organ, or cell expresses the GLUT; and
wherein when administered to the subject, the compound is delivered to a greater extent to the specific tissue, organ, or cell compared with tissues, organs, or cells that do not express the GLUT.

In some embodiments, R is an inhibitory oligonucleotide molecule.

In another aspect, provided herein is a method of reducing or inhibiting a target molecule expression and/or activity in a specific tissue, organ, or cell of a subject in need thereof comprising administering to the subject a therapeutically effective amount of a compound comprises the structure of Formula 17:

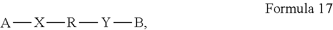

wherein:
R is an inhibitory oligonucleotide molecule;
A is a hydrophilic moiety;
B is a hydrophobic moiety; and
X and Y are independently a covalent bond or a first linker; and
wherein a ligand molecule for a glucose transporter (GLUT), or functional analog thereof is covalently linked to a terminus of A, a terminus of B, or a combination thereof via a second linker or a bond;
wherein the specific tissue, organ, or cell expresses the GLUT;
wherein the compound reduces or inhibits expression and/or activity of the target molecule in the specific tissue, organ, or cell to a greater extent compared with a target molecule expression and/or activity in a tissue, organ, or cell that do not express the GLUT.

In another aspect, provided herein is a compound for reducing or inhibiting a target molecule expression and/or activity in a specific tissue, organ, or cell of a subject in need thereof, comprising the structure of Formula 17:

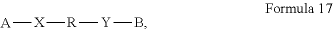

wherein:
R is an inhibitory oligonucleotide molecule;
A is a hydrophilic moiety;
B is a hydrophobic moiety; and
X and Y are independently a covalent bond or a first linker; and
wherein a ligand molecule for a glucose transporter (GLUT), or functional analog thereof is covalently linked to a terminus of A, a terminus of B, or a combination thereof via a second linker or a bond; and
wherein the specific tissue, organ, or cell expresses the GLUT;
wherein the compound reduces or inhibits expression and/or activity of the target molecule in the specific tissue, organ, or cell to a greater extent compared with a target molecule expression and/or activity in a tissue, organ, or cell that do not express the GLUT.

In some embodiments, the second linker is —NH(CH₂)ₙCONH—CH₂—CH(OH)—CH₂—, an ether linkage, or Compound 4:

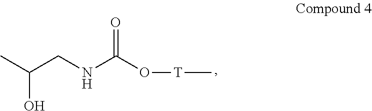

wherein T is 1-15 repetitive Compound 1:

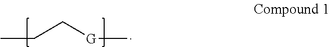

wherein G is selected from the group consisting of CH2, O, S and NH.

In some embodiments, the compound comprises the structure of Formula 18 or Formula 19:

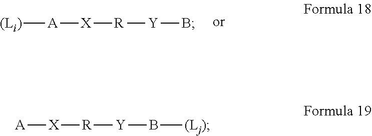

and
wherein L is the ligand molecule for a GLUT, or functional analog thereof;
wherein i and j are independently an integer of 0 through 10; and
wherein when i is 0, j is an integer of 1 through 10, and when j is 0, i is an integer of 1 through 10.

In some embodiments, the ligand molecule for a GLUT is a sugar.

In some embodiments, the sugar is selected from the group consisting of fructose, galactose, glucosamine, glucose, mannose, xylose, dehydroascorbic acid, and trehalose.

In some embodiments, the ligand molecule for a GLUT is selected from the group consisting of fructose, galactose, glucosamine, glucose, mannose, xylose, dehydroascorbic acid, trehalose, and urate.

In some embodiments, the GLUT is selected from the group consisting of GLUT1-GLUT14.

In some embodiments, the ligand molecule for a GLUT is fructose and the GLUT is selected from the group consisting of GLUT2, GLUT5, GLUT7, GLUT8, GLUT9, GLUT11, and GLUT12.

In some embodiments, the ligand molecule for a GLUT is galactose and the GLUT is selected from the group consisting of GLUT1, GLUT2, GLUT3, GLUT4, and GLUT14.

In some embodiments, the ligand molecule for a GLUT is glucose and the GLUT is selected from the group consisting of GLUT1, GLUT2, GLUT3, GLUT4, GLUT6, GLUT7, GLUT8, GLUT9, GLUT10, GLUT11, and GLUT12.

In some embodiments, the ligand molecule for a GLUT is mannose and the GLUT is selected from the group consisting of GLUT1, GLUT2, GLUT3, and GLUT4.

In some embodiments, the ligand molecule for a GLUT is xylose and the GLUT is GLUT3.

In some embodiments, the ligand molecule for a GLUT is dehydroascorbic acid and the GLUT is GLUT4.

In some embodiments, the ligand molecule for a GLUT is trehalose and the GLUT is GLUT8.

In some embodiments, the ligand molecule for a GLUT is urate and the GLUT is GLUT9.

In some embodiments, R is a double stranded oligonucleotide molecule or a single stranded oligonucleotide molecule.

In some embodiments, R reduces or inhibits expression and/or activity of a target molecule.

In some embodiments, the target molecule is any one selected from the group consisting of BACE1-AS, BACE1, presenilin1 (PS1), ROCK-II, 12 PP-2A, ACAT-1, Nogo receptor, mutant presenilin1 (L392V PS-1), APP, Tau, VDAC1, α-synuclein, Huntingtin (Htt), Notch1, LINGO-1, NR4A2, TRIF, caspase-2, CaMKII, glial fibrillary acidic protein (GFAP), Vimentin, EphB3, iNOS, Nischarin, and RhoA.

In some embodiments, the inhibitory oligonucleotide molecule is an siRNA, an shRNA, a miRNA, an amiRNA, an antisense oligonucleotide molecule, an RNAi agent, or any combination thereof.

In some embodiments, R is a double strand oligonucleotide molecule comprising a sense strand and an antisense strand comprising a sequence complementary to the sense strand.

In some embodiments, the sense strand or anti-sense strand is 19 to 31 nucleotides in length.

In some embodiments, at least one phosphate group is linked to the 5′ terminus of the anti-sense strand.

In some embodiments, the compound comprises the structure of Formula 2:

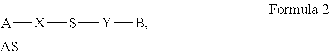

where S is the sense strand, and AS is the anti-sense strand.

In some embodiments, R is the antisense oligonucleotide molecule consisting of from 5 to 50 oligonucleotides, 10 to 40 oligonucleotides, or 15 to 30 oligonucleotides.

In some embodiments, R is DNA, RNA, or a combination thereof.

In some embodiments, R comprises a backbone modification comprising a phosphorothioate linkage, a phosphorodiamidate linkage, a boranophosphate linkage, a methyl phosphonate linkage, or any combination thereof.

In some embodiments, R comprises a locked nucleic acid, a peptide nucleic acid, a unlocked nucleic acid, or any combination thereof.

In some embodiments, R comprises a 2′-O-methyl moiety, a 2′-fluoro moiety, a 2′-O-methoxyethyl moiety, a 2′-methyl moiety, a 2′-methoxyl moiety, a 2′-amine moiety, a 2′-O-propyl moiety, a 2′-O-2-methylthioethyl moiety, a 2′-O-3-aminopropyl moiety, a 2′-O-3-dimethylaminopropyl moiety, a 2′-O—N-methylacetamino moiety, or a 2′-O-dimethylamidooxyethyl moiety.

In some embodiments, R comprises a modified sugar moiety.

In some embodiments, the hydrophilic moiety is any one selected from the group consisting of polyethylene glycol (PEG), polyvinyl pyrrolidone and polyoxazoline.

In some embodiments, the compound comprises the structure of Formula 7 or Formula 8:

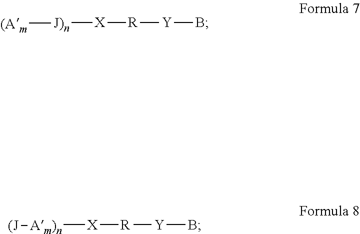

wherein:
A′ is a monomer hydrophilic moiety,
J is selected from the group comprised of PO₃⁻, SO₃⁻ and CO₂⁻, and links A′ to each other or links A′ to X,
m is an integer of 1 to 15, and
n is an integer of 1 to 10; and
wherein A′ is any one selected from the group consisting of Compound 1, Compound 2, and Compound 3:

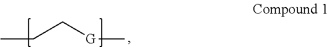

wherein G is selected from the group comprised of O, S and NH;

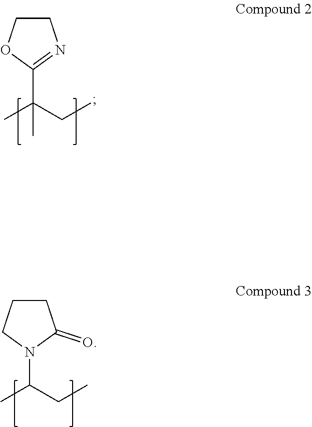

In some embodiments, the molecular weight of the hydrophilic moiety is 200 to 10,000.

In some embodiments, the hydrophobic moiety is any one selected from the group consisting of a steroid derivative, a glyceride derivative, glycerol ether, poplypropylene glycol, unsaturated or saturated C₁₂-C₅₀, diacylphosphatidylcholine, a fatty acid, a phospholipid, a lipopolyamine, a lipid, tocopherol, and tocotrienol.

In some embodiments, the steroid derivative is any one selected from the group consisting of cholesterol, cholestanol, cholic acid, cholesteryl formate, cotestanyl formate, and colistanylamine.

In some embodiments, the glyceride derivative is any one selected from the group consisting of monoglyceride, diglyceride and triglyceride.

In some embodiments, the molecular weight of the hydrophobic moiety is 250 to 1,000.

In some embodiments, X and Y are independently a non-degradable bond or a degradable bond.

In some embodiments, the non-degradable bond is an amide bond or a phosphorylated bond.

In some embodiments, the degradable bond is any one selected from the group consisting of a disulfide bond, an acid-degradable bond, an ester bond, an anhydride bond, a biodegradable bond, and an enzyme degradable bond.

In some embodiments, the compound is formulated as a nanoparticle.

In some embodiments, the compound is formulated as a composition.

In some embodiments, the composition is formulated as a pharmaceutical composition, wherein the pharmaceutical composition further comprises a pharmaceutically acceptable excipient, carrier, or diluent.

In some embodiments, the specific tissue, organ, or cell that expresses the GLUT comprises brain, blood, brain, intestine, liver, kidney, pancreas, embryos, testis, placenta, muscle, heart, fat, adipose tissue, spleen, colon, prostate, testis, blastocysts, skeletal muscle, white adipose tissue, brown adipose tissue, or any combination thereof.

In some embodiments, (i) the GLUT is GLUT1, and the specific tissue, organ, or cell that expresses the GLUT comprises brain, blood, or a combination thereof;
(ii) the GLUT is GLUT2, and the specific tissue, organ, or cell that expresses the GLUT comprises brain, intestine, liver, kidney, pancreas, or any combination thereof;
(iii) the GLUT is GLUT3, and the specific tissue, organ, or cell that expresses the GLUT comprises brain, embryos, testis, placenta, or any combination thereof;
(iv) the GLUT is GLUT4, and the specific tissue, organ, or cell that expresses the GLUT comprises brain, muscle, heart, fat, adipose tissue, or any combination thereof;
(v) the GLUT is GLUT5, and the specific tissue, organ, or cell that expresses the GLUT comprises brain, intestine, testis, muscle, kidney, fat, or any combination thereof;
(vi) the GLUT is GLUT6, and the specific tissue, organ, or cell that expresses the GLUT comprises brain, spleen, or a combination thereof;
(vii) the GLUT is GLUT7, and the specific tissue, organ, or cell that expresses the GLUT comprises intestine, colon, prostate, testis, prostate gland, or any combination thereof;
(viii) the GLUT is GLUT8, and the specific tissue, organ, or cell that expresses the GLUT comprises brain, testis, liver, spleen, fat, blastocysts, or any combination thereof;
(ix) the GLUT is GLUT9, and the specific tissue, organ, or cell that expresses the GLUT comprises liver, kidney, intestine, colon, or any combination thereof;
(x) the GLUT is GLUT10, and the specific tissue, organ, or cell that expresses the GLUT comprises liver, pancreas, fat, skeletal muscle, heart, adipose tissue, placenta, kidney, or any combination thereof;
(xi) the GLUT is GLUT11, and the specific tissue, organ, or cell that expresses the GLUT comprises heart, muscle, kidney, pancreas, placenta, or any combination thereof;
(xii) the GLUT is GLUT12, and the specific tissue, organ, or cell that expresses the GLUT comprises brain, muscle, heart, fat, pancreas, prostate, adipose tissue, placenta, kidney, or any combination thereof;
(xiii) the GLUT is GLUT13, and the specific tissue, organ, or cell that expresses the GLUT comprises brain, white adipose tissue, brown adipose tissue, kidney, or any combination thereof; or
(xiv) the GLUT is GLUT14, and the specific tissue, organ, or cell that expresses the GLUT

In some embodiments, (i) the GLUT is GLUT1, and the specific tissue, organ, or cell that expresses the GLUT comprises blood-brain barrier, astrocyte, erythrocyte, or any combination thereof;
(ii) the GLUT is GLUT2, and the specific tissue, organ, or cell that expresses the GLUT comprises astrocyte, gastrointestinal tract, small intestine, intestinal absorptive epithelial cells, hepatocytes, pancreatic B-cells, brain nuclei, nucleus tractus solitatius, motor nucleus of the vagus, paraventricular hypothalamic nucleus, lateral hypothalamic area, arcuate nucleus, and olfactory bulb, or any combination thereof;
(iii) the GLUT is GLUT3, and the specific tissue, organ, or cell that expresses the GLUT comprises neuron, blood-brain barrier, astrocyte, sperm, spermatozoa, fibroblast, platelet, retinal endothelial cell, white blood cell, or any combination thereof;
(iv) the GLUT is GLUT4, and the specific tissue, organ, or cell that expresses the GLUT comprises neuron, adipocyte, skeletal muscle cell, cardiomyocyte, hypothalamus, cerebellum, cortex, and hippocampus, or a combination thereof;
(v) the GLUT is GLUT5, and the specific tissue, organ, or cell that expresses the GLUT comprises microglia, blood-brain barrier, small intestine, apical membrane of the enterocytes, plasma membrane of mature spermatozoa, skeletal muscle, or any combination thereof;
(vi) the GLUT is GLUT6, and the specific tissue, organ, or cell that expresses the GLUT comprises leukocyte, peripheral leukocyte, germinal cell of the testis, or any combination thereof;
(vii) the GLUT is GLUT7, and the specific tissue, organ, or cell that expresses the GLUT comprises small intestine;
(viii) the GLUT is GLUT8, and the specific tissue, organ, or cell that expresses the GLUT comprises neuron, brown adipose tissue, cerebellum, adrenal gland, spermatocyte, mature spermatozoa, or any combination thereof;
(ix) the GLUT is GLUT9, and the specific tissue, organ, or cell that expresses the GLUT comprises small intestine, leukocyte, chondrocyte, or any combination thereof;
(x) the GLUT is GLUT10, and the specific tissue, organ, or cell that expresses the GLUT comprises white adipose;
(xi) the GLUT is GLUT12, and the specific tissue, organ, or cell that expresses the GLUT comprises astrocyte, prostate gland, small intestine, chondrocyte, or any combination thereof; or
(xii) the GLUT is GLUT13, and the specific tissue, organ, or cell that expresses the GLUT comprises neuron.

In some embodiments, the specific tissue, organ, or cell that expresses the GLUT

In some embodiments, the cancer is selected from the group consisting of brain cancer, breast cancer, cervix cancer, colon cancer, kidney cancer, lung cancer, ovary cancer, prostate cancer, skin cancer, thyroid cancer, glioblastoma, pancreas cancer, leukemia, gastric cancer, colorectal cancer, and any combination thereof.

In some embodiments, (i) the GLUT is GLUT1, and the cancer is selected from the group consisting of brain cancer, breast cancer, cervix cancer, colon cancer, kidney cancer, lung cancer, ovary cancer, prostate cancer, skin cancer, thyroid cancer, and any combination thereof;
(ii) the GLUT is GLUT3, and the cancer is selected from the group consisting of brain cancer, breast cancer, colon cancer, cervix cancer, kidney cancer, lung cancer, glioblastoma, and any combination thereof;
(iii) the GLUT is GLUT5, and the cancer is selected from the group consisting of breast cancer, colon cancer, pancreas cancer, ovary cancer, lung cancer, leukemia, and any combination thereof;
(iv) the GLUT is GLUT10, and the cancer is leukemia;
(v) the GLUT is GLUT12, and the cancer is breast cancer; or
(vi) the GLUT is GLUT14, and the cancer is brain cancer.

In another aspect, provided herein is a method of increasing blood brain barrier penetration of a compound comprising the structure of Formula 20 in a subject in need thereof comprising:

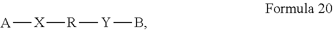

wherein:
R is an oligonucleotide molecule;
A is a hydrophilic moiety;
B is a hydrophobic moiety; and
X and Y are independently a covalent bond or a first linker,
(i) covalently linking a ligand molecule for a glucose transporter (GLUT), or functional analog thereof via a second linker or a bond to a terminus of A, a terminus of B, or a combination thereof of the compound comprising the structure of Formula 20; and
(ii) administering to the subject;
wherein the compound exhibits blood brain barrier penetration in the subject to a greater extent compared with a compound having the same structure without the ligand molecule for a GLUT, or functional analog thereof linked.

In another aspect, provided herein is a compound with increased blood brain barrier penetration in a subject in need thereof, comprising the structure of Formula 20:

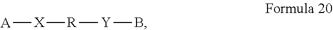

wherein:
R is an oligonucleotide molecule;
A is a hydrophilic moiety;
B is a hydrophobic moiety; and
X and Y are independently a covalent bond or a first linker,
wherein a ligand molecule for a glucose transporter (GLUT), or functional analog thereof is covalently linked to a terminus of A, a terminus of B, or a combination thereof via a second linker or a bond; and
wherein when administered to the subject, the compound exhibits blood brain barrier penetration in the subject to a greater extent compared with a compound having the same structure without the ligand molecule for a GLUT, or functional analog thereof linked.

In some embodiments, the second linker is —NH(CH₂)ₙCONH—CH₂—CH(OH)—CH₂—, an ether linkage, or Compound 4:

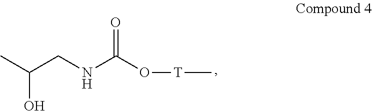

wherein T is 1-15 repetitive Compound 1:

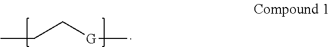

wherein G is selected from the group consisting of CH2, O, S and NH.

In some embodiments, the compound comprises the structure of Formula 18 or Formula 19:

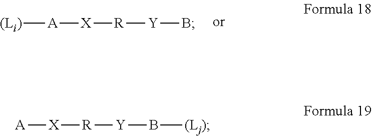

and
wherein L is the ligand molecule for a GLUT, or functional analog thereof;
wherein i and j are independently an integer of 0 through 10; and
wherein when i is 0, j is an integer of 1 through 10, and when j is 0, i is an integer of 1 through 10.

In some embodiments, the ligand molecule for a GLUT is a sugar.

In some embodiments, the sugar is selected from the group consisting of fructose, galactose, glucosamine, glucose, mannose, xylose, dehydroascorbic acid, and trehalose.

In some embodiments, the GLUT is selected from the group consisting of GLUT1, GLUT2, GLUT3, GLUT4, GLUT5, GLUT6, GLUT8, GLUT12, and GLUT13.

In some embodiments, the ligand molecule for a GLUT is fructose and the GLUT is selected from the group consisting of GLUT2, GLUT5, GLUT8, and GLUT12.

In some embodiments, the ligand molecule for a GLUT is galactose and the GLUT is selected from the group consisting of GLUT1, GLUT2, GLUT3, and GLUT4.

In some embodiments, the ligand molecule for a GLUT is glucose and the GLUT is selected from the group consisting of GLUT1, GLUT2, GLUT3, GLUT4, GLUT6, GLUT8, and GLUT12.

In some embodiments, the ligand molecule for a GLUT is mannose and the GLUT is selected from the group consisting of GLUT1, GLUT2, GLUT3, and GLUT4.

In some embodiments, the ligand molecule for a GLUT is xylose and the GLUT is GLUT3.

In some embodiments, the ligand molecule for a GLUT is dehydroascorbic acid and the GLUT is GLUT4.

In some embodiments, the ligand molecule for a GLUT is trehalose and the GLUT is GLUT8.

In some embodiments, R is a double stranded oligonucleotide molecule or a single stranded oligonucleotide molecule.

In some embodiments, R reduces or inhibits expression and/or activity of a target molecule.

In some embodiments, the target molecule is any one selected from the group consisting of BACE1-AS, BACE1, presenilin1 (PS1), ROCK-II, 12 PP-2A, ACAT-1, Nogo receptor, mutant presenilin1 (L392V PS-1), APP, Tau, VDAC1, α-synuclein, Huntingtin (Htt), Notch1, LINGO-1, NR4A2, TRIF, caspase-2, CaMKII, glial fibrillary acidic protein (GFAP), Vimentin, EphB3, iNOS, Nischarin, and RhoA.

In some embodiments, the inhibitory oligonucleotide molecule is an siRNA, an shRNA, a miRNA, an amiRNA, an antisense oligonucleotide molecule, an RNAi agent, or any combination thereof.

In some embodiments, R is a double strand oligonucleotide molecule comprising a sense strand and an antisense strand comprising a sequence complementary to the sense strand.

In some embodiments, the sense strand or anti-sense strand is 19 to 31 nucleotides in length.

In some embodiments, at least one phosphate group is linked to the 5′ terminus of the anti-sense strand.

In some embodiments, the compound comprises the structure of Formula 2:

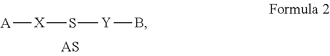

where S is the sense strand, and AS is the anti-sense strand.

In some embodiments, R is the antisense oligonucleotide molecule consisting of from 5 to 50 oligonucleotides, 10 to 40 oligonucleotides, or 15 to 30 oligonucleotides.

In some embodiments, R is DNA, RNA, or a combination thereof.

In some embodiments, R comprises a backbone modification comprising a phosphorothioate linkage, a phosphorodiamidate linkage, a boranophosphate linkage, a methyl phosphonate linkage, or any combination thereof.

In some embodiments, R comprises a locked nucleic acid, a peptide nucleic acid, a unlocked nucleic acid, or any combination thereof.

In some embodiments, R comprises a 2′-O-methyl moiety, a 2′-fluoro moiety, a 2′-O-methoxyethyl moiety, a 2′-methyl moiety, a 2′-methoxyl moiety, a 2′-amine moiety, a 2′-O-propyl moiety, a 2′-O-2-methylthioethyl moiety, a 2′-O-3-aminopropyl moiety, a 2′-O-3-dimethylaminopropyl moiety, a 2′-O—N-methylacetamino moiety, or a 2′-O-dimethylamidooxyethyl moiety.

In some embodiments, R comprises a modified sugar moiety.

In some embodiments, the hydrophilic moiety is any one selected from the group consisting of polyethylene glycol (PEG), HEG (hexaethylene glycol), polyvinyl pyrrolidone and polyoxazoline.

In some embodiments, the compound comprises the structure of Formula 7 or Formula 8:

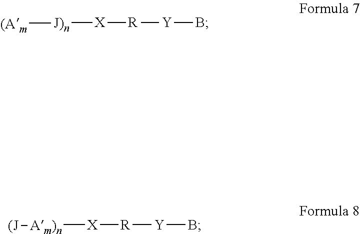

wherein:
A′ is a monomer hydrophilic moiety,
J is selected from the group comprised of PO₃⁻, SO₃⁻ and CO₂⁻, and links A′ to each other or links
A′ to X,
m is an integer of 1 to 15, and
n is an integer of 1 to 10; and
   wherein A′ is any one selected from the group consisting of Compound 1, Compound 2, and Compound 3:

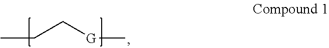

wherein G is selected from the group comprised of O, S and NH;

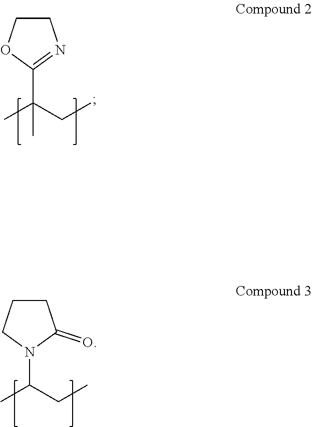

In some embodiments, the molecular weight of the hydrophilic moiety is 200 to 10,000.

In some embodiments, the hydrophobic moiety is any one selected from the group consisting of a steroid derivative, a glyceride derivative, glycerol ether, poplypropylene glycol, unsaturated or saturated C₁₂-C₅₀, diacylphosphatidylcholine, a fatty acid, a phospholipid, a lipopolyamine, a lipid, tocopherol, and tocotrienol.

In some embodiments, the steroid derivative is any one selected from the group consisting of cholesterol, cholestanol, cholic acid, cholesteryl formate, cotestanyl formate, and colistanylamine.

In some embodiments, the glyceride derivative is any one selected from the group consisting of monoglyceride, diglyceride and triglyceride.

In some embodiments, the molecular weight of the hydrophobic moiety is 250 to 1,000.

In some embodiments, X and Y are independently a non-degradable bond or a degradable bond.

In some embodiments, the non-degradable bond is an amide bond or a phosphorylated bond.

In some embodiments, the degradable bond is any one selected from the group consisting of a disulfide bond, an acid-degradable bond, an ester bond, an anhydride bond, a biodegradable bond, and an enzyme degradable bond.

In some embodiments, the blood brain barrier penetration of the compound is mediated via a GLUT-mediated ligand uptake into a cell and a GLUT-mediated ligand efflux across the blood brain barrier.

In some embodiments, the compound is formulated as a nanoparticle.

In some embodiments, the compound is formulated as a composition.

In some embodiments, the composition is formulated as a pharmaceutical composition, wherein the pharmaceutical composition further comprises a pharmaceutically acceptable excipient, carrier, or diluent.

### BRIEF DESCRIPTION OF THE DRAWINGS

FIG. 1 is a diagram showing the Pathway through GLUT. In some embodiments, receptor-targeting chemistry is combined with SAMiRNA™ technology to enable central nervous system (CNS) delivery of siRNA and effective K_{D}.

FIG. 2 is a scheme showing that an exemplary embodiment of BBB targeting ligand on the hydrophilic end enhances intravenous siRNA delivery by over 20 folds. CNS-SAMiRNA™ platform incorporates a BBB-targeting ligand to the existing SAMiRNA™ platform, which doubly conjugates the ends of the RNA sense strand with hydrophilic and hydrophobic moieties to enable self-assembly into nanoparticles.

FIG. 3 illustrates the DMTr-Protection, Tatra-acetylation, DMTr-deprotection and 6-alcohol protection (trifluoromethanesulfonate) process of D-(+)-Glucose of Example 1.

FIG. 4 illustrates the Amine TFA-protection (6-Aminohexnoic acid) process Example 2.

FIG. 5 illustrates the Amine TFA-Protection (3-amino-1,2-propanediol), DMT-Protection (3-amino-1,2-propanediol) and TFA-Deprotection (3-amino-1,2-propanediol) process of Example 3.

FIG. 6 illustrates the Amide coupling (6-Aminohexnoicacid+3-amino-1,2-propanediol) and Amine-TFA deprotection (6-Aminohexnoicacid+3-amino-1,2-propanediol) process of Example 4.

FIG. 7 illustrates the Amine addition, Amine-TFA Protection, Succinylation, CPG coupling and Capping process of Example 5.

FIG. 8 illustrates the structure of a sense strand conjugated with glucose synthesized according to Example 6.

FIG. 9 illustrates the micelle qNANO size distribution of Example 7.

FIG. 10 illustrates Immunocytochemistry (ICC) results for entry of particles into neuro2A cells following treatment of the particles at varying glucose-SAMiRNA™-cy5/SAMIRNA™-cy5 ratios under culture medium conditions having different glucose content.

FIGS. 11A-11D show longer residence time in the brain means enhanced bioavailability potentially more favorable dosing schedule. FIG. 11A is a scheme showing an exemplary study design. FIG. 11B shows ex vivo fluorescence brain images after intravenous (IV) administration of Cy5-labeled CNS-SAMiRNA™, acquired by IVIS-200, Perkin Elmer. The results of IVIS200 measurement of cy5 fluorescence was obtained from the brain tissues extracted at different dates following mouse lateral tail vein administration of the glucose-SAMIRNA™-cy5 of Example 9-2. FIG. 11C shows average radiant efficiency of ex vivo images. For instance, the graph analyzes mean radiant efficiency by date compared to seconds to minutes for typical RNAi. FIG. 11D shows quantitative analysis of CNS-SAMiRNA™ (“Ligand-SAMiRNA™”) in the whole brain tissue compared to non-targeting SAMiRNA™ (“SAMIRNA™”). The graph exhibits percentage delivery versus amount of glucose-SAMIRNA™-cy5 administered per unit gram brain tissue in Example 9-3.

FIG. 12A shows photos of confocal imaging results of cy5 fluorescence at the sagittal section side of brain tissue extracted at different dates following mouse lateral tail vein administration of glucose-SAMiRNA™-cy5 in Example 9-4. Bar=1000 μm. FIG. 12B shows magnified confocal images of cy5 fluorescence at the sagittal section side of brain tissue extracted at 2 days after mouse lateral tail vein administration of glucose-SAMiRNA™-cy5 in Example 9-4. Bar=1000 μm (Upper panel). 40 μm (Lower panel), 10 μm (boxes in Lower panel); acquired by DragonFly, Andor. FIG. 12C shows photos of results for each part of cy5 fluorescence at the sagittal section side of brain tissue extracted at 2 days after mouse lateral tail vein administration of glucose-SAMiRNA™-cy5 in Example 9-4 as shown in FIG. 12B, Upper panel. Name of each part: 1; Olfactory bulb, 2; Septum, 3; Ventricle 3v, 4; Hippocam-pus, 5; Cerebral cortex white, 6; Cerebral cortex gray, 7; Thalamus, 8; Hypothalamus, 9; Midbrain, 10; Ventricle 4v, 11; Ventricle 4v, 12; Choroid plexus 4v, 13; Cerebellal nu-clei, 14; Cerebellal cortex, 15; Pons, 16; Medulla.

FIG. 13 shows potential for wide range of CNS indications enabled by effective delivery and distribution of an exemplary compound as provided herein in the brain. IV delivery of an exemplary compound as provided herein across BBB distributes all across and especially to the deepest parts of the brain (top panel) compared with intrathecal delivery of a comparative control compound via cerebrospinal fluid (CSF) that largely distributes to the outer parts of the brain (bottom panel). Name of each part: 1; Olfactory bulb, 2; Septum, 3; Ventricle 3v, 4; Hippocampus, 5; Cerebral cortex white, 6; Cerebral cortex gray, 7; Thalamus, 8; Hypothalamus, 9; Midbrain, 10; Ventricle 4v, 11; Ventricle 4v, 12; Choroid plexus 4v, 13; Cerebellal nu-clei, 14; Cerebellal cortex, 15; Pons, 16; Medulla.

FIG. 14 shows potential for wide range of CNS indications enabled by effective delivery and distribution of an exemplary compound as provided herein in the brain. Left panel shows image of distribution with name of each part: 1; Olfactory bulb, 2; Septum, 3; Ventricle 3v, 4; Hippocampus, 5; Cerebral cortex white, 6; Cerebral cortex gray, 7; Thalamus, 8; Hypothalamus, 9; Midbrain, 10; Ventricle 4v, 11; Ventricle 4v, 12; Choroid plexus 4v, 13; Cerebellal nu-clei, 14; Cerebellal cortex, 15; Pons, 16; Medulla. Right panel shows plot of fluoresce intensity for each of the 16 tissues at day 2.

FIGS. 15A-15B show immunofluorescent confocal images of astrocytes, BCECs, and Cy5-labeled CNS-SAMiRNA™ (FIG. 15A; hippocampus region, FIG. 15B; hypothalamus region for verifying BBB penetration). White arrows, SAMiRNA™; White dotted line, BBB. FIG. 15A show that an exemplary embodiment of the CNS platform as provided herein crosses the BBB to deliver to hippocampus, the deep region in temporal brain for learning and memory. FIG. 15B show good delivery of an exemplary embodiment of the CNS platform as provided herein to hypothalamus of the forebrain responsible for autonomous functions, sleep and emotions. FIGS. 15C-15D show immunofluorescent confocal images of neurons (FIG. 15C; cortex, FIG. 15D; hypothalamus) of brain tissue extracted after mouse lateral tail vein administration of glucose-SAMiRNA™-cy5 in Example 9-5, demonstrating neuronal delivery of an exemplary embodiment of the CNS platform as provided herein, confirmed in hypothalamus and cerebral cortex, responsible for consciousness. FIG. 15C shows immunofluorescent images of neuron, and Cy5-labeled CNS-SAMiRNA™ in the cerebral cortex region. FIG. 15D shows hypothalamus region for verifying CNS-SAMiRNA™ neuronal co-localization. Scale bar, 200 or 50 μm; Section, 50 μm; Acquisition, Dragonfly, Andor; White dotted line, neuron. FIG. 15A. Hippocampus, astrocyte; green, BCEC; yellow, nucleus; blue, red; glu-SAMiRNA™. FIG. 15B. Hypothalamus, astrocyte; green, BCEC; yellow, nucleus; blue, red; glu-SAMiRNA™. FIG. 15C. Cortex, neuron; green, blue; nucleus, red; glu-SAMiRNA™. FIG. 15D. Hypothalamus, neuron; green, blue; nucleus, red; glu-SAMiRNA™. FIG. 15E. 3V choroid plexus, astrocyte; green, BCEC; yellow, nucleus; blue, red; glu-SAMiRNA™. FIG. 15F. 3V, astrocyte; green, BCEC; yellow, nucleus; blue, red; glu-SAMiRNA™.

FIG. 16 shows graphs representing results of stem-loop qPCR relative quantification for residual glucose-SAMiRNA™ for each part of brain tissue extracted at different dates following mouse lateral vein administration of glucose-SAMiRNA™ of Example 9-6. The initial PK as shown data suggest two route of delivery—via BBB & CSF.

FIG. 17 shows that proof of concept (PoC) study on an exemplary embodiment of CNS platform as provided herein targeting neural marker or housekeeping gene confirms knockdown (K_{D}) in key brain disease areas by IV or subcutaneous (SC) administration in normal mice. The exemplary embodiment of CNS platform as provided herein shows impressive inhibition of Map2, neural marker or Gapdh, housekeeping gene in the key brain regions via both IV and SC administration. n, 3; P-value, t-test. Left panel shows image of distribution with name of each part: 1; Olfactory bulb, 2; Septum, 3; Ventricle 3v, 4; Hippocampus, 5; Cerebral cortex white, 6; Cerebral cortex gray, 7; Thalamus, 8; Hypothalamus, 9; Midbrain, 10; Ventricle 4v, 11; Ventricle 4v, 12; Choroid plexus 4v, 13; Cerebellal nu-clei, 14; Cerebellal cortex, 15; Pons, 16; Medulla. Right panel shows quantification of the images in the left panel.

FIG. 18 shows that region-specific inhibition (K_{D}) by an exemplary compound as provided herein is due to region-specific delivery of the exemplary compound as provided. Region-specific delivery is focused on key brain disease areas and inhibition (K_{D}) is induced by the focused delivery of an exemplary compound as provided herein.

FIG. 19 shows that region-specific delivery of an exemplary compound as provided herein is due to region-specific energy consumption. Energy consumption by area shows different pattern, and the relatively high consumption was found in the regions of the cortex, hippocampus, and cerebellum, where more delivery of and inhibition by CNS-SAMiRNA™ were observed. Top Panel, 18 fluorodeoxyglucose (FDG) uptake positron emission tomography (PET) quantification by region. SUV, standardized uptake value; Cort, cortex; Hypo, hypothalamus; STR, striatum; HC, hippocampus; cBS, caudal brainstem; Cere, cerebellum; mean with SEM. Bottom panel, Glutamate metabolite analysis by mouse brain region using LC-MS/MS.

FIG. 20 shows that PoC studies of an exemplary CNS platform as provided herein confirms delivery to key Alzheimer's disease (AD) regions in deep brain and efficacy in key AD proteins. Alzheimer's disease often starts in and around the hippocampus. Thus, one of the first noticeable symptoms is memory loss.

FIG. 21 shows that PoC studies of an exemplary CNS platform as provided herein confirms delivery to key AD regions in deep brain and efficacy in key AD proteins. Alzheimer's disease is most often associated with Aβ and tau accumulation. Notable pathways include BACE-1 for Aβ and MAPT for tau and phosphorylated tau.

FIGS. 22A-22D show the results of the primary qPCR screening for human BACE1 target (593 species) (FIG. 22A), human MAPT target (443 species) (FIG. 22B), mouse Bace1 target (42 species) (FIG. 22C), and mouse Mapt target (126 species) (FIG. 22D).

FIGS. 22E-22H show the results of of the secondary qPCR screening for 12 species of human BACE1 target (FIG. 22E), 12 species of human MAPT target (FIG. 22F), 19 species of mouse Bace1 target (FIG. 22G), and 12 species of mouse Mapt target (FIG. 22H).

FIG. 22I shows that an exemplary embodiment of the compound as provided herein, BACE1-MAPT siRNA cocktail using an exemplary CNS platform as provided herein targets both Aβ and tau, the two representative genes for Alzheimer's disease, confirming target genes knockdown.

FIG. 23 shows that BACE1 knockdown by Glut-SAMiRNA targeting BACE1 prevents Aβ peptide accumulation in CA1 and subiculum, in the deep brain region hippocampus.

FIG. 24 shows that MAPT knockdown by Glut-SAMiRNA targeting MAPT results in approximately 70% tau peptide accumulation in the subiculum, preventing phosphorylation of tau protein.

FIG. 25 shows that phosphorylated tau is effectively regulated as a result of tau protein inhibition via the MAPT knockdown pathway.

FIG. 26 shows suppression of Key AD-associated genes result in mouse cognitive behavior improvement up to normal level (n=10). Left Panel, Y Maze Spontaneous Alternation Test is useful in assessing hippocampal damage, quantifying the cognitive deficits, and evaluating the effects of drugs on cognition. Right graph, SAMiRNA™ candidate recovered cognitive deficits of the AD mouse to normal levels.

FIG. 27 shows that the toxicity evaluation of an exemplary CNS platform as provided herein upon 4-week repeated IV administration demonstrated no systemic toxicity.

FIG. 28 shows that the toxicity evaluation of an exemplary CNS platform as provided herein upon 4-week repeated IV administration demonstrated minimum hepatotoxicity, as assessed by the Alkaline phosphatase (ALP) level in the serum of 3×Tg mice.

FIG. 29 shows that the toxicity evaluation of an exemplary CNS platform as provided herein upon 4-week repeated IV administration demonstrated minimum hepatotoxicity, as assessed by the Alanine aminotransferase (ALT) level and the Aspartate aminotransferase (AST) level in the serum of 3×Tg mice.

FIG. 30 shows that the toxicity evaluation of an exemplary CNS platform as provided herein upon 4-week repeated IV administration demonstrated minimum hepatotoxicity, as assessed by the Total bilirubin (T-bil) level and the Ammonia (NH3) level in the serum of 3×Tg mice.

FIG. 31 shows that the toxicity evaluation of an exemplary CNS platform as provided herein upon 4-week repeated IV administration demonstrated no kidney toxicity, as assessed by the lactic acid (Lac) level in the serum of 3×Tg mice.

FIG. 32 shows that the toxicity evaluation of an exemplary CNS platform as provided herein upon 4-week repeated IV administration demonstrated no kidney toxicity, as assessed by the Blood Urea Nitrogen (BUN) level and the Creatinine (Crea) level.

FIG. 33 shows that the 4-week repeated IV administration confirmed that an exemplary CNS platform did not affect lipid metabolism, as assessed by the Lipase (Lip) level in the serum of 3×Tg mice.

FIG. 34 shows that the 4-week repeated IV administration confirmed that an exemplary CNS platform did not affect lipid metabolism, as assessed by the High density lipoprotein (HDL) level and the Low density lipoprotein (LDL) level in the serum of 3×Tg mice.

FIG. 35 shows that the 4-week repeated IV administration confirmed that an exemplary CNS platform did not affect lipid metabolism, as assessed by the Total cholesterol (T-Chol) level and the Triglycerides (TG) level in the serum of 3×Tg mice.

FIG. 36 shows that the 4-week repeated IV administration confirmed that an exemplary CNS platform had no effect in calcium homeostasis, as assessed by the Ca level and the P level in the serum of 3×Tg mice.

FIG. 37 shows that biodistribution of an exemplary CNS platform as provided herein shows significant delivery to the brain (1.4% injected does (ID)/g brain), as well as in liver and kidney. In some embodiments, as with transferrin, glucose receptor also shows some off target effects, but has the significant advantage of superior delivery through blood glucose monitoring. In some embodiment, selection of target gene can be important for safety and efficacy of therapeutic development.

FIGS. 38A-38C shows the effects of an exemplary compound SRN-008 (compound comprising Glut-SAMiRNA targeting BACE1 and MAPT) as provided herein on cognitive decline in 3×Tg mice. The Y-maze test was performed 24 h after final administration. FIG. 38A is a scheme showing an exemplary study design. FIG. 38B shows spontaneous alterations and FIG. 38C shows total arm entry during 8-min sessions measured. The vehicle group was treated with PBS. Values are expressed as the mean±S.E.M. #p<0.05 indicate significant differences compared with the vehicle-treated B6129F1/J mice and *p<0.05 indicates significant differences compared with the vehicle-treated 3×Tg mice.

FIGS. 39A-39F show the effects of an exemplary compound SRN-008 treatment on Aβ, human tau, and phosphorylated tau accumulation in the CA1 and subiculum of 3×Tg mice. FIGS. 39A and 39B show 4G8-positive area significantly decreased in the CA1 (FIG. 39A) and the subiculum (FIG. 39B) of SRN-008-treated 3×Tg mice compared to the control. FIGS. 39C and 39D show HT7-positive area significantly decreased in the CA1 (FIG. 39C) and the subiculum (FIG. 39D) of SRN-008-treated 3×Tg mice compared to the control. FIGS. 39E and 39F show AT8-positive area significantly reduced in the CA1 (FIG. 39E) and the subiculum (FIG. 39F) of SRN-008-treated 3×Tg mice compared to the control. Values are expressed as the mean±S.E.M. Scale bar=50 μm*** p<0.001 indicates significant differences compared with the vehicle-treated 3×Tg mice.

FIGS. 40A-40F show exemplary links or linkages and exemplary compounds comprising links or linkages. FIG. 40A show an exemplary linker-NH(CH₂)ₙCONH—CH₂—CH(OH)—CH₂—, and FIG. 40B show exemplary embodiments of the compound as provided herein. In some embodiments, the N linker replaces the OH groups at C₆ position of glucose. FIG. 40C show an exemplary linker hexaethylene glycol, and FIG. 40D show an exemplary embodiment of the compound as provided herein. FIG. 40E show an exemplary linkage, ether linkage where R and R′ represent any alkyl or aryl substituent, and FIG. 40F show an exemplary embodiment of the compound as provided herein. In some embodiments, Glucose is introduced via an ether linkage at the C6 position to preserve the binding ability to GLUT1 because the OH groups at positions C1, C3 and C4 of glucose can be essential for its interaction with GLUT1. In some embodiments, the exemplary embodiment of the compounds as provided herein is a self-assembled supramolecular nanocarrier with a surface featuring properly configured glucose. In some embodiment, a PEG-poly(α,β-aspartic acid) block copolymer (PEG-PAsp) with glucose conjugated to the α-end of the PEG segment via a bond with the C6-O moiety (Gluc (6)-PEG-PAsp).

FIGS. 41A-41C represents the immunocytochemistry (ICC) results confirmed by confocal imaging, depicting the improved cellular uptake efficiency of glu-SAMiRNA-cy5 compared to SAMiRNA-cy5 in A549 lung cancer cell line, according to treatment time. FIG. 41A shows a representative image. FIG. 41B display quantified fluorescence graphs from five randomly selected regions at 1 h. FIG. 41C display quantified fluorescence graphs from five randomly selected regions at 20 h.

FIG. 42 illustrates the IC₅₀ graph obtained from RT-qPCR analysis, confirming the enhanced gene inhibition efficacy of glu-SAMiRNA compared to SAMiRNA in A549 lung cancer cell line.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Unless otherwise defined herein, all technical and scientific terms used in the present embodiment shall have the same meaning as ordinarily understood by a person skilled in the technical field to which the present disclosure belongs. Generally, the nomenclatures used in the present disclosure are well-known and commonly used in the art.

### Definition

As used herein, the singular includes the plural unless specifically stated otherwise. It must be noted that, as used herein, the singular forms “a,” “an” and “the” include plural referents unless the context clearly dictates otherwise.

As used herein, the term “or” means “and/or” unless stated otherwise. Furthermore, the term “including” as well as other forms, such as “include”, “includes,” and “included,” is not limiting.

As used herein, reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used herein, the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed herein can be implemented with respect to any method or composition of the present disclosure, and vice versa. Furthermore, compositions of the present disclosure can be used to achieve methods of the present disclosure.

As used herein, the term “about” in relation to a reference numerical value and its grammatical equivalents can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

As used herein, the terms “subject”, “patient,” and “individual”, include animals (e.g., vertebrates, amphibians, fish, mammals, cats, dogs, horses, pigs, cows, sheep, rodents, rabbits, squirrels, bears, primates (e.g., chimpanzees, gorillas, and humans)), which are capable of suffering from a disease or a disorder as described herein. “Subject” may also be a cell, a population of cells, a tissue, an organ, or an organism, preferably to human and constituents thereof. In some embodiments, the cell is a eukaryotic cell. In some embodiments, the cell is a mammalian cell. In some embodiments, the cell is a human cell. In some embodiments, the cell is an animal cell. The subject is preferably a mammal in need of such treatment, e.g., a subject that has been diagnosed with or has a risk of or have a predisposition of developing a disorder as described herein. The mammal can be, e.g., any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

As used herein, a “control” or “standard control” refers to a sample, measurement, or value that serves as a reference, usually a known reference, for comparison to a test sample, measurement, or value. For example, a test sample can be taken from a subject having a given disease and compared with a known normal (non-diseased) individual (e.g., a standard control subject). A standard control can also represent an average measurement or value gathered from a population of similar individuals (e.g., standard control subjects) that do not have a given disease (i.e., standard control population), e.g., healthy individuals with a similar medical background, same age, weight, etc. A standard control value can also be obtained from the same individual, e.g., from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g., RNA levels, protein levels, specific cell types, specific bodily fluids, specific tissues, brain tissue, etc).

As used herein, the terms “prevent,” “preventing,” and “prevention,” refer to a decrease in the occurrence of pathology of a disease or a disorder as described herein in a subject. The prevention may be complete, e.g., the total absence of pathology of a disease or a disorder as described herein in a subject. The prevention may also be partial, such that the occurrence of pathology a disease or a disorder as described herein in a subject is less than that which would have occurred without the present disclosure.

As used herein, the terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The present disclosure is directed towards treating a patient's suffering from a disease or a disorder as described herein. The term “prophylaxis” is used herein to refer to a measure or measures taken for the prevention or partial prevention of a disease or condition.

By “treating or preventing a disease or a disorder” is meant ameliorating any of the conditions or signs or symptoms associated with the disorder or the disease before or after it has occurred including, for example, neuronal cell death, inflammation, and motor deficits. For example, alleviating a symptom of a disorder may involve reducing visible areas of neuronal cell death relative to an untreated control. As compared with an equivalent untreated control, such reduction or degree of prevention is at least 3%, 5%, 10%, 15%, 20%, 25%, 30%, 35%, 40%, 45%, 50%, 55%, 60%, 65%, 70%, 75%, 80%, 85%, 90%, 95%, 96%, 96\hat{ }, 98%, 99%, or 100% as measured by any standard technique. A patient who is being treated for a disease or a disorder as described herein, for example, a neurological disease or cancer, is one who a medical practitioner has diagnosed as having such a condition. Diagnosis may be by any suitable means. Diagnosis and monitoring may involve, for example, detecting the presence of destroyed or dying neurons in a biological sample (e.g., tissue biopsy, blood test, or urine test), detecting the presence of plaques, detecting the level of a surrogate marker of the neurologic disorder in a biological sample, or detecting symptoms or signs associated with the neurologic disorder. Diagnosis and monitoring may involve, for example, detecting the presence of cancer cells in a biological sample (e.g., tissue biopsy, blood test, or urine test), detecting the presence of tumors, detecting the level of a surrogate marker of cancer in a biological sample, or detecting symptoms or signs associated with cancer. A patient in whom the development of a neurologic disorder or cancer is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

As used herein, the terms “disease” and “disorder” are interchangeably used.

As used herein, the terms “blood brain barrier” and “BBB” are used interchangeably, referring to a permeable barrier which closely regulates and severely restricts exchange between blood and brain tissue. In some embodiments, blood-brain barrier components include endothelial cells that form the deepest intima of all blood vessels, dense junctions between adjacent endothelial cells structurally associated with the BBB, basement membrane of endothelial cells, and enlarged foot processes of nearby astrocytes covering almost all of the exposed outer surface of blood vessels.

As used herein, the phrase “therapeutically effective amounts” used herein refers to the amount of agent, e.g., the compound as provided herein, the nanoparticles as provided herein, the composition as provided herein, or the pharmaceutical composition as provided herein, needed to treat, ameliorate, or prevent a targeted disease or condition. In some embodiments, a “therapeutically effective amount” is the amount of the compound as provided herein, the nanoparticles as provided herein, the composition as provided herein, or the pharmaceutical composition as provided herein sufficient to provide a beneficial effect or to otherwise reduce a detrimental non-beneficial event to the individual to whom the composition is administered. The exact dose will depend on the purpose of the treatment, and will be determined by one skilled in the art using known techniques (see, e.g. Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the term “inhibitory oligonucleotide molecule” refers to a nucleic acid molecule that can inhibit the expression and/or activity of a target nucleic acid molecule, e.g., an RNA molecule. Exemplary inhibitory oligonucleotide molecules include, but are not limited to RNAi agent, shRNA, siRNA, miRNA, amiRNA, and antisense oligonucleotide molecule. In some embodiments, the inhibitory oligonucleotide molecule comprises a region that is substantially complementary to at least part the targeted nucleic acid molecule, e.g., targeted mRNA transcript.

As used herein, the term “RNA interference (RNAi)” refers to a biological process in which RNA molecules are involved in sequence-specific suppression of gene expression and/or activity by double-stranded RNA, through translational or transcriptional repression.

As used herein, the term “RNAi agent” refers to an agent that induces or promotes RNA interference. In some embodiments, an RNAi agent induces or promotes RNA interference in vivo, in vitro, in cells, in tissues or in subjects.

As used herein, the term “shRNA”, also known as “short hairpin RNA” or “small hairpin RNA” refers to an artificial RNA molecule with a tight hairpin turn that can be used to silence target gene expression and/or activity via RNA interference.

As used herein, the term “siRNA”, also known as “small interfering RNA” “short interfering RNA”, or “silencing RNA” refers to a class of double-stranded RNA at first non-coding RNA molecules, typically 20-24 (normally 21) base pairs in length, and operating within the RNA interference pathway. In some embodiments, siRNA interferes with the expression and/or activity of specific genes with complementary nucleotide sequences by degrading mRNA after transcription, preventing translation

As used herein, the term “miRNA” or “microRNA” or “miR” refers to a small, single-stranded, non-coding RNA molecules containing 21 to 23 nucleotides, which are involved in RNA silencing and post-transcriptional regulation of gene expression. In some embodiments, miRNA is found in plants, animals and some viruses. As used herein, the term “amiRNA” refers to an artificial microRNA.

As used herein, the term “Lᵢ” or “Lⱼ” refers to a polysaccharide comprising “i” or “j” number of monosaccharides. For example, L2 refers to a disaccharide and L3 refers to a trisaccharide.

As used herein, the term “antisense oligonucleotide molecule” refers to a single strand of DNA or RNA that are complementary to a chosen sequence. In some embodiments, the antisense oligonucleotide molecule prevents protein translation of certain messenger RNA strands by binding to them, in a process called hybridization. In some embodiment, the antisense oligonucleotide molecule can be used to target a specific, complementary (coding or non-coding) RNA.

As used herein, the term “isolated,” refers to altered or removed material from the natural state or from its original or native environment. For example, a naturally-occurring polynucleotide or polypeptide present in a living animal is not isolated, but the same polynucleotide or polypeptide, separated by human intervention from some or all of the co-existing materials in the natural system, is isolated. An isolated polynucleotide (ribonucleic acid (RNA), deoxyribonucleic acid (DNA)), or polypeptide is free of the genes/nucleic acids or sequences/amino acids that flank it in its naturally-occurring state.

The term “operably linked” refers to functional linkage between two moieties or two nucleotide sequence. For example, the first moiety or the first nucleotide sequence is operably linked to the second moiety or the second nucleotide sequence when the first moiety or the first nucleotide sequence is placed in a functional relationship with the second moiety or the second nucleotide sequence.

As used herein, the terms “nucleic acid,” “nucleotide,” “polynucleotide,” or “oligonucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The nucleic acid may be a recombinant polynucleotide, or a polynucleotide of genomic, cDNA, semisynthetic, or synthetic origin which either does not occur in nature or is linked to another polynucleotide in a non-natural arrangement. The following abbreviations for the commonly occurring nucleic acid bases are used. “A” refers to adenosine, “C” refers to cytosine, “G” refers to guanosine, “T” refers to thymidine, and “U” refers to uridine.

As used herein, the terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

As used herein, the term “identity” or “homology” are used interchangeably. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm, for example, using the Needleman and Wunsch ((1970) J. Mol. Biol. 48:444-453) algorithm which has been incorporated into the GAP program in the GCG software package; using either a Blossum 62 matrix or a PAM250 matrix with a gap weight of 16, 14, 12, 10, 8, 6, or 4 and a length weight of 1, 2, 3, 4, 5, or 6; using the GAP program in the GCG software package with a NWSgapdna.CMP matrix and a gap weight of 40, 50, 60, 70, or 80 and a length weight of 1, 2, 3, 4, 5, or 6; using the algorithm of E. Meyers and W. Miller ((1989) CABIOS, 4:11-17) which has been incorporated into the ALIGN program (version 2.0), with a PAM120 weight residue table, a gap length penalty of 12 and a gap penalty of 4; or using the NBLAST and XBLAST programs (version 2.0) of Altschul, et al. (1990) J. Mol. Biol. 215:403-10 with the NBLAST program, score=100, wordlength=12 or with the XBLAST program, score=50, wordlength=3.

As used herein, the term “analog” or “functional analog” refers to a molecule that comprises a substantially similar structure, and is capable of having one or more activities of the reference molecule. In some embodiments, the functional analog have at least 40% 50%, 60%, 70%, 80%, 90%, 95%, 96%, 97%, 98%, 99% or 100% activity of the reference molecule.

As used herein, the term “monosaccharide,” or “simple sugar” or “sugar” refers to the simplest forms of sugar and the most basic units from which all carbohydrates are built.

As used herein, the term “SAMiRNA™” refers to a self-assembled micelle inhibitory RNA, which is a nanoparticle-type siRNA.

In some embodiments, SAMiRNA™ comprises the structure of Formula 1:

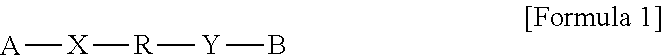

wherein:
A is a hydrophilic moiety,
B is a hydrophobic moiety, and
X and Y each independently represent a simple covalent bond or linked-mediated covalent bond, and
R refers to a double strand oligonucleotide comprising a sense strand and an antisense strand comprising a sequence complementary thereto.

As used herein, the terms “CNS platform” or “brain platform” are used interchangeably and refer to the compound as provided herein, the nanoparticle as provided herein, the composition as provided herein, or the pharmaceutical composition as provided herein, in which a ligand molecule for a glucose transporter (GLUT), or functional analog thereof is covalently linked to a hydrophilic moiety or a hydrophobic moiety of a self-assembled micelle oligonucleotide molecule, e.g., SAMiRNA™, wherein the blood brain barrier penetration and/or the brain delivery of the CNS platform or the brain platform is increased compared with a self-assembled micelle oligonucleotide molecule that does not comprise a ligand molecule for a GLUT, or functional analog thereof linked.

### Oligonucleotide Compounds Comprising GLUT Ligand, Hydrophilic and Hydrophobic Moieties

In an aspect, provided herein, inter alia, is a compound comprising the structure of Formula 16:

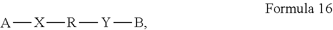

wherein:
R is an oligonucleotide molecule;
A is a hydrophilic moiety;
B is a hydrophobic moiety; and
X and Y are independently a covalent bond or a first linker;
wherein a ligand molecule for a glucose transporter (GLUT), or functional analog thereof is covalently linked to a terminus of A, a terminus of B, or a combination thereof via a second linker;
wherein the second linker is —NH(CH₂)ₙCONH—CH₂—CH(OH)—CH₂—; and
wherein R modulates the expression level and/or activity level of a target molecule.

In an aspect, provided herein, inter alia, is a compound comprising the structure of Formula 16:

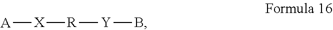

wherein:
R is an oligonucleotide molecule;
A is a hydrophilic moiety;
B is a hydrophobic moiety; and
X and Y are independently a covalent bond or a first linker;
wherein a ligand molecule for a glucose transporter (GLUT), or functional analog thereof is covalently linked to a terminus of A, a terminus of B, or a combination thereof.

In some embodiments, the compound comprises the structure of Formula 16:

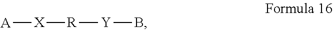

wherein:
R is an oligonucleotide molecule;
A is a hydrophilic moiety;
B is a hydrophobic moiety; and
X and Y are independently a covalent bond or a first linker;
wherein a ligand molecule for a glucose transporter (GLUT), or functional analog thereof is covalently linked to a terminus of A, a terminus of B, or a combination thereof via a second linker; and
wherein the second linker is —NH(CH₂)ₙCONH—CH₂—CH(OH)—CH₂—.

In another aspect, provided herein is a compound comprising the structure of Formula 16:

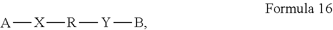

wherein:
R is an oligonucleotide molecule;
A is a hydrophilic moiety;
B is a hydrophobic moiety; and
X and Y are independently a covalent bond or a first linker;
wherein a ligand molecule for a glucose transporter (GLUT), or functional analog thereof is covalently linked to a terminus of A, a terminus of B, or a combination thereof via a second linker or a bond; and
wherein R modulates the expression level and/or activity level of a target molecule. In some embodiments, the second linker is —NH(CH₂)ₙCONH—CH₂—CH(OH)—CH₂—, an ether linkage, or Compound 4:

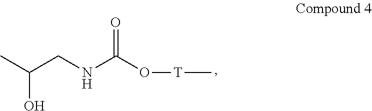

wherein T is 1-15 repetitive Compound 1:

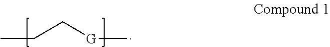

wherein G is selected from the group consisting of CH₂, O, S and NH.

In some embodiments, the second linker is any one listed in Table 7.

In another aspect, provided herein is a compound comprising the structure of Formula 16:

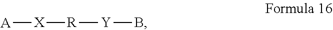

wherein:
R is an oligonucleotide molecule;
A is a hydrophilic moiety;
B is a hydrophobic moiety; and
X and Y are independently a covalent bond or a first linker;
wherein a ligand molecule for a glucose transporter (GLUT), or functional analog thereof is covalently linked to a terminus of A, a terminus of B, or a combination thereof via a second linker or a bond. In some embodiments, the second linker is —NH(CH₂)ₙCONH—CH₂—CH(OH)—CH₂—, an ether linkage, or Compound 4:

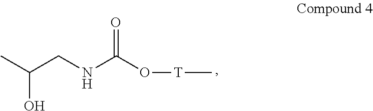

wherein T is 1-15 repetitive Compound 1:

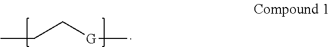

wherein G is selected from the group consisting of CH₂, O, S and NH.

In some embodiments, the second linker is any one listed in Table 7.

In some embodiments, R reduces or inhibits the expression and/or activity of a target molecule. In some embodiments, R reduces or inhibits the expression of a target molecule. In some embodiments, R reduces or inhibits the activity of a target molecule.

In some embodiments, R reduces or inhibits the expression of a target molecule by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 99.5%, about 99.9%, or 100% compared with the expression of the target molecule in a control tissue, organ, cell, or subject that is not treated with the compound as provided herein. In some embodiments, R reduces or inhibits the activity of a target molecule by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 99.5%, about 99.9%, or 100% compared with the expression of the target molecule in a control tissue, organ, cell, or subject that is not treated with the compound as provided herein.

In some embodiments, the compound comprises the structure of Formula 14 or Formula 15:

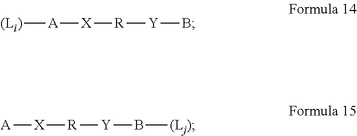

and
wherein L is the ligand molecule for a GLUT, or functional analog thereof;
wherein i and j are independently an integer of 0 through 10; and
wherein when i is 0, j is an integer of 1 through 10, and when j is 0, i is an integer of 1 through 10.

In some embodiments, i is an integer of 0 through 10. In some embodiments, i is an integer of 0 through 100, 0 through 95, 0 through 90, 0 through 85, 0 through 80, 0 through 75, 0 through 70, 0 through 70, 0 through 65, 0 through 60, 0 through 55, 0 through 50, 0 through 45, 0 through 40, 0 through 35, 0 through 30, 0 through 25, 0 through 20, 0 through 19, 0 through 18, 0 through 17, 0 through 16, 0 through 15, 0 through 14, 0 through 13, 0 through 12, 0 through 11, 0 through 10, 0 through 9, 0 through 8, 0 through 7, 0 through 6, 0 through 5, 0 through 4, 0 through 3, or 0 through 2. In some embodiments, i is an integer of 1 through 100, 2 through 100, 3 through 100, 4 through 100, 5 through 100, 6 through 100, 7 through 100, 8 through 100, 9 through 100, 10 through 100, 11 through 100, 12 through 100, 13 through 100, 14 through 100, 15 through 100, 16 through 100, 17 through 100, 18 through 100, 19 through 100, 20 through 100, 25 through 100, 30 through 100, 35 through 100, 40 through 100, 45 through 100, 50 through 100, 55 through 100, 60 through 100, 65 through 100, 70 through 100, 75 through 100, 80 through 100, 85 through 100, 90 through 100, or 95 through 100. In some embodiments, i is an integer of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments, j is an integer of 0 through 10. In some embodiments, j is an integer of 0 through 100, 0 through 95, 0 through 90, 0 through 85, 0 through 80, 0 through 75, 0 through 70, 0 through 70, 0 through 65, 0 through 60, 0 through 55, 0 through 50, 0 through 45, 0 through 40, 0 through 35, 0 through 30, 0 through 25, 0 through 20, 0 through 19, 0 through 18, 0 through 17, 0 through 16, 0 through 15, 0 through 14, 0 through 13, 0 through 12, 0 through 11, 0 through 10, 0 through 9, 0 through 8, 0 through 7, 0 through 6, 0 through 5, 0 through 4, 0 through 3, or 0 through 2. In some embodiments, j is an integer of 1 through 100, 2 through 100, 3 through 100, 4 through 100, 5 through 100, 6 through 100, 7 through 100, 8 through 100, 9 through 100, 10 through 100, 11 through 100, 12 through 100, 13 through 100, 14 through 100, 15 through 100, 16 through 100, 17 through 100, 18 through 100, 19 through 100, 20 through 100, 25 through 100, 30 through 100, 35 through 100, 40 through 100, 45 through 100, 50 through 100, 55 through 100, 60 through 100, 65 through 100, 70 through 100, 75 through 100, 80 through 100, 85 through 100, 90 through 100, or 95 through 100. In some embodiments, j is an integer of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments, i is an integer of 0, and j is an integer of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, i is an integer of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and j is an integer of 0.

In some embodiments, the ligand molecule for a GLUT is a sugar. In some embodiments, the sugar is selected from the group consisting of fructose, galactose, glucosamine, glucose, mannose, xylose, dehydroascorbic acid, and trehalose. In some embodiments, the ligand molecule for a GLUT is selected from the group consisting of fructose, galactose, glucosamine, glucose, mannose, xylose, dehydroascorbic acid, trehalose, and urate.

In some embodiments, the GLUT is selected from the group consisting of GLUT1, GLUT2, GLUT 3, GLUT 4, GLUT 5, GLUT 6, GLUT 7, GLUT 8, GLUT 9, GLUT 10, GLUT 11, GLUT 12, GLUT 13, and GLUT14. In some embodiments, the GLUT is selected from any one of the GLUT listed in Table 3, Table 4, and Table 5. In some embodiments, the GLUT includes any of the recombinant or naturally-occurring forms of GLUT or variants or homologs thereof that have or maintain GLUT activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring GLUT. In some embodiments, GLUT is substantially identical to the protein identified by the UniProt reference number as listed in Table 3 or a variant or homolog having substantial identity thereto.

In some embodiments, the ligand molecule for a GLUT is fructose and the GLUT is selected from the group consisting of GLUT2, GLUT5, GLUT7, GLUT8, GLUT9, GLUT11, and GLUT12. In some embodiments, the ligand molecule for a GLUT is galactose and the GLUT is selected from the group consisting of GLUT1, GLUT2, GLUT3, GLUT4, and GLUT14. In some embodiments, the ligand molecule for a GLUT is glucose and the GLUT is selected from the group consisting of GLUT1, GLUT2, GLUT3, GLUT4, GLUT6, GLUT7, GLUT8, GLUT9, GLUT10, GLUT11, and GLUT12. In some embodiments, the ligand molecule for a GLUT is mannose and the GLUT is selected from the group consisting of GLUT1, GLUT2, GLUT3, and GLUT4. In some embodiments, the ligand molecule for a GLUT is xylose and the GLUT is GLUT3. In some embodiments, the ligand molecule for a GLUT is dehydroascorbic acid and the GLUT is GLUT4. In some embodiments, the ligand molecule for a GLUT is trehalose and the GLUT is GLUT8. In some embodiments, the ligand molecule for a GLUT is urate and the GLUT is GLUT9. In some embodiments, the ligand molecule for a GLUT is any one selected from the ligands listed in Table 3, and the GLUT is the corresponding GLUT as listed in Table 3.

In some embodiments, R is a double stranded oligonucleotide molecule or a single stranded oligonucleotide molecule. In some embodiments, R is an inhibitory oligonucleotide molecule. In some embodiments, the inhibitory oligonucleotide molecule reduces or inhibits expression and/or activity of the target molecule. In some embodiments, the target molecule is a transcript of a gene associated with a disease.

In some embodiments, the target molecule is a transcript of a gene associated with a neural disease. In some embodiments, the target molecule is over expressed or activated in a patient having the neural disease. In some embodiments, the target molecule is a transcript of a gene associated with Alzheimer's disease. In some embodiments, the target molecule is a transcript of a gene disclosed in Table 6A. In some embodiments, the target molecule is a transcript of a gene associated with Parkinson's disease. In some embodiments, the target molecule is a transcript of a gene disclosed in Table 6B. In some embodiments, the target molecule is a transcript of a gene associated with Huntington's disease. In some embodiments, the target molecule is a transcript of a gene disclosed in Table 6C. In some embodiments, the target molecule is a transcript of a gene associated with Multiple sclerosis. In some embodiments, the target molecule is a transcript of a gene disclosed in Table 6D. In some embodiments, the target molecule is a transcript of a gene associated with Spinal cord injury. In some embodiments, the target molecule is a transcript of a gene disclosed in Table 6E.

In some embodiments, the target molecule is a transcript of any one selected from the group consisting of BACE1-AS, BACE1, presenilin1 (PS1), ROCK-II, 12 PP-2A, ACAT-1, Nogo receptor, mutant presenilin1 (L392V PS-1), APP, Tau, VDAC1, α-synuclein, Huntingtin (Htt), Notch1, LINGO-1, NR4A2, TRIF, caspase-2, CaMKII, glial fibrillary acidic protein (GFAP), Vimentin, EphB3, iNOS, Nischarin, and RhoA.

In some embodiments, the target molecule is a transcript of a gene associated with cancer. In some embodiments, the target molecule is over expressed or activated in a cancer cell.

In some embodiments, the inhibitory oligonucleotide molecule is an siRNA, an shRNA, a miRNA, an amiRNA, an antisense oligonucleotide molecule, an RNAi agent, or any combination thereof. In some embodiments, R is a double strand oligonucleotide molecule comprising a sense strand and an antisense strand comprising a sequence complementary to the sense strand. In some embodiments, R is a double stranded oligonucleotide comprising a DNA/DNA, DNA/RNA, or RNA/RNA hybrid. In some embodiments, R comprises a DNA/RNA duplex.

In some embodiments, the sense strand or anti-sense strand is 19 to 31 nucleotides in length. In some embodiments, the sense strand or anti-sense strand is 1 to 100, 1 to 95, 1 to 90, 1 to 85, 1 to 80, 1 to 75, 1 to 70, 1 to 65, 1 to 60, 1 to 55, 1 to 50, 1 to 45, 1 to 40, 1 to 35, 1 to 30, 1 to 25, 1 to 20, 1 to 15, or 1 to 10 nucleotides in length. In some embodiments, the sense strand or anti-sense strand is 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100 nucleotides in length. In some embodiments, the sense strand or anti-sense strand is 5 to 45, 10 to 40, 15 to 35, 16 to 34, 17 to 33, 18 to 32, 19 to 31, 20 to 30, 21 to 29, 22 to 28, 23 to 27, 24 to 26 nucleotides in length. In some embodiments, the sense strand or anti-sense strand is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the sense strand or anti-sense strand comprises 19 to 31 nucleotides. In some embodiments, the sense strand or anti-sense strand comprises 1 to 100, 1 to 95, 1 to 90, 1 to 85, 1 to 80, 1 to 75, 1 to 70, 1 to 65, 1 to 60, 1 to 55, 1 to 50, 1 to 45, 1 to 40, 1 to 35, 1 to 30, 1 to 25, 1 to 20, 1 to 15, or 1 to 10 nucleotides. In some embodiments, the sense strand or anti-sense strand comprises 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100 nucleotides. In some embodiments, the sense strand or anti-sense strand comprises 5 to 45, 10 to 40, 15 to 35, 16 to 34, 17 to 33, 18 to 32, 19 to 31, 20 to 30, 21 to 29, 22 to 28, 23 to 27, 24 to 26 nucleotides. In some embodiments, the sense strand or anti-sense strand comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides.

In some embodiments, at least one phosphate group is linked to the 5′ terminus of the anti-sense strand.

In some embodiments, the compound comprise the structure of Formula 2:

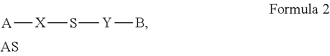

where S is the sense strand, and AS is the anti-sense strand.

In some embodiments, R is the antisense oligonucleotide molecule consisting of from 5 to 50 oligonucleotides, 10 to 40 oligonucleotides, or 15 to 30 oligonucleotides.

In some embodiments, R is the antisense oligonucleotide molecule consisting of from 1 to 200 oligonucleotides, from 5 to 200 oligonucleotides, from 10 to 200 oligonucleotides, from 15 to 200 oligonucleotides, from 20 to 200 oligonucleotides, from 25 to 200 oligonucleotides, from 30 to 200 oligonucleotides, from 35 to 200 oligonucleotides, from 40 to 200 oligonucleotides, from 45 to 200 oligonucleotides, from 50 to 200 oligonucleotides, from 55 to 200 oligonucleotides, from 60 to 200 oligonucleotides, from 65 to 200 oligonucleotides, from 70 to 200 oligonucleotides, from 75 to 200 oligonucleotides, from 80 to 200 oligonucleotides, from 85 to 200 oligonucleotides, from 90 to 200 oligonucleotides, from 95 to 200 oligonucleotides, from 100 to 200 oligonucleotides, from 105 to 200 oligonucleotides, from 110 to 200 oligonucleotides, from 115 to 200 oligonucleotides, from 120 to 200 oligonucleotides, from 125 to 200 oligonucleotides, from 130 to 200 oligonucleotides, from 135 to 200 oligonucleotides, from 140 to 200 oligonucleotides, from 145 to 200 oligonucleotides, from 150 to 200 oligonucleotides, from 155 to 200 oligonucleotides, from 160 to 200 oligonucleotides, from 165 to 200 oligonucleotides, from 160 to 200 oligonucleotides, from 175 to 200 oligonucleotides, from 180 to 200 oligonucleotides, from 185 to 200 oligonucleotides, from 190 to 200 oligonucleotides, or from 195 to 200 oligonucleotides. In some embodiments, R is the antisense oligonucleotide molecule consisting of from 1 to 195 oligonucleotides, from 1 to 190 oligonucleotides, from 1 to 185 oligonucleotides, from 1 to 180 oligonucleotides, from 1 to 175 oligonucleotides, from 1 to 170 oligonucleotides, from 1 to 165 oligonucleotides, from 1 to 160 oligonucleotides, from 1 to 155 oligonucleotides, from 1 to 150 oligonucleotides, from 1 to 145 oligonucleotides, from 1 to 140 oligonucleotides, from 1 to 135 oligonucleotides, from 1 to 130 oligonucleotides, from 1 to 125 oligonucleotides, from 1 to 120 oligonucleotides, from 1 to 115 oligonucleotides, from 1 to 110 oligonucleotides, from 1 to 105 oligonucleotides, from 1 to 100 oligonucleotides, from 1 to 95 oligonucleotides, from 1 to 90 oligonucleotides, from 1 to 85 oligonucleotides, from 1 to 80 oligonucleotides, from 1 to 75 oligonucleotides, from 1 to 70 oligonucleotides, from 1 to 65 oligonucleotides, from 1 to 60 oligonucleotides, from 1 to 55 oligonucleotides, from 1 to 50 oligonucleotides, from 1 to 45 oligonucleotides, from 1 to 40 oligonucleotides, from 1 to 35 oligonucleotides, from 1 to 30 oligonucleotides, from 1 to 25 oligonucleotides, from 1 to 20 oligonucleotides, from 1 to 15 oligonucleotides, or from 1 to 10 oligonucleotides. In some embodiments, R is the antisense oligonucleotide molecule consisting of 5 to 45 oligonucleotides, 10 to 40 oligonucleotides, 15 to 30 oligonucleotides, or 20 to 25 oligonucleotides. In some embodiments, R is the antisense oligonucleotide molecule consisting of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 oligonucleotides.

In some embodiments, R is DNA, RNA, or a combination thereof. In some embodiments, R comprises DNA/DNA, RNA/RNA, or DNA/RNA hybrid.

In some embodiments, R comprises a backbone modification comprising a phosphorothioate linkage, a phosphorodiamidate linkage, a boranophosphate linkage, a methyl phosphonate linkage, or any combination thereof.

In some embodiments, R comprises a locked nucleic acid, a peptide nucleic acid, a unlocked nucleic acid, or any combination thereof.

In some embodiments, R comprises a 2′-O-methyl moiety, a 2′-fluoro moiety, a 2′-O-methoxyethyl moiety, a 2′-methyl moiety, a 2′-methoxyl moiety, a 2′-amine moiety, a 2′-O-propyl moiety, a 2′-O-2-methylthioethyl moiety, a 2′-O-3-aminopropyl moiety, a 2′-O-3-dimethylaminopropyl moiety, a 2′-O—N-methylacetamino moiety, or a 2′-O-dimethylamidooxyethyl moiety.

In some embodiments, R comprises a modified sugar moiety.

In some embodiments, the hydrophilic moiety is any one selected from the group consisting of polyethylene glycol (PEG), polyvinyl pyrrolidone and polyoxazoline. In some embodiments, the hydrophilic moiety comprises HEG (hexaethylene glycol). In some embodiments, the hydrophilic moiety is HEG (hexaethylene glycol). In some embodiments, the hydrophilic moiety comprises or consist of hexaethyleneglycol-(—PO₃⁻hexaethyleneglycol)₃.

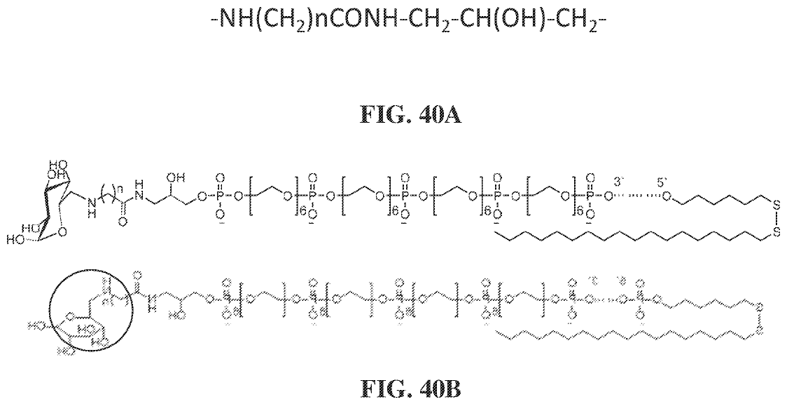

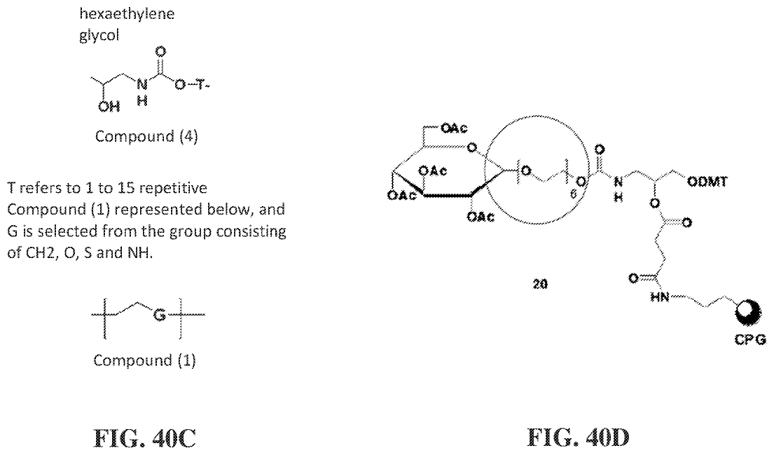

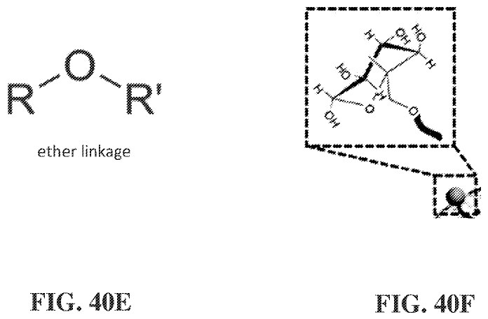

In some embodiments, R comprises nucleotide modification. In some embodiments, the modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the T position or 4′ position) or replacement of the sugar, as well as (d) backbone modifications, including modification or replacement of the phosphodiester linkages. In some embodiments, modified nucleotide backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. In some embodiments, various salts, mixed salts and free acid forms are also included. In some embodiments, modified nucleotide backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. In some embodiments, these include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH₂ component parts, and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂—, and —N(CH₃)—CH₂—CH₂— (wherein the native phosphodiester backbone is represented as —O—P—O—CH₂—). In some embodiments, exemplary links or linkages and exemplary compounds comprising links or linkages are as shown in FIGS. 40A-40F.

In some embodiments, in other nucleotide mimetics suitable or contemplated for use herein, both the sugar and the internucleoside linkage, i.e., the backbone, of the nucleotide units are replaced with novel groups. In some embodiments, the base units are maintained for hybridization with an appropriate nucleic acid target compound. In some embodiments, one such oligomeric compound is a peptide nucleic acid (PNA). In PNA compounds, the sugar backbone of a nucleotide is replaced with an amide containing backbone, in particular an aminoethylglycine backbone. The nucleobases are retained and are bound directly or indirectly to aza nitrogen atoms of the amide portion of the backbone. In some embodiments, the oligonucleotide molecule can also be modified to include one or more locked nucleic acids (LNA). A locked nucleic acid is a nucleotide having a modified ribose moiety in which the ribose moiety comprises an extra bridge connecting the 2′ and 4′ carbons. In some embodiments, this structure effectively “locks” the ribose in the 3′-endo structural conformation. In some embodiments, the addition of locked nucleic acids to the oligonucleotide molecule can increase the stability in serum, and to reduce off-target effects.

In some embodiments, the modified nucleotides can also contain one or more substituted sugar moieties. In some embodiments, R can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In some embodiments, exemplary suitable modifications include O[(CH₂)ₙO]mCH₃, O(CH₂)nOCH₃, O(CH₂)nNH2, O(CH₂)nCH₃, O(CH₂)nONH₂, and O(CH₂)nON[(CH₂)nCH₃)]₂, wherein n and m are from 1 to about 10. In some embodiments, R may include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a nucleotide cleaving group, a reporter group, an intercalator, a group for improving the pharmacokinetic properties of an oligonucleotide molecule, or a group for improving the pharmacodynamic properties of an oligonucleotide molecule, and other substituents having similar properties. In some embodiments, the modification includes a 2′ methoxyethoxy (2′-O—CH₂CH₂OCH₃, also known as 2′-O-(2-methoxyethyl) or 2′-MOE), i.e., an alkoxy-alkoxy group. Another exemplary modification includes, but is not limited to, 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

In some embodiments, other modifications include 2′-methoxy (2′-OCH₃), 2′-aminopropoxy (2-OCH₂CH₂CH₂NH₂) and 2′-fluoro (2′-F). In some embodiments, similar modifications can also be made at other positions on the nucleotide, particularly the 3′ position of the sugar on the 3′ terminal nucleotide or in 2′-5′ linked double-stranded oligonucleotides and the 5′ position of 5′ terminal nucleotide. In some embodiments, R may also have sugar mimetics such as cyclobutyl moieties in place of the pentofuranosyl sugar.

In some embodiments, R can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). In some embodiments, modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. In some embodiments, these nucleobases may be useful for increasing the binding affinity of the oligonucleotide molecule. In some embodiments, these include 5-substituted pyrimidines, 6-azapyrimidines and N-2, N-6 and O-6 substituted purines, including 2-aminopropyladenine, 5-propynyluracil and 5-propynylcytosine, and 5-methylcytosine substitutions to increase nucleic acid duplex stability.

The preparation of the modified nucleic acids, backbones, and nucleobases described above are well known in the art.

In some embodiments, the compound comprises the structure of Formula 7 or Formula 8:

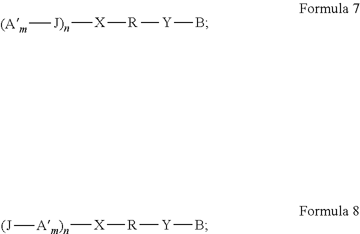

wherein:
A′ is a monomer hydrophilic moiety,
J is selected from the group comprised of PO₃⁻, SO₃⁻ and CO₂⁻, and links A′ to each other or links A′ to X,
m is an integer of 1 to 15, and
n is an integer of 1 to 10; and
wherein A′ is any one selected from the group consisting of Compound 1, Compound 2, and Compound 3:

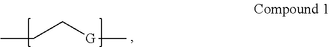

wherein G is selected from the group comprised of O, S and NH;

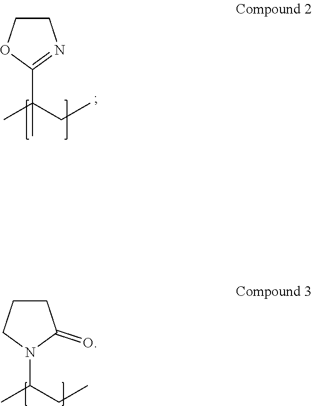

In some embodiments, m is an integer of 1 to 15. In some embodiments, m is an integer of 1 through 100, 1 through 95, 1 through 90, 1 through 85, 1 through 80, 1 through 75, 1 through 70, 1 through 70, 1 through 65, 1 through 60, 1 through 55, 1 through 50, 1 through 45, 1 through 40, 1 through 35, 1 through 30, 1 through 25, 1 through 20, 1 through 19, 1 through 18, 1 through 17, 1 through 16, 1 through 15, 1 through 14, 1 through 13, 1 through 12, 1 through 11, 1 through 10, 1 through 9, 1 through 8, 1 through 7, 1 through 6, 1 through 5, 1 through 4, 1 through 3, or 1 through 2. In some embodiments, m is an integer of 1 through 100, 2 through 100, 3 through 100, 4 through 100, 5 through 100, 6 through 100, 7 through 100, 8 through 100, 9 through 100, 10 through 100, 11 through 100, 12 through 100, 13 through 100, 14 through 100, 15 through 100, 16 through 100, 17 through 100, 18 through 100, 19 through 100, 20 through 100, 25 through 100, 30 through 100, 35 through 100, 40 through 100, 45 through 100, 50 through 100, 55 through 100, 60 through 100, 65 through 100, 70 through 100, 75 through 100, 80 through 100, 85 through 100, 90 through 100, or 95 through 100. In some embodiments, m is an integer of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments, n is an integer of 1 to 10. In some embodiments, n is an integer of 1 through 100, 1 through 95, 1 through 90, 1 through 85, 1 through 80, 1 through 75, 1 through 70, 1 through 70, 1 through 65, 1 through 60, 1 through 55, 1 through 50, 1 through 45, 1 through 40, 1 through 35, 1 through 30, 1 through 25, 1 through 20, 1 through 19, 1 through 18, 1 through 17, 1 through 16, 1 through 15, 1 through 14, 1 through 13, 1 through 12, 1 through 11, 1 through 10, 1 through 9, 1 through 8, 1 through 7, 1 through 6, 1 through 5, 1 through 4, 1 through 3, or 1 through 2. In some embodiments, n is an integer of 1 through 100, 2 through 100, 3 through 100, 4 through 100, 5 through 100, 6 through 100, 7 through 100, 8 through 100, 9 through 100, 10 through 100, 11 through 100, 12 through 100, 13 through 100, 14 through 100, 15 through 100, 16 through 100, 17 through 100, 18 through 100, 19 through 100, 20 through 100, 25 through 100, 30 through 100, 35 through 100, 40 through 100, 45 through 100, 50 through 100, 55 through 100, 60 through 100, 65 through 100, 70 through 100, 75 through 100, 80 through 100, 85 through 100, 90 through 100, or 95 through 100. In some embodiments, n is an integer of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In some embodiments, the molecular weight of the hydrophilic moiety is 200 to 10,000.

In some embodiments, the molecular weight of the hydrophilic moiety is 10 to 50,000, 10 to 45,000, 10 to 40,000, 10 to 35,000, 10 to 30,000, 10 to 25,000, 10 to 20,000, 10 to 15,000, 10 to 10,000, 10 to 9,500, 10 to 9,000, 10 to 8,500, 10 to 8,000, 10 to 7,500, 10 to 7,000, 10 to 6,500, 10 to 6,000, 10 to 5,500, 10 to 5,000, 10 to 4,500, 10 to 4,000, 10 to 3,500, 10 to 3,000, 10 to 2,500, 10 to 2,000, 10 to 1,500, 10 to 1,000, 10 to 950, 10 to 900, 10 to 850, 10 to 800, 10 to 750, 10 to 700, 10 to 650, 10 to 600, 10 to 550, 10 to 500, 10 to 450, 10 to 400, 10 to 350, 10 to 300, 10 to 250, 10 to 200, 10 to 150, 10 to 150, or 10 to 100. In some embodiments, the molecular weight of the hydrophilic moiety is 50 to 50,000, 100 to 50,000, 150 to 50,000, 200 to 50,000, 250 to 50,000, 300 to 50,000, 350 to 50,000, 400 to 50,000, 450 to 50,000, 500 to 50,000, 550 to 50,000, 600 to 50,000, 650 to 50,000, 700 to 50,000, 750 to 50,000, 800 to 50,000, 850 to 50,000, 900 to 50,000, 950 to 50,000, 1,000 to 50,000, 1,500 to 50,000, 2,000 to 50,000, 2,500 to 50,000, 3,000 to 50,000, 3,500 to 50,000, 4,000 to 50,000, 4,500 to 50,000, 5,000 to 50,000, 5,500 to 50,000, 6,000 to 50,000, 6,500 to 50,000, 7,000 to 50,000, 7,500 to 50,000, 8,000 to 50,000, 8,500 to 50,000, 9,000 to 50,000, 10,000 to 50,000, 15,000 to 50,000, 20,000 to 50,000, 25,000 to 50,000, 30,000 to 50,000, 35,000 to 50,000, 40,000 to 50,000, or 45,000 to 50,00. In some embodiments, the molecular weight of the hydrophilic moiety is 10 to 30,000, 50 to 25,000, 100 to 20,000, 150 to 15,000, 200 to 10,000, 250 to 5,000, or 500 to 1,000.

In some embodiments, the hydrophobic moiety is any one selected from the group consisting of a steroid derivative, a glyceride derivative, glycerol ether, poplypropylene glycol, unsaturated or saturated C₁₂-C₅₀, diacylphosphatidylcholine, a fatty acid, a phospholipid, a lipopolyamine, a lipid, tocopherol, and tocotrienol. In some embodiments, the steroid derivative is any one selected from the group consisting of cholesterol, cholestanol, cholic acid, cholesteryl formate, cotestanyl formate, and colistanylamine. In some embodiments, the glyceride derivative is any one selected from the group consisting of monoglyceride, diglyceride and triglyceride.

In some embodiments, the molecular weight of the hydrophobic moiety is 250 to 1,000. In some embodiments, the molecular weight of the hydrophobic moiety is 10 to 5,000, 50 to 5,000, 100 to 5,000, 150 to 5,000, 200 to 5,000, 250 to 5,000, 300 to 5,000, 350 to 5,000, 400 to 5,000, 450 to 5,000, 500 to 5,000, 550 to 5,000, 600 to 5,000, 650 to 5,000, 700 to 5,000, 750 to 5,000, 800 to 5,000, 850 to 5,000, 900 to 5,000, 1,000 to 5,000, 1,500 to 5,000, 2,000 to 5,000, 2,500 to 5,000, 3,000 to 5,000, 3,500 to 5,000, 4,000 to 5,000, or 4,500 to 5,000. In some embodiments, the molecular weight of the hydrophobic moiety is 10 to 4,500, 10 to 4,000, 10 to 3,500, 10 to 3,000, 10 to 2,500, 10 to 2,000, 10 to 1,500, 10 to 1,000, 10 to 950, 10 to 900, 10 to 850, 10 to 800, 10 to 750, 10 to 700, 10 to 650, 10 to 600, 10 to 550, 10 to 500, 10 to 450, 10 to 400, 10 to 350, 10 to 300, 10 to 250, 10 to 200, 10 to 150, 10 to 100, 10 to 90, 10 to 80, 10 to 70, 10 to 60, 10 to 50, 10 to 40, 10 to 30, or 10 to 20. In some embodiments, the molecular weight of the hydrophobic moiety is 10 to 3,500, 50 to 3,000, 100 to 2,500, 150 to 2,000, 200 to 1,500, 250 to 1,000, 300 to 950, 350 to 900, 400 to 850, 450 to 800, 500 to 750, 550 to 700, or 600 to 650.

In some embodiments, X and Y are independently a non-degradable bond or a degradable bond. In some embodiments, the non-degradable bond is an amide bond or a phosphorylated bond. In some embodiments, the degradable bond is any one selected from the group consisting of a disulfide bond, an acid-degradable bond, an ester bond, an anhydride bond, a biodegradable bond, and an enzyme degradable bond.

In another aspect, provided herein is a nanoparticle comprising the compound as provided herein. In another aspect, provided herein is a composition comprising the compound as provided herein or the nanoparticle as provided herein. In another aspect, provided herein is a pharmaceutical composition comprising the compound as provided herein, the nanoparticle as provided herein, or the composition as provided herein, and a pharmaceutically acceptable excipient, carrier, or diluent.

### Target Molecules

In some embodiments, the target molecule is any one selected from the group consisting of BACE1-AS, BACE1, presenilin1 (PS1), ROCK-II, 12 PP-2A, ACAT-1, Nogo receptor, mutant presenilin1 (L392V PS-1), APP, Tau, VDAC1, α-synuclein, Huntingtin (Htt), Notch1, LINGO-1, NR4A2, TRIF, caspase-2, CaMKII, glial fibrillary acidic protein (GFAP), Vimentin, EphB3, iNOS, Nischarin, and RhoA.

In some embodiments, the target molecule is BACE1-AS and the compound as provided herein modulates expression and/or activity of BACE1-AS by targeting an mRNA transcribed from a BACE1-AS genomic sequence (Exemplary sequence: ENSG00000278768). In some embodiments, the target molecule is BACE1-AS and the compound as provided herein reduces or inhibits expression and/or activity of BACE1-AS by targeting an mRNA transcribed from a BACE1-AS genomic sequence (Exemplary sequence: ENSG00000278768). In some embodiments, the compound as provided herein may target any region of a BACE1-AS mRNA.

In some embodiments, the target molecule is Beta-secretase 1 (BACE1) and the compound as provided herein modulates expression and/or activity of BACE1 (UniProt No. for BACE1 protein: P56817) by targeting an mRNA transcribed from a BACE1 genomic sequence (Exemplary sequence: ENSG00000186318). In some embodiments, the target molecule is Beta-secretase 1 (BACE1) and the compound as provided herein reduces or inhibits expression and/or activity of BACE1 (UniProt No. for BACE1 protein: P56817) by targeting an mRNA transcribed from a BACE1 genomic sequence (Exemplary sequence: ENSG00000186318). In some embodiments, the compound as provided herein may target any region of a BACE1 mRNA.

In some embodiments, the target molecule is presenilin 1 (PS1) and the compound as provided herein modulates expression and/or activity of presenilin 1 (PS1) (UniProt No. for presenilin 1 (PS1) protein: P49768) by targeting an mRNA transcribed from a presenilin 1 (PS1) genomic sequence (Exemplary sequence: ENSG00000080815). In some embodiments, the target molecule is presenilin 1 (PS1) and the compound as provided herein reduces or inhibits expression and/or activity of presenilin 1 (PS1) (UniProt No. for presenilin 1 (PS1) protein: P49768) by targeting an mRNA transcribed from a presenilin 1 (PS1) genomic sequence (Exemplary sequence: ENSG00000080815). In some embodiments, the compound as provided herein may target any region of a presenilin 1 (PS1) mRNA.

In some embodiments, the target molecule is Rho-associated protein kinase II (ROCK-II) and the compound as provided herein modulates expression and/or activity of ROCK-II (UniProt No. for ROCK-II protein: 075116) by targeting an mRNA transcribed from a ROCK-II genomic sequence (Exemplary sequence: ENSG00000134318). In some embodiments, the target molecule is Rho-associated protein kinase II (ROCK-II) and the compound as provided herein reduces or inhibits expression and/or activity of ROCK-II (UniProt No. for ROCK-II protein: O75116) by targeting an mRNA transcribed from a ROCK-II genomic sequence (Exemplary sequence: ENSG00000134318). In some embodiments, the compound as provided herein may target any region of a ROCK-II mRNA.

In some embodiments, the target molecule is 12 PP-2A (Protein SET) and the compound as provided herein modulates expression and/or activity of 12 PP-2A (UniProt No. for 12 PP-2A protein: Q01105) by targeting an mRNA transcribed from a 12 PP-2A genomic sequence (Exemplary sequence: ENSG00000119335). In some embodiments, the target molecule is 12 PP-2A (Protein SET) and the compound as provided herein reduces or inhibits expression and/or activity of I2 PP-2A (UniProt No. for 12 PP-2A protein: Q01105) by targeting an mRNA transcribed from a 12 PP-2A genomic sequence (Exemplary sequence: ENSG00000119335). In some embodiments, the compound as provided herein may target any region of a I2 PP-2A mRNA.

In some embodiments, the target molecule is Acetyl-CoA acetyltransferase, mitochondrial (ACAT-1) and the compound as provided herein modulates expression and/or activity of ACAT-1 (UniProt No. for ACAT-1 protein: P24752) by targeting an mRNA transcribed from a ACAT-1 genomic sequence (Exemplary sequence: ENSG00000075239). In some embodiments, the target molecule is Acetyl-CoA acetyltransferase, mitochondrial (ACAT-1) and the compound as provided herein reduces or inhibits expression and/or activity of ACAT-1 (UniProt No. for ACAT-1 protein: P24752) by targeting an mRNA transcribed from a ACAT-1 genomic sequence (Exemplary sequence: ENSG00000075239). In some embodiments, the compound as provided herein may target any region of a ACAT-1 mRNA.

In some embodiments, the target molecule is Nogo receptor (Reticulon 4 receptor (RTN4R)) and the compound as provided herein modulates expression and/or activity of Nogo receptor (UniProt No. for Nogo receptor protein: Q9BZR6) by targeting an mRNA transcribed from a Nogo receptor genomic sequence (Exemplary sequence: ENSG00000040608). In some embodiments, the target molecule is Nogo receptor (Reticulon 4 receptor (RTN4R)) and the compound as provided herein reduces or inhibits expression and/or activity of Nogo receptor (UniProt No. for Nogo receptor protein: Q9BZR6) by targeting an mRNA transcribed from a Nogo receptor genomic sequence (Exemplary sequence: ENSG00000040608). In some embodiments, the compound as provided herein may target any region of a Nogo receptor mRNA.

In some embodiments, the target molecule is mutant presenilin 1 (L392V PS-1), i.e., presenilin 1 comprising the L392V mutation, and the compound as provided herein modulates expression and/or activity of mutant presenilin 1 (L392V PS-1) by targeting an mRNA transcribed from a presenilin 1 genomic sequence comprising the L392V mutation. In some embodiments, the target molecule is mutant presenilin 1 (L392V PS-1), i.e., presenilin 1 comprising the L392V mutation, and the compound as provided herein reduces or inhibits expression and/or activity of mutant presenilin 1 (L392V PS-1) by targeting an mRNA transcribed from a presenilin 1 genomic sequence comprising the L392V mutation. In some embodiments, the compound as provided herein may target any region of a mutant presenilin 1 (L392V PS-1) mRNA.

In some embodiments, the target molecule is APP (amyloid-beta precursor protein) and the compound as provided herein modulates expression and/or activity of APP (UniProt No. for APP protein: P05067) by targeting an mRNA transcribed from a APP genomic sequence (Exemplary sequence: ENSG00000142192). In some embodiments, the target molecule is APP (amyloid-beta precursor protein) and the compound as provided herein reduces or inhibits expression and/or activity of APP (UniProt No. for APP protein: P05067) by targeting an mRNA transcribed from a APP genomic sequence (Exemplary sequence: ENSG00000142192). In some embodiments, the compound as provided herein may target any region of a APP mRNA.

In some embodiments, the target molecule is Tau (tubulin associated unit) and the compound as provided herein modulates expression and/or activity of Tau (UniProt No. for Tau protein: P10636) by targeting an mRNA transcribed from a Tau genomic sequence (Exemplary sequence: ENSG00000186868; ENSG00000276155; or ENSG00000277956). In some embodiments, the target molecule is Tau (tubulin associated unit) and the compound as provided herein reduces or inhibits expression and/or activity of Tau (UniProt No. for Tau protein: P10636) by targeting an mRNA transcribed from a Tau genomic sequence (Exemplary sequence: ENSG00000186868; ENSG00000276155; or ENSG00000277956). In some embodiments, the compound as provided herein may target any region of a Tau mRNA.

In some embodiments, the target molecule is Voltage-dependent anion-selective channel 1 (VDAC1) and the compound as provided herein modulates expression and/or activity of VDAC1 (UniProt No. for VDAC1 protein: P21796) by targeting an mRNA transcribed from a VDAC1 genomic sequence (Exemplary sequence: ENSG00000213585). In some embodiments, the target molecule is Voltage-dependent anion-selective channel 1 (VDAC1) and the compound as provided herein reduces or inhibits expression and/or activity of VDAC1 (UniProt No. for VDAC1 protein: P21796) by targeting an mRNA transcribed from a VDAC1 genomic sequence (Exemplary sequence: ENSG00000213585). In some embodiments, the compound as provided herein may target any region of a VDAC1 mRNA.

In some embodiments, the target molecule is α-synuclein and the compound as provided herein modulates expression and/or activity of α-synuclein (UniProt No. for α-synuclein protein: P37840) by targeting an mRNA transcribed from a α-synuclein genomic sequence (Exemplary sequence: ENSG00000145335). In some embodiments, the target molecule is α-synuclein and the compound as provided herein reduces or inhibits expression and/or activity of α-synuclein (UniProt No. for α-synuclein protein: P37840) by targeting an mRNA transcribed from a α-synuclein genomic sequence (Exemplary sequence: ENSG00000145335). In some embodiments, the compound as provided herein may target any region of a α-synuclein mRNA.

In some embodiments, the target molecule is Huntingtin (Htt) and the compound as provided herein modulates expression and/or activity of Htt (UniProt No. for Htt protein: P42858) by targeting an mRNA transcribed from a Htt genomic sequence (Exemplary sequence: ENSG00000197386). In some embodiments, the target molecule is Huntingtin (Htt) and the compound as provided herein reduces or inhibits expression and/or activity of Htt (UniProt No. for Htt protein: P42858) by targeting an mRNA transcribed from a Htt genomic sequence (Exemplary sequence: ENSG00000197386). In some embodiments, the compound as provided herein may target any region of a Htt mRNA.

In some embodiments, the target molecule is Neurogenic locus notch homolog protein 1 (Notch1) and the compound as provided herein modulates expression and/or activity of Notch1 (UniProt No. for Notch1 protein: P46531) by targeting an mRNA transcribed from a Notch1 genomic sequence (Exemplary sequence: ENSG00000148400). In some embodiments, the target molecule is Neurogenic locus notch homolog protein 1 (Notch1) and the compound as provided herein reduces or inhibits expression and/or activity of Notch1 (UniProt No. for Notch1 protein: P46531) by targeting an mRNA transcribed from a Notch1 genomic sequence (Exemplary sequence: ENSG00000148400). In some embodiments, the compound as provided herein may target any region of a Notch1 mRNA.

In some embodiments, the target molecule is Leucine rich repeat and Immunoglobin-like domain-containing protein 1 (LINGO-1) and the compound as provided herein modulates expression and/or activity of LINGO-1 (UniProt No. for LINGO-1 protein: Q96FE5) by targeting an mRNA transcribed from a LINGO-1 genomic sequence (Exemplary sequence: ENSG00000169783). In some embodiments, the target molecule is Leucine rich repeat and Immunoglobin-like domain-containing protein 1 (LINGO-1) and the compound as provided herein reduces or inhibits expression and/or activity of LINGO-1 (UniProt No. for LINGO-1 protein: Q96FE5) by targeting an mRNA transcribed from a LINGO-1 genomic sequence (Exemplary sequence: ENSG00000169783). In some embodiments, the compound as provided herein may target any region of a LINGO-1 mRNA.

In some embodiments, the target molecule is nuclear receptor 4A2 (NR4A2) and the compound as provided herein modulates expression and/or activity of NR4A2 (UniProt No. for NR4A2 protein: P43354) by targeting an mRNA transcribed from a NR4A2 genomic sequence (Exemplary sequence: ENSG00000153234). In some embodiments, the target molecule is nuclear receptor 4A2 (NR4A2) and the compound as provided herein reduces or inhibits expression and/or activity of NR4A2 (UniProt No. for NR4A2 protein: P43354) by targeting an mRNA transcribed from a NR4A2 genomic sequence (Exemplary sequence: ENSG00000153234). In some embodiments, the compound as provided herein may target any region of a NR4A2 mRNA.

In some embodiments, the target molecule is TIR-domain-containing adapter-inducing interferon-β (TRIF) and the compound as provided herein modulates expression and/or activity of TRIF (UniProt No. for TRIF protein: Q8IUC6) by targeting an mRNA transcribed from a TRIF genomic sequence (Exemplary sequence: ENSG00000127666). In some embodiments, the target molecule is TIR-domain-containing adapter-inducing interferon-β (TRIF) and the compound as provided herein reduces or inhibits expression and/or activity of TRIF (UniProt No. for TRIF protein: Q8IUC6) by targeting an mRNA transcribed from a TRIF genomic sequence (Exemplary sequence: ENSG00000127666). In some embodiments, the compound as provided herein may target any region of a TRIF mRNA.

In some embodiments, the target molecule is caspase-2 and the compound as provided herein modulates expression and/or activity of caspase-2 (UniProt No. for caspase-2 protein: P42575) by targeting an mRNA transcribed from a caspase-2 genomic sequence (Exemplary sequence: ENSG00000106144). In some embodiments, the target molecule is caspase-2 and the compound as provided herein reduces or inhibits expression and/or activity of caspase-2 (UniProt No. for caspase-2 protein: P42575) by targeting an mRNA transcribed from a caspase-2 genomic sequence (Exemplary sequence: ENSG00000106144). In some embodiments, the compound as provided herein may target any region of a caspase-2 mRNA.

In some embodiments, the target molecule is Ca2+/calmodulin-dependent protein kinase II, (CaMKII), e.g., Ca2+/calmodulin-dependent protein kinase II alpha, (CaMKII alpha) and/or Ca2+/calmodulin-dependent protein kinase II beta, (CaMKII beta), and the compound as provided herein modulates expression and/or activity of CaMKII alpha (UniProt No. for CaMKII alpha protein: Q9UQM7) and/or CaMKII beta (UniProt No. for CaMKII beta protein: Q13554) by targeting an mRNA transcribed from a CaMKII alpha genomic sequence (Exemplary sequence: ENSG00000058404) or a CaMKII beta genomic sequence (Exemplary sequence: ENSG00000106144). In some embodiments, the target molecule is Ca2+/calmodulin-dependent protein kinase II, (CaMKII), e.g., Ca2+/calmodulin-dependent protein kinase II alpha, (CaMKII alpha) and/or Ca2+/calmodulin-dependent protein kinase II beta, (CaMKII beta), and the compound as provided herein reduces or inhibits expression and/or activity of CaMKII alpha (UniProt No. for CaMKII alpha protein: Q9UQM7) and/or CaMKII beta (UniProt No. for CaMKII beta protein: Q13554) by targeting an mRNA transcribed from a CaMKII alpha genomic sequence (Exemplary sequence: ENSG00000058404) or a CaMKII beta genomic sequence (Exemplary sequence: ENSG00000106144). In some embodiments, the compound as provided herein may target any region of a CaMKII alpha mRNA or a CaMKII beta mRNA.

In some embodiments, the target molecule is glial fibrillary acidic protein (GFAP) and the compound as provided modulates expression and/or activity of GFAP (UniProt No. for GFAP protein: P14136) by targeting an mRNA transcribed from a GFAP genomic sequence (Exemplary sequence: ENSG00000131095). In some embodiments, the target molecule is glial fibrillary acidic protein (GFAP) and the compound as provided herein reduces or inhibits expression and/or activity of GFAP (UniProt No. for GFAP protein: P14136) by targeting an mRNA transcribed from a GFAP genomic sequence (Exemplary sequence: ENSG00000131095). In some embodiments, the compound as provided herein may target any region of a GFAP mRNA.

In some embodiments, the target molecule is Vimentin and the compound as provided herein modulates expression and/or activity of Vimentin (UniProt No. for Vimentin protein: P08670) by targeting an mRNA transcribed from a Vimentin genomic sequence (Exemplary sequence: ENSG00000026025). In some embodiments, the target molecule is Vimentin and the compound as provided herein reduces or inhibits expression and/or activity of Vimentin (UniProt No. for Vimentin protein: P08670) by targeting an mRNA transcribed from a Vimentin genomic sequence (Exemplary sequence: ENSG00000026025). In some embodiments, the compound as provided herein may target any region of a Vimentin mRNA.

In some embodiments, the target molecule is Ephrin type-B receptor 3 (EphB3) and the compound as provided herein modulates expression and/or activity of EphB3 (UniProt No. for EphB3 protein: P54753) by targeting an mRNA transcribed from a EphB3 genomic sequence (Exemplary sequence: ENSG00000182580). In some embodiments, the target molecule is Ephrin type-B receptor 3 (EphB3) and the compound as provided herein reduces or inhibits expression and/or activity of EphB3 (UniProt No. for EphB3 protein: P54753) by targeting an mRNA transcribed from a EphB3 genomic sequence (Exemplary sequence: ENSG00000182580). In some embodiments, the compound as provided herein may target any region of a EphB3 mRNA.

In some embodiments, the target molecule is inducible Nitric oxide synthase (iNOS) and the compound as provided herein modulates expression and/or activity of iNOS (UniProt No. for iNOS protein: P35228) by targeting an mRNA transcribed from an iNOS genomic sequence (Exemplary sequence: ENSG00000007171). In some embodiments, the target molecule is inducible Nitric oxide synthase (iNOS) and the compound as provided herein reduces or inhibits expression and/or activity of iNOS (UniProt No. for iNOS protein: P35228) by targeting an mRNA transcribed from an iNOS genomic sequence (Exemplary sequence: ENSG00000007171). In some embodiments, the compound as provided herein may target any region of an iNOS mRNA.

In some embodiments, the target molecule is Nischarin and the compound as provided herein modulates expression and/or activity of Nischarin (UniProt No. for Nischarin protein: Q9Y211) by targeting an mRNA transcribed from a Nischarin genomic sequence (Exemplary sequence: ENSG00000010322). In some embodiments, the target molecule is Nischarin and the compound as provided herein reduces or inhibits expression and/or activity of Nischarin (UniProt No. for Nischarin protein: Q9Y211) by targeting an mRNA transcribed from a Nischarin genomic sequence (Exemplary sequence: ENSG00000010322). In some embodiments, the compound as provided herein may target any region of a Nischarin mRNA.

In some embodiments, the target molecule is Transforming protein RhoA (RhoA) and the compound as provided herein modulates expression and/or activity of RhoA (UniProt No. for RhoA protein: P61586) by targeting an mRNA transcribed from a RhoA genomic sequence (Exemplary sequence: ENSG00000067560). In some embodiments, the target molecule is Transforming protein RhoA (RhoA) and the compound as provided herein reduces or inhibits expression and/or activity of RhoA (UniProt No. for RhoA protein: P61586) by targeting an mRNA transcribed from a RhoA genomic sequence (Exemplary sequence: ENSG00000067560). In some embodiments, the compound as provided herein may target any region of a RhoA mRNA.

In some embodiments, the target molecule is any one of the target molecule listed in Table 6A, Table 6B, Table 6C, Table 6D, and Table 6E. In some embodiments, the compound as provided herein modulates expression and/or activity of any one of the target molecule listed in Table 6A, Table 6B, Table 6C, Table 6D, and Table 6E. In some embodiments, the compound as provided herein reduces or inhibits expression and/or activity of any one of the target molecule listed in Table 6A, Table 6B, Table 6C, Table 6D, and Table 6E. In some embodiments, the compound as provided herein modulates expression and/or activity of any one of the target molecule listed in Table 6A, Table 6B, Table 6C, Table 6D, and Table 6E by targeting an mRNA transcribed from a genomic sequence of any one of the target molecule listed in Table 6A, Table 6B, Table 6C, Table 6D, and Table 6E. In some embodiments, the compound as provided herein reduces or inhibits expression and/or activity of any one of the target molecule listed in Table 6A, Table 6B, Table 6C, Table 6D, and Table 6E by targeting an mRNA transcribed from a genomic sequence of any one of the target molecule listed in Table 6A, Table 6B, Table 6C, Table 6D, and Table 6E. In some embodiments, the compound as provided herein may target any region of an mRNA of any one of the target molecule listed in Table 6A, Table 6B, Table 6C, Table 6D, and Table 6E.

In some embodiments, R modulates the expression and/or activity of a target molecule. In some embodiments, R modulates the expression of a target molecule. In some embodiments, R modulates the activity of a target molecule.

In some embodiments, R modulates the expression of a target molecule by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 99.5%, about 99.9%, or 100% compared with the expression of the target molecule in a control tissue, organ, cell, or subject that is not treated with the compound as provided herein. In some embodiments, R modulates the activity of a target molecule by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 99.5%, about 99.9%, or 100% compared with the expression of the target molecule in a control tissue, organ, cell, or subject that is not treated with the compound as provided herein. In some embodiments, the expression of the target molecule is modulated by the compound as provided herein by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared to the expression of the target molecule in a control tissue, organ, cell, or subject that is not treated with the compound as provided herein. In some embodiments, the activity of the target molecule is modulated by the compound as provided herein by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared to the activity of the target molecule in a control tissue, organ, cell, or subject that is not treated with the compound as provided herein.

In some embodiments, R reduces or inhibits the expression and/or activity of a target molecule. In some embodiments, R reduces or inhibits the expression of a target molecule. In some embodiments, R reduces or inhibits the activity of a target molecule.

In some embodiments, R reduces or inhibits the expression of a target molecule by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 99.5%, about 99.9%, or 100% compared with the expression of the target molecule in a control tissue, organ, cell, or subject that is not treated with the compound as provided herein. In some embodiments, R reduces or inhibits the activity of a target molecule by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 99.5%, about 99.9%, or 100% compared with the expression of the target molecule in a control tissue, organ, cell, or subject that is not treated with the compound as provided herein. In some embodiments, the expression of the target molecule is reduces or inhibits by the compound as provided herein by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared to the expression of the target molecule in a control tissue, organ, cell, or subject that is not treated with the compound as provided herein. In some embodiments, the activity of the target molecule is reduces or inhibits by the compound as provided herein by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared to the activity of the target molecule in a control tissue, organ, cell, or subject that is not treated with the compound as provided herein.

In some embodiments, the double strand oligonucleotide structure includes sense and antisense pair targeting hu-BACE1 selected from: #081, #001, #091, #131, #135, #177, #175, #093, #056, #009, #002, and #072, or combinations thereof (or a pair selected from Table 2C, Table 2D, Table 2E, Table 2F, and Table 2G). In some embodiments, the double strand oligonucleotide structure includes sense and antisense pairs selected from: AGGCACCACCCCTTGGAAC (SEQ ID NO: 22) and GUUCCAAGGGGUGGUGCCU (SEQ ID NO: 23), CCAGCACATACCGGGACCT (SEQ ID NO: 24) and AGGUCCCGGUAUGUGCUGG (SEQ ID NO: 25), GGTGAGGTTACCAACCAGT (SEQ ID NO: 26) and ACUGGUUGGUAACCUCACC (SEQ ID NO: 27), CCCGAAAACGAATTGGCTT (SEQ ID NO: 28) and AAGCCAAUUCGUUUUCGGG (SEQ ID NO: 29), TGGCTTTGCTGTCAGCGCT (SEQ ID NO: 30) and AGCGCUGACAGCAAAGCCA (SEQ ID NO: 31), TACTCTTGGTCACCTCAAA (SEQ ID NO: 32) and UUUGAGGUGACCAAGAGUA (SEQ ID NO: 33), AATACTCTTGGTCACCTCA (SEQ ID NO: 34) and UGAGGUGACCAAGAGUAUU (SEQ ID NO: 35), CCAACCAGTCCTTCCGCAT (SEQ ID NO: 36) and AUGCGGAAGGACUGGUUGG (SEQ ID NO: 37), ATCCGGCGGGAGTGGTATT (SEQ ID NO: 38) and AAUACCACUCCCGCCGGAU (SEQ ID NO: 39), CCTCCGGAAGGGTGTGTAT (SEQ ID NO: 40) and CAGCACATACCGGGACCTC (SEQ ID NO: 42); or GGGTGGAGATCAATGGACA (SEQ ID NO: 44) and UGUCCAUUGAUCUCCACCC (SEQ ID NO: 45), or combinations thereof.

In some embodiments, the oligonucleotide includes a sense strand having a sequence with at least 80% (e.g., at least 85%, at least 90%, at least 95%, or at least 99%) sequence identity to a sequence selected from: AGGCACCACCCCTTGGAAC (SEQ ID NO: 22), CCAGCACATACCGGGACCT (SEQ ID NO: 24), GGTGAGGTTACCAACCAGT (SEQ ID NO: 26), CCCGAAAACGAATTGGCTT (SEQ ID NO: 28), TGGCTTTGCTGTCAGCGCT (SEQ ID NO: 30, TACTCTTGGTCACCTCAAA (SEQ ID NO: 32), AATACTCTTGGTCACCTCA (SEQ ID NO: 34), CCAACCAGTCCTTCCGCAT (SEQ ID NO: 36), ATCCGGCGGGAGTGGTATT (SEQ ID NO: 38), CCTCCGGAAGGGTGTGTAT (SEQ ID NO: 40), CAGCACATACCGGGACCTC (SEQ ID NO: 42), or GGGTGGAGATCAATGGACA (SEQ ID NO: 44), or combinations thereof. In some embodiments, the oligonucleotide includes a sense strand having a sequence with at least 80% (e.g., at least 85%, at least 90%, at least 95%, or at least 99%) sequence identity to a sequence selected from: AGGCACCACCCCTTGGAAC (SEQ ID NO: 22), CCAGCACATACCGGGACCT (SEQ ID NO: 24), GGTGAGGTTACCAACCAGT (SEQ ID NO: 26), and TACTCTTGGTCACCTCAAA (SEQ ID NO: 32), or combinations thereof.

In some embodiments, the double strand oligonucleotide structure includes sense and antisense pairs targeting hu-MAPT selected from: #059, #326, #024, #310, #319, #102, #103, #327, #286, #118, #111, and #091, or combinations thereof (or a pair selected from Table 2C, Table 2D, Table 2E, Table 2F, and Table 2G). In some embodiments, the double strand oligonucleotide structure includes sense and antisense pairs selected from: GGGCTGATGGTAAAACGAA (SEQ ID NO: 46) and UUCGUUUUACCAUCAGCCC (SEQ ID NO: 47), CTGGTCTGGCTTGCGGCGC (SEQ ID NO: 48) and GCGCCGCAAGCCAGACCAG (SEQ ID NO: 49, GGAAGATCACGCTGGGACG (SEQ ID NO: 50) and CGUCCCAGCGUGAUCUUCC (SEQ ID NO: 51, AGCTAAGGAGGCCGTTCAG (SEQ ID NO: 52) and CUGAACGGCCUCCUUAGCU (SEQ ID NO: 53), GGATTAGGACTGAAGCGAT (SEQ ID NO: 54) and AUCGCUUCAGUCCUAAUCC (SEQ ID NO: 55), CCGTACTCCACCCAAGTCG (SEQ ID NO: 56) and CGACUUGGGUGGAGUACGG (SEQ ID NO: 57), CGTACTCCACCCAAGTCGC (SEQ ID NO: 58) and GCGACUUGGGUGGAGUACG (SEQ ID NO: 59), CTGGCTTGCGGCGCGAGGA (SEQ ID NO: 60) and UCCUCGCGCCGCAAGCCAG (SEQ ID NO: 61), TTCTAACCCACCCTCACGA (SEQ ID NO: 62) and UCGUGAGGGUGGGUUAGAA (SEQ ID NO: 63), AAGTCCAAGATCGGCTCCA (SEQ ID NO: 64) and UGGAGCCGAUCUUGGACUU (SEQ ID NO: 65), TCGCCGTCTTCCGCCAAGA (SEQ ID NO: 66) and UCUUGGCGGAAGACGGCGA (SEQ ID NO: 67), or GGATCGCAGCGGCTACAGC (SEQ ID NO: 68) and GCUGUAGCCGCUGCGAUCC (SEQ ID NO: 69), or combinations thereof.

In some embodiments, the oligonucleotide includes a sense strand having a sequence with at least 80% (e.g., at least 85%, at least 90%, at least 95%, or at least 99%) sequence identity to a sequence selected from: GGGCTGATGGTAAAACGAA (SEQ ID NO: 46), CTGGTCTGGCTTGCGGCGC (SEQ ID NO: 48), GGAAGATCACGCTGGGACG (SEQ ID NO: 50), AGCTAAGGAGGCCGTTCAG (SEQ ID NO: 52), GGATTAGGACTGAAGCGAT (SEQ ID NO: 54), CCGTACTCCACCCAAGTCG (SEQ ID NO: 56), CGTACTCCACCCAAGTCGC (SEQ ID NO: 58), CTGGCTTGCGGCGCGAGGA (SEQ ID NO: 60), TTCTAACCCACCCTCACGA (SEQ ID NO: 62), AAGTCCAAGATCGGCTCCA (SEQ ID NO: 64), TCGCCGTCTTCCGCCAAGA (SEQ ID NO: 66) or GGATCGCAGCGGCTACAGC (SEQ ID NO: 68), or combinations thereof. In some embodiments, the oligonucleotide includes a sense strand having a sequence with at least 80% (e.g., at least 85%, at least 90%, at least 95%, or at least 99%) sequence identity to a sequence selected from: GGGCTGATGGTAAAACGAA (SEQ ID NO: 46), CTGGTCTGGCTTGCGGCGC (SEQ ID NO: 48), GGAAGATCACGCTGGGACG (SEQ ID NO: 50), AGCTAAGGAGGCCGTTCAG (SEQ ID NO: 52), and CCGTACTCCACCCAAGTCG (SEQ ID NO: 56), or combinations thereof.

In some embodiments, a nanoparticle is formed with two or more compounds, wherein the two or more compounds comprise two or more oligonucleotides where one of the oligonucleotides includes sense and antisense pairs selected from: #081, #001, #091, #131, #135, #177, #175, #093, #056, #009, #002, and #072, or a combination thereof (or a pair selected from Table 2C, Table 2D, Table 2E, Table 2F, and Table 2G) and one of the oligonucleotides includes #059, #326, #024, #310, #319, #102, #103, #327, #286, #118, #111, and #091, or a combination thereof (or a pair selected from Table 2C, Table 2D, Table 2E, Table 2F, and Table 2G), or a combination thereof.

In some embodiments, a nanoparticle is formed with two or more compounds, wherein the two or more compounds comprise two or more oligonucleotides where one of the oligonucleotides includes sense and antisense pairs selected from: AGGCACCACCCCTTGGAAC (SEQ ID NO: 22) and GUUCCAAGGGGUGGUGCCU (SEQ ID NO: 23), CCAGCACATACCGGGACCT (SEQ ID NO: 24) and AGGUCCCGGUAUGUGCUGG (SEQ ID NO: 25), GGTGAGGTTACCAACCAGT (SEQ ID NO: 26) and ACUGGUUGGUAACCUCACC (SEQ ID NO: 27), CCCGAAAACGAATTGGCTT (SEQ ID NO: 28) and AAGCCAAUUCGUUUUCGGG (SEQ ID NO: 29), TGGCTTTGCTGTCAGCGCT (SEQ ID NO: 30) and AGCGCUGACAGCAAAGCCA (SEQ ID NO: 31), TACTCTTGGTCACCTCAAA (SEQ ID NO: 32) and UUUGAGGUGACCAAGAGUA (SEQ ID NO: 33), AATACTCTTGGTCACCTCA (SEQ ID NO: 34) and UGAGGUGACCAAGAGUAUU (SEQ ID NO: 35), CCAACCAGTCCTTCCGCAT (SEQ ID NO: 36) and AUGCGGAAGGACUGGUUGG (SEQ ID NO: 37), ATCCGGCGGGAGTGGTATT (SEQ ID NO: 38) and AAUACCACUCCCGCCGGAU (SEQ ID NO: 39), CCTCCGGAAGGGTGTGTAT (SEQ ID NO: 40) and CAGCACATACCGGGACCTC (SEQ ID NO: 42); or GGGTGGAGATCAATGGACA (SEQ ID NO: 44) and UGUCCAUUGAUCUCCACCC (SEQ ID NO: 45), or combinations thereof, and one of the oligonucleotides includes sense and antisense pairs selected from: GGGCTGATGGTAAAACGAA (SEQ ID NO: 46) and UUCGUUUUACCAUCAGCCC (SEQ ID NO: 47), CTGGTCTGGCTTGCGGCGC (SEQ ID NO: 48) and GCGCCGCAAGCCAGACCAG (SEQ ID NO: 49), GGAAGATCACGCTGGGACG (SEQ ID NO: 50) and CGUCCCAGCGUGAUCUUCC (SEQ ID NO: 51), AGCTAAGGAGGCCGTTCAG (SEQ ID NO: 52) and CUGAACGGCCUCCUUAGCU (SEQ ID NO: 53, GGATTAGGACTGAAGCGAT (SEQ ID NO: 54) and AUCGCUUCAGUCCUAAUCC (SEQ ID NO: 55), CCGTACTCCACCCAAGTCG (SEQ ID NO: 56) and CGACUUGGGUGGAGUACGG (SEQ ID NO: 57), CGTACTCCACCCAAGTCGC (SEQ ID NO: 58) and GCGACUUGGGUGGAGUACG (SEQ ID NO: 59), CTGGCTTGCGGCGCGAGGA (SEQ ID NO: 60) and UCCUCGCGCCGCAAGCCAG (SEQ ID NO: 61), TTCTAACCCACCCTCACGA (SEQ ID NO: 62) and UCGUGAGGGUGGGUUAGAA (SEQ ID NO: 63), AAGTCCAAGATCGGCTCCA (SEQ ID NO: 64) and UGGAGCCGAUCUUGGACUU (SEQ ID NO: 65, TCGCCGTCTTCCGCCAAGA (SEQ ID NO: 66) and UCUUGGCGGAAGACGGCGA (SEQ ID NO: 67), or GGATCGCAGCGGCTACAGC (SEQ ID NO: 68) and GCUGUAGCCGCUGCGAUCC (SEQ ID NO: 69), or combinations thereof.

In some embodiments, a nanoparticle is formed with two or more compounds, wherein the two or more compounds comprise two or more oligonucleotides where one of the oligonucleotides includes a sense strand having a sequence with at least 80% (e.g., at least 85%, at least 90%, at least 95%, or at least 99%) sequence identity to a sequence selected from: AGGCACCACCCCTTGGAAC (SEQ ID NO: 22), CCAGCACATACCGGGACCT (SEQ ID NO: 24), GGTGAGGTTACCAACCAGT (SEQ ID NO: 26), CCCGAAAACGAATTGGCTT (SEQ ID NO: 28), TGGCTTTGCTGTCAGCGCT (SEQ ID NO: 30), TACTCTTGGTCACCTCAAA (SEQ ID NO: 32), AATACTCTTGGTCACCTCA (SEQ ID NO: 34), CCAACCAGTCCTTCCGCAT (SEQ ID NO: 36), ATCCGGCGGGAGTGGTATT (SEQ ID NO: 38), CCTCCGGAAGGGTGTGTAT (SEQ ID NO: 40), CAGCACATACCGGGACCTC (SEQ ID NO: 42) and GGGTGGAGATCAATGGACA (SEQ ID NO: 44), or combinations thereof, and one of the oligonucleotides includes a sense strand having a sequence with at least 80% (e.g., at least 85%, at least 90%, at least 95%, or at least 99%) sequence identity to a sequence selected from: GGGCTGATGGTAAAACGAA (SEQ ID NO: 46), CTGGTCTGGCTTGCGGCGC (SEQ ID NO: 48), GGAAGATCACGCTGGGACG (SEQ ID NO: 50), AGCTAAGGAGGCCGTTCAG (SEQ ID NO: 52), GGATTAGGACTGAAGCGAT (SEQ ID NO: 54), CCGTACTCCACCCAAGTCG (SEQ ID NO: 56), CGTACTCCACCCAAGTCGC (SEQ ID NO: 58), CTGGCTTGCGGCGCGAGGA (SEQ ID NO: 60), TTCTAACCCACCCTCACGA (SEQ ID NO: 62), AAGTCCAAGATCGGCTCCA (SEQ ID NO: 64), TCGCCGTCTTCCGCCAAGA (SEQ ID NO: 66) or GGATCGCAGCGGCTACAGC (SEQ ID NO: 68), or combinations thereof.

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to AGGCACCACCCCTTGGAAC (SEQ ID NO: 22) (sense strand of hu-BACE1 pair #81) and an antisense strand having a sequence with at least 80% (e.g., at least 85%, at least 90%, at least 95%, or at least 99%) sequence identity to GUUCCAAGGGGUGGUGCCU (SEQ ID NO: 23) (antisense strand of hu-BACE1 pair #81).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to AGGCACCACCCCUUGGAAC (SEQ ID NO: 136) (sense strand of hu-BACE1 pair #81) and an antisense strand having a sequence with at least 80% (e.g., at least 85%, at least 90%, at least 95%, or at least 99%) sequence identity to GUUCCAAGGGGUGGUGCCU (SEQ ID NO: 23) (antisense strand of hu-BACE1 pair #81).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to AGGCACCACCCCTTGGAAC (SEQ ID NO: 22) (sense strand of hu-BACE1 pair #81) and an antisense strand having a sequence with at least 80% (e.g., at least 85%, at least 90%, at least 95%, or at least 99%) sequence identity to GTTCCAAGGGGTGGTGCCT (SEQ ID NO: 137) (antisense strand of hu-BACE1 pair #81).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to AGGCACCACCCCUUGGAAC (SEQ ID NO: 136) (sense strand of hu-BACE1 pair #81) and an antisense strand having a sequence with at least 80% (e.g., at least 85%, at least 90%, at least 95%, or at least 99%) sequence identity to GTTCCAAGGGGTGGTGCCT (SEQ ID NO: 137) (antisense strand of hu-BACE1 pair #81).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to GGAAGATCACGCTGGGACG (SEQ ID NO: 50) (sense strand of hu-MAPT pair #24) and an antisense strand having a sequence with at least 80% (e.g., at least 85%, at least 90%, at least 95%, or at least 99%) sequence identity to CGUCCCAGCGUGAUCUUCC (SEQ ID NO: 51) (antisense strand of hu-MAPT pair #24).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to GGAAGAUCACGCUGGGACG (SEQ ID NO: 164) (sense strand of hu-MAPT pair #24) and an antisense strand having a sequence with at least 80% (e.g., at least 85%, at least 90%, at least 95%, or at least 99%) sequence identity to CGTCCCAGCGTGATCTTCC (SEQ ID NO: 165) (antisense strand of hu-MAPT pair #24).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to GGAAGAUCACGCUGGGACG (SEQ ID NO: 164) (sense strand of hu-MAPT pair #24) and an antisense strand having a sequence with at least 80% (e.g., at least 85%, at least 90%, at least 95%, or at least 99%) sequence identity to CGUCCCAGCGUGAUCUUCC (SEQ ID NO: 51) (antisense strand of hu-MAPT pair #24).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to GGAAGATCACGCTGGGACG (SEQ ID NO: 50) (sense strand of hu-MAPT pair #24) and an antisense strand having a sequence with at least 80% (e.g., at least 85%, at least 90%, at least 95%, or at least 99%) sequence identity to CGTCCCAGCGTGATCTTCC (SEQ ID NO: 165) (antisense strand of hu-MAPT pair #24).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to AGGCACCACCCCTTGGAAC (SEQ ID NO: 22) (sense strand of BACE1 pair #81) and a second oligonucleotide includes an antisense strand having a sequence with at least 80% (e.g., at least 85%, at least 90%, at least 95%, or at least 99%) sequence identity GGAAGATCACGCTGGGACG (SEQ ID NO: 50) (sense strand for MAPT #24).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to AGGCACCACCCCUUGGAAC (SEQ ID NO: 136) (sense strand of BACE1 pair #81) and a second oligonucleotide includes an antisense strand having a sequence with at least 80% (e.g., at least 85%, at least 90%, at least 95%, or at least 99%) sequence identity GGAAGATCACGCTGGGACG (SEQ ID NO: 50) (sense strand for MAPT #24).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to AGGCACCACCCCUUGGAAC (SEQ ID NO: 136) (sense strand of BACE1 pair #81) and a second oligonucleotide includes an antisense strand having a sequence with at least 80% (e.g., at least 85%, at least 90%, at least 95%, or at least 99%) sequence identity GGAAGAUCACGCUGGGACG (SEQ ID NO: 164) (sense strand for MAPT #24).

In some embodiments, a nanoparticle is formed with one or more compounds, wherein the one or more compounds include one or more double stranded oligonucleotides where a first oligonucleotide includes a sense strand having a sequence with at least 80% (e.g., at least 85%, at least 90%, at least 95%, or at least 99%) sequence identity to AGGCACCACCCCTTGGAAC (SEQ ID NO: 22) (sense strand of BACE1 pair #81) and a second oligonucleotide includes an antisense strand having a sequence with at least 80% (e.g., at least 85%, at least 90%, at least 95%, or at least 99%) sequence identity GGAAGAUCACGCUGGGACG (SEQ ID NO: 164) (sense strand for MAPT #24).

In some embodiments, the double strand oligonucleotide structure includes sense and antisense pairs selected from: #035, #024, #032, #034, #006, #003, #028, #030, #029, #022, #023, #002, #004, #017, #015, #033, #027, #026, and #025, or a combination thereof (or a pair selected from Table 2C, Table 2D, Table 2E, Table 2F, and Table 2G). In some embodiments, two or more compounds form a particle and they comprise two or more oligonucleotides where one of the oligonucleotides includes sense and antisense pairs selected from: AACGTCACTGTGCGTGCCA (SEQ ID NO: 70) and UGGCACGCACAGUGACGUU (SEQ ID NO: 71), CGGGAGACCGACGAGGAAT (SEQ ID NO: 72) and AUUCCUCGUCGGUCUCCCG (SEQ ID NO: 73), GGAGGAGCCTGGCCGGAGA (SEQ ID NO: 74) and UCUCCGGCCAGGCUCCUCC (SEQ ID NO: 75), TCCGGCTGCCCCTTCGCAG (SEQ ID NO: 76) and CUGCGAAGGGGCAGCCGGA (SEQ ID NO: 77), GGAGCCCGGAGTCCCTGTC (SEQ ID NO: 78) and GACAGGGACUCCGGGCUCC (SEQ ID NO: 79), TGCCTAGGTGCTGGGAGCT (SEQ ID NO: 80) and AGCUCCCAGCACCUAGGCA) (SEQ ID NO: 81), CGGGCAGTAGTAACTTTGC (SEQ ID NO: 82) and GCAAAGUUACUACUGCCCG (SEQ ID NO: 83), GCTGTCCAGCACATATCGA (SEQ ID NO: 84) and UCGAUAUGUGCUGGACAGC (SEQ ID NO: 85), ACCCTTTCCTGCATCGCTA (SEQ ID NO: 86) and UAGCGAUGCAGGAAAGGGU (SEQ ID NO: 87), AGGGAACCCATCTCGGCAT (SEQ ID NO: 88) and AUGCCGAGAUGGGUUCCCU (SEQ ID NO: 89), GCATCCCTCATGGCCCCAA (SEQ ID NO: 90) and UUGGGGCCAUGAGGGAUGC (SEQ ID NO: 91), ACAAGCCTTTCCGCCTCCC (SEQ ID NO: 92) and GGGAGGCGGAAAGGCUUGU (SEQ ID NO: 93), CTGGATTATGGTGGCCTGA (SEQ ID NO: 94) and UCAGGCCACCAUAAUCCAG (SEQ ID NO: 95), CAGTCCTGATGCCGCCGAG (SEQ ID NO: 96) and CUCGGCGGCAUCAGGACUG (SEQ ID NO: 97), CGCCGCCTGGACCGGGAAC (SEQ ID NO: 98) and GUUCCCGGUCCAGGCGGCG (SEQ ID NO: 99), GGGCACCGACCTGGTGAGC (SEQ ID NO: 100) and GCUCACCAGGUCGGUGCCC (SEQ ID NO: 101, TATGTGGAGATGACCGTAG (SEQ ID NO: 102) and CUACGGUCAUCUCCACAUA (SEQ ID NO: 103), GAGGGGAAAGTCCGGCCAG (SEQ ID NO: 104) and CUGGCCGGACUUUCCCCUC (SEQ ID NO: 105), or CCCTACACCCAGGGCAAGT (SEQ ID NO: 106) and ACUUGCCCUGGGUGUAGGG (SEQ ID NO: 107), or combinations thereof.

In some embodiments, the oligonucleotide includes a sense strand having a sequence with at least 80% (e.g., at least 85%, at least 90%, at least 95%, or at least 99%) sequence identity to a sequence selected from SEQ ID NO: 70, 72, 74, 76, 78, 80, 82, 84, 86, 88, 90, 92, 94, 96, 98, 100, 102, 104, and 106. In some embodiments, the oligonucleotide includes a sense strand having a sequence with at least 80% (e.g., at least 85%, at least 90%, at least 95%, or at least 99%) sequence identity to a sequence selected from: AACGTCACTGTGCGTGCCA (SEQ ID NO: 70), CGGGAGACCGACGAGGAAT (SEQ ID NO: 72), GGAGGAGCCTGGCCGGAGA SEQ ID NO: 74), TCCGGCTGCCCCTTCGCAG (SEQ ID NO: 76), and GGAGCCCGGAGTCCCTGTC (SEQ ID NO: 78), or combinations thereof.

In some embodiments, the double strand oligonucleotide structure includes sense and antisense pairs selected from: #038, #089, #001, #101, #096, #102, #121, #103, #011, #097, #008, and #095, or a combination thereof (or a pair selected from Table 2C, Table 2D, Table 2E, Table 2F, and Table 2G). In some embodiments, a compound comprises two or more oligonucleotides where one of the oligonucleotides includes sense and antisense pairs selected from: TGGGACTTTAGGGCTAACC (SEQ ID NO: 108) and GGUUAGCCCUAAAGUCCCA (SEQ ID NO: 109), TGGGGTGATCAGCCCAAAA (SEQ ID NO: 110) and UUUUGGGCUGAUCACCCCA (SEQ ID NO: 111), CGCCGGCCTCCAGAACGCG (SEQ ID NO: 112) and CGCGUUCUGGAGGCCGGCG (SEQ ID NO: 113), ATTGCTTGCGTTGTGGGGG (SEQ ID NO: 114) and CCCCCACAACGCAAGCAAU (SEQ ID NO: 115), GGTGGGCTAGATAGATAGG (SEQ ID NO: 116) and CCUAUCUAUCUAGCCCACC (SEQ ID NO: 117), GGAGGAGGAACACATTAAG (SEQ ID NO: 118) and CUUAAUGUGUUCCUCCUCC (SEQ ID NO: 119), GGGCATGATCTCCAGTGCT (SEQ ID NO: 120) and AGCACUGGAGAUCAUGCCC (SEQ ID NO: 121), AAAGGGAGGTCTTGGAGTG (SEQ ID NO: 122) and CACUCCAAGACCUCCCUUU (SEQ ID NO: 123), GGCAAAACCGGGGCGAAGA (SEQ ID NO: 124) and UCUUCGCCCCGGUUUUGCC (SEQ ID NO: 125), TACAATTGGCCTGGTTCCT (SEQ ID NO: 126) and AGGAACCAGGCCAAUUGUA (SEQ ID NO: 127), GCTCGTGTGGCCAGCAAAG (SEQ ID NO: 128) and CUUUGCUGGCCACACGAGC (SEQ ID NO: 129), or GAGCCAATGTCTTTGGGTG (SEQ ID NO: 130) and CACCCAAAGACAUUGGCUC (SEQ ID NO: 131).

In some embodiments, the oligonucleotide includes a sense strand having a sequence with at least 80% (e.g., at least 85%, at least 90%, at least 95%, or at least 99%) sequence identity to a sequence selected from: 108, 110, 112, 114, 116, 118, 120, 122, 124, 126, 128, and 130. In some embodiments, the oligonucleotide includes a sense strand having a sequence with at least 80% (e.g., at least 85%, at least 90%, at least 95%, or at least 99%) sequence identity to a sequence selected from: TGGGACTTTAGGGCTAACC (SEQ ID NO: 108), TGGGGTGATCAGCCCAAAA (SEQ ID NO: 110), CGCCGGCCTCCAGAACGCG (SEQ ID NO: 112), ATTGCTTGCGTTGTGGGGG (SEQ ID NO: 114), and GGTGGGCTAGATAGATAGG (SEQ ID NO: 116).

In some embodiments, the double strand oligonucleotide includes an oligonucleotide having a sense strand selected from Table 2C. In some embodiments, the double strand oligonucleotide includes an oligonucleotide having an antisense sense strand selected from Table 2C.

In some embodiments, the double strand oligonucleotide includes an oligonucleotide having a sense strand selected from Table 2D. In some embodiments, the double strand oligonucleotide includes an oligonucleotide having an antisense sense strand selected from Table 2D.

In some embodiments, the double strand oligonucleotide includes an oligonucleotide having a sense strand selected from Table 2E. In some embodiments, the double strand oligonucleotide includes an oligonucleotide having an antisense sense strand selected from Table 2E.

In some embodiments, the double strand oligonucleotide includes an oligonucleotide having a sense strand selected from Table 2F. In some embodiments, the double strand oligonucleotide includes an oligonucleotide having an antisense sense strand selected from Table 2F.

In some embodiments, the double strand oligonucleotide includes an oligonucleotide having a sense strand selected from Table 2G. In some embodiments, the double strand oligonucleotide includes an oligonucleotide having an antisense sense strand selected from Table 2G.

### Double Strand Oligonucleotide Structure

In an aspect, provided herein is a double strand oligonucleotide structure comprising the structure of Formula 1 below:

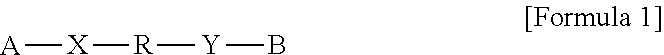

In Formula 1, A is a hydrophilic moiety, B is a hydrophobic moiety, and X and Y each independently represent a simple covalent bond or linked-mediated covalent bond, and R refers to a double strand oligonucleotide comprising a sense strand and an antisense strand comprising a sequence complementary thereto, and a monosaccharide is bonded to the A or B terminal.

The monosaccharide may be, for example, glucose, fructose, or galactose, but may specifically be glucose.

The monosaccharide may specifically be bonded to the A terminal of hydrophilic moiety A. For example, the monosaccharide may be glucose, with the glucose bonded to the A terminal of hydrophilic moiety A.

Accordingly, the present disclosure may be structure comprising a ligand, for example a monosaccharide, for improving BBB permeability of the structures of Formula 1 through Formula 3′, Formula 6 and Formula 7 illustrated below, and may have the structure of Formula 14.

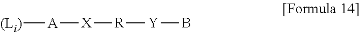

In Formula 14 above, A, B, X and Y are as defined in Formula 1 above, L is a monosaccharide, i is an integer of 1 through 10, specifically, an integer of 2 through 8, and more specifically 6.

The A and Lᵢ may be joined by a linker. The linker may, for example, have the Formula of NH(CH₂)ₙCONH—CH₂—CH(OH)—CH₂—.

Specifically, the double strand oligonucleotide structure according to the present disclosure may be an oligonucleotide structure comprising a hydrophilic and hydrophobic group connected by a linker to the number 6 carbon of glucose, and may be self-assembled.

The oligonucleotide structure is capable of self-assembly, and “self-assembly” means that the structure may self-assemble into groups having an organized structure or pattern exhibiting a particular functionality.

As used herein, “double strand oligonucleotide” refers to a nucleic acid molecule which is able to mediate RNA inhibition or gene silencing through “siRNA”, and is thereby able to specifically inhibit the expression and/or activity of a target gene.

As used herein, “specificity” refers to the ability to specifically inhibit a target gene only, without impacting other genes in the cell.

In some embodiments, the double strand oligonucleotide structure comprises a sense strand and an anti-sense strand comprising a sequence complementary thereto.

In some embodiments, the sense strand or anti-sense strand may be characterized in that it is comprised of 19 to 31 nucleotides, but is not limited hereto. In some cases, the sense strand or antisense strand may additionally comprise 19 to 31 nucleotides, but is not limited hereto.

In some embodiments, the sense or anti-sense strand may be characterized in that they are independently DNA or RNA, and may, for example, comprise RNA/RNA, DNA/DNA, or DNA/RNA hybrid sequences.

In some embodiments, the sense or anti-sense strand may be characterized in that it comprises chemical modifications, and the chemical modifications may be at least one of, but are not limited to:
Modifications wherein a hydroxy group (—OH) at the 2′ carbon position of a sugar structure in the nucleotide is substituted by any one selected from the group comprised of a methyl group (—CH₃), a methoxy group (—OCH₃), an amine group (—NH₂), a fluorine (—F), an O-2-methoxyethyl group, an O-propyl group, an O-2-methylthioethyl group, an O-3-aminopropyl group, an O-3-dimethylaminopropyl group, an O—N-methylacetamino group and an O-dimethylamidooxyethyl group;
Modifications wherein the oxygen of a sugar structure in the nucleotide is substituted by sulfur;
Modifications wherein the nucleotide bond becomes any one selected from the group comprised of a phosphorothioate bond, a boranophosphate bond and a methyl phosphonate bond; and,
Modifications to a PNA (peptide nucleic acid), LNA (locked nucleic acid) or UNA (unlocked nucleic acid) form.

In some embodiments, the chemical modification may be for improving in vivo stability, or may contribute to imparting nuclease resistance and reducing non-specific immune response.

In some embodiments, the double strand oligonucleotide structure be characterized in that at least one phosphate group is bonded to the 5′ terminal of the anti-sense strand, and preferably characterized in that 1 to 3 phosphate groups are bonded, but the present disclosure is not limited hereto.

In some embodiments, the double strand oligonucleotide as provided herein is a concept encompassing all substances having RNAi activity, and that the target gene-specific double strand oligonucleotide includes target gene-specific shRNAs, etc. is self-evident to a person skilled in the art. That is, the oligonucleotide may be characterized in that it is siRNA, shRNA or miRNA.

In some embodiments, the double strand oligonucleotide according to the present disclosure may include, at the 3′ terminal of one or both strands, an overhang which is a structure comprising one or more unpaired nucleotides.

In some embodiments, the form of the double strand oligonucleotide according to the present disclosure is preferably DNA-RNA hybrid, siRNA (short interfering RNA), shRNA (short hairpin RNA) and miRNA (microRNA), etc., but the present disclosure is not limited hereto, and includes single strand miRNA inhibitors able to function as antagonists to miRNA.

In some embodiments, further, in the Formula 14 above, the hydrophilic moiety A may be a hydrophilic block according to Formula 4 and Formula 5 below.

In some embodiments, the double strand oligonucleotide structure according to the present disclosure may comprise the structure of Formula 2 below:

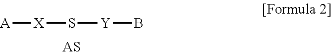

In some embodiments, in Formula 2, S and AS refer to the sense strand and anti-sense strand of the double strand oligonucleotide, and the definitions of A, B, X and Y are identical to those of Formula 1.

In some embodiments, more preferably, the pharmaceutical composition according to the present disclosure comprises the structure of Formula 3 below:

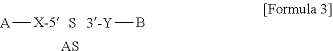

In some embodiments, alternatively, the double strand oligonucleotide according to the present disclosure comprises the structure of Formula 3′ below:

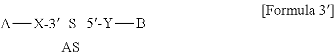

In some embodiments, in Formula 3 and Formula 3′ above, the definitions of A, B, X, Y, S and AS are identical to those of Formula 2, and 5′ and 3′ refer to the 5′ and 3′ terminals of the sense strand.

In some embodiments, the molecular weight of the hydrophilic moiety may be 200 to 10,000, but is not limited hereto.

In some embodiments, the hydrophilic moiety may be selected from the group comprised of polyethylene glycol (PEG), polyvinyl pyrrolidone and polyoxazoline, but is not limited hereto.

In some embodiments, the hydrophilic moiety may be characterized in that it has the structure of Formula 4 or Formula 5 below:

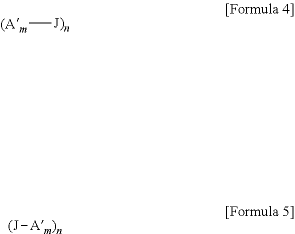

In some embodiments, in Formula 4 above, A′ refers to a hydrophilic moiety monomer, J refers to a linker connecting a number m of hydrophilic monomers to each other or a number m of hydrophilic monomer to siRNAs, m is an integer of 1 to 15, and n is an integer of 1 to 10, where the hydrophilic monomer A′ is any one compound selected from among Compound 1 through Compound 3 below, and the linker (J) is selected from the group comprised of PO₃⁻, SO₃ and CO₂;

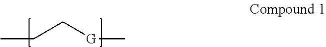

In some embodiments, in Compound 1 above, G is selected from the group comprised of CH₂, O, S and NH;

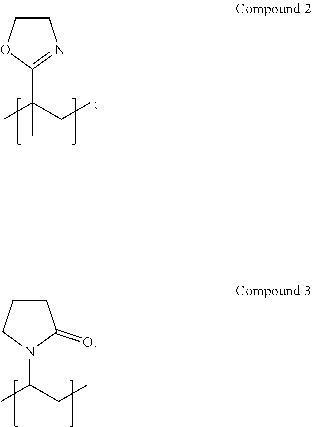

In some embodiments, in a case where there is a hydrophilic block as in Formula 4 or Formula 5 above, the double strand oligonucleotide structure according to the present disclosure may have the structure of Formula 6 or Formula 7 below.

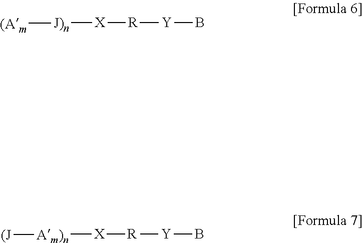

In some embodiments, in Formula 6 and Formula 7 above, the definitions of X, R, Y and B are identical to those of Formula 1, and the definitions of A′, J, m and n are identical to those of Formula 4 and Formula 5.

In some embodiments, in Formula 4 and Formula 5 above, the hydrophilic monomer (A′) may be any monomer of a nonionic hydrophilic polymer which suits the object of the present disclosure, and may preferably be a monomer selected from among Compound 1 through Compound 3 stated in Table 1, more preferably the monomer of Compound 1, and even more preferably the G of Compound 1 may be selected from among O, S and NH.

In some embodiments, in particular, among hydrophilic monomers, the monomer represented by Compound 1 is particularly suitable for the preparation of the structure according to the present disclosure as various functional groups may be introduced, as it has excellent bio-compatibility with good bioaffinity and low induction of immune response, improves the in vivo stability of the double strand oligonucleotide included in the structures according to Formula 6 or Formula 7, and is able to improve delivery efficiency.

TABLE 9
Hydrophilic monomer structures in the present disclosure

| Compound 1 | Compound 2 | Compound 3 |
|---|---|---|
| 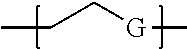 G is O, S or NH | 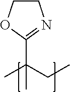 | 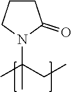 |

In some embodiments, preferably, in Formula 4 through Formula 7 above, the total molecular weight of the hydrophilic moiety is in the range of 1,000 to 2,000. Accordingly, for example, in a case using a substance where hexaethylene glycol, that is, G, according to Compound 1 in Formula 6 and Formula 7 is O, and m is 6, the molecular weight of the hexaethylene glycol is 344, therefor the number of repetitions is preferably 3 to 5. In particular, the present disclosure is characterized in that the number of repetitions of the hydrophilic group represented by (A′m-J), or (J-A′ m) n in Formula 4 or Formula 5, that is, the hydrophilic block, may be an appropriate number represented by n. The hydrophilic monomer A and linker J included in the respective hydrophilic blocks may be identical or different between the respective hydrophilic blocks. That is, in a case where 3 hydrophilic blocks are used (n=3), different hydrophilic monomers may be used in all hydrophilic blocks by using a hydrophilic monomer according to Compound 1 in the first block, a hydrophilic monomer according to Compound 2 in the second block, and a hydrophilic monomer according to Compound 3 in the third block, or, the same hydrophilic monomer selected from among the hydrophilic monomers of Compound 1 through Compound 3 may be used in all of the hydrophilic blocks. Likewise, for the linkers mediating the bonding of the hydrophilic monomers, the same linker may be used across all hydrophilic blocks, or different linkers may be used for each hydrophilic block. Further, the number m of hydrophilic monomers may be the same across all hydrophilic blocks, or may be different. That is, different numbers of hydrophilic monomers may be used, with 3 hydrophilic monomers connected (m=3) in the first hydrophilic block, 5 hydrophilic monomers connected (m=5) in the second hydrophilic block, and 4 hydrophilic monomers connected (m=4) in the third hydrophilic block, or, the same number of hydrophilic monomers may be used across all of the hydrophilic blocks.

In some embodiments, further, where in the present disclosure, the linker (J) is preferably selected from the group comprised of —PO₃⁻—, —SO₃— and —CO₂—, the present disclosure is not limited hereto, and it shall be self-evident to a person skilled in that art that any linker which suits the object of the present disclosure may be used depending on the hydrophilic monomer used.

In some embodiments, in Formula 1-Formula 3, Formula 6 and Formula 7 above, the hydrophobic moiety (B) plays the role of forming a nanoparticle comprised of a double strand oligonucleotide structure through a hydrophobic interaction. Preferably, the molecular weight of the hydrophobic moiety is 250 to 1,000, and may be, but is not limited to, any one selected from the group comprised of steroid derivatives, glyceride derivatives, glycerol ether, poplypropylene glycol, unsaturated or saturated hydrocarbon of C₁₂ through C₅₀, diacylphosphatidylcholine, fatty acid, phospholipid, lipopolyamine, lipid, tocopherol and tocotrienol, and it shall be self-evident to a person skilled in the art that any hydrophobic moiety suitable for the object of the present disclosure may be used.

In some embodiments, the steroid derivative may be selected from the group comprised of cholesterol, cholestanol, cholic acid, cholesteryl formate, cotestanyl formate, and colistanylamine, and the glyceride derivative may be mono-, di- and tri-glyceride, wherein the fatty acid of glyceride is preferably an unsaturated or saturated fatty acids of C₁₂ to C₅₀.

In some embodiments, in particular, among the hydrophobic moieties, saturated or unsaturated hydrocarbons or cholesterol are preferable in that they have the advantage of being easily combined in the synthesis step of the oligonucleotide structure according to the present disclosure, and most preferable is a C₂₄ hydrocarbon, particular a form comprising a disulfide bond. In some embodiments, the hydrophobic moiety comprises C₂₄ (C₆—S—S—C₁₈).

In some embodiments, the hydrophobic material is bound to the distal end of the hydrophilic material, and may be attached to any position of the sense strand or antisense strand of the miRNA.

In some embodiments, the hydrophilic moiety of Formula 1 to Formula 3′, Formula 6, Formula 7 and Formula 14, the hydrophilic moiety block or the hydrophobic moiety and the double strand oligonucleotide are joined by a simple covalent bond or a linker-mediated covalent bond (X or Y). There is no particular restriction on the linker insofar as it bonds covalently between the hydrophilic moiety or hydrophobic moiety and the terminal of the double strand oligonucleotide, and provides a bond which may be degraded under certain environments as needed. Therefore, as the linker, any compound which bonds to activate the double strand oligonucleotide and/or the hydrophilic moiety (or hydrophobic moiety) in the process of preparing the double strand oligonucleotide structure according to the present disclosure may be used. The covalent bond may be either a non-degradable bond or a degradable bond. Here, the non-degradable bond includes an amide bond or a phosphorylated bond, and the degradable bond includes a disulfide bond, an acid-degradable bond, an ester bond, an anhydride bond, a biodegradable bond, or an enzyme degradable bond, but is not limited hereto.

In some embodiments, in the structure comprising a double strand oligonucleotide according to the present disclosure, an additional amine or polyhistidine group may be introduced at the terminal opposite the terminal bonded to the hydrophilic moiety in the structure.

In some embodiments, this is to facilitate the introduction of the carrier of the structure comprising a double strand oligonucleotide according to the present disclosure into a cell and its endosomal escape. That introduction of an amine group and polyhistidine groups may be used to facilitate the introduction of carriers such as quantum dot, dendrimer and liposome into cells and their endosomal escape, and their benefits, have been reported.

In some embodiments, specifically, the primary amine group modified at the end or outside of the carrier forms a conjugate by electrostatic interaction with a negatively charged gene while being protonated at in vivo pH. It is known that the carrier can be protected from degradation of lysosomes as endosomal escape is facilitated due to an internal tertiary amine having a buffering effect at the low pH of endosomes after introduction into cells. (Inhibition of gene transfer and expression using a polymer-based hybrid material. Polymer Sci. Technol., Vol. 23, No. 3, pp. 254-259).

In some embodiments, as histidine, a non-essential amino acid, has an imidazole ring (pK_{R} 6.04) at the residue (—R), it has the effect of increasing buffering capacity at endosomes and lysosomes. It is well known that a histidine modification may be used to improve endosomal escape efficiency in non-viral gene carriers such as liposomes (Novel histidine-conjugated galactosylated cationic liposomes for efficient hepatocyte selective gene transfer in human hepatoma HepG2 cells. J. Controlled Release 118, pp. 262-270).

In some embodiments, the amine group or polyhistidine group may be connected to the hydrophilic moiety or the hydrophilic block through one or more linkers.

In some embodiments, in a case where an amine group or polyhistidine group is introduced into the hydrophilic moiety of the double strand oligonucleotide structure according to Formula 1 of the present disclosure, the structure of Formula 8 may be had.

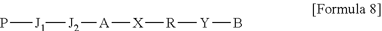

In some embodiments, in Formula 8, the definitions for A, B, R, X and Y are identical to those for Formula 1.

In some embodiments, P is an amine group or polyhistidine group, and J₁ and J₂ are linkers, where J₁ and J₂ may independently be selected from among a simple covalent bond, O₃⁻, SO₃, CO₂, C₂₋₁₂ alkyl, alkenyl and alkynyl, but are not limited hereto. It shall be self-evident to a person skilled in the art that J₁ and J₂ may be any linker suitable for the object of the present disclosure, depending on the type of hydrophilic moiety used.

In some embodiments, preferably, in a case where an amine group has been introduced, J₂ is a simple covalent bond or PO₃⁻, and J₁ is preferably a C₆ alkyl, but these are not limited hereto.

In some embodiments, further, in a case where a polyhistidine group has been introduced, J₂ is preferably a simple covalent bond or PO₃ and J₁ is preferably Compound 4, but these are not limited hereto.

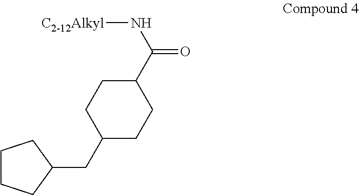

In some embodiments, further, in a case where the hydrophilic moiety of the double strand oligonucleotide according to Formula 8 is the hydrophilic block according to Formula 5 or Formula 6, and an amine group or polyhistidine group is introduced into the same, the structure of Formula 9 or Formula 10 may be had.

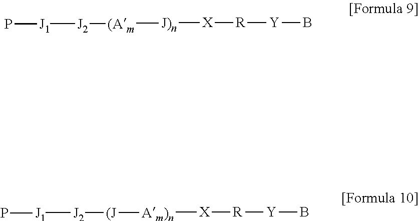

In some embodiments, in Formula 9 and Formula 10 above, the definitions of X, R, Y, B, A′, J, m and n are identical to those of Formula 4 and Formula 5, while the definitions of P, J₁ and J₂ are identical to those of Formula 8.

In some embodiments, in particular, in Formula 9 and Formula 10 above, the hydrophilic moiety is preferably bonded to the 3′ terminal of the sense strand of the double strand oligonucleotide, in which case Formula 8 through Formula 10 above may have the form of Formula 11 through Formula 13 below.

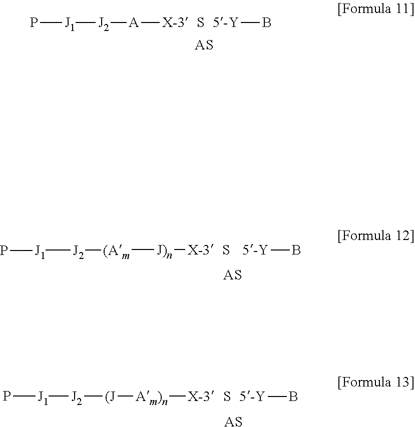

In some embodiments, in Formula 11-Formula 13 above, the definitions of X, R, Y, B, A, A′, J, m, n, P, J₁ and J₂ are identical to those of Formula 8 through Formula 10 above, and 5′ and 3′ represent the 5′ terminal and 3′ terminal of the sense strand of a target gene-specific double strand oligonucleotide.

In some embodiments, as the amine group which may be introduced into the present disclosure, a primary to tertiary amine may be used, but use of a primary amine is particularly preferable. The introduced amine group may exist in the form of an amine salt. For example, the salt of the primary amine may exist in the form of NH₃⁺.

In some embodiments, in addition, the polyhistidine group that can be introduced in the present disclosure preferably includes 3 to 10 histidines, more preferably 5 to 8, and most preferably 6 histidines. Additionally, one or more cysteines may be included in addition to histidine.

In some embodiments, meanwhile, if the double strand oligonucleotide structure comprising the target gene-specific double strand oligonucleotide according to the present disclosure and the nanoparticles formed therefrom are provided with a targeting moiety, this can efficiently promote delivery to the target cell, thereby delivering to a target cell and exhibiting strong target gene expression control effect even at relatively low dose administration.

In another aspect, provided herein is a nanoparticle comprising a double strand oligonucleotide structure. In the present disclosure, the double strand oligonucleotide structure may be characterized in that it forms self-assembled nanoparticles of 10 to 100 nm in size and having neutral charge in an aqueous solution for administration.

In some embodiments, the nanoparticle may be characterized in that is comprised of a mix of double helix oligonucleotide structures comprising different sequences. In some embodiments, the nanoparticle comprises a mix of double helix oligonucleotide structures comprising different sequences specific to different target molecules (e.g., different mRNA transcripts). In some embodiments, the nanoparticle comprises a mix of double helix oligonucleotide structures comprising different sequences specific different sites on a single target molecule (e.g., different target sites on a single mRNA transcript).

In some embodiments, the nanoparticle comprises a mix of double helix oligonucleotide structures comprising two different sequences. In some embodiments, the nanoparticle comprises a mix of double helix oligonucleotide structures comprising three or more different sequences.

When the nanoparticle comprises a mix of double helix oligonucleotide structures comprising different sequences on different target molecules, the different target molecules can be molecules involved in the same disease. When the nanoparticle comprises a mix of double helix oligonucleotide structures comprising different sequences on different target molecules, the different target molecules can be molecules involved in the same biological process (e.g., same molecular pathway).

In some embodiments, the nanoparticle comprises a mix of double helix oligonucleotide structures comprising two different sequences, wherein the two different sequences target two different target molecules associated with Alzheimer's disease (see e.g., Table 6A). In some embodiments, the nanoparticle comprises a mix of double helix oligonucleotide structures comprising two different sequences, wherein the two different sequences target two different target molecules associated with Parkinson's disease (see e.g., Table 6B). In some embodiments, the nanoparticle comprises a mix of double helix oligonucleotide structures comprising two different sequences, wherein the two different sequences target two different target molecules associated with Huntington's disease (see e.g., Table 6C). In some embodiments, the nanoparticle comprises a mix of double helix oligonucleotide structures comprising two different sequences, wherein the two different sequences target two different target molecules associated with Multiple sclerosis (see e.g., Table 6D). In some embodiments, the nanoparticle comprises a mix of double helix oligonucleotide structures comprising two different sequences, wherein the two different sequences target two different target molecules associated with Spinal cord injury (see e.g., Table 6E).

In another aspect, provided herein is a composition for blood brain barrier permeation, the composition comprising a double strand oligonucleotide structure.

### Methods of Treatment

In another aspect, provided herein is a method of preventing or treating a disorder in a subject in need thereof comprising administering to the subject a therapeutically effective amount of the compound as provided herein, the nanoparticle as provided herein, the composition as provided herein, or a pharmaceutical composition as provided herein,
wherein the disorder is associated with expression and/or activity of one or more pathological genes,
wherein the target molecule is the one or more pathological genes, and
wherein the administration reduces or inhibits expression and/or activity of the target molecule in the subject compared with a target molecule expression and/or activity in a subject prior to or in the absence of administration of the compound as provided herein, the nanoparticle as provided herein, the composition as provided herein, or a pharmaceutical composition as provided herein, thereby, alleviating or delaying one or more signs or symptoms of the disorder in the subject relative to a subject prior to or in the absence of the administration of the compound as provided herein, the nanoparticle as provided herein, the composition as provided herein, or a pharmaceutical composition as provided herein.

In another aspect, provided herein is a method of alleviating or delaying one or more signs or symptoms of a disorder in a subject in need thereof comprising administering a therapeutically effective amount of the compound as provided herein, the nanoparticle as provided herein, the composition as provided herein, or a pharmaceutical composition as provided herein to the subject,
wherein the disorder is associated with expression and/or activity of one or more pathological genes,
wherein the target molecule is the one or more pathological genes, and
wherein the administration reduces or inhibits expression and/or activity of the target molecule in the subject compared with a target molecule expression and/or activity in a subject prior to or in the absence of the administration of the compound of as provided herein, the nanoparticle as provided herein, the composition as provided herein, or a pharmaceutical composition as provided herein.

In another aspect, provided herein is a method of treating a disorder in a subject in need thereof comprising administering to the subject a therapeutically effective amount of a compound comprising the structure of Formula 17:

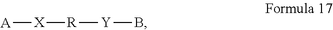

wherein:
R is an inhibitory oligonucleotide molecule;
A is a hydrophilic moiety;
B is a hydrophobic moiety; and
X and Y are independently a covalent bond or a first linker; and
wherein a ligand molecule for a glucose transporter (GLUT), or functional analog thereof is covalently linked to a terminus of A, a terminus of B, or a combination thereof via a second linker or a bond,
wherein the disorder is associated with expression and/or activity of one or more pathological genes,
wherein the target molecule is the one or more pathological genes, and
wherein the administration reduces or inhibits expression and/or activity of the target molecule in the subject compared with a target molecule expression and/or activity in a subject prior to or in the absence of the administration of the compound, thereby, alleviating or delaying one or more signs or symptoms of the disorder in the subject relative to a subject prior to or in the absence of the administration of the compound.

A method of alleviating or delaying one or more signs or symptoms of a disorder in a subject in need thereof comprising administering to the subject a therapeutically effective amount of a compound comprising the structure of Formula 17:

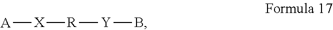

wherein:
R is an inhibitory oligonucleotide molecule;
A is a hydrophilic moiety;
B is a hydrophobic moiety; and
X and Y are independently a covalent bond or a first linker; and
wherein a ligand molecule for a glucose transporter (GLUT), or functional analog thereof is covalently linked to a terminus of A, a terminus of B, or a combination thereof via a second linker or a bond, and
wherein the administration reduces or inhibits expression and/or activity of the target molecule in the subject compared with a target molecule expression and/or activity in a subject prior to or in the absence of the administration of the compound, thereby, alleviating or delaying one or more signs or symptoms of the disorder in the subject relative to a subject prior to or in the absence of the administration of the compound.

In some embodiments, the administration of the compound as provided herein, the nanoparticle as provided herein, the composition as provided herein, or a pharmaceutical composition as provided herein alleviates or delays one or more signs or symptoms of the disorder in the subject relative to a subject prior to or in the absence of such administration. In some embodiments, the administration of the compound as provided herein, the nanoparticle as provided herein, the composition as provided herein, or a pharmaceutical composition as provided herein alleviates one or more signs or symptoms of the disorder in the subject relative to a subject prior to or in the absence of such administration. In some embodiments, the administration of the compound as provided herein, the nanoparticle as provided herein, the composition as provided herein, or a pharmaceutical composition as provided herein delays one or more signs or symptoms of the disorder in the subject relative to a subject prior to or in the absence of such administration.

In some embodiments, the administration of the compound as provided herein, the nanoparticle as provided herein, the composition as provided herein, or a pharmaceutical composition as provided herein alleviates one or more signs or symptoms of the disorder in the subject by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% relative to a subject prior to or in the absence of such administration. In some embodiments, the administration of the compound as provided herein, the nanoparticle as provided herein, the composition as provided herein, or a pharmaceutical composition as provided herein alleviates one or more signs or symptoms of the disorder in the subject by at least about 1.1-folds, about 1.2-folds, about 1.3-folds, about 1.4-folds, about 1.5-folds, about 1.6-folds, 1.7-folds, about 1.8-folds, about 1.9-folds, about 2.5-folds, about 3-folds, about 3.5-folds, about 4-folds, about 4.5-folds, about 5-folds, about 5.5-folds, about 6-folds, 6.5-folds, about 7-folds, about 7.5-folds, about 8-folds, about 8.5-folds, about 9-folds, about 9.5-folds, about 10-folds, about 20-folds, about 30-folds, about 40-folds, about 50-folds, about 60-folds, about 70-folds, about 80-folds, about 90-folds, about 100-folds, about 200-folds, about 300-folds, about 400-folds, about 500-folds, about 600-folds, about 700-folds, about 800-folds, about 900-folds, about 1000-folds, about 1500-folds, about 2000-folds, about 2500-folds, about 3000-folds, about 3500-folds, about 4000-folds, about 4500-folds, about 5000-folds, about 5500-folds, about 6000-folds, about 6500-folds, about 7000-folds, about 7500-folds, about 8000-folds, about 8500-folds, about 9000-folds, about 9500-folds, about 10000-folds-folds, about 20000-folds, about 30000-folds, about 40000-folds, or about 50000-folds relative to a subject prior to or in the absence of such administration.

In some embodiments, the administration of the compound as provided herein, the nanoparticle as provided herein, the composition as provided herein, or a pharmaceutical composition as provided herein delays one or more signs or symptoms of the disorder in the subject by at least about 5 min, 10 min, 15 min, 20 min, 25 min, 30 min, 35 min, 40 min, 45 min, 50 min, 55 min, 1 hr, 1.5 hr, 2 hr, 2.5 hr, 3 hr, 3.5 hr, 4 hr, 4.5 hr, 5 hr, 5.5 hr, 6 hr, 6.5 hr, 7 hr, 7.5 hr, 8 hr, 8.5 hr, 9 hr, 9.5 hr, 10 hr, 10.5 hr, 11 hr, 11.5 hr, 12 hr, 13 hr, 14 hr, 15 hr, 16 hr, 17 hr, 18 hr, 19 hr, 20 hr, 21 hr, 22 hr, 23 hr, 24 hr, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 12 months, 1 yr, 2 yrs, 3 yrs, 4 yrs, 5 yrs, 6 yrs, 7 yrs, 8 yrs, 9 yrs, 10 yrs, 11 yrs, 12 yrs, 13 yrs, 14 yrs, 15 yrs, 16 yrs, 17 yrs, 18 yrs, 19 yrs, 20 yrs, 21 yrs, 22 yrs, 23 yrs, 24 yrs, 25 yrs, 26 yrs, 27 yrs, 28 yrs, 29 yrs, or 30 yrs relative to a subject prior to or in the absence of such administration.

In some embodiments, the disorder is a neurological disorder. The compound of as provided herein, the nanoparticle as provided herein, the composition as provided herein, or a pharmaceutical composition as provided herein may be used in compositions for treating or improving neurological disease (Alzheimer's disease, dementia, Parkinson's disease, etc.), and is expected to be useful as a composition for preventing and treating neurological disease by improving cognitive function/memory by inhibiting deposition of amyloid beta (AB), inhibiting brain inflammation, and inhibiting tau phosphorylation.

In some embodiments, the neurological disorder is Alzheimer's Disease (AD), stroke, dementia, muscular dystrophy (MD), multiple sclerosis (MS), amyotrophic lateral sclerosis (ALS), cystic fibrosis, Angel-man syndrome, Liddle syndrome, Parkinson's Disease, Pick's Disease, Pagets Disease, cancer, or traumatic brain damage.

In some embodiments, the neurological disorder is Alzheimer's Disease (AD), stroke, dementia, muscular dystrophy (MD), multiple sclerosis (MS), amyotrophic lateral sclerosis (ALS), cystic fibrosis, Angel-man syndrome, Liddle syndrome, Parkinson's Disease, Pick's Disease, Pagets Disease, cancer, tauopathies, age-related macular degeneration, or traumatic brain damage.

In some embodiments, the disorder is Alzheimer's disease, and the target molecule is BACE1-AS, BACE1, presenilin1 (PS1), ROCK-II, 12 PP-2A, ACAT-1, Nogo receptor, mutant presenilin1 (L392V PS-1), APP, Tau, VDAC1, or any combination thereof. In some embodiments, the disorder is Parkinson's disease, and the target molecule is α-synuclein. In some embodiments, the disorder is Huntington's disease, and the target molecule is Huntingtin (Htt). In some embodiments, the disorder is multiple sclerosis, and the target molecule is Notch1, LINGO-1, NR4A2, TRIF, caspase-2, CaMKII, or any combination thereof. In some embodiments, the disorder is spinal cord injury, and the target molecule is glial fibrillary acidic protein (GFAP), Vimentin, EphB3, iNOS, Nischarin, RhoA, or any combination thereof.

In some embodiments, the disorder is any one of the diseases or disorders listed in Table 6A, Table 6B, Table 6C, Table 6D, Table 6E, or Table 5, and the target molecule is any one of the corresponding target molecules listed in Table 6A, Table 6B, Table 6C, Table 6D, or Table 6E.

In some embodiments, the disorder is cancer. In some embodiments, the cancer is brain cancer, breast cancer, cervix cancer, colon cancer, kidney cancer, lung cancer, ovary cancer, prostate cancer, skin cancer, thyroid cancer, glioblastoma, pancreas cancer, leukemia, gastric cancer, colorectal cancer, or any combination thereof.

In some embodiments, the cancer is glioblastoma, non-small cell lung carcinoma, prostate carcinoma, pancreatic ductal adenocarcinoma, metastatic gastric adenocarcinoma, invasive ductal carcinoma, or colorectal carcinoma.

In some embodiments, the cancer is prostate cancer, lung cancer, renal cancer, stomach cancer, colon cancer, ovarian cancer, bladder cancer, breast cancer, cervical cancer, esophageal cancer, testicular cancer, liver cancer, pancreatic cancer, rectal cancer, thyroid cancer, uterine cancer, skin cancer, muscle cancer, cartilage cancer, bone cancer, endothelial cancer, epithelial cancer, dermal cancer, basal cancer, retinal cancer, skin cancer, or brain cancer.

In some embodiments, the cancer is leukemia, lymphoma, or myeloma. In some embodiments, the cancer is B-cell acute lymphoid leukemia (B-ALL), T-cell acute lymphoid leukemia (T-ALL), acute lymphoblastic leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, or preleukemia.

In some embodiments, the one or more signs or symptoms comprises Aβ peptide accumulation in the brain, tau peptide accumulation in the brain, phosphorylated tau accumulation in the brain, tau protein phosphorylation, a hippocampal damage, a cognitive deficit, or any combination thereof. In some embodiments, the Aβ peptide accumulation occurs in the CA1 and subiculum of the brain of the subject. In some embodiments, the tau peptide accumulation occurs in the CA1 and subiculum of the brain of the subject. In some embodiments, the phosphorylated tau accumulation occurs in the CA1 and subiculum of the brain of the subject.

In some embodiments, the administration of the compound as provided herein, the nanoparticle as provided herein, the composition as provided herein, or a pharmaceutical composition as provided herein results in any one of the effects listed in Table 6A, Table 6B, Table 6C, Table 6D, or Table 6E, or any combination thereof relative to a subject prior to or in the absence of such administration.

In some embodiments, upon the administration of the compound as provided herein, the nanoparticle as provided herein, the composition as provided herein, or a pharmaceutical composition as provided herein, the subject shows no or minimum systemic toxicity comparable to a control subject prior to or in the absence of the administration. In some embodiments, upon the administration of the compound as provided herein, the nanoparticle as provided herein, the composition as provided herein, or a pharmaceutical composition as provided herein, the subject shows systemic toxicity at the level of at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% relative to a control subject prior to or in the absence of the administration.

In some embodiments, upon the administration of the compound as provided herein, the nanoparticle as provided herein, the composition as provided herein, or a pharmaceutical composition as provided herein, the subject shows no or minimum hepatotoxicity comparable to a control subject prior to or in the absence of the administration.

In some embodiments, upon the administration of the compound as provided herein, the nanoparticle as provided herein, the composition as provided herein, or a pharmaceutical composition as provided herein, the subject shows hepatotoxicity at the level of at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% relative to a control subject prior to or in the absence of the administration.

In some embodiments, the hepatotoxicity is assessed by the alkaline phosphatase (ALP) level, by the alanine aminotransferase (ALT) level, the aspartate aminotransferase (AST) level, the total bilirubin (T-bil) level, the ammonia (NH₃) level, or any combination thereof in the subject. In some embodiments, the hepatotoxicity is assessed by the alkaline phosphatase (ALP) level, by the alanine aminotransferase (ALT) level, the aspartate aminotransferase (AST) level, the total bilirubin (T-bil) level, the ammonia (NH₃) level, or any combination thereof in a sample from the subject. In some embodiments, the hepatotoxicity is assessed by the alkaline phosphatase (ALP) level, by the alanine aminotransferase (ALT) level, the aspartate aminotransferase (AST) level, the total bilirubin (T-bil) level, the ammonia (NH₃) level, or any combination thereof in a blood sample from the subject.

In some embodiments, upon the administration of the compound as provided herein, the nanoparticle as provided herein, the composition as provided herein, or a pharmaceutical composition as provided herein, the alkaline phosphatase (ALP) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the alkaline phosphatase (ALP) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the alanine aminotransferase (ALT) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration.

In some embodiments, upon the administration of the compound as provided herein, the nanoparticle as provided herein, the composition as provided herein, or a pharmaceutical composition as provided herein, the alanine aminotransferase (ALT) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the aspartate aminotransferase (AST) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the aspartate aminotransferase (AST) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the total bilirubin (T-bil) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the total bilirubin (T-bil) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the ammonia (NH3) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the ammonia (NH3) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the subject shows no or minimum kidney toxicity comparable to a control subject prior to or in the absence of the administration.

In some embodiments, upon the administration of the compound as provided herein, the nanoparticle as provided herein, the composition as provided herein, or a pharmaceutical composition as provided herein, the subject shows kidney toxicity at the level of at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% relative to a control subject prior to or in the absence of the administration.

In some embodiments, the kidney toxicity is assessed by the lactic acid (Lac) level, the blood urea nitrogen (BUN) level, the creatinine (Crea) level, or any combination thereof in the subject. In some embodiments, the kidney toxicity is assessed by the lactic acid (Lac) level, the blood urea nitrogen (BUN) level, the creatinine (Crea) level, or any combination thereof in a sample from the subject. In some embodiments, the kidney toxicity is assessed by the lactic acid (Lac) level, the blood urea nitrogen (BUN) level, the creatinine (Crea) level, or any combination thereof in a blood sample from the subject.

In some embodiments, upon the administration of the compound as provided herein, the nanoparticle as provided herein, the composition as provided herein, or a pharmaceutical composition as provided herein, the lactic acid (Lac) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the lactic acid (Lac) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the blood urea nitrogen (BUN) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the blood urea nitrogen (BUN) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the creatinine (Crea) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the creatinine (Crea) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the subject shows no or minimum altered lipid metabolism comparable to a control subject prior to or in the absence of the administration.

In some embodiments, upon the administration of the compound as provided herein, the nanoparticle as provided herein, the composition as provided herein, or a pharmaceutical composition as provided herein, the subject shows altered lipid metabolism at the level of at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% relative to a control subject prior to or in the absence of the administration.

In some embodiments, the lipid metabolism is assessed by the lipase (Lip) level, the high density lipoprotein (HDL) level, the low density lipoprotein (LDL) level, the total cholesterol (T-Chol) level, the triglycerides (TG) level, or any combination thereof in the subject. In some embodiments, the lipid metabolism is assessed by the lipase (Lip) level, the high density lipoprotein (HDL) level, the low density lipoprotein (LDL) level, the total cholesterol (T-Chol) level, the triglycerides (TG) level, or any combination thereof in a sample from the subject. In some embodiments, the lipid metabolism is assessed by the lipase (Lip) level, the high density lipoprotein (HDL) level, the low density lipoprotein (LDL) level, the total cholesterol (T-Chol) level, the triglycerides (TG) level, or any combination thereof in a blood sample from the subject.

In some embodiments, upon the administration of the compound as provided herein, the nanoparticle as provided herein, the composition as provided herein, or a pharmaceutical composition as provided herein, the lipase (Lip) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the lipase (Lip) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the high density lipoprotein (HDL) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the high density lipoprotein (HDL) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the low density lipoprotein (LDL) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the low density lipoprotein (LDL) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the total cholesterol (T-Chol) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the total cholesterol (T-Chol) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the triglycerides (TG) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the triglycerides (TG) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the subject shows no or minimum altered calcium homeostasis comparable to a control subject prior to or in the absence of the administration.

In some embodiments, upon the administration of the compound as provided herein, the nanoparticle as provided herein, the composition as provided herein, or a pharmaceutical composition as provided herein, the subject shows altered calcium homeostasis at the level of at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% relative to a control subject prior to or in the absence of the administration.

In some embodiments, the calcium homeostasis is assessed by the calcium (Ca) level, the phosphorus (P) level, or a combination thereof in the subject. In some embodiments, the calcium homeostasis is assessed by the calcium (Ca) level, the phosphorus (P) level, or a combination thereof in a sample from the subject. In some embodiments, the calcium homeostasis is assessed by the calcium (Ca) level, the phosphorus (P) level, or a combination thereof in a blood sample from the subject.

In some embodiments, upon the administration of the compound as provided herein, the nanoparticle as provided herein, the composition as provided herein, or a pharmaceutical composition as provided herein, the calcium (Ca) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the calcium (Ca) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, upon the administration of the compound as provided herein, the nanoparticle as provided herein, the composition as provided herein, or a pharmaceutical composition as provided herein, the phosphorus (P) level in the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a control subject prior to or in the absence of the administration. In some embodiments, upon the administration of the compound as provided herein, the nanoparticle as provided herein, the composition as provided herein, or a pharmaceutical composition as provided herein, the phosphorus (P) level in a sample from the subject is at least about 100%, about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 85%, about 80%, about 75%, about 70%, about 65%, about 60% compared with the level in a sample from a control subject prior to or in the absence of the administration.

In some embodiments, the blood brain barrier penetration of the compound in the brain of the subject is increased compared with the blood brain barrier penetration of a compound having the same structure without the ligand molecule for a GLUT, or functional analog thereof linked.

In some embodiments, the blood brain barrier penetration of the compound in the brain of the subject is increased by at least about 10%, about 20%, about 30%, about 40%, about 50%, about 55%, about 60%, about 65%, about 70%, about 75%, about 80%, about 85%, about 90%, about 95%, about 100%, 150%, about 200%, about 250%, about 300%, about 350%, about 400%, about 450%, about 500%, about 550%, about 600%, about 650%, about 700%, about 800%, about 850%, about 900%, about 1000%, about 1500%, about 2000%, about 2500%, about 3000%, about 3500%, about 4000%, about 4500%, about 5000%, about 5500%, about 6000%, about 6500%, about 7000%, about 7500%, about 8000%, about 8500%, about 9000%, about 9500%, about 10000%, about 15000%, about 20000%, about 25000%, about 30000%, about 35000%, about 40000%, about 45000%, about 50000%, about 55000%, about 60000%, about 65000%, about 70000%, about 75000%, about 80000%, about 85000%, about 90000%, about 100000%, or more compared with the blood brain barrier penetration of a compound having the same structure without the ligand molecule for a GLUT, or functional analog thereof linked. In some embodiments, the blood brain barrier penetration of the compound in the brain of the subject is increased by at least about 1.1-folds, about 1.2-folds, about 1.3-folds, about 1.4-folds, about 1.5-folds, about 1.6-folds, 1.7-folds, about 1.8-folds, about 1.9-folds, about 2.5-folds, about 3-folds, about 3.5-folds, about 4-folds, about 4.5-folds, about 5-folds, about 5.5-folds, about 6-folds, 6.5-folds, about 7-folds, about 7.5-folds, about 8-folds, about 8.5-folds, about 9-folds, about 9.5-folds, about 10-folds, about 20-folds, about 30-folds, about 40-folds, about 50-folds, about 60-folds, about 70-folds, about 80-folds, about 90-folds, about 100-folds, about 200-folds, about 300-folds, about 400-folds, about 500-folds, about 600-folds, about 700-folds, about 800-folds, about 900-folds, about 1000-folds, about 1500-folds, about 2000-folds, about 2500-folds, about 3000-folds, about 3500-folds, about 4000-folds, about 4500-folds, about 5000-folds, about 5500-folds, about 6000-folds, about 6500-folds, about 7000-folds, about 7500-folds, about 8000-folds, about 8500-folds, about 9000-folds, about 9500-folds, about 10000-folds-folds, about 20000-folds, about 30000-folds, about 40000-folds, about 50000-folds, 60000-folds, about 70000-folds, about 80000-folds, about 90000-folds, about 100000-folds, or more compared with the blood brain barrier penetration of a compound having the same structure without the ligand molecule for a GLUT, or functional analog thereof linked.

In some embodiments, the retention time and/or the distribution of the compound in the brain of the subject is increased compared with the retention time and/or the distribution of a compound having the same structure without the ligand molecule for a GLUT, or functional analog thereof linked.

In some embodiments, the retention time and/or the distribution of the compound in the brain of the subject is increased by at least about 10%, about 20%, about 30%, about 40%, about 50%, about 55%, about 60%, about 65%, about 70%, about 75%, about 80%, about 85%, about 90%, about 95%, about 100%, 150%, about 200%, about 250%, about 300%, about 350%, about 400%, about 450%, about 500%, about 550%, about 600%, about 650%, about 700%, about 800%, about 850%, about 900%, about 1000%, about 1500%, about 2000%, about 2500%, about 3000%, about 3500%, about 4000%, about 4500%, about 5000%, about 5500%, about 6000%, about 6500%, about 7000%, about 7500%, about 8000%, about 8500%, about 9000%, about 9500%, about 10000%, about 15000%, about 20000%, about 25000%, about 30000%, about 35000%, about 40000%, about 45000%, about 50000%, about 55000%, about 60000%, about 65000%, about 70000%, about 75000%, about 80000%, about 85000%, about 90000%, about 100000%, or more compared with the retention time and/or the distribution of a compound having the same structure without the ligand molecule for a GLUT, or functional analog thereof linked. In some embodiments, the retention time and/or the distribution of the compound in the brain of the subject is increased by at least about 1.1-folds, about 1.2-folds, about 1.3-folds, about 1.4-folds, about 1.5-folds, about 1.6-folds, 1.7-folds, about 1.8-folds, about 1.9-folds, about 2.5-folds, about 3-folds, about 3.5-folds, about 4-folds, about 4.5-folds, about 5-folds, about 5.5-folds, about 6-folds, 6.5-folds, about 7-folds, about 7.5-folds, about 8-folds, about 8.5-folds, about 9-folds, about 9.5-folds, about 10-folds, about 20-folds, about 30-folds, about 40-folds, about 50-folds, about 60-folds, about 70-folds, about 80-folds, about 90-folds, about 100-folds, about 200-folds, about 300-folds, about 400-folds, about 500-folds, about 600-folds, about 700-folds, about 800-folds, about 900-folds, about 1000-folds, about 1500-folds, about 2000-folds, about 2500-folds, about 3000-folds, about 3500-folds, about 4000-folds, about 4500-folds, about 5000-folds, about 5500-folds, about 6000-folds, about 6500-folds, about 7000-folds, about 7500-folds, about 8000-folds, about 8500-folds, about 9000-folds, about 9500-folds, about 10000-folds-folds, about 20000-folds, about 30000-folds, about 40000-folds, about 50000-folds, 60000-folds, about 70000-folds, about 80000-folds, about 90000-folds, about 100000-folds, or more compared with the retention time and/or the distribution of a compound having the same structure without the ligand molecule for a GLUT, or functional analog thereof linked.

In some embodiments, the compound is more specifically delivered to one or more tissues, organs, or cells that express the GLUT compared with tissues, organs, or cells that do not express the GLUT.

In some embodiments, the compound is delivered to one or more tissues, organs, or cells that express the GLUT to a greater extent by at least about 10%, about 20%, about 30%, about 40%, about 50%, about 55%, about 60%, about 65%, about 70%, about 75%, about 80%, about 85%, about 90%, about 95%, about 100%, 150%, about 200%, about 250%, about 300%, about 350%, about 400%, about 450%, about 500%, about 550%, about 600%, about 650%, about 700%, about 800%, about 850%, about 900%, about 1000%, about 1500%, about 2000%, about 2500%, about 3000%, about 3500%, about 4000%, about 4500%, about 5000%, about 5500%, about 6000%, about 6500%, about 7000%, about 7500%, about 8000%, about 8500%, about 9000%, about 9500%, about 10000%, about 15000%, about 20000%, about 25000%, about 30000%, about 35000%, about 40000%, about 45000%, about 50000%, about 55000%, about 60000%, about 65000%, about 70000%, about 75000%, about 80000%, about 85000%, about 90000%, about 100000%, or more compared with tissues, organs, or cells that do not express the GLUT. In some embodiments, the compound is delivered to one or more tissues, organs, or cells that express the GLUT to a greater extent by at least about 1.1-folds, about 1.2-folds, about 1.3-folds, about 1.4-folds, about 1.5-folds, about 1.6-folds, 1.7-folds, about 1.8-folds, about 1.9-folds, about 2.5-folds, about 3-folds, about 3.5-folds, about 4-folds, about 4.5-folds, about 5-folds, about 5.5-folds, about 6-folds, 6.5-folds, about 7-folds, about 7.5-folds, about 8-folds, about 8.5-folds, about 9-folds, about 9.5-folds, about 10-folds, about 20-folds, about 30-folds, about 40-folds, about 50-folds, about 60-folds, about 70-folds, about 80-folds, about 90-folds, about 100-folds, about 200-folds, about 300-folds, about 400-folds, about 500-folds, about 600-folds, about 700-folds, about 800-folds, about 900-folds, about 1000-folds, about 1500-folds, about 2000-folds, about 2500-folds, about 3000-folds, about 3500-folds, about 4000-folds, about 4500-folds, about 5000-folds, about 5500-folds, about 6000-folds, about 6500-folds, about 7000-folds, about 7500-folds, about 8000-folds, about 8500-folds, about 9000-folds, about 9500-folds, about 10000-folds-folds, about 20000-folds, about 30000-folds, about 40000-folds, about 50000-folds, 60000-folds, about 70000-folds, about 80000-folds, about 90000-folds, about 100000-folds, or more compared with tissues, organs, or cells that do not express the GLUT.

In some embodiments, the target molecule expression and/or activity is modulated in one or more tissues, organs, or cells that express the GLUT to a greater extent compared with tissues, organs, or cells that do not express the GLUT. In some embodiments, the target molecule expression is modulated in one or more tissues, organs, or cells that express the GLUT to a greater extent compared with tissues, organs, or cells that do not express the GLUT. In some embodiments, the target molecule expression is modulated in one or more tissues, organs, or cells that express the GLUT to a greater extent by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared with tissues, organs, or cells that do not express the GLUT.

In some embodiments, the target molecule activity is modulated in one or more tissues, organs, or cells that express the GLUT to a greater extent compared with tissues, organs, or cells that do not express the GLUT. In some embodiments, the target molecule activity is modulated in one or more tissues, organs, or cells that express the GLUT to a greater extent by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared with tissues, organs, or cells that do not express the GLUT.

In some embodiments, the target molecule expression and/or activity is reduced or inhibited in one or more tissues, organs, or cells that express the GLUT to a greater extent compared with tissues, organs, or cells that do not express the GLUT.

In some embodiments, the target molecule expression is reduced or inhibited in one or more tissues, organs, or cells that express the GLUT to a greater extent compared with tissues, organs, or cells that do not express the GLUT. In some embodiments, the target molecule expression is reduced or inhibited in one or more tissues, organs, or cells that express the GLUT to a greater extent compared with tissues, organs, or cells that do not express the GLUT. In some embodiments, the target molecule expression is modulated in one or more tissues, organs, or cells that express the GLUT to a greater extent by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared with tissues, organs, or cells that do not express the GLUT.

In some embodiments, the target molecule activity is reduced or inhibited in one or more tissues, organs, or cells that express the GLUT to a greater extent compared with tissues, organs, or cells that do not express the GLUT. In some embodiments, the target molecule activity is reduced or inhibited in one or more tissues, organs, or cells that express the GLUT to a greater extent by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared with tissues, organs, or cells that do not express the GLUT.

In some embodiments, the one or more tissues, organs, or cells that express the GLUT comprises brain, blood, brain, intestine, liver, kidney, pancreas, embryos, testis, placenta, muscle, heart, fat, adipose tissue, spleen, colon, prostate, testis, blastocysts, skeletal muscle, white adipose tissue, brown adipose tissue, or any combination thereof.

In some embodiments, (i) the GLUT is GLUT1, and the one or more tissues, organs, or cells that express the GLUT comprises brain, blood, or a combination thereof;
(ii) the GLUT is GLUT2, and the one or more tissues, organs, or cells that express the GLUT comprises brain, intestine, liver, kidney, pancreas, or any combination thereof;
(iii) the GLUT is GLUT3, and the one or more tissues, organs, or cells that express the GLUT comprises brain, embryos, testis, placenta, or any combination thereof;
(iv) the GLUT is GLUT4, and the one or more tissues, organs, or cells that express the GLUT comprises brain, muscle, heart, fat, adipose tissue, or any combination thereof;
(v) the GLUT is GLUT5, and the one or more tissues, organs, or cells that express the GLUT comprises brain, intestine, testis, muscle, kidney, fat, or any combination thereof;
(vi) the GLUT is GLUT6, and the one or more tissues, organs, or cells that express the GLUT comprises brain, spleen, or a combination thereof;
(vii) the GLUT is GLUT7, and the one or more tissues, organs, or cells that express the GLUT comprises intestine, colon, prostate, testis, prostate gland, or any combination thereof;
(viii) the GLUT is GLUT8, and the one or more tissues, organs, or cells that express the GLUT comprises brain, testis, liver, spleen, fat, blastocysts, or any combination thereof;
(ix) the GLUT is GLUT9, and the one or more tissues, organs, or cells that express the GLUT comprises liver, kidney, intestine, colon, or any combination thereof;
(x) the GLUT is GLUT10, and the one or more tissues, organs, or cells that express the GLUT comprises liver, pancreas, fat, skeletal muscle, heart, adipose tissue, placenta, kidney, or any combination thereof;
(xi) the GLUT is GLUT11, and the one or more tissues, organs, or cells that express the GLUT comprises heart, muscle, kidney, pancreas, placenta, or any combination thereof;
(xii) the GLUT is GLUT12, and the one or more tissues, organs, or cells that express the GLUT comprises brain, muscle, heart, fat, pancreas, prostate, adipose tissue, placenta, kidney, or any combination thereof;
(xiii) the GLUT is GLUT13, and the one or more tissues, organs, or cells that express the GLUT comprises brain, white adipose tissue, brown adipose tissue, kidney, or any combination thereof; or
(xiv) the GLUT is GLUT14, and the one or more tissues, organs, or cells that express the GLUT comprises testis.

In some embodiments, (i) the GLUT is GLUT1, and the one or more tissues, organs, or cells that express the GLUT comprises blood-brain barrier, astrocyte, erythrocyte, or any combination thereof;
(ii) the GLUT is GLUT2, and the one or more tissues, organs, or cells that express the GLUT comprises astrocyte, gastrointestinal tract, small intestine, intestinal absorptive epithelial cells, hepatocytes, pancreatic B-cells, brain nuclei, nucleus tractus solitatius, motor nucleus of the vagus, paraventricular hypothalamic nucleus, lateral hypothalamic area, arcuate nucleus, and olfactory bulb, or any combination thereof;
(iii) the GLUT is GLUT3, and the one or more tissues, organs, or cells that express the GLUT comprises neuron, blood-brain barrier, astrocyte, sperm, spermatozoa, fibroblast, platelet, retinal endothelial cell, white blood cell, or any combination thereof;
(iv) the GLUT is GLUT4, and the one or more tissues, organs, or cells that express the GLUT comprises neuron, adipocyte, skeletal muscle cell, cardiomyocyte, hypothalamus, cerebellum, cortex, and hippocampus, or a combination thereof;
(v) the GLUT is GLUT5, and the one or more tissues, organs, or cells that express the GLUT comprises microglia, blood-brain barrier, small intestine, apical membrane of the enterocytes, plasma membrane of mature spermatozoa, skeletal muscle, or any combination thereof;
(vi) the GLUT is GLUT6, and the one or more tissues, organs, or cells that express the GLUT comprises leukocyte, peripheral leukocyte, germinal cell of the testis, or any combination thereof;
(vii) the GLUT is GLUT7, and the one or more tissues, organs, or cells that express the GLUT comprises small intestine;
(viii) the GLUT is GLUT8, and the one or more tissues, organs, or cells that express the GLUT comprises neuron, brown adipose tissue, cerebellum, adrenal gland, spermatocyte, mature spermatozoa, or any combination thereof;
(ix) the GLUT is GLUT9, and the one or more tissues, organs, or cells that express the GLUT comprises small intestine, leukocyte, chondrocyte, or any combination thereof;
(x) the GLUT is GLUT10, and the one or more tissues, organs, or cells that express the GLUT comprises white adipose;
(xi) the GLUT is GLUT12, and the one or more tissues, organs, or cells that express the GLUT comprises astrocyte, prostate gland, small intestine, chondrocyte, or any combination thereof; or
(xii) the GLUT is GLUT13, and the one or more tissues, organs, or cells that express the GLUT comprises neuron.

In some embodiments, the one or more tissues, organs, or cells that express the GLUT comprises a cancer cell.

In some embodiments, the cancer is selected from the group consisting of brain cancer, breast cancer, cervix cancer, colon cancer, kidney cancer, lung cancer, ovary cancer, prostate cancer, skin cancer, thyroid cancer, glioblastoma, pancreas cancer, leukemia, gastric cancer, colorectal cancer, and any combination thereof.

In some embodiments, (i) the GLUT is GLUT1, and the cancer is selected from the group consisting of brain cancer, breast cancer, cervix cancer, colon cancer, kidney cancer, lung cancer, ovary cancer, prostate cancer, skin cancer, thyroid cancer, and any combination thereof;
(ii) the GLUT is GLUT3, and the cancer is selected from the group consisting of brain cancer, breast cancer, colon cancer, cervix cancer, kidney cancer, lung cancer, glioblastoma, and any combination thereof;
(iii) the GLUT is GLUT5, and the cancer is selected from the group consisting of breast cancer, colon cancer, pancreas cancer, ovary cancer, lung cancer, leukemia, and any combination thereof;
(iv) the GLUT is GLUT10, and the cancer is leukemia;
(v) the GLUT is GLUT12, and the cancer is breast cancer; or
(vi) the GLUT is GLUT14, and the cancer is brain cancer.

In some embodiments, the one or more tissues, organs, or cells that express the GLUT comprises any one of the tissue, organ, or cell listed in Table 4 or Table 5, or any combination thereof.

In some embodiments, (i) the target molecule is BACE1-AS, and the administration results in improvement of memory and learning behaviors;
(ii) the target molecule is BACE1, and the administration results in decrease of amyloid plaque rate, improvement in neuropathological and behavioral signs, suppression of BACE1 expression and/or activity, reduction of plaque load in the cerebral cortex and hippocampus, increase in the level of synaptophysin, rescue of memory loss, or any combination thereof;
(iii) the target molecule is presenilin1 (PS1), and the administration results in reduction of the level of Aβ 42;
(iv) the target molecule is ROCK-II, and the administration results in promotion of axonal regeneration;
(v) the target molecule is I2 PP-2A, and the administration results in decrease of the level of Aβ and APP and phosphorylated tau, improvement of memory and learning ability, or a combination thereof;
(vi) the target molecule is ACAT-1, and the administration results in reductio of the enzymatic process of APP, enhancement of the level of free cholesterol, or a combination thereof;
(vii) the target molecule is Nogo receptor, and the administration results in promotion of the regeneration and repair of cholinergic neurons;
(viii) the target molecule is mutant presenilin1 (L392V PS-1), BACE1, or a combination thereof, and the administration results in decrease of the level of amyloid plaque;
(ix) the target molecule is APP, Tau, VDAC1, or a combination thereof, and the administration results in improvement in synaptic activity and mitochondrial function;
(x) the target molecule is α-synuclein (SNCA), and the administration results in reduction of the level of SNCA, reduction of hSNCA-mediated behavioral deficits, improvement in motor dysfunction, protection of cells from death via apoptosis, reduction of α-syn accumulation, amelioration of inflammatory pathology, or any combination thereof;
(xi) the target molecule is Htt, and the administration results in inhibition of the Htt expression and/or activity, reduction of size and number of neuronal intranuclear inclusions (NIIs), improvement of movement deficits, survival of neurons, decreasing the level of mutant htt protein, or any combination thereof;
(xii) the target molecule is T-bet, and the administration results in regulation of interferon (IFN), prevention of the onset of disease, or a combination thereof;
(xiii) the target molecule is Notch1, and the administration results in improved motor function, repair in histopathological sections, or a combination thereof;
(xiv) the target molecule is LINGO-1, and the administration results in improvement of memory and learning behaviors;
(xv) the target molecule is NR4A2, and the administration results in inhibition of the pathogenic potentials of IFN and IL-17;
(xvi) the target molecule is TRIF, and the administration results in alleviation of the severity of the disease via the inhibition of interleukin and cytokine release;
(xvii) the target molecule is caspase-2, and the administration results in inhibition of nerve cell loss, decrease in retinal nerve fiber layer (RNFL) thickness, increased survival of retinal ganglion cells (RGC), or any combination thereof;
(xviii) the target molecule is CaMKII, and the administration results in reduction mechanical and thermal hypersensitivity, reduction of the evoked and non-evoked pain, or a combination thereof;
(xix) the target molecule is GFAP, Vimentin, or a combination thereof, and the administration results in improvement of urinary function;
(xx) the target molecule is EphB3, and the administration results in improvement of axonal regeneration, improvement of the motor function, or a combination thereof;
(xxi) the target molecule is iNOS, and the administration results in improvement of the secondary damage following spinal cord injury;
(xxii) the target molecule is Nischarin, and the administration results in improvement of motor function; or
(xxiii) the target molecule is RhoA, and the administration results in improvement in walking, declining of allodynia, or a combination thereof.

In some embodiments, the target molecule is any one of the target molecules listed in listed in Table 6A, Table 6B, Table 6C, Table 6D, or Table 6E, or any combination thereof, and the administration of the compound as provided herein, the nanoparticle as provided herein, the composition as provided herein, or a pharmaceutical composition as provided herein results in any one of the effects listed in Table 6A, Table 6B, Table 6C, Table 6D, or Table 6E, or any combination thereof compared with a subject prior to or in the absence of such administration.

In some embodiments, a single dose of the compound as provided herein may be 1 μg/kg to 100 mg/kg, preferably 5 μg/kg to 50 mg/kg, administered once weekly or 1 to 3 times weekly, but the dose and administration intervals are not limited hereto. In some embodiments, a single dose of the compound as provided herein may be about 1 μg/kg to about 100 mg/kg, about 1 μg/kg to about 90 mg/kg, about 1 μg/kg to about 80 mg/kg, about 1 μg/kg to about 70 mg/kg, about 1 μg/kg to about 60 mg/kg, about 1 μg/kg to about 50 mg/kg, about 1 μg/kg to about 40 mg/kg, about 1 μg/kg to about 30 mg/kg, about 1 μg/kg to about 20 mg/kg, about 1 μg/kg to about 10 mg/kg, about 1 μg/kg to about 5 mg/kg, about 1 μg/kg to about 1 mg/kg, about 1 μg/kg to about 900 μg/kg, about 1 μg/kg to about 800 μg/kg, about 1 μg/kg to about 700 μg/kg, about 1 μg/kg to about 600 μg/kg, about 1 μg/kg to about 500 μg/kg, about 1 μg/kg to about 400 μg/kg, about 1 μg/kg to about 300 μg/kg, about 1 μg/kg to about 200 μg/kg, about 1 μg/kg to about 100 μg/kg, about 1 μg/kg to about 90 μg/kg, about 1 μg/kg to about 80 μg/kg, about 1 μg/kg to about 70 μg/kg, about 1 μg/kg to about 60 μg/kg, about 1 μg/kg to about 50 μg/kg, about 1 μg/kg to about 40 μg/kg, about 1 μg/kg to about 30 μg/kg, about 1 μg/kg to about 20 μg/kg, about 1 μg/kg to about 10 μg/kg, or about 1 μg/kg to about 5 μg/kg. In some embodiments, a single dose of the compound as provided herein may be about 1 μg/kg to about 100 mg/kg, about 5 μg/kg to about 100 mg/kg, about 10 μg/kg to about 100 mg/kg, about 20 μg/kg to about 100 mg/kg, about 30 μg/kg to about 100 mg/kg, about 40 μg/kg to about 100 mg/kg, 50 μg/kg to about 100 mg/kg, about 60 μg/kg to about 100 mg/kg, about 70 μg/kg to about 100 mg/kg, about 80 μg/kg to about 100 mg/kg, about 90 μg/kg to about 100 mg/kg, about 100 μg/kg to about 100 mg/kg, about 200 μg/kg to about 100 mg/kg, about 300 μg/kg to about 100 mg/kg, about 400 μg/kg to about 100 mg/kg, about 500 μg/kg to about 100 mg/kg, about 600 μg/kg to about 100 mg/kg, about 700 μg/kg to about 100 mg/kg, about 800 μg/kg to about 100 mg/kg, about 900 μg/kg to about 100 mg/kg, about 1 mg/kg to about 100 mg/kg, about 5 mg/kg to about 100 mg/kg, about 10 mg/kg to about 100 mg/kg, about 20 mg/kg to about 100 mg/kg, about 30 mg/kg to about 100 mg/kg, about 40 mg/kg to about 100 mg/kg, about 50 mg/kg to about 100 mg/kg, about 60 mg/kg to about 100 mg/kg, about 70 mg/kg to about 100 mg/kg, about 80 mg/kg to about 100 mg/kg, about 90 mg/kg to about 100 mg/kg, or about 100 mg/kg to about 100 mg/kg. In some embodiments, a single dose of the compound as provided herein may be about 1 μg/kg to about 100 mg/kg, about 5 μg/kg to about 50 mg/kg, about 10 μg/kg to about 10 mg/kg, about 50 μg/kg to about 1 mg/kg, or about 100 μg/kg to about 500 μg/kg.

### Methods of Targeted Delivery and Target Molecule Knock-Down on the Target

In another aspect, provided herein is a method of targeted delivery of a compound comprising the structure of Formula 20 to a specific tissue, organ, or cell in a subject in need thereof comprising:

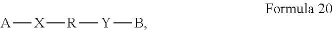

wherein:
R is an oligonucleotide molecule;
A is a hydrophilic moiety;
B is a hydrophobic moiety; and
X and Y are independently a covalent bond or a first linker,
(i) covalently linking a ligand molecule for a glucose transporter (GLUT), or functional analog thereof via a second linker or a bond to a terminus of A, a terminus of B, or a combination thereof of the compound comprising the structure of Formula 20; and
(ii) administering to the subject;
wherein the specific tissue, organ, or cell expresses the GLUT; and
wherein the compound is delivered to a greater extent to the specific tissue, organ, or cell compared with tissues, organs, or cells that do not express the GLUT.

In another aspect, provided herein is a compound for targeted delivery to a specific tissue, organ, or cell in a subject in need thereof, comprising the structure of Formula 20:

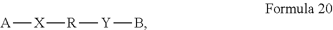

wherein:
R is an oligonucleotide molecule;
A is a hydrophilic moiety;
B is a hydrophobic moiety; and
X and Y are independently a covalent bond or a first linker,
wherein a ligand molecule for a glucose transporter (GLUT), or functional analog thereof is covalently linked to a terminus of A, a terminus of B, or a combination thereof via a second linker or a bond; and
wherein the specific tissue, organ, or cell expresses the GLUT; and
wherein when administered to the subject, the compound is delivered to a greater extent to the specific tissue, organ, or cell compared with tissues, organs, or cells that do not express the GLUT.

In some embodiments, the compound is delivered to the specific tissue, organ, or cell expresses the GLUT to a greater extent by at least about 10%, about 20%, about 30%, about 40%, about 50%, about 55%, about 60%, about 65%, about 70%, about 75%, about 80%, about 85%, about 90%, about 95%, about 100%, 150%, about 200%, about 250%, about 300%, about 350%, about 400%, about 450%, about 500%, about 550%, about 600%, about 650%, about 700%, about 800%, about 850%, about 900%, about 1000%, about 1500%, about 2000%, about 2500%, about 3000%, about 3500%, about 4000%, about 4500%, about 5000%, about 5500%, about 6000%, about 6500%, about 7000%, about 7500%, about 8000%, about 8500%, about 9000%, about 9500%, about 10000%, about 15000%, about 20000%, about 25000%, about 30000%, about 35000%, about 40000%, about 45000%, about 50000%, about 55000%, about 60000%, about 65000%, about 70000%, about 75000%, about 80000%, about 85000%, about 90000%, about 100000%, or more compared with tissues, organs, or cells that do not express the GLUT. In some embodiments, the compound is delivered to the specific tissue, organ, or cell expresses the GLUT to a greater extent by at least about 1.1-folds, about 1.2-folds, about 1.3-folds, about 1.4-folds, about 1.5-folds, about 1.6-folds, 1.7-folds, about 1.8-folds, about 1.9-folds, about 2.5-folds, about 3-folds, about 3.5-folds, about 4-folds, about 4.5-folds, about 5-folds, about 5.5-folds, about 6-folds, 6.5-folds, about 7-folds, about 7.5-folds, about 8-folds, about 8.5-folds, about 9-folds, about 9.5-folds, about 10-folds, about 20-folds, about 30-folds, about 40-folds, about 50-folds, about 60-folds, about 70-folds, about 80-folds, about 90-folds, about 100-folds, about 200-folds, about 300-folds, about 400-folds, about 500-folds, about 600-folds, about 700-folds, about 800-folds, about 900-folds, about 1000-folds, about 1500-folds, about 2000-folds, about 2500-folds, about 3000-folds, about 3500-folds, about 4000-folds, about 4500-folds, about 5000-folds, about 5500-folds, about 6000-folds, about 6500-folds, about 7000-folds, about 7500-folds, about 8000-folds, about 8500-folds, about 9000-folds, about 9500-folds, about 10000-folds-folds, about 20000-folds, about 30000-folds, about 40000-folds, about 50000-folds, 60000-folds, about 70000-folds, about 80000-folds, about 90000-folds, about 100000-folds, or more compared with tissues, organs, or cells that do not express the GLUT.

In some embodiments, R is an inhibitory oligonucleotide molecule.

In another aspect, provided herein is a method of reducing or inhibiting a target molecule expression and/or activity in a specific tissue, organ, or cell of a subject in need thereof comprising administering to the subject a therapeutically effective amount of a compound comprises the structure of Formula 17:

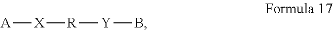

wherein:
R is an inhibitory oligonucleotide molecule;
A is a hydrophilic moiety;
B is a hydrophobic moiety; and
X and Y are independently a covalent bond or a first linker; and
wherein a ligand molecule for a glucose transporter (GLUT), or functional analog thereof is covalently linked to a terminus of A, a terminus of B, or a combination thereof via a second linker or a bond;
wherein the specific tissue, organ, or cell expresses the GLUT;
wherein the compound reduces or inhibits expression and/or activity of the target molecule in the specific tissue, organ, or cell to a greater extent compared with a target molecule expression and/or activity in a tissue, organ, or cell that do not express the GLUT.

In another aspect, provided herein is a compound for reducing or inhibiting a target molecule expression and/or activity in a specific tissue, organ, or cell of a subject in need thereof, comprising the structure of Formula 17:

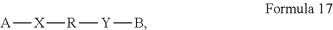

wherein:
R is an inhibitory oligonucleotide molecule;
A is a hydrophilic moiety;
B is a hydrophobic moiety; and
X and Y are independently a covalent bond or a first linker; and
wherein a ligand molecule for a glucose transporter (GLUT), or functional analog thereof is covalently linked to a terminus of A, a terminus of B, or a combination thereof via a second linker or a bond; and wherein the specific tissue, organ, or cell expresses the GLUT;
wherein the compound reduces or inhibits expression and/or activity of the target molecule in the specific tissue, organ, or cell to a greater extent compared with a target molecule expression and/or activity in a tissue, organ, or cell that do not express the GLUT.

In some embodiments, the compound reduces or inhibits expression of the target molecule in the specific tissue, organ, or cell to a greater extent compared with a target molecule expression in a tissue, organ, or cell that do not express the GLUT. In some embodiments, the compound reduces or inhibits expression of the target molecule in the specific tissue, organ, or cell to a greater extent by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared with a target molecule expression in a tissue, organ, or cell that do not express the GLUT. In some embodiments, the compound reduces or inhibits activity of the target molecule in the specific tissue, organ, or cell to a greater extent compared with a target molecule activity in a tissue, organ, or cell that do not express the GLUT. In some embodiments, the compound reduces or inhibits activity of the target molecule in the specific tissue, organ, or cell to a greater extent by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared with a target molecule activity in a tissue, organ, or cell that do not express the GLUT.

In the compound modulates expression and/or activity of the target molecule in the specific tissue, organ, or cell to a greater extent compared with a target molecule expression and/or activity in a tissue, organ, or cell that do not express the GLUT. In some embodiments, the compound modulates expression of the target molecule in the specific tissue, organ, or cell to a greater extent compared with a target molecule expression in a tissue, organ, or cell that do not express the GLUT. In some embodiments, the compound modulates expression of the target molecule in the specific tissue, organ, or cell to a greater extent by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared with a target molecule expression in a tissue, organ, or cell that do not express the GLUT. In some embodiments, the compound modulates activity of the target molecule in the specific tissue, organ, or cell to a greater extent compared with a target molecule activity in a tissue, organ, or cell that do not express the GLUT. In some embodiments, the compound modulates activity of the target molecule in the specific tissue, organ, or cell to a greater extent by at least about 10%, about 20%, about 30%, about 40%, about 50%, about 55%, about 60%, about 65%, about 70%, about 75%, about 80%, about 85%, about 90%, about 95%, about 96%, about 97%, about 98%, about 99%, about 99.5%, about 99.9%, or about 100% compared with a target molecule activity in a tissue, organ, or cell that do not express the GLUT.

In some embodiments, the second linker is —NH(CH₂)ₙCONH—CH₂—CH(OH)—CH₂—, an ether linkage, or Compound 4:

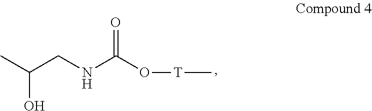

wherein T is 1-15 repetitive Compound 1:

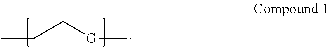

wherein G is selected from the group consisting of CH₂, O, S and NH.

In some embodiments, the second linker is any one listed in Table 7.

In some embodiments, the specific tissue, organ, or cell that expresses the GLUT comprises brain, blood, brain, intestine, liver, kidney, pancreas, embryos, testis, placenta, muscle, heart, fat, adipose tissue, spleen, colon, prostate, testis, blastocysts, skeletal muscle, white adipose tissue, brown adipose tissue, or any combination thereof.

In some embodiments, (i) the GLUT is GLUT1, and the specific tissue, organ, or cell that expresses the GLUT comprises brain, blood, or a combination thereof;
(ii) the GLUT is GLUT2, and the specific tissue, organ, or cell that expresses the GLUT comprises brain, intestine, liver, kidney, pancreas, or any combination thereof;
(iii) the GLUT is GLUT3, and the specific tissue, organ, or cell that expresses the GLUT comprises brain, embryos, testis, placenta, or any combination thereof;
(iv) the GLUT is GLUT4, and the specific tissue, organ, or cell that expresses the GLUT comprises brain, muscle, heart, fat, adipose tissue, or any combination thereof;
(v) the GLUT is GLUT5, and the specific tissue, organ, or cell that expresses the GLUT comprises brain, intestine, testis, muscle, kidney, fat, or any combination thereof;
(vi) the GLUT is GLUT6, and the specific tissue, organ, or cell that expresses the GLUT comprises brain, spleen, or a combination thereof;
(vii) the GLUT is GLUT7, and the specific tissue, organ, or cell that expresses the GLUT comprises intestine, colon, prostate, testis, prostate gland, or any combination thereof;
(viii) the GLUT is GLUT8, and the specific tissue, organ, or cell that expresses the GLUT comprises brain, testis, liver, spleen, fat, blastocysts, or any combination thereof;
(ix) the GLUT is GLUT9, and the specific tissue, organ, or cell that expresses the GLUT comprises liver, kidney, intestine, colon, or any combination thereof;
(x) the GLUT is GLUT10, and the specific tissue, organ, or cell that expresses the GLUT comprises liver, pancreas, fat, skeletal muscle, heart, adipose tissue, placenta, kidney, or any combination thereof;
(xi) the GLUT is GLUT11, and the specific tissue, organ, or cell that expresses the GLUT comprises heart, muscle, kidney, pancreas, placenta, or any combination thereof;
(xii) the GLUT is GLUT12, and the specific tissue, organ, or cell that expresses the GLUT comprises brain, muscle, heart, fat, pancreas, prostate, adipose tissue, placenta, kidney, or any combination thereof;
(xiii) the GLUT is GLUT13, and the specific tissue, organ, or cell that expresses the GLUT comprises brain, white adipose tissue, brown adipose tissue, kidney, or any combination thereof; or
(xiv) the GLUT is GLUT14, and the specific tissue, organ, or cell that expresses the GLUT comprises testis.

In some embodiments, (i) the GLUT is GLUT1, and the specific tissue, organ, or cell that expresses the GLUT comprises blood-brain barrier, astrocyte, erythrocyte, or any combination thereof;
(ii) the GLUT is GLUT2, and the specific tissue, organ, or cell that expresses the GLUT comprises astrocyte, gastrointestinal tract, small intestine, intestinal absorptive epithelial cells, hepatocytes, pancreatic B-cells, brain nuclei, nucleus tractus solitatius, motor nucleus of the vagus, paraventricular hypothalamic nucleus, lateral hypothalamic area, arcuate nucleus, and olfactory bulb, or any combination thereof;
(iii) the GLUT is GLUT3, and the specific tissue, organ, or cell that expresses the GLUT comprises neuron, blood-brain barrier, astrocyte, sperm, spermatozoa, fibroblast, platelet, retinal endothelial cell, white blood cell, or any combination thereof;
(iv) the GLUT is GLUT4, and the specific tissue, organ, or cell that expresses the GLUT comprises neuron, adipocyte, skeletal muscle cell, cardiomyocyte, hypothalamus, cerebellum, cortex, and hippocampus, or a combination thereof;
(v) the GLUT is GLUT5, and the specific tissue, organ, or cell that expresses the GLUT comprises microglia, blood-brain barrier, small intestine, apical membrane of the enterocytes, plasma membrane of mature spermatozoa, skeletal muscle, or any combination thereof;
(vi) the GLUT is GLUT6, and the specific tissue, organ, or cell that expresses the GLUT comprises leukocyte, peripheral leukocyte, germinal cell of the testis, or any combination thereof;
(vii) the GLUT is GLUT7, and the specific tissue, organ, or cell that expresses the GLUT comprises small intestine;
(viii) the GLUT is GLUT8, and the specific tissue, organ, or cell that expresses the GLUT comprises neuron, brown adipose tissue, cerebellum, adrenal gland, spermatocyte, mature spermatozoa, or any combination thereof;
(ix) the GLUT is GLUT9, and the specific tissue, organ, or cell that expresses the GLUT comprises small intestine, leukocyte, chondrocyte, or any combination thereof;
(x) the GLUT is GLUT10, and the specific tissue, organ, or cell that expresses the GLUT comprises white adipose;
(xi) the GLUT is GLUT12, and the specific tissue, organ, or cell that expresses the GLUT comprises astrocyte, prostate gland, small intestine, chondrocyte, or any combination thereof; or
(xii) the GLUT is GLUT13, and the specific tissue, organ, or cell that expresses the GLUT comprises neuron.

In some embodiments, the specific tissue, organ, or cell that expresses the GLUT comprises cancer cells.

In some embodiments, the cancer is selected from the group consisting of brain cancer, breast cancer, cervix cancer, colon cancer, kidney cancer, lung cancer, ovary cancer, prostate cancer, skin cancer, thyroid cancer, glioblastoma, pancreas cancer, leukemia, gastric cancer, colorectal cancer, and any combination thereof.

In some embodiments, (i) the GLUT is GLUT1, and the cancer is selected from the group consisting of brain cancer, breast cancer, cervix cancer, colon cancer, kidney cancer, lung cancer, ovary cancer, prostate cancer, skin cancer, thyroid cancer, and any combination thereof;
(ii) the GLUT is GLUT3, and the cancer is selected from the group consisting of brain cancer, breast cancer, colon cancer, cervix cancer, kidney cancer, lung cancer, glioblastoma, and any combination thereof;
(iii) the GLUT is GLUT5, and the cancer is selected from the group consisting of breast cancer, colon cancer, pancreas cancer, ovary cancer, lung cancer, leukemia, and any combination thereof;
(iv) the GLUT is GLUT10, and the cancer is leukemia;
(v) the GLUT is GLUT12, and the cancer is breast cancer; or
(vi) the GLUT is GLUT14, and the cancer is brain cancer.

In some embodiments, the specific tissue, organ, or cell that expresses the GLUT comprises any one of the tissue, organ, or cell listed in Table 4 or Table 5, or any combination thereof.

### Methods of Improving Blood Brain Barrier Penetration

In another aspect, provided herein is a method of increasing blood brain barrier penetration of a compound comprising the structure of Formula 20 in a subject in need thereof comprising:

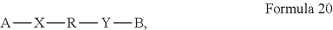

wherein:
R is an oligonucleotide molecule;
A is a hydrophilic moiety;
B is a hydrophobic moiety; and
X and Y are independently a covalent bond or a first linker,
(i) covalently linking a ligand molecule for a glucose transporter (GLUT), or functional analog thereof via a second linker or a bond to a terminus of A, a terminus of B, or a combination thereof of the compound comprising the structure of Formula 20; and
(ii) administering to the subject;
wherein the compound exhibits blood brain barrier penetration in the subject to a greater extent compared with a compound having the same structure without the ligand molecule for a GLUT, or functional analog thereof linked.

In another aspect, provided herein is a compound with increased blood brain barrier penetration in a subject in need thereof, comprising the structure of Formula 20:

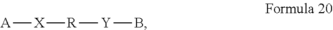

wherein:
R is an oligonucleotide molecule;
A is a hydrophilic moiety;
B is a hydrophobic moiety; and
X and Y are independently a covalent bond or a first linker,
wherein a ligand molecule for a glucose transporter (GLUT), or functional analog thereof is covalently linked to a terminus of A, a terminus of B, or a combination thereof via a second linker or a bond; and
wherein when administered to the subject, the compound exhibits blood brain barrier penetration in the subject to a greater extent compared with a compound having the same structure without the ligand molecule for a GLUT, or functional analog thereof linked.

In some embodiments, the compound exhibits blood brain barrier penetration in the subject to a greater extent by at least about 10%, about 20%, about 30%, about 40%, about 50%, about 55%, about 60%, about 65%, about 70%, about 75%, about 80%, about 85%, about 90%, about 95%, about 100%, 150%, about 200%, about 250%, about 300%, about 350%, about 400%, about 450%, about 500%, about 550%, about 600%, about 650%, about 700%, about 800%, about 850%, about 900%, about 1000%, about 1500%, about 2000%, about 2500%, about 3000%, about 3500%, about 4000%, about 4500%, about 5000%, about 5500%, about 6000%, about 6500%, about 7000%, about 7500%, about 8000%, about 8500%, about 9000%, about 9500%, about 10000%, about 15000%, about 20000%, about 25000%, about 30000%, about 35000%, about 40000%, about 45000%, about 50000%, about 55000%, about 60000%, about 65000%, about 70000%, about 75000%, about 80000%, about 85000%, about 90000%, about 100000%, or more compared with a compound having the same structure without the ligand molecule for a GLUT, or functional analog thereof linked.

In some embodiments, the compound exhibits blood brain barrier penetration in the subject to a greater extent by at least about 1.1-folds, about 1.2-folds, about 1.3-folds, about 1.4-folds, about 1.5-folds, about 1.6-folds, 1.7-folds, about 1.8-folds, about 1.9-folds, about 2.5-folds, about 3-folds, about 3.5-folds, about 4-folds, about 4.5-folds, about 5-folds, about 5.5-folds, about 6-folds, 6.5-folds, about 7-folds, about 7.5-folds, about 8-folds, about 8.5-folds, about 9-folds, about 9.5-folds, about 10-folds, about 20-folds, about 30-folds, about 40-folds, about 50-folds, about 60-folds, about 70-folds, about 80-folds, about 90-folds, about 100-folds, about 200-folds, about 300-folds, about 400-folds, about 500-folds, about 600-folds, about 700-folds, about 800-folds, about 900-folds, about 1000-folds, about 1500-folds, about 2000-folds, about 2500-folds, about 3000-folds, about 3500-folds, about 4000-folds, about 4500-folds, about 5000-folds, about 5500-folds, about 6000-folds, about 6500-folds, about 7000-folds, about 7500-folds, about 8000-folds, about 8500-folds, about 9000-folds, about 9500-folds, about 10000-folds-folds, about 20000-folds, about 30000-folds, about 40000-folds, about 50000-folds, 60000-folds, about 70000-folds, about 80000-folds, about 90000-folds, about 100000-folds, or more compared with a compound having the same structure without the ligand molecule for a GLUT, or functional analog thereof linked.

In some embodiments, the blood brain barrier penetration of the compound in the brain of the subject is increased by at least about 10%, about 20%, about 30%, about 40%, about 50%, about 55%, about 60%, about 65%, about 70%, about 75%, about 80%, about 85%, about 90%, about 95%, about 100%, 150%, about 200%, about 250%, about 300%, about 350%, about 400%, about 450%, about 500%, about 550%, about 600%, about 650%, about 700%, about 800%, about 850%, about 900%, about 1000%, about 1500%, about 2000%, about 2500%, about 3000%, about 3500%, about 4000%, about 4500%, about 5000%, about 5500%, about 6000%, about 6500%, about 7000%, about 7500%, about 8000%, about 8500%, about 9000%, about 9500%, about 10000%, about 15000%, about 20000%, about 25000%, about 30000%, about 35000%, about 40000%, about 45000%, about 50000%, about 55000%, about 60000%, about 65000%, about 70000%, about 75000%, about 80000%, about 85000%, about 90000%, about 100000%, or more compared with the blood brain barrier penetration of a compound having the same structure without the ligand molecule for a GLUT, or functional analog thereof linked.

In some embodiments, the blood brain barrier penetration of the compound in the brain of the subject is increased by at least about 1.1-folds, about 1.2-folds, about 1.3-folds, about 1.4-folds, about 1.5-folds, about 1.6-folds, 1.7-folds, about 1.8-folds, about 1.9-folds, about 2.5-folds, about 3-folds, about 3.5-folds, about 4-folds, about 4.5-folds, about 5-folds, about 5.5-folds, about 6-folds, 6.5-folds, about 7-folds, about 7.5-folds, about 8-folds, about 8.5-folds, about 9-folds, about 9.5-folds, about 10-folds, about 20-folds, about 30-folds, about 40-folds, about 50-folds, about 60-folds, about 70-folds, about 80-folds, about 90-folds, about 100-folds, about 200-folds, about 300-folds, about 400-folds, about 500-folds, about 600-folds, about 700-folds, about 800-folds, about 900-folds, about 1000-folds, about 1500-folds, about 2000-folds, about 2500-folds, about 3000-folds, about 3500-folds, about 4000-folds, about 4500-folds, about 5000-folds, about 5500-folds, about 6000-folds, about 6500-folds, about 7000-folds, about 7500-folds, about 8000-folds, about 8500-folds, about 9000-folds, about 9500-folds, about 10000-folds-folds, about 20000-folds, about 30000-folds, about 40000-folds, about 50000-folds, 60000-folds, about 70000-folds, about 80000-folds, about 90000-folds, about 100000-folds, or more compared with the blood brain barrier penetration of a compound having the same structure without the ligand molecule for a GLUT, or functional analog thereof linked.

In some embodiments, the second linker is —NH(CH₂)ₙCONH—CH₂—CH(OH)—CH₂—, an ether linkage, or Compound 4:

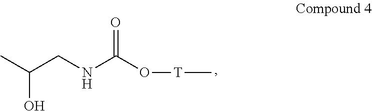

wherein T is 1-15 repetitive Compound 1:

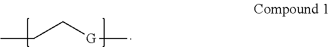

wherein G is selected from the group consisting of CH₂, O, S and NH.

In some embodiments, the second linker is any one listed in Table 7.

In some embodiments, the ligand molecule for a GLUT is a sugar. In some embodiments, the sugar is selected from the group consisting of fructose, galactose, glucosamine, glucose, mannose, xylose, dehydroascorbic acid, and trehalose.

In some embodiments, the GLUT is selected from the group consisting of GLUT1, GLUT2, GLUT3, GLUT4, GLUT5, GLUT6, GLUT8, GLUT12, and GLUT13.

In some embodiments, the ligand molecule for a GLUT is fructose and the GLUT is selected from the group consisting of GLUT2, GLUT5, GLUT8, and GLUT12. In some embodiments, the ligand molecule for a GLUT is galactose and the GLUT is selected from the group consisting of GLUT1, GLUT2, GLUT3, and GLUT4. In some embodiments, the ligand molecule for a GLUT is glucose and the GLUT is selected from the group consisting of GLUT1, GLUT2, GLUT3, GLUT4, GLUT6, GLUT8, and GLUT12. In some embodiments, the ligand molecule for a GLUT is mannose and the GLUT is selected from the group consisting of GLUT1, GLUT2, GLUT3, and GLUT4. In some embodiments, the ligand molecule for a GLUT is xylose and the GLUT is GLUT3. In some embodiments, the ligand molecule for a GLUT is dehydroascorbic acid and the GLUT is GLUT4. In some embodiments, the ligand molecule for a GLUT is trehalose and the GLUT is GLUT8. In some embodiments, the ligand molecule for a GLUT is any one selected from the ligands listed in Table 3, and the GLUT is the corresponding GLUT as listed in Table 3.

In some embodiments, the blood brain barrier penetration of the compound is mediated via a GLUT-mediated ligand uptake into a cell and a GLUT-mediated ligand efflux across the blood brain barrier.

In some embodiments, when administered to a subject, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% of the compound administered penetrates the blood brain barrier of the subject. In some embodiments, when administered to a subject, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% of the compound administered is delivered to the brain of the subject.

### Treatment of Alzheimer's Disease Using Double Strand Oligonucleotide Structure

In an aspects, provided herein is a double strand oligonucleotide structure for blood brain barrier permeability and a composition for preventing or treating neurological disorder comprising the same. In some embodiments, provided herein is a double strand oligonucleotide structure for blood brain barrier permeability wherein glucose is conjugated through a linker, and a composition for preventing or treating neurological disorder comprising the same.

In an aspect, an object of the present disclosure is to provide a double strand oligonucleotide structure comprising a double strand oligonucleotide wherein simple sugars are bonded by a ligand.

In another aspect, another object of the present disclosure is to provide a composition for carriage through the blood brain barrier, the composition comprising the double strand oligonucleotide structure.

Yet in another aspect, another object of the present disclosure is to provide a composition for preventing or treating neurological disorder comprising the double strand oligonucleotide structure.

To achieve the above-stated object, in some embodiments, provided herein is a double strand oligonucleotide structure comprising the structure of Formula 1 below:

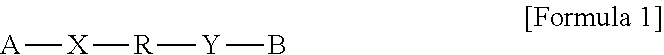

In Formula 1, A is a hydrophilic substance, B is a hydrophobic substance, and X and Y each independently represent a simple covalent bond or linked-mediated covalent bond, and R refers to a double strand oligonucleotide comprising a sense strand and an antisense strand comprising a sequence complementary thereto,
And a monosaccharide is bonded to the A or B terminal.

In another aspect, provided herein is a nanoparticle comprising the double strand oligonucleotide structure.

In another aspect, provided herein is a composition for trans-blood brain barrier transport, the composition comprising the double strand oligonucleotide structure.

In another aspect, provided herein is a composition for preventing or treating neurological disorder comprising the double strand oligonucleotide structure.

In some embodiments, the double strand oligonucleotide structure as provided herein comprises, as a nanoparticle structure for delivery to brain tissue, a ligand which effectively permeates the blood brain barrier. Alzheimer's disease is a chronic illness wherein degeneration of brain cells gradually reduces cognitive function include memory, causing difficulty in everyday life. The double strand oligonucleotide structure according to the present disclosure may be used in compositions for treating or improving neurological disease (Alzheimer's disease, dementia, Parkinson's disease, etc.), and is expected to be useful as a composition for preventing and treating neurological disease by improving cognitive function/memory by inhibiting deposition of amyloid beta (Aβ), inhibiting brain inflammation, and inhibiting tau phosphorylation.

In some embodiments, the neurological disorder is Alzheimer's disease. The Alzheimer's disease medications currently being marked can be classified into AChE inhibitors (substance names donepezil, rivastigmine, galantamine), which inhibit the breaking down of the neurotransmitter acetylcholine, and NMDA receptor antagonists (substance name memantine), which prevents binding of glutamate with receptors leading to hyperactivity. These are mostly drugs which improve symptoms, with most patents expiring since 2009 and generic drugs released. Whereas therapeutic agents that temporarily increase concentrations of neurotransmitters, such as AChEI, to exhibit improved cognitive function were the mainstream until recently, the presence of pipelines targeting the amyloid beta (Aβ) and Tau proteins which are strongly suspected to be the cause of the illness is increasing. Of these, Aducanumab from Biogen was the first antibody drug, currently in Phase 3 clinical trials.

In some embodiments, the compound as provided herein, e.g., the double strand oligonucleotide structure as provided herein, as a gene therapy agent, fundamentally blocks the generation of amyloid beta and Tau proteins that are the root cause of the illness, and has substantial technical benefits compared to Aducanumab, which is currently the most promising drug. In some embodiments, unlike antibody drugs that block etiological proteins after they are created, gene therapy agents block the synthesis of etiological proteins beforehand, and theoretically have an efficiency at least approximately 1,000 times greater. The only reason why therapeutic agents have not been developed despite their outstanding inhibition efficacy is delivery to the etiological site. Whereas RNA is easily broken down and has the disadvantage of low in vivo stability, SAMiRNA™-based technology has already been proven to have resolved this problem. Additionally, positive delivery results indicating efficient permeation of the blood brain barrier, which is the second barrier as well as the in vivo stability have been also demonstrated.

In another aspect, provided herein is a composition for preventing or treating neurological disorder comprising the double strand oligonucleotide structure.

In some embodiments, non-limiting examples of the neurological disorder may include Alzheimer's Disease (AD), stroke, dementia, muscular dystrophy (MD), multiple sclerosis (MS), amyotrophic lateral sclerosis (ALS), cystic fibrosis, Angelman syndrome, Liddle syndrome, Parkinson's Disease, Pick's Disease, Pagets Disease, cancer and traumatic brain damage.

In some embodiments, the composition of the present disclosure, e.g., the double strand oligonucleotide structure as provided herein, may be prepared for administration to include, in addition to the active ingredient described in the above, one or more pharmaceutically acceptable carriers. A pharmaceutically acceptable carrier must be compatible with the active ingredient of the present disclosure, and may be saline, sterile water, Ringer's solution, buffered saline, dextrose solution, maltodextrin solution, glycerol, ethanol, and mixtures of two or more of these components. These may be mixed with other conventional additives such as antioxidants, buffers, and bacteriostats as needed. In addition, diluents, dispersants, surfactants, binders and lubricants may be additionally added to form an injectable formulation such as an aqueous solution, suspension, emulsion, and the like. In particular, it is preferable to provide the formulation in a lyophilized form. For the preparation of a freeze-dried formulation, a method commonly known in the art to which the present disclosure pertains may be used, and a stabilizer for freeze-drying may be added. Furthermore, the composition may be preferably formulated according to each disease or component by using a method disclosed in Remington's Pharmaceutical Science (Remington's Pharmaceutical Science, Mack Publishing company, Easton PA) by an appropriate method in the art.

In some embodiments, the composition of the present disclosure, e.g., the double strand oligonucleotide structure as provided herein, is preferably administered parenterally, and the dosage varies depending on the patient's weight, age, sex, health status, diet, administration time, method, excretion rate, or severity of disease, etc., and may be readily decided by a person skilled in the art.

In some embodiments, a single dose of the composition according to the present disclosure, e.g., the double strand oligonucleotide structure as provided herein, may be 1 μg/kg to 100 mg/kg, preferably 5 μg/kg to 50 mg/kg, administered once weekly or 1 to 3 times weekly, but the dose and administration intervals are not limited hereto. In some embodiments, a single dose of the composition according to the present disclosure, e.g., the double strand oligonucleotide structure as provided herein, may be about 1 μg/kg to about 100 mg/kg, about 1 μg/kg to about 90 mg/kg, about 1 μg/kg to about 80 mg/kg, about 1 μg/kg to about 70 mg/kg, about 1 μg/kg to about 60 mg/kg, about 1 μg/kg to about 50 mg/kg, about 1 μg/kg to about 40 mg/kg, about 1 μg/kg to about 30 mg/kg, about 1 μg/kg to about 20 mg/kg, about 1 μg/kg to about 10 mg/kg, about 1 μg/kg to about 5 mg/kg, about 1 μg/kg to about 1 mg/kg, about 1 μg/kg to about 900 μg/kg, about 1 μg/kg to about 800 μg/kg, about 1 μg/kg to about 700 μg/kg, about 1 μg/kg to about 600 μg/kg, about 1 μg/kg to about 500 μg/kg, about 1 μg/kg to about 400 μg/kg, about 1 μg/kg to about 300 μg/kg, about 1 μg/kg to about 200 μg/kg, about 1 μg/kg to about 100 μg/kg, about 1 μg/kg to about 90 μg/kg, about 1 μg/kg to about 80 μg/kg, about 1 μg/kg to about 70 μg/kg, about 1 μg/kg to about 60 μg/kg, about 1 μg/kg to about 50 μg/kg, about 1 μg/kg to about 40 μg/kg, about 1 μg/kg to about 30 μg/kg, about 1 μg/kg to about 20 μg/kg, about 1 μg/kg to about 10 μg/kg, or about 1 μg/kg to about 5 μg/kg. In some embodiments, a single dose of the composition according to the present disclosure, e.g., the double strand oligonucleotide structure as provided herein, may be about 1 μg/kg to about 100 mg/kg, about 5 μg/kg to about 100 mg/kg, about 10 μg/kg to about 100 mg/kg, about 20 μg/kg to about 100 mg/kg, about 30 μg/kg to about 100 mg/kg, about 40 μg/kg to about 100 mg/kg, 50 μg/kg to about 100 mg/kg, about 60 μg/kg to about 100 mg/kg, about 70 μg/kg to about 100 mg/kg, about 80 μg/kg to about 100 mg/kg, about 90 μg/kg to about 100 mg/kg, about 100 μg/kg to about 100 mg/kg, about 200 μg/kg to about 100 mg/kg, about 300 μg/kg to about 100 mg/kg, about 400 μg/kg to about 100 mg/kg, about 500 μg/kg to about 100 mg/kg, about 600 μg/kg to about 100 mg/kg, about 700 μg/kg to about 100 mg/kg, about 800 μg/kg to about 100 mg/kg, about 900 μg/kg to about 100 mg/kg, about 1 mg/kg to about 100 mg/kg, about 5 mg/kg to about 100 mg/kg, about 10 mg/kg to about 100 mg/kg, about 20 mg/kg to about 100 mg/kg, about 30 mg/kg to about 100 mg/kg, about 40 mg/kg to about 100 mg/kg, about 50 mg/kg to about 100 mg/kg, about 60 mg/kg to about 100 mg/kg, about 70 mg/kg to about 100 mg/kg, about 80 mg/kg to about 100 mg/kg, about 90 mg/kg to about 100 mg/kg, or about 100 mg/kg to about 100 mg/kg. In some embodiments, a single dose of the composition according to the present disclosure, e.g., the double strand oligonucleotide structure as provided herein, may be about 1 μg/kg to about 100 mg/kg, about 5 μg/kg to about 50 mg/kg, about 10 μg/kg to about 10 mg/kg, about 50 μg/kg to about 1 mg/kg, or about 100 μg/kg to about 500 μg/kg.

### Pharmaceutical Compositions

In an aspect, provided herein are the pharmaceutical compositions comprising the compound as provided herein or the composition as provided herein. In some embodiments, the pharmaceutical compositions as provided herein may further comprise one or more pharmaceutically acceptable carriers, diluents or excipients. In some embodiments, the pharmaceutical compositions as provided herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some embodiments, the pharmaceutical composition further comprises a pharmaceutically acceptable excipient. Examples of pharmaceutically acceptable carriers, diluents or excipients include, but are not limited to, antifoaming agents, antioxidants, binders, carriers or carrier materials, dispersing agents, viscosity modulating agents, diluents, filling agents, lubricants, glidants, plasticizers, solubilizers, stabilizers, suspending agents, surfactants, viscosity enhancing agents, and wetting agents. Pharmaceutical compositions may be sterile and/or pyrogen-free. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

In some embodiments, the pharmaceutical compositions as provided herein are formulated to be compatible with its intended route of administration for the methods as provided herein. The pharmaceutical compositions as provided herein may be administered in a manner appropriate to the disease to be treated (or prevented). The quantity and frequency of administration will be determined by such factors as the condition of the patient, and the type and severity of the patient's disease, although appropriate dosages may be determined by clinical trials.

In some embodiments, the compound as provided herein, the composition as provided herein, or the pharmaceutical compositions as provided herein is administered orally or parenterally. In some embodiments, the compound as provided herein, the composition as provided herein, or the pharmaceutical compositions as provided herein is administered is administered by injection. In some embodiments, The administration can be systemic administration or local administration. In some embodiments, the compound as provided herein, the composition as provided herein, or the pharmaceutical compositions as provided herein is administered is administered intravenously, intraarterially, intraperitoneally, intradermally, intracranially, intrathecally, intralymphaticly, subcutaneously, or intramuscularly. In some embodiments, the administration of the compound as provided herein, the composition as provided herein, or the pharmaceutical compositions as provided herein may be carried out in any convenient manner, including by aerosol inhalation, injection, ingestion, transfusion, implantation or transplantation. In some embodiments, the compound as provided herein, the composition as provided herein, or the pharmaceutical compositions as provided herein may be administered to a patient trans arterially, subcutaneously, intradermally, intratumorally, intranodally, intramedullary, intramuscularly, by intravenous (i.v.) injection, or intraperitoneally.

Pharmaceutical compositions suitable for injectable use include sterile aqueous solutions (where water soluble) or dispersions and sterile powders for the extemporaneous preparation of sterile injectable solutions or dispersion. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. For example, depending on the injection site, the vehicle may contain water, synthetic or vegetable oil, and/or organic co-solvents. In certain instances, such as with lyophilized product or a concentrate, the parenteral formulation would be reconstituted or diluted prior to administration. pH can be adjusted with acids or bases, such as hydrochloric acid or sodium hydroxide. Depot formulations, providing controlled or sustained release of a composition described herein, may include injectable suspensions of nano/micro particles or nano/micro or non-micronized crystals.

For oral administration, the compositions can be formulated in liquid or solid dosage forms and as instant or controlled/sustained release formulations. Suitable dosage forms for oral ingestion by a subject include powders, tablets, pills, granules, dragees, hard and soft shell capsules, liquids, gels, syrups, slurries, suspensions, emulsions and the like. Oral compositions generally include an inert diluent or an edible carrier. The tablets, granules, pills, capsules, troches and the like can contain any of the following ingredients, or compounds of a similar nature: a binder such as micro-crystalline cellulose, gum tragacanth or gelatin; an excipient such as starch or lactose; dissolution retardant; anti-adherants; cationic exchange resin; wetting agents; antioxidants; preservatives; a disintegrating agent such as alginic acid, Primogel, or corn starch; a lubricant such as magnesium stearate or Sterotes; a glidant such as colloidal silicon dioxide; a preservative; a colorant; a sweetening agent such as sugars such as dextrose, sucrose or saccharin; or a flavoring agent such as peppermint, methyl salicylate, or orange flavoring, each of these being synthetic and/or natural.

For administration by inhalation, the compositions are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams, emulsion, a solution, a suspension, or a foam, as generally known in the art. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustments; use of complexing agents and other techniques, such as iontophoresis, may be used to regulate skin penetration of the active ingredient. The compositions may also be formulated in rectal compositions, such as suppositories (e.g., with conventional suppository bases such as cocoa butter and other glycerides) or retention enemas.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

The dosage of the treatments to be administered to a patient will vary with the precise nature of the condition being treated and the recipient of the treatment. The scaling of dosages for human administration can be performed according to art-accepted practices. In some embodiments, the dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Adjustment and manipulation of established dosages (e.g., frequency and duration) are well within the ability of those skilled in the art. The treatment, such as those disclosed herein, can be administered to the subject on a daily, twice daily, biweekly, monthly or any applicable basis that is therapeutically effective. In some embodiments, the treatment is only on an as-needed basis, e.g., upon appearance of signs or symptoms of a disease or a disorder as descried herein.

The amount of the compound in the composition should also be in therapeutically effective amounts. The phrase “therapeutically effective amounts” used herein refers to the amount of agent needed to treat, ameliorate, or prevent a targeted disease or condition. An effective initial method to determine a “therapeutically effective amount” may be by carrying out cell culture assays (for example, using neuronal cells or cancer cells) or using animal models (for example, mice, rats, rabbits, dogs or pigs), or as measured using the methods known in the art. A dose may be formulated in animal models to achieve a concentration range that includes the IC50 (i.e., the concentration of the composition which achieves a half-maximal inhibition of symptoms) as determined in cell culture. Such information can be used to more accurately determine useful doses in humans. As is well known in the art, effective amounts for use in humans can also be determined from animal models. For example, a dose for humans can be formulated to achieve a concentration that has been found to be effective in animals. In addition to determining the appropriate concentration range for a composition described herein to be therapeutically effective, animal models may also yield other relevant information such as preferable routes of administration that will give maximum effectiveness. Such information may be useful as a basis for patient administration. The dosage in humans can be adjusted by monitoring effectiveness and adjusting the dosage upwards or downwards, as described above. Adjusting the dose to achieve maximal efficacy in humans based on the methods described herein and other methods is well within the capabilities of the ordinarily skilled artisan.

Toxicity and therapeutic efficacy of the compound as provided herein or the composition as provided herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects (the ratio LD50/ED50) is the therapeutic index. Agents that exhibit high therapeutic indices are preferred. The dosage of agents lies preferably within a range of circulating concentrations that include the ED50 with little or no toxicity. While agents that exhibit toxic side effects may be used, care should be taken to design a delivery system that targets such agents to the site of affected tissue in order to minimize potential damage to uninfected cells and, thereby, reduce side effects.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals, e.g., pet and live-stock animals, such as cattle, pigs, horses, sheep, cats, dogs, mice, and/or rats; and/or birds, including commercially relevant birds such as poultry, chickens, ducks, geese, and/or turkeys.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present disclosure is dictated by and directly dependent on the unique characteristics of the compositions and the particular therapeutic effect to be achieved, and the limitations inherent in the art of compounding such an agent for the treatment of subjects.

### Kits

In an aspect, provided herein are kits including the compounds as described herein, the compositions as described herein, or the pharmaceutical compositions as described herein. The kits can further include one or more additional therapeutic regimens or agents for treating the diseases or the disorders as described herein.

Also disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include the compounds as described herein, the compositions as described herein, or the pharmaceutical compositions as described herein, and optionally in addition, with therapeutic regimens or agents described herein. Such kits optionally include an identifying description or label or instructions relating to its use in the methods described herein.

A kit typically includes labels listing contents and/or instructions for use, and package inserts with instructions for use. A set of instructions will also typically be included.

In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiment, a label is used to indicate that the contents are to be used for a specific therapeutic application. The label also indicates directions for use of the contents, such as in the methods described herein.

### EXAMPLES

In the following, the present disclosure will be described in further detail through examples. These examples are intended only to illustrate the present disclosure in more detail, and it shall be self-evident to those skilled in the art that the scope of the present disclosure is not limited by these examples. Therefore, it shall be said that the real scope of the present disclosure is defined by the appended claims and their equivalents.

According to a recent survey conducted under the Second Basic Plan for Fosterage of Health and Medical Service Technology, dementia was found to be the most important of illnesses that need to be overcome. Dementia involves the highest medical cost burdens (34.3%), most suffering on the part of the patient and family members (54.8%), highest uncertainty of cause and lack of treatment alternatives (26.4%), while also being the second most threatening illness (22.5%) only after cancer in terms of mortality and cure rates. Recent positive news from global clinical trials by pharmaceutical companies is increasing the possibility of preventing and managing Alzheimer's disease through drug therapy, and it is forecast that demand for early diagnosis and treatment will increase.

Meanwhile, in a normal situation, the blood brain barrier (hereinafter BBB) serves as a primary barrier to prevent ingress of foreign material and helps to maintain health of the human body, but in pathological conditions, the BBB functions as a barrier which obstructs treatment. Therapeutic agents to treat brain disease or maintain the current state are also blocked by the BBB. A very low percentage of 95% of drugs pass through the BBB, and are unable to effectively act on the brain. Due to this reason, patients with various brain diseases have limited choice in drug therapy. One method which may be employed to treat brain disease is to develop oil-soluble drugs. This is because oil-soluble substances are able to more readily pass through the double phospholipid structure of the cell membrane. However, this applies not only to the brain but also to other organs, meaning substantial side effects in organs other than the target organ. Further, oil-soluble substances have the advantage of being able to pass through the cell membrane of endothelial cells, but also the disadvantage of being excreted quickly through the efflux pumps in the membrane. Due to the difficulties posed by the BBB in treating patients of various brain diseases, many research teams are studying various methods for effectively administering drugs through the BBB.

Advances in nanotechnology have enabled the development of nanoparticles as precise carriers of drugs of various shapes and size. These nanoparticles are promising carriers of various therapeutic agents to parts of the body afflicted by illness, in particular to solid tumors through their EPR (enhanced permeability and retention) effect. However, in the case of blood vessels with low permeability, delivery of biologically active substances through nanoparticles remains a challenge. Specifically, the brain is thoroughly protected by the BBB, which is comprised of BCECs (brain capillary endothelial cells), pericytes, and astrocytes. Only hydrophobic molecules having a molecular weight less than 450 MW are known to be able to passively penetrate the BBB, and numerous attempts to treat central nervous system disease have been obstructed by the BBB. Accordingly, development of nanoparticles able to actively permeate the BBB is crucial.

Many attempts to transport nanoparticles to be brain have been reported. Recently, in the case of nanoparticles coated with peptide able to recognize transferrin receptors on BCECs, the rate of accumulation in the brain did not exceed 1.0% dose/g-brain. Whereas nanoparticles bound to apolipoprotein E were able to permeate the BBB and reach the neurons, important drug delivery effects were not reported.

The present disclosure provides that SAMiRNA™ (confirmed to reach solid tumors through the EPR effect) conjugated with glucose is able to achieve delivery to brain tissue.

### Example 1. DMTr-Protection, Tatra-Acetylation, DMTr-Deprotection, and 6-Alcohol Protection (Trifluoromethanesulfonate) Process of D-(+)-Glucose

### Background for Examples

### Step 1: DMTr-Protection

20.0 g of D-(+)-Glucose (111.0 mmol, 1.0 eq), and 370 ml 0.3 M Pyridine, were stirred at 60° C., followed by addition of 77 ml TEA (555.0 mmol, 5.0 eq). 45.1 g DMT-Cl (133 mmol, 1.2 eq) was dissolved in 133 ml 1M pyridine, then stirred for 20 hours at 60° C. The pyridine was vacuum concentrated and extracted, desiccated using 45 g Na₂SO₄, vacuum concentrated, isolated using a column, then vacuum concentrated to obtain Product-1.

### Step 2: Tatra-Acetylation

57 g of the compound from Step 1 (118.21 mmol, 1.0 eq) and 394 ml 0.3M pyridine were stirred at room temperature, then 247 ml TEA (1.77 mol, 15.0 eq) was added, followed by stirring with acetic anhydride for 20 hours at room temperature. The Pyridine was vacuum concentrated and extracted, desiccated using 45 g Na₂SO₄, dried, vacuum concentrated, isolated using a column, then vacuum desiccated to obtain Product-2.

### Step 3: DMTr-Deprotection

23 g of the compound from Step 2 (35.37 mmol, 1.0 eq) and 120 ml 0.3M MC were stirred at room temperature, then stirred at with 8.1 ml TFA (106.11 mmol, 3.0 eq) for 1 hour at room temperature, vacuum concentrated, isolated with a column, vacuum concentrated, and vacuum desiccated to obtain Product-3.

### Step 4:6-Alcohol Protection (Trifluoromethanesulfonate)

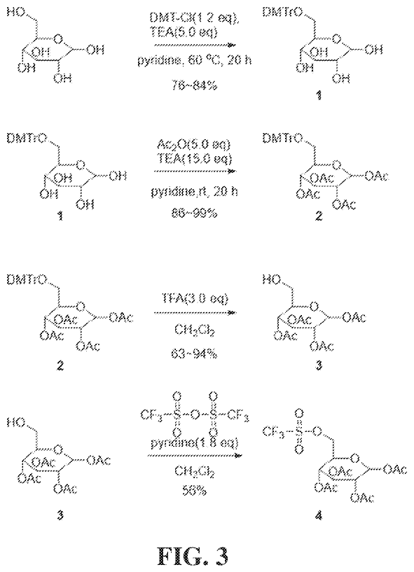

3.4 g of the compound from Step 3 (9.77 mmol, 1.0 eq) and 33 ml 0.3M MC were stirred at room temperature, followed by chilling with 1.4 ml pyridine (17.59 mmol, 1.8 eq), followed by dropping with 2.1 ml Tf₂O (12.7 mmol, 1.3 eq), extraction, desiccation with 15 g Na₂SO₄, filtration, vacuum concentration, isolation using a column, vacuum concentration, and vacuum desiccation to obtain Product-4 (FIG. 3).

### Example 2. Amine TFA-Protection (6-Aminohexnoic Acid) Process

### Step 5: Amine TFA-Protection (6-Aminohexnoic Acid)

30 g 6-Aminohexnoic acid (228.69 mmol, 1.0 eq) and 762 ml 0.3M MeOH were stirred at room temperature, stirred for 20 hours with 95.6 ml TEA (686.07 mmol, 3.0 eq) and 32.7 ml Ethyl Trifluoroacetic acid (274.43 mmol, 1.2 eq), vacuum concentrated, isolated using a column, vacuum concentrated, and stirred at pH 2 with con. HCl and filtered to obtain Product-5 (FIG. 4).

### Example 3. Amine TFA-Protection (3-amino-1,2-propanediol), DMT-Protection (3-amino-1,2-propanediol) and TFA-Deprotection (3-amino-1,2-propanediol) Process

Step 6: Amine TFA-Protection (3-amino-1,2-propanediol)

30.0 g 3-amino-1,2, propanediol (319.39 mmol, 1.0 eq) and 603 g DMF (0.5M) were stirred, then stirred with 42 g TEA (415.21 mmol, 1.3 eq) and 136 g Ethyl Trifluoroacetate (958.18 mmol, 3.0 eq) at room temperature for 1 day, vacuum concentrated and vacuum desiccated to obtain Product-6.

Step 7: DMT-Protection (3-amino-1,2-propanediol)

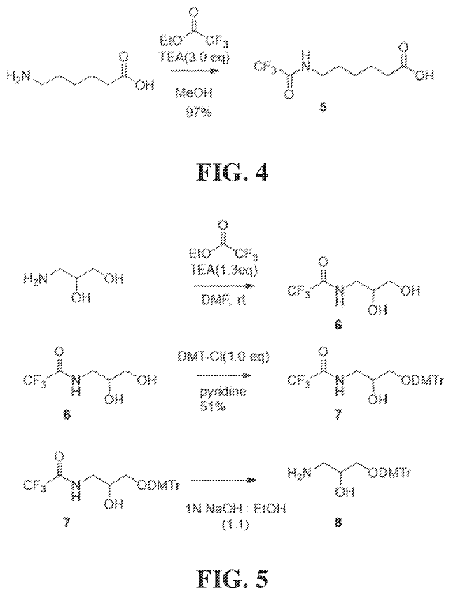

61.6 g of the compound [from Step 6] (329.27 mmol, 1.0 eq) and 98 g Pyridine were vacuum concentrated and vacuum desiccated, and stirred with 644 g Pyridine (0.5 M), followed by addition of 111.56 g DMT-Cl (329.27 mmol, 1.0 eq), stirring for 1 day at room temperature, vacuum concentration, extraction, desiccation, filtration, vacuum concentration, isolation using a column, vacuum concentration, and desiccation to obtain Product-7 (FIG. 5).

Step 8: TFA-Deprotection (3-amino-1,2-propanediol)

67.8 g of the compound from Step 7 (138.51 mmol, 1.0 eq) and 329 g EtOH (0.33M, EtOH: 1N NaOH=1:1) were stirred, stirred for 3 hours at room temperature with 416 g 1N NaOH (415.54 mmol, 3.0 eq), extracted, desiccated, filtered, vacuum concentrated, and vacuum desiccated to obtain Product-8.

### Example 4. Amide Coupling (6-Aminohexnoicacid+3-amino-1,2-propanediol) and Amine-TFA Deprotection (6-Aminohexnoicacid+3-amino-1,2-propanediol) Process

Step 9: Amide Coupling (6-Aminohexnoicacid+3-amino-1,2-propanediol)

5.0 g of the compound from Step 5 (22.02 mmol, 1.0 eq) was dissolved in 74 ml 0.3M MC, then stirred for 10 minutes after adding 5.9 g DCC (28.62 mmol, 1.3 eq) and 3.87 g HOBt (28.62 g, 1.3 eq), then stirred for 20 hours at room temperature with 6.1 ml TEA (44.04 mmol, 2.0 eq) and 11.26 g of the compound from Step 8 (28.62 mmol, 1.3 eq), concentrated, purified with a column, and vacuum desiccated to obtain Product-9.

Step 10: Amine-TFA Deprotection (6-Aminohexnoicacid+3-amino-1,2-propanediol)

12.4 g of the compound from Step 9 (20.59 mmol, 1.0 eq) was stirred over heat for 6 hours with 100 ml MeOH and 200 ml NH₄OH, concentrated, and vacuum desiccated to obtain Product-10.

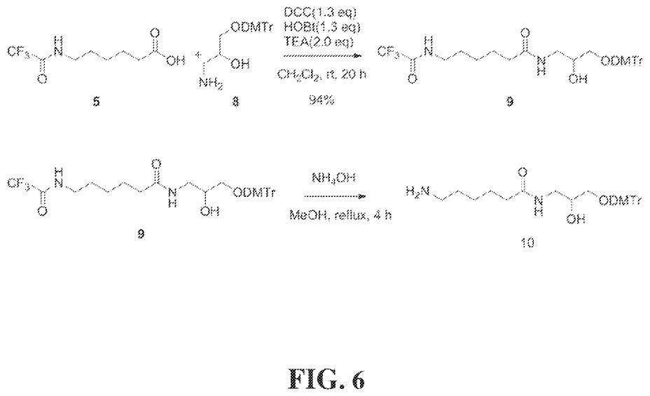

FIG. 6 illustrates the Amide coupling (6-Aminohexnoicacid+3-amino-1,2-propanediol) and Amine-TFA deprotection (6-Aminohexnoicacid+3-amino-1,2-propanediol) process of Example 4.

### Example 5. Amine Addition, Amine-Acetyl Protection, Succinylation, CPG Coupling and Capping Process

### Step 11: Amine Addition

2.3 g of the compound from Step 4 (4.80 mmol, 1.0 eq) was dissolved in 20 ml MC, stirred at room temperature with 2.5 ml DIPEA (14.37 mmol, 3.0 eq) and 2.7 g of the compound from Step 10 (5.28 mmol, 1.1 eq), extracted, desiccated, filtered, vacuum concentrated, isolated using a column, vacuum concentrated, and vacuum desiccated to obtain Product-11.

### Step 12: Amine-Acetyl Protection

300 mg of the compound from Step 11 (0.6 mmol, 1.0 eq) was dissolved in 8 ml pyridine, chilled, stirred for 30 minutes at −5° C.~0° C. after adding 85 μl TMSCl (1.08 mmol, 3.0 eq), stirred for 4 hours at room temperature with 251 ul TEA (1.8 mmol, 5.0 eq) and 26 μl AcCl (0.36 mmol, 1.0 eq), extracted, desiccated, filtered, vacuum concentrated, isolated using a column, vacuum concentrated, and desiccated to obtain Product-12.

### Step 13: Succinylation

172 mg of the compound from Step 12 (0.20 mmol, 1.0 eq) was stirred with 10 ml Pyridine, then stirred for 20 hours at 50° C. with 59 mg Succinic anhydride (0.98 mmol, 5.0 eq) and 12 mg DMAP (0.10 mmol, 0.5 eq), followed by vacuum concentration, extraction, desiccation, filtration, vacuum concentration, isolation using a column, vacuum concentration, and vacuum desiccation to obtain Product-13.

### Step 14: CPG Coupling and Capping

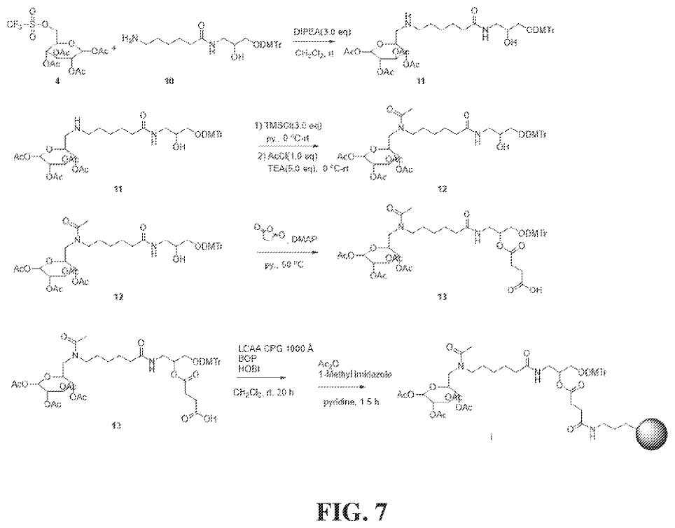

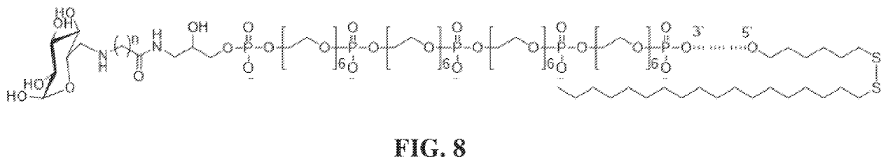

100 mg of the compound from Step 13 (0.10 mmol, 1.0 eq), 2.13 g activation LCAA-CPG (1000 Å), 66 mg BOP (0.15 mmol, 1.5 eq), 20 mg HOBt (0.15 mmol, 1.5 eq), 16 ml MC (0.01 M), and 105 ul TEA (0.75 mmol, 7.5 eq) were reacted for 1 day at 30° C., filtered, washed and vacuum desiccated. The product was reacted for 1.5 hours with 30.5 mol Pyridine (1.1 M), 2.2 ml 1-Methylimidazole (28.03 mmol, 1.0 eq) and 2.65 ml Acetic anhydride (28.03 mmol, 1.0 eq) for 1.5 hours, filtered, washed and vacuum desiccated to obtain Product-14 (FIG. 7).

### Example 6. Synthesis of SAMiRNA™ Structure Comprising Glucose-Conjugated Double Strand Oligonucleotide

The double strand oligonucleotide structure (SAMiRNA™) prepared in the present disclosure has the structure of the following Formula.
C₂₄-5′ S 3′-(hexaethyleneglycol-PO₃⁻)₄-glucose
3′ AS 5′-PO₄

The sense strand of the double strand oligonucleotide structure was prepared using the product of Example 5 as a support. 4 DMT hexaethylene glycol phosphoramidate hydrophilic monomers were continuously bonded through the above reaction, followed by RNA or DNA synthesis, and additional bonding at the 5′ terminal of C₂₄ (C₆—S—S—C₁₈) with a disulfide bond as a hydrophobic substance. Thereby, the sense strand of glucose-SAMiRNA™ where glucose-(—PO₃⁻ hexaethyleneglycol)₄ is bonded to the 3′ terminal, and C₂₄ (C₆—S—S—C₁₈) is bonded to the 5′ terminal was prepared.

After completion of synthesis, the RNA single strand and oligo (DNA or RNA)-polymer structure synthesized by treating with 28% (v/v) ammonia in a water bath at 60° C. was detached from the CPG, and the protecting residue was removed through a deprotection reaction. The RNA single strand and oligo (DNA or RNA)-polymer structure where the protecting residue was removed was treated in an oven at 70° C. with N-methylpyrolidone, triethylamine and treithylaminetrihydrofluoride at a volumetric ratio of 10:3:4 to remove 2′TBDMS (tert-butyldimethylsilyl). From the above reactants, high performance liquid chromatography (HPLC) was used to isolate the RNA single strand, oligo (DNA or RNA)-polymer structure and ligand-bonded oligo (DNA or RNA)-polymer structure. Molecular weights were measured using MALDI-TOF mass spectrometry (MALDI TOF-MS, SHIMADZU, Japan) to verify a match with the base sequence to be synthesized and the oligo-polymer structure. Then, to prepare the respective double strand oligonucleotide structures, equal amounts of sense strand and anti-sense strand were mixed and placed in 1× annealing buffer (30 mM HEPES, 100 mM potassium acetate, 2 mM magnesium acetate, pH 7.0-7.5), reacted for 3 minutes in a 90° C. water bath, then reacted again at 37° C. to prepare the intended SAMiRNA™. Annealing of the prepared double strand oligonucleotide RNA structures was confirmed using electrophoresis. The sense strand after completion of synthesis has the structure of FIG. 8. The structure of 3′ glucose-linker-hexaethyleneglycol×4-sense nucleic acid strand-C₆S—S—C₁₈ 5′ is had.

### Example 7. Preparation of Nanoparticles and Particle Size Analysis

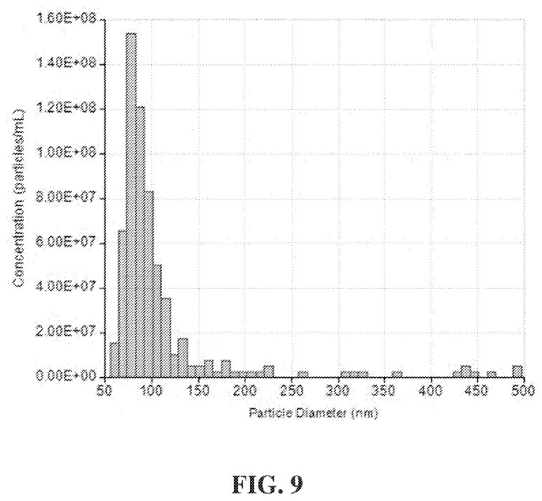

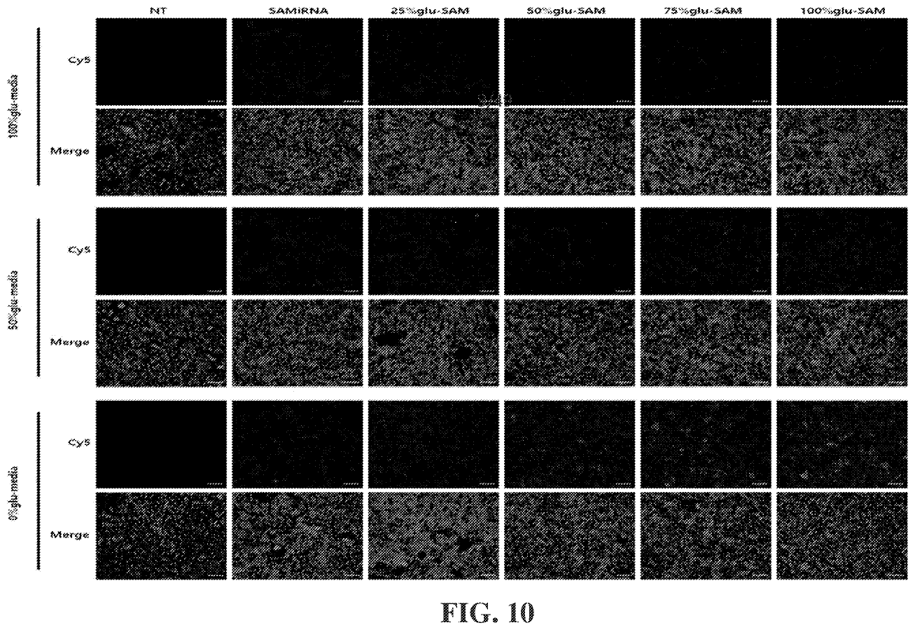

The glucose-conjugated SAMiRNA™ synthesized in Example 6 was dissolved in 1× Dulbecco's Phosphate Buffered Saline (DPBS) and freeze-dried for 5 days in a freeze-dryer (LGJ-100F). The freeze-dried nanoparticle powder was dissolved in deionized distilled water and homogenized for use in the experiment for the present disclosure. For particle size analysis of the prepared SAMiRNA™ nanoparticles, Zetasizer Nano ZS and qNANO Gold were used. Results of nanoparticle size and polydispersity index measurement for glucose-SAMiRNA™ are as shown in Table 1A, and the size distribution graph is as shown in FIG. 9.

TABLE 1A
Particle size measurements for Example 7

| Instrument | The qNano Gold |
|---|---|
| Nanopore size | NP 100 |
| Calibration particle | CPC 100 |
| Mean | 110 |
| Mode | 78 |
| Maximum | 495 |
| Minimum | 60 |
| Raw Concentration | 6.26e+08 |
| PDI | 0.289 |

### Example 8. Efficiency of Cell Entry by Glucose-SAMiRNA™ Nanoparticles

Neuro2a mouse neuroblastoma cells were cultured for a day in normal glucose concentration (4.5 g/L), ½ concentration (2.25 g/L) or glucose-free medium with 10% FBS, then treated for 1 day with 1 uM of SAMiRNA™-cy5 or SAMiRNA™ bonded with 25, 50, 75 or 100% glucose under like culture conditions as the previous day. After 1 day, the cells were washed with PBS, fixed for 5 minutes using 4% para-formaldehyde, then mounting using mounting media with DAPI for fluorescent imaging using confocal equipment. Blue, DAPI; Red, Cy5; Magnification, ×100; Bar, 100 um. (LSM 880, Zeiss).

In the results, as shown in FIG. 10, it was found that efficiency of cell entry was improved as the glucose conjugation % of SAMiRNA™ increased in the glucose-free medium. Thereby it was indirectly proven that the entry of glucose-SAMiRNA™ into cells is dependent on cells' glucose receptors.

### Example 9. Evaluation of Glucose-SAMiRNA™ Nanoparticle Delivery in Mouse

### Example 9-1. Preparation of Animal Model and Administration of Nanoparticle

To observe delivery of glucose-SAMiRNA™ nanoparticle at dates following administration to different parts of the brain, Balb/c 6-week-old female mice purchased from Nara biotech were divided into the following 3 groups: (1) Balb/c+Vehicle (n=2), (2) Balb/c+glucose-SAMiRNA™ 40 mpk (n=1, N=7), and (3) Balb/c+glucose-SAMiRNA™-cy5 40 mpk (n=1, N=7). The mice were fasted for 24 hours prior to administration, then glucose-SAMIRNA™ or glucose-SAMiRNA™-cy5 dissolved in PBS were administered to groups (2) and (3), respectively. The same amount of PBS was administered to group (1). 3 hours after administration, 200 ul of 20% (w/v) glucose was administered to all groups by intraperitoneal injection. For performance of Examples 9-2 through 9-5 on group (3), mice in each group were anesthetized at 1, 2, 3, 4, 5, 6 and 7 days after administration, PBS perfusion was performed through the heart to remove any residual particles in the blood vessels, and perfusion fixation was carried out using 10% neutral buffered formalin. For performance of Example 9-6 and PK quantification through stem-loop qRT-PCR on group (2), mice in each group were anesthetized at 1, 2, 3, 4, 5, 6 and 7 days after administration, PBS perfusion was performed through the heart to remove any residual particles in the blood vessels. Then serum, CSF and brain tissue were sampled, with the brain tissue divided into the 6 following regions: (1; left hemisphere, 2; right olfactory groove, 3; right cerebral cortex, 4; right sub-cortex region-hippocampus, thalamus, hypothalamus, midbrain, choroid plexus 3V, 5; right cerebellum, choroid plexus 4V, 6; right medulla-pons).

### Example 9-2. Ex-Vivo Brain Imaging

To evaluate biofluorescence in forebrain tissue by day following administration of glucose-SAMiRNA™, brain tissue obtained from group (3) of Example 9-1 was scanned using the IVIS 200 imaging system (Xenogen, Caliper Life Sciences). Cy5 excitation (λex=640 nm) and emission (λem=700 nm) filter was used, and the strength of fluorescence for each brain tissue was quantified using the Living Image software package (Caliper Life Sciences). Brain emission efficiency was measured (photons/second/cm2/sr)/uW/cm2), this indicating emission/illumination power density.

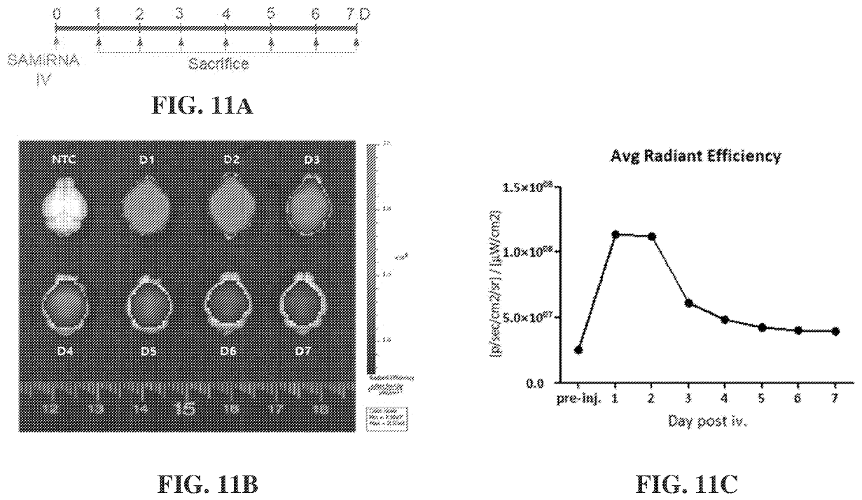

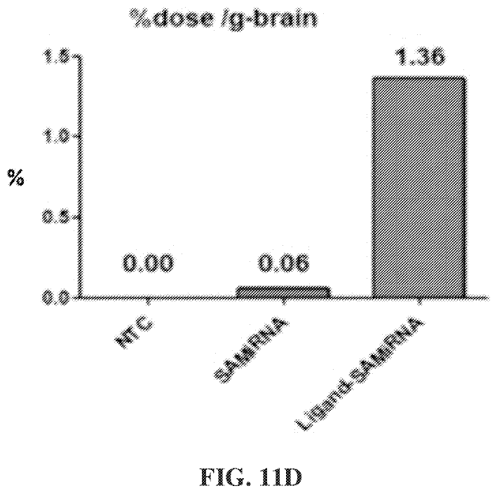

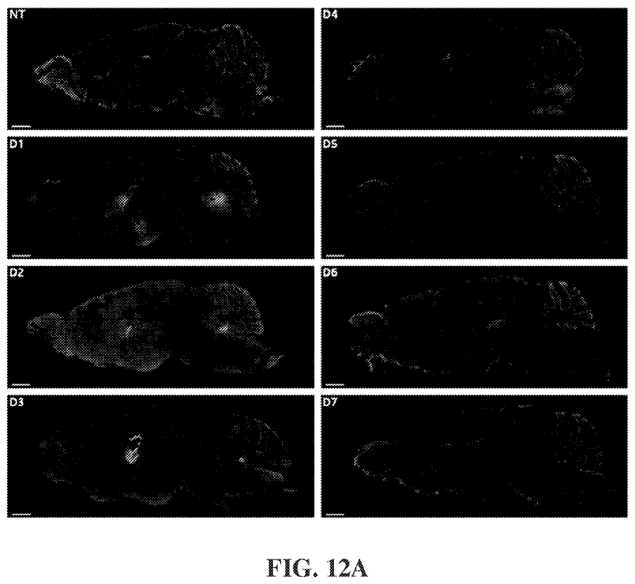

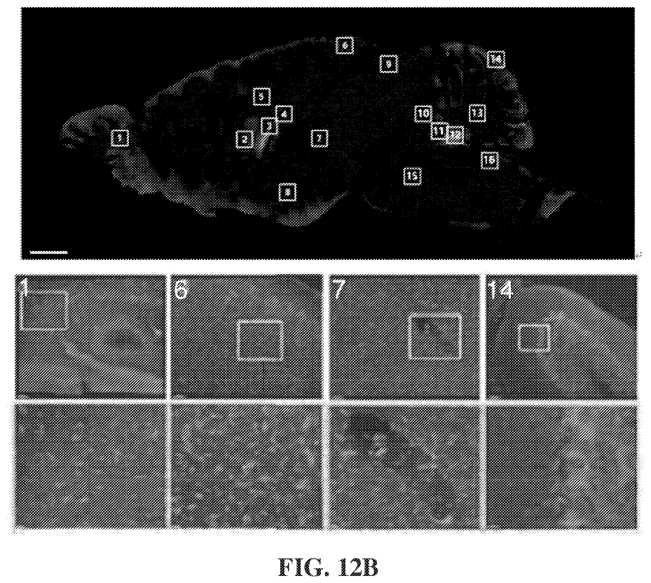

Results of imaging are as shown in FIG. 11B and FIG. 11C. As shown in the quantification analysis graph, the strongest fluorescent signals were observed on the first and second days after administration, with a gradually decreasing trend.

### Example 9-3. Quantification Analysis

For quantification analysis of the glucose-SAMiRNA™-cy5 delivered to the brain, the left hemisphere of the samples from 1 day after administration imaged in Example 9-2 was washed with D-PBS(−). Excess moisture was removed before weighing and homogenization in lysis buffer (Promega Corporation). The amount of glucose-SAMiRNA™-cy5 accumulated was quantified using an HPLC fluorescence detector (Shimazu).

In the results, shown in FIG. 11D, the amount of glucose-SAMiRNA™-cy5 delivered to the brain was measured to be 1.36% of the amount administered per unit gram brain tissue.

### Example 9-4. Immunofluorescence Imaging

To examine the delivery of the glucose-SAMiRNA™ to each part of the brain, the brain tissue after ex-vivo brain imaging in Example 9-2 was additionally fixed for 20 hours at 4° C. using cold 4% paraformaldehyde in 0.1M phosphate buffer, then immersed in an anti-freeze solution of 30% sucrose in PBS. The settled tissues were removed and placed in OCT solution, then tissue molds frozen in liquid nitrogen were sliced along the sagittal plane into 30 μm slices at −25° C. using a cryostat (Leica Biosystems). The sliced tissues were mounted using a mountain solution containing DAPI, and fluorescent signals were imaged (Blue; nucleus, Red; cy5, Device; DragonFly).

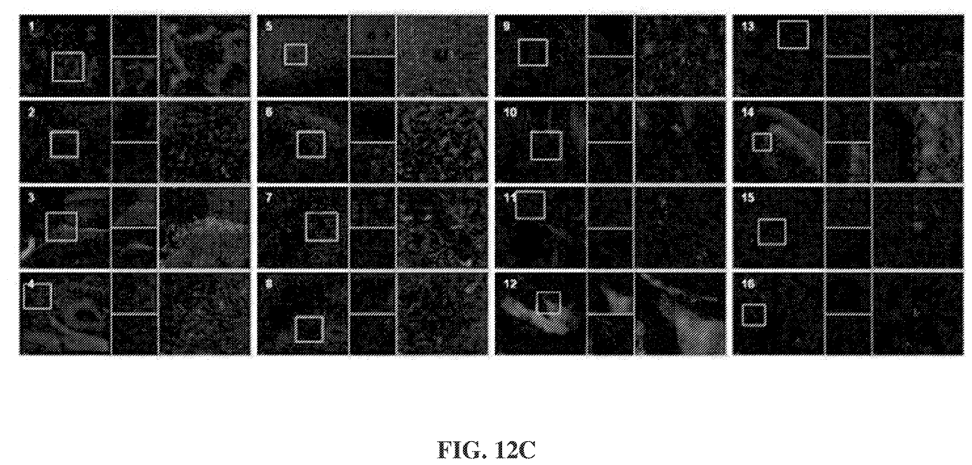

In the results, as shown in FIG. 12A, the strongest fluorescence was observed at the choroid plexus until the 4th day after administration, with the most even distribution across all brain tissue on the second day and a tendency for slow general reduction in fluorescence from day 5 onwards.

To examine each region of the brain more closely on day 2, when the most even distribution was achieved, 16 brain tissues were imaged under magnification.

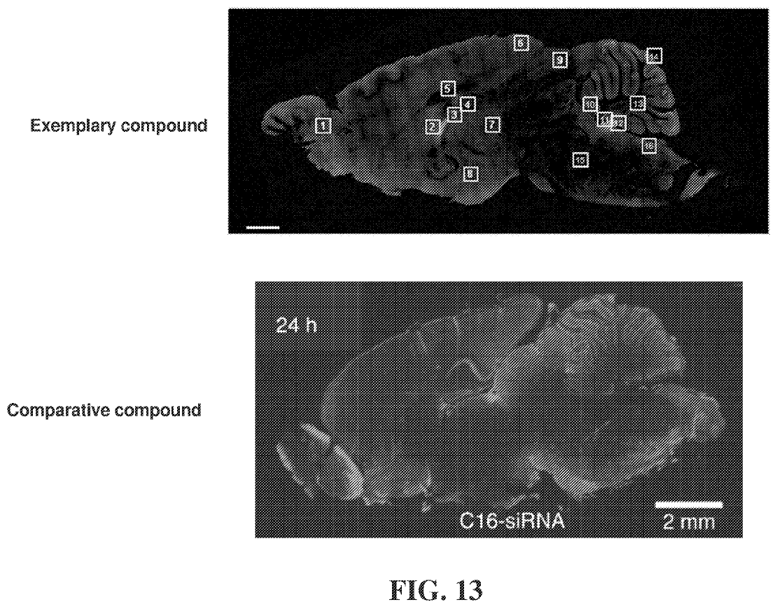

Different results were observed for each region, as shown in FIG. 12B and FIG. 12C. First, in the gray matter with generally high distribution of cell nuclei (4; Hippocampus, 5; Cerebral cortex white, 6; Cerebral cortex gray, 7; Thalamus, 8; Hypothalamus, 9; Midbrain, 13; Cerebellal nuclei, 14; Cerebellal cortex), relatively greater amounts of fluorescent signal were observed. On the other hand, in the white matter with relatively less cell nuclei, relatively less fluorescence was observed (1; Olfactory bulb, 2; Septum, 15; Pons, 16; Medulla). Also, strong fluorescence was observed in the cerebral cortex and hippocampus, which are reported as the key regions for occurrence of Alzheimer's disease (4; Hippocampus, 5; Cerebral cortex white, 6; Cerebral cortex gray). From these results, it was confirmed that relatively greater amounts of glucose-SAMiRNA™ were delivered to gray matter regions with greater distribution of cell nuclei and more active glucose intake, including Alzheimer's Disease sites. FIG. 13 shows an exemplary compound compared to a comparative compound, thereby showing potential for wide range of CNS indications enabled by effective delivery and distribution of an exemplary compound as provided herein in the brain.

### Example 9-5. Immunofluorescence Staining of Tissue

To observe the passage of glucose-SAMiRNA™ delivered into brain tissue through cerebral blood vessels and entry into cerebral neurons, the day 2 samples among the tissue slices of Example 9-4 were permeabilizated for 30 minutes using 0.3% Triton X-100, washed 3 times for 5 minutes using PBS, then blocked for 30 minutes using 0.5 mg/mL bovine serum albumin (PBS). Then, the tissues were washed with PBS 3 times for 5 minutes, then incubated overnight at 4° C. with one of the following primary antibodies: Using Rabbit anti-GFAP antibody (1:200; Abcam), Mouse anti-Tuj1 (1:100; Covance), and Rat anti-PCAM (1:200; BD) reactions were carried out using PBS containing 0.5 mg/mL BSA and 0.3% Triton X-100 at individual dilution concentrations. Then, the slices were incubated for 50 minutes with a secondary antibody at room temperature: Alexa 488-marked anti-Rabbit IgG, 568-marked anti-Rat IgG, and 488-marked anti-Mouse IgG (Thermo Fisher Scientific) were diluted using PBS containing 1:500 0.3% Triton X-100. The stained tissues were placed on slides, and cover slipped using a fluoroshield mounting medium containing 4,6,-diamidino-2-phenylindol (DAPI; Sigma-Aldrich). To quantify immune response, images were captured using a Dragonfly microscope (Andor).

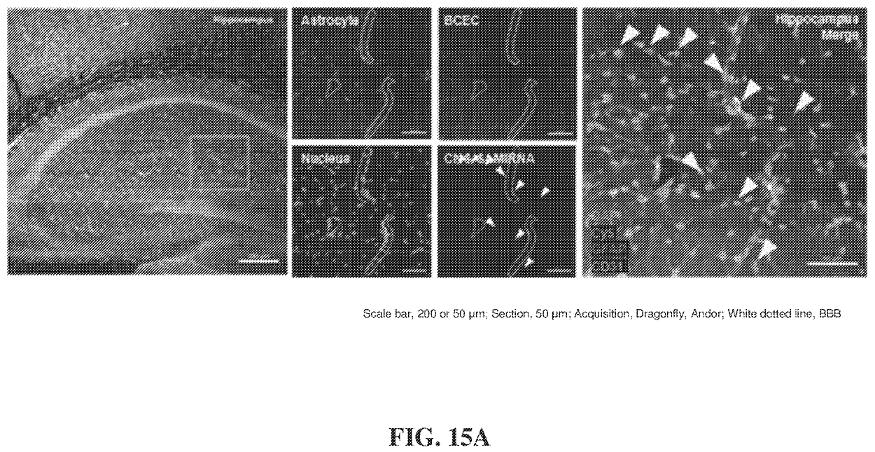

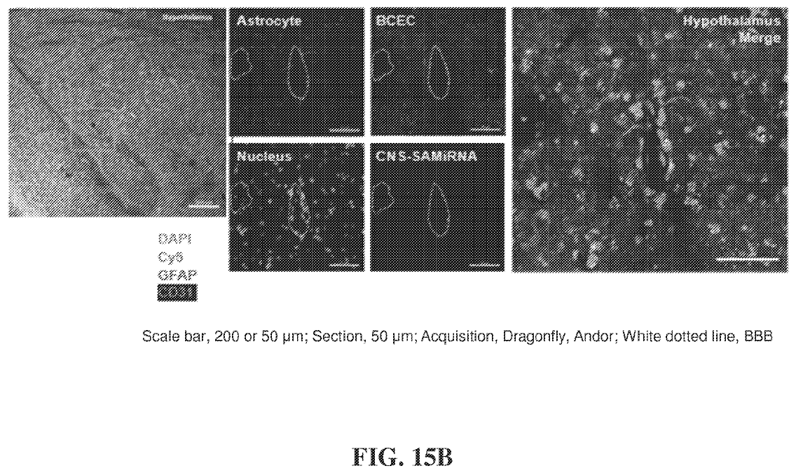

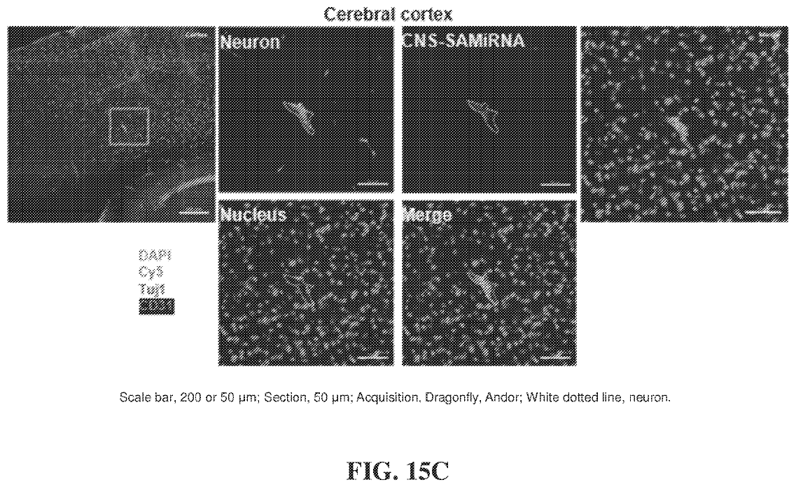

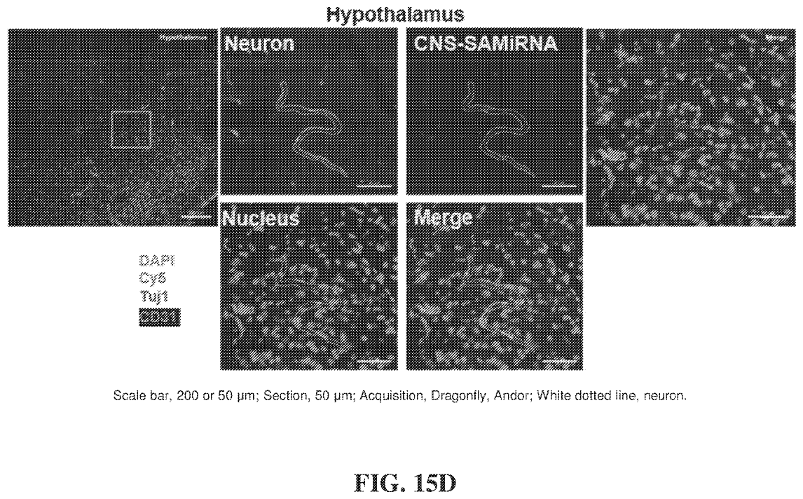

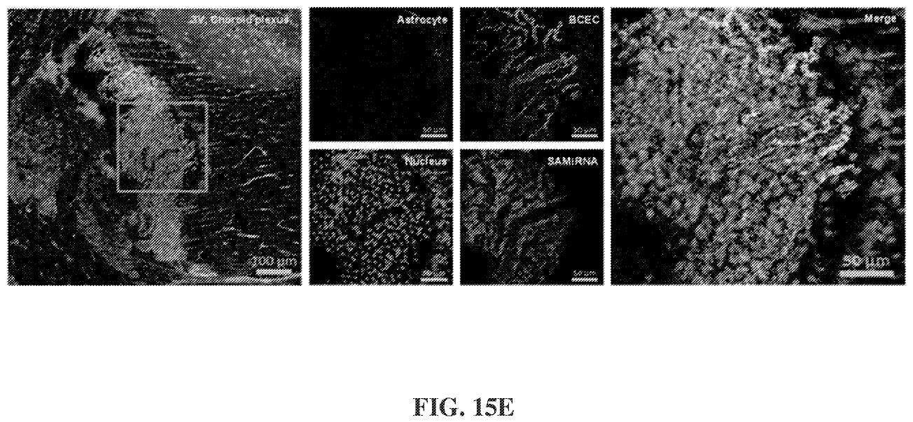

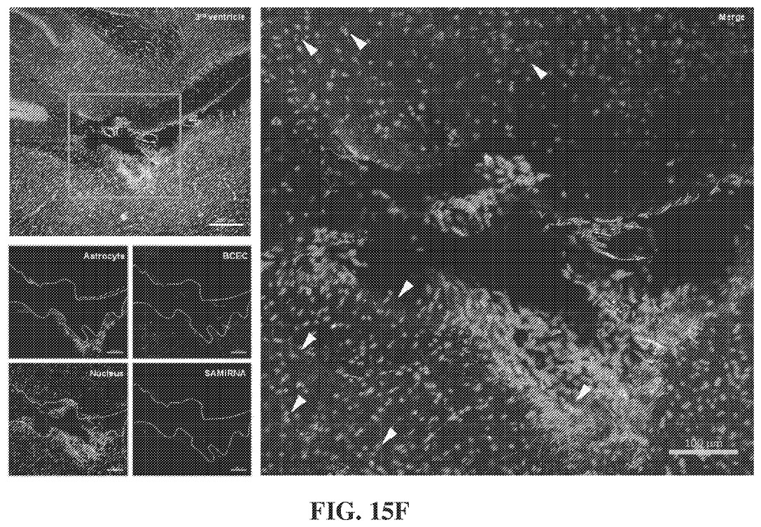

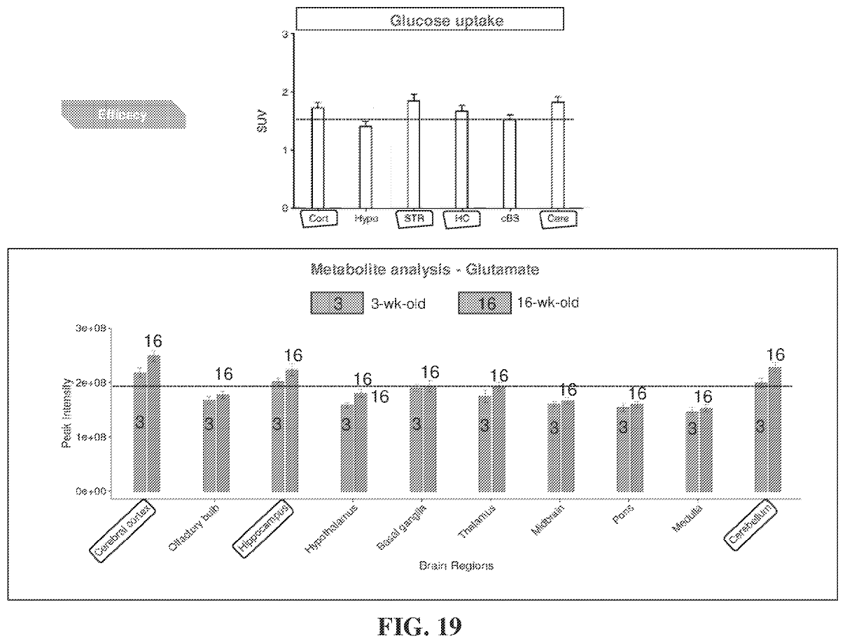

In the results, as shown in FIG. 15A-FIG. 15F, glucose-SAMiRNA™ was observed outside the blood vessels and the surrounding astrocytes (FIG. 15A; hippocampus, FIG. 15B; hypothalamus). Also, co-localization with brain neurons was observed (FIG. 15C; cortex, FIG. 15D; hypothalamus). Further, observing the magnified choroid plexus of FIG. 12A, FIG. 12B, and FIG. 12C, deposition on the ependymocytes around the blood vessels was verified (FIG. 15E; 3^{rd} ventricle choroid plexus), and imaging of the surrounding ventricles verified transport past the CSF-brain barrier into the parenchyma (FIG. 15F; 3^{rd} ventricle). FIG. 19 shows glutamate metabolism in the indicated brain regions, thereby identifying a non-limiting list of brain regions for use in targeting with a glucose-conjugated SAMiRNA™.

### Example 9-6. PK Study

For comparative quantification of the glucose-SAMiRNA™ delivered into the brain tissue by brain region and days after administration, the serum, cereobrospinal fluid, and brain tissue samples from 6 different regions in group (2) of Example 9-1 were used to carry out the following comparative quantification test.

The tissue samples were added to NP-40 lysis buffer (GENDEPOT) with RNase inhibitor (ambion) and Proteinase inhibitor cocktail (Roche), then homogenized using a homogemizer (IKA). The homogenized samples were shredded once more using a QIAshredder (QIAGEN). Ago 2 immunoprecipitation was carried out on the tissue lysate using MagListo™ Protein G Kit with anti-mouse AGO2 antibody (Sigma) attached. Absolute quantification was performed on the obtained RISC-loaded siRNA using stem-loop RT-qPCR. After first converting to cDNA using a Taqman MicroRNA Reverse transcription kit (ABI), Mygenie™ 96 and antisense RT primer, AccuPower® 2× GreenStar™ qPCR Master Mix, 600 nM qPCR primer and Exicycler™ 96 were used to carry out qPCR. The reference material used for quantification was used to spike a non-treated sample, and used for analysis through the same process. The RT and qPCR primer sequences used for analysis are as shown in Table 1B.

TABLE 1B
RT and qPCR primer sequences

| | | SEQ ID |
|---|---|---|
| | Sequence | NO: |
| RT primer | 5′- | 1 |
| | GTCGTATCCAGTGCAGGGTCCGAGGTATT | |
| | CGCACTGGATACGACACGGGA-3′ | |
| Forward | 5′-GCGATGGCATGCACAGTC-3′ | 2 |
| Reverse | 5′-GTGCAGGGTCCGAGGT-3′ | 3 |

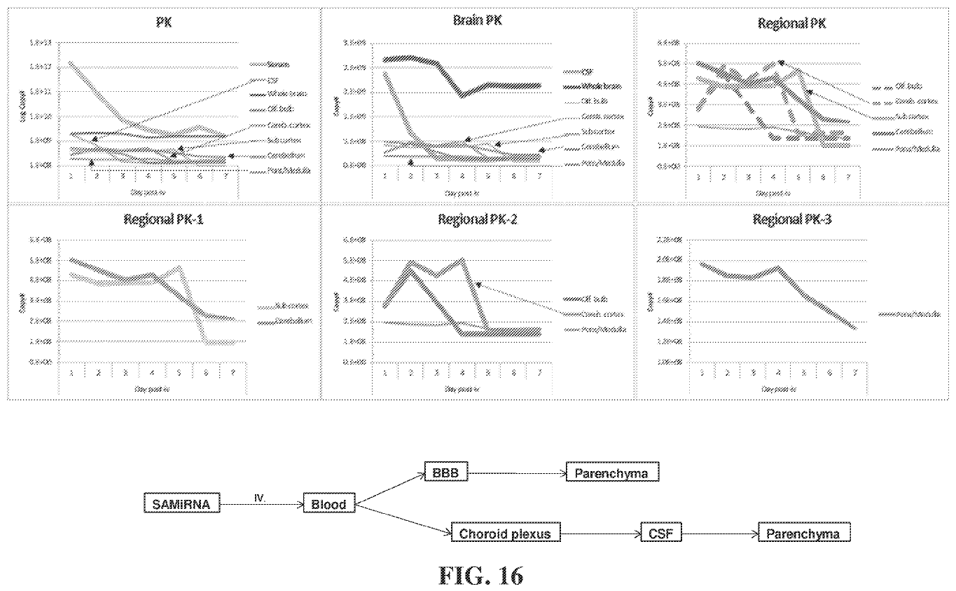

In the results, the greatest amount was measured in the serum (purple) as shown in FIG. 16 (Upper, left graph), with a slow decreasing trend from the 4th day after administration onward (A). Magnifying the portion excluding the serum (dotted line box beneath A), the largest amount was measured in the forebrain and cerebrospinal fluid, with a tendency for levels to hold until the 4^{th} day after administration in the forebrain (black) and a rapid decreasing tendency on the 2^{nd} day after administration in the case of the cerebrospinal fluid (gray). Examining the regions for each part of the brain (dotted box beneath B), as shown in C, relatively high amounts of glucose-SAMIRNA™ were detected from the first day after administration in the sub cortex (blue), which is the sample group including the choroid plexus, hippocampus, thalamus, hypothalamus, and midbrain, and the cerebellum (light blue), with levels holding until day 5. In the sample groups not including the choroid plexus, the cerebral cortex (red) and olfactory groove (orange), the relative amount which was low on day 1 increased starting on day 2, with levels holding until day 3 and day 4, respectively. In contrast, in the medulla and pons, with nerve fiber bundles and relatively few cell nuclei, relatively low amounts delivered were observed compared to other regions over 7 days.

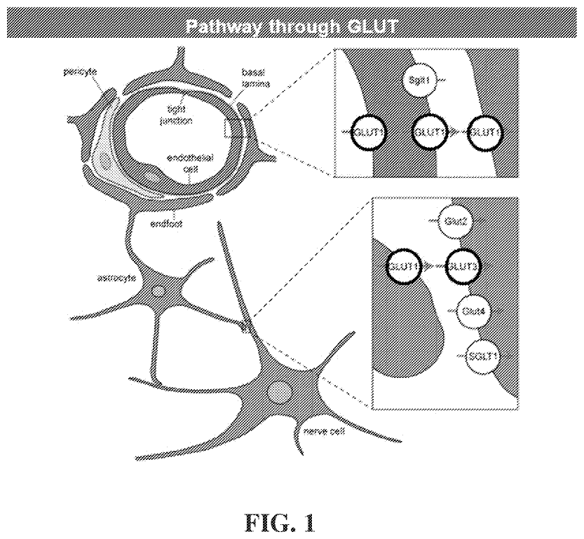

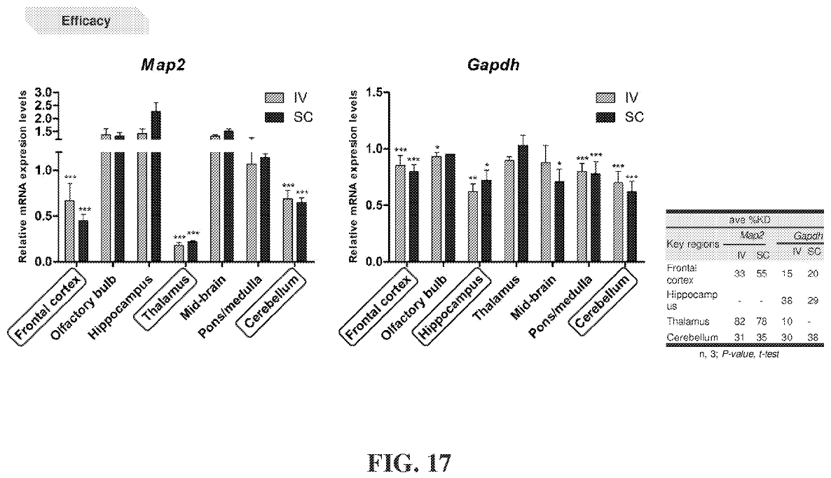

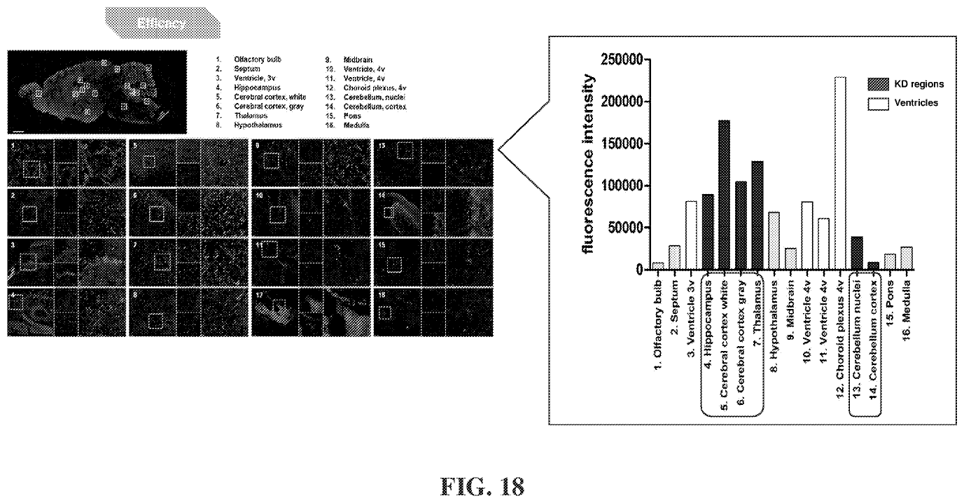

Analyzing these results as shown in FIG. 12A, FIG. 12B, FIG. 12C, and FIGS. 15A-15F, 16, 17, 18, and 19, glucose-conjugated SAMiRNA™ administered through the lateral tail vein directly passed through the cerebrovascular BBB (FIG. 15A, FIG. 15B) and was delivered to cerebral nerve cells (FIG. 15C, FIG. 15D), and was strongly deposited on the ependymocytes of the choroid plexus early on after administration (Days 1 through 3) (FIG. 12A, FIG. 12B, FIG. 12C, FIG. 15E). As shown in FIG. 17 and FIG. 18, SAMiRNA™ targeting Map2 passed through the BBB and knocked down expression of Map 2 and that achieving knockdown has. The primary function of these cells is to circulate nutrition from the blood vessels through whole blood tissue through production and secretion of cerebrospinal fluid. Also, the barrier between the cerebrospinal fluid and parenchyma (brain-CSF barrier), unlike the tight junctions of the BBB, is comprised of gap junctions. Accordingly, a pathway wherein the [glucose-conjugated SAMIRNA™] circulates in the cerebrospinal fluid (see FIG. 1) which is secreted actively by the ependymocytes of the choroid plexus whereon high levels of deposition were confirmed, then passes through the brain-CSF barrier which is comprised of gap junctions into the brain tissue can be assumed, and observing the tissues around the 3^{rd} ventricle, substantial infiltration of glucose-SAMiRNA™ into the tissue can be seen (FIG. 15F). As shown in FIG. 16 (Upper, middle graph), this can be supported by the reduction in the relatively high amounts of glucose-SAMiRNA™ detected in the cerebrospinal fluid, which begins on day 2 after administration, and the increase in levels in the cerebral cortex and olfactory bulb occurring at the same time, as shown in FIG. 16 (Upper, right graph). This region was dissected not to include the choroid plexus. In contrast, in the subcortex and cerebellum, which include the choroid plexus, relatively high amounts of glucose-SAMiRNA™ were measured from day 1. Ultimately, the glucose-conjugated SAMiRNA™ administered through the lateral tail vein was confirmed to directly permeate the BBB (FIG. 15A, FIG. 15B) and be delivered to brain nerve cells, and to be simultaneously delivered to parenchymal tissues through cerebrospinal fluid circulation by the ependymocytes of the choroid plexus.

### Example 10. Effective Intravenous Delivery of siRNA Across BBB Using CNS-SAMiRNA™ Platform Improved Alzheimer's Disease (AD)-Associated Markers and Behavioral Scores in 3×Tg-AD Mice

### Example 10-1. Brain Delivery Poses a Major Challenge in RNAi Therapeutic Development for CNS Indications Like AD

Alzheimer's disease (AD) is a common, progressive neurodegenerative disease in which abnormal proteins such as amyloid plaques and neurofibrillary tangles accumulate in the brain, leading to the destruction of neuronal cells and loss of cognitive functions.

Developing therapies for CNS diseases such as AD has been a major challenge due to difficulties in getting drugs to cross the blood-brain barrier (BBB). BBB helps maintain the complex physiology of the brain by selectively passing only the essentials for maintaining brain function.

Most therapeutic classes, including small interference RNA (siRNA), do not cross the BBB efficiently enough to achieve therapeutic efficacy. siRNA is an exciting class of oligonucleotide-based therapies that harnesses the innate viral defense mechanism to knock down protein expression. Previously, safety, stability, bioavailability, manufacturing and delivery of siRNA was analyzed with the proprietary siRNA delivery platform technology, SAMiRNA™.

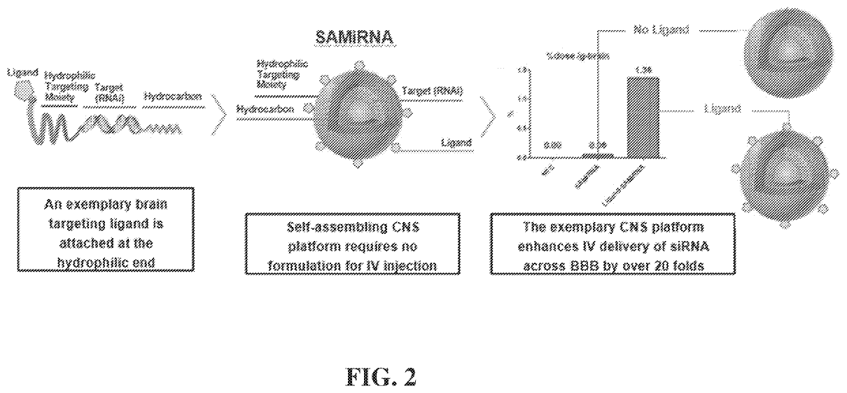

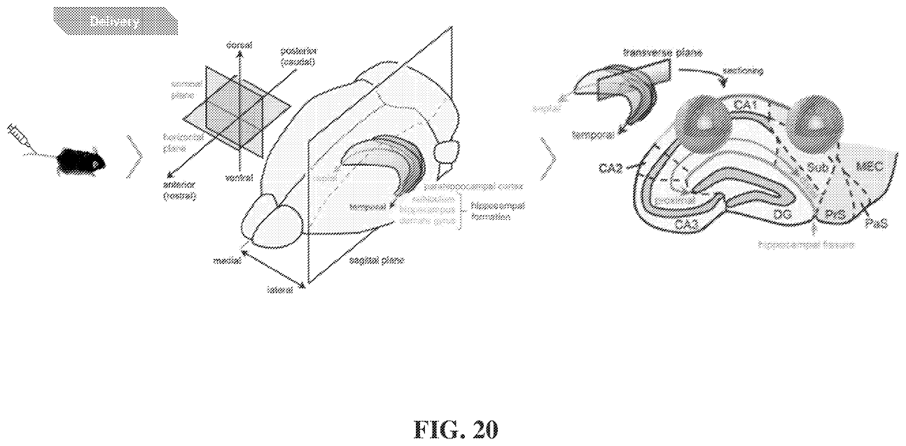

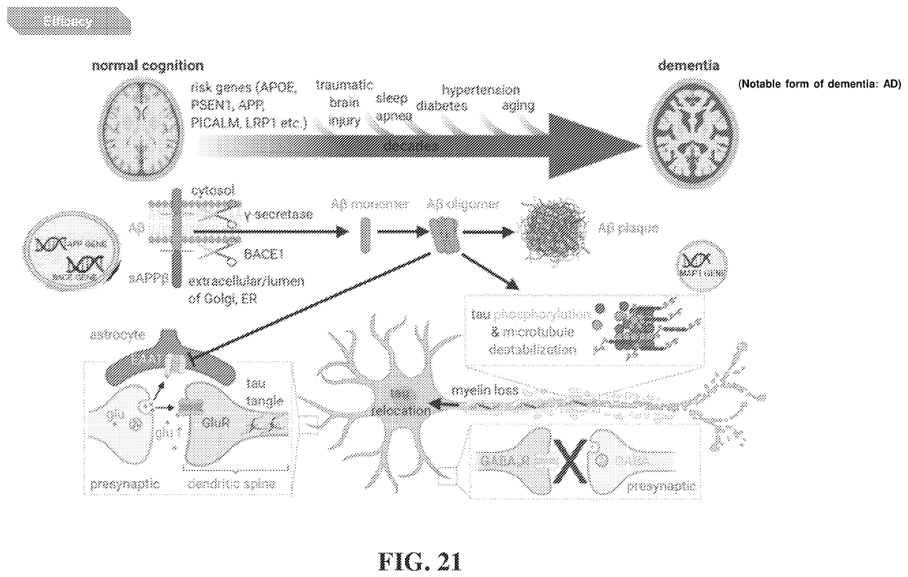

To overcome the challenges around delivery across the BBB, herein a targeting ligand was incorporated onto the SAMiRNA™ platform to enhance intravenous (IV) delivery of RNAi across BBB by 20 folds (see FIG. 1 and FIG. 2). FIG. 20 and FIG. 21 represent non-limiting example schematics of the workflow and mechanism of action of the compounds and methods of use as described herein. A therapeutic candidate SRN-008, an exemplary compound as provided herein, for AD using this platform improved the behavior and the AD-associated markers in 3×Tg AD mouse model (FIG. 2).

### Example 10-2. Methods

### Immunofluorescence Labeling

To investigate immunoreactivity, floating brain sections were each rinsed three times for 5 minutes. The brain tissues were permeabilized with 0.3% triton X-100 for 30 minutes, washed 3 times for 5 minutes with PBS, and blocked with a 0.5 mg/mL bovine serum albumin (BSA) for 30 minutes. Then, the tissues washed 3 times for 5 minutes with PBS and incubated with primary antibodies overnight at 4° C. Subsequently, sections were incubated with the following secondary antibodies for 50 min at room temperature. Immunostained tissues were mounted on slides and cover-slipped with the Fluoroshield mounting medium with DAPI.

Animals and administration. 3×Tg mice purchased at Jackson Laboratory (Bar Harbor) express three familial AD mutations (APPSwedish, MAPTP301L and PSEN1M246V). 8-month-old female 3×Tg mice and 10-week-old female B6129F1/J mice were used in this study and divided into three groups: (1) B6129F1/J+Vehicle (n=9), (2) 3×Tg+Vehicle (n=10), and (3) 3×Tg+SRN-008 (n=10). We IV administered SRN-008 at 40 mg/kg once a week for 4 weeks. SRN-008 was dissolved in PBS, and the same amount of PBS was administered to the vehicle group.

Behavioral memory impairment test. Short-term spatial memory was examined using a Y-maze 24 h after the final administration of an exemplary compound as provided herein, SRN-008. The Y-maze is a three-arm maze with equal angles between all arms (30 cm long×3 cm wide×14 cm high). The floor and walls of the maze were constructed using black acrylic. Mice were placed in the center of the Y-maze and allowed to explore each arm freely. Spontaneous alternation and the total number of arm entries were manually counted for each mouse during the 8 min session. The total number of arm entries was used as an indicator of locomotor activity. Spontaneous alternation was defined as successive entries into all arms (i.e., ABC, CBA, or BAC) without repetitions (i.e., ABA or CBC). The percentage of alternations was calculated as [(the number of alterations)/(the total number of arms entered-2)]×100.

Image acquisition and analysis. To quantify immunoreactivity, images were captured with a Zeiss LSM 700 microscope (Carl Zeiss AG) and analyzed using ImageJ software (National Institutes of Health). The analysis was performed blinded to the hippocampal formation of each animal's brain section. To measure the 4G8, AT8, and HT7 signals, we quantified the area fractions of the immune-positive signals in the CA1 and subiculum.

Statistical analysis. All analyses were randomly conducted in a blinded manner for each group. Statistical analyses were performed using GraphPad Prism 7.0 software (GraphPad Software, Inc.). Data are presented as mean±standard error of the mean. The significance of differences between the groups was determined by an independent t-test or one-way analysis of variance, with either Tukey's post hoc test. Differences were considered statistically significant at p<0.05.

### Example 10-3. CNS-SAMIRNA™ Platform Significantly Enhances IV Delivery of siRNA Across Blood-Brain Barrier (BBB)

### Immunofluorescence Labeling

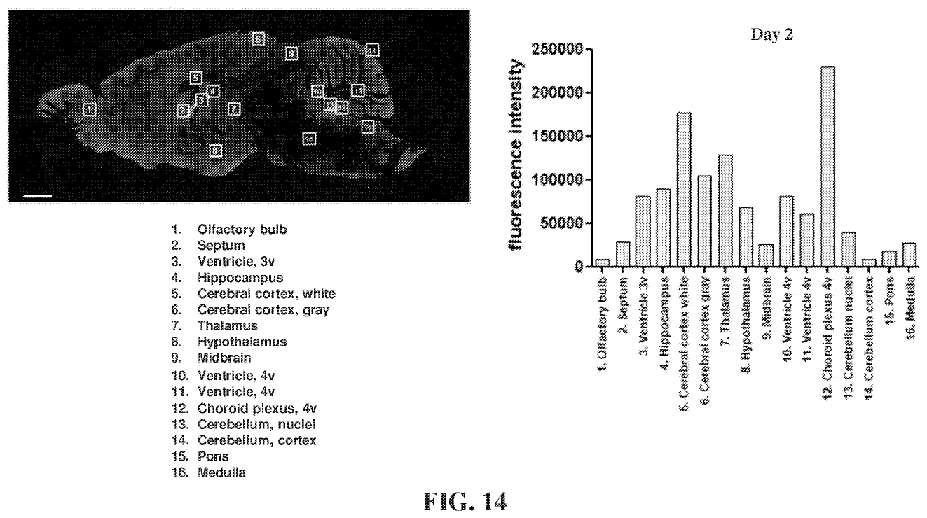

The delivery of fluorescently tagged CNS-SAMIRNA™ construct to the healthy mice brain was confirmed by confocal imaging and quantitative HPLC. Fluorescence could be detected in the brain region for over a week and 1.36% of the dose per g-brain could be delivered compared to 0.06% with no BBB-targeting ligand. Higher depositions were observed in gray matter where cell bodies are concentrated, such as hippocampus (#4), cerebral cortex (#6), and thalamus (#7). In the cerebellar cortex (#14), deposition appeared to concentrate in purkinje cells (FIGS. 11A-11D, FIGS. 12A-12C, FIGS. 15A-15D; FIG. 17). Additional data is shown in FIG. 14.

### Example 10-4. Candidate SAMiRNA Selection

### Example 10-4A: Selection of Candidate Sequences

SAMiRNA-based drug high-throughput screening involves applying a 1-base or 2-base sliding window algorithm to the entire mRNA transcriptome to generate all possible candidate sequences. Homology filtering is then performed to eliminate unnecessary candidate sequences, followed by evaluating the degree of gene expression inhibition for all selected SAMiRNAs. For this study, a 1-base or 2-base sliding window algorithm was applied to human and mouse genes of interest, resulting in 19-base candidate sequences. Homology filtering was performed to select the most promising candidate sequences for further analysis of their inhibitory effects.

### Example 10-4B: Intracellular Treatment of Nanoparticles

To discover SAMiRNAs that inhibit the expression of human BACE1 and MAPT genes, human neuroblastoma cell line SH-SY5Y A549 (KCLB #22266, Korean Cell Line Bank) was used. For the discovery of mouse Bace1 SAMiRNAs, B16-F0 mouse melanoma cell line (ATCC® CRL-6322, Manassas, VA, USA) was utilized, and for the discovery of mouse Mapt SAMiRNAs, 4T1 mouse breast cancer cell line (ATCC® CRL-2539, Manassas, VA, USA) was employed. SH-SY5Y and 4T1 cells were cultured in Gibco™ RPMI-1640 medium (Thermo, US) supplemented with 10% Fetal Bovine Serum (Hyclone, US) and 1% Penicillin-Streptomycin (Hyclone, US), while B16-F0 cells were cultured in Gibco™ DMEM medium under the same conditions at 37° C. with 5% CO2. SH-SY5Y cells were seeded in a 24-well plate (Costar, US) at a density of 1×10\hat{ }5 cells/well using the same medium. The next day, SAMiRNAs, which were homogenized in deionized distilled water as described in Example 7.1, were diluted to a concentration of 5 μM in 1×DPBS and added to the cells. The cells were treated with SAMiRNAs for 24 hours and maintained at 37° C. with 5% CO2.

### Example 10-4C: Analysis of Target mRNA Expression Inhibition Efficacy within Cells

Total RNA was extracted from the SAMiRNA-treated cell lines described in Example B. Subsequently, cDNA was synthesized, and real-time PCR was performed to relatively quantify the mRNA expression levels of the target genes. For mRNA expression analysis of the target genes, AccuPower® Dual-HotStart RT-qPCR kits (Bioneer, Korea) were prepared with specific forward primers (300 nM), reverse primers (300 nM), and probes (300 nM) for each target in each well (Table 2A, primer and hydrolysis probe sequences used in high-throughput screening (HTS) experiments). The performance of the prepared kits was validated by generating a standard curve using A549 cell total RNA to determine PCR amplification efficiency (Table 2B). The RT-qPCR reaction protocol involved incubation at 95° C. for 5 minutes, followed by 45 cycles of denaturation at 95° C. for 5 seconds and annealing/extension at 58° C. for 15 seconds, with fluorescence detection at each cycle. The SAMiRNA-treated 24-well plate (Costar, US) was subjected to automated RNA extraction and one-step RT-qPCR using the ExiStation™ HTKorea, a fully automated system that incorporates the HT DNA/RNA extraction kit (Bioneer, Korea), target-specific primers and probes, and the AccuPower® Dual-HotStart RT-qPCR kit (Bioneer, Korea) according to the automated program.

TABLE 2A
Primer and hydrolysis probe sequences used in high-throughput screening (HTS)
experiments for each target.

| | F primer | R primer | |
|---|---|---|---|
| Name | sequence | sequence | Probe sequence |
| BACE1 | TGAAGTGCTGG | GATCCTGTCCA | [TAMRA]ACACCCATCCGGCG |
| | CCTCTGT (SEQ | TTGATCTCCAC | GGAGTGGT[EBQ] (SEQ ID NO: 6) |
| | ID NO: 4) | (SEQ ID NO: 5) | |
| MAPT | ACCACAGCCAC | AGCCATCCTG | [TAMRA]CGCTGTCCTCTCCCG |
| | CTTCTCC (SEQ | GTTCAAAGTTC | TCCTCGCC[EBQ] (SEQ ID NO: 9) |
| | ID NO: 7) | (SEQ ID NO: 8) | |
| HPRT1 | TGCAGACTTTG | CTGGCTTATAT | [TEXAS- |
| | CTTTCCTTG | CCAACACTTCG | RED]CCAAAGATGGTCAAGGT |
| | (SEQ ID NO: 10) | (SEQ ID NO: 11) | CGCAAG[EBQ] (SEQ ID NO: 12) |
| Bace1 | GCAAAAACCCT | CTGCTCAGGCC | [FAM]CGCCTCCCCAGCCTGCC |
| | TTGGCTTTGA | ACCATAATC | TAG[EBQ] (SEQ ID NO: 15) |
| | (SEQ ID NO: 13) | (SEQ ID NO: 14) | |
| Mapt | CGCCTTCTGTC | CTCCAGCATG | [FAM]TGGCTGACCCTCGCCA |
| | GATTATCAGG | GTCTTCCATT | GGAGT[EBQ] (SEQ ID NO: 18) |
| | (SEQ ID NO: 16) | (SEQ ID NO: 17) | |
| Tfrc | AAACTTGCCCA | ATGAAAGGTA | [TAMRA] |
| | AGTATTCTCAG | TCCCTCCAACC | TGCCAGCTGGACTGCAGGCG |
| | (SEQ ID NO: 19) | (SEQ ID NO: 20) | ACT[EBQ] (SEQ ID NO: 21) |

TABLE 2B
PCR amplification efficiency for each
target as determined by standard curve analysis.
Table 2B.

| | | | | Effi- |
|---|---|---|---|---|
| Gene | Dye | Dynamic range | Slope/R\hat{ }2 | ciency |
| BACE1 | TAMRA- | 5X~0.00244X | Y = −0.2836X + | 92% |
| | EBQ | | 8.4383/R2 = 0.9975 | |
| MAPT | TAMRA- | 5X~0.00244X | Y = −0.2782X + | 90% |
| | EBQ | | 8.4754/R2 = 0.9965 | |
| HPRT1 | TEXAS- | 5X~0.00244X | Y = −0.2794X + | 90% |
| | RED-EBQ | | 7.1147/R2 = 0.9978 | |
| Bace1 | FAM-EBQ | 5X~0.01953125X | Y = −0.2948X + | 97% |
| | | | 8.7934/R2 = 0.9965 | |
| Mapt | FAM-EBQ | 5X~0.01953125X | Y = −0.2948X + | 97% |
| | | | 8.7934/R2 = 0.9965 | |
| Tfrc | TAMRA- | 5X~0.01953125X | Y = −0.3004X + | 100% |
| | EBQ | | 8.1207/R2 = 0.9965 | |

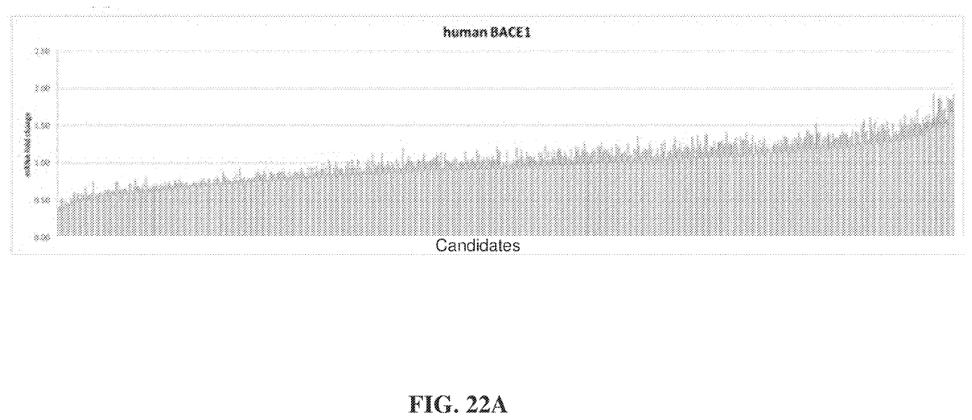

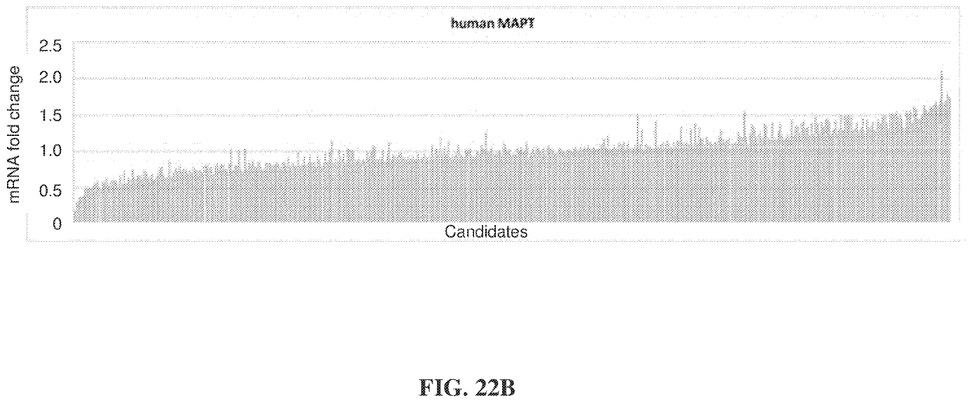

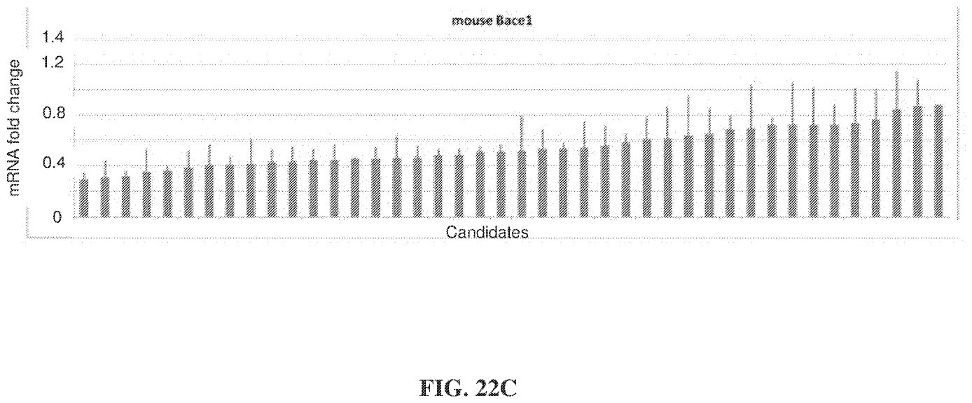

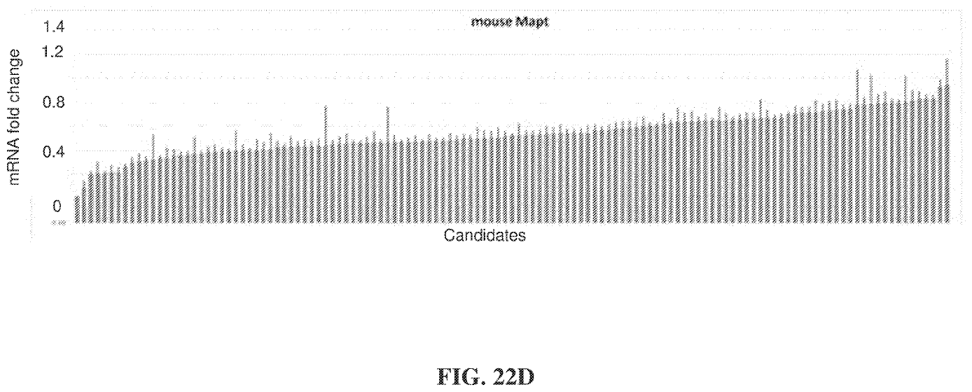

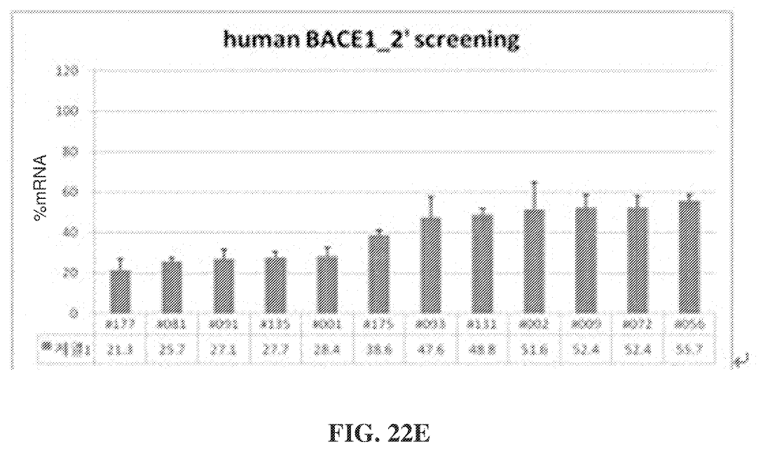

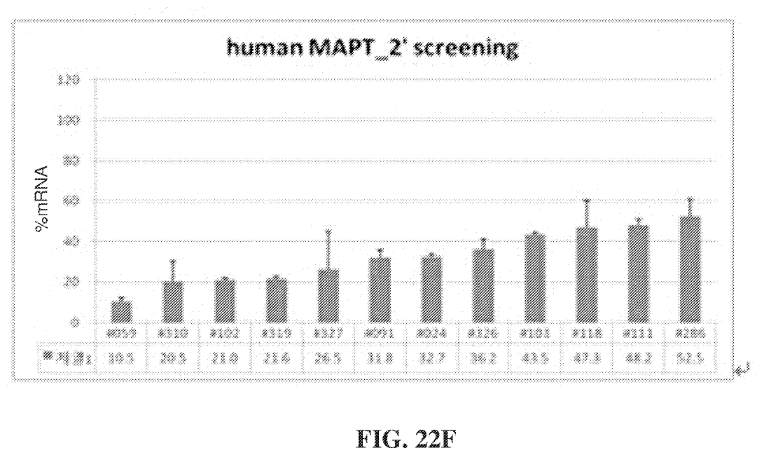

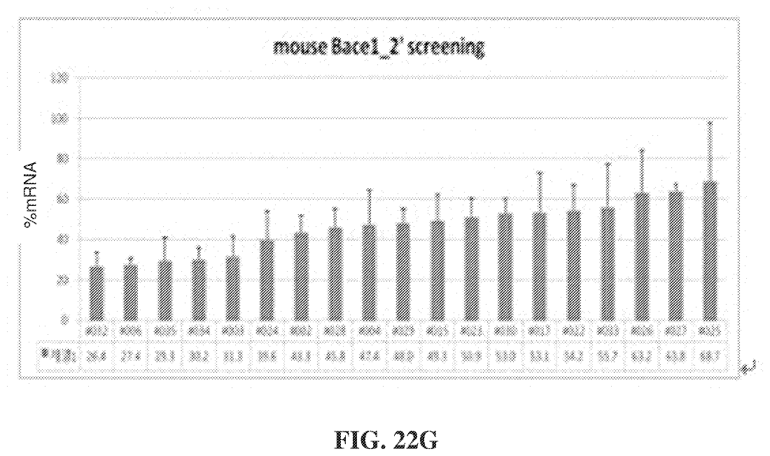

The Ct values of the two target genes obtained from the qPCR array were analyzed using the 2(−Delta Delta C(T)) Method (Livak K J, Schmittgen T D. 2001). Analysis of relative gene expression data using real-time quantitative PCR and the 2(-Delta Delta C(T)) Method. Methods. December; 25 (4): 402-8] to calculate the relative quantity (%) of each target mRNA in the experimental group compared to the control group. Based on the results shown in FIG. 22A-22D, a total of 55 SAMIRNAs, comprising 12 sequences for human BACE1, 12 sequences for human MAPT, 19 sequences for mouse Bace1, and 12 sequences for mouse Mapt, with each sequence having a sense strand, were selected as the most efficient SAMiRNAs for further investigation (Table 2C). Table 2D-2F provide additional embodiments where sense is RNA and antisense is DNA (Table 2D), sense is RNA and antisense is RNA (Table 2E), and sense is DNA and antisense is DNA (Table 2F).

TABLE 2C
Selected species from the results of the primary screening where sense strand is DNA
and antisense strand is RNA.

| | | | | SEQ | | SEQ |
|---|---|---|---|---|---|---|
| | Pair | | | ID | | ID |
| Target | # | position | Sense (DNA) | NO: | Antisense (RNA) | NO: |
| hu-BACE1 | #081 | 1460~1478 | AGGCACCACCCCTTGG | 22 | GUUCCAAGGGGUGGUG | 23 |
| (NM_012104.4) | | | AAC | | CCU | |
| | #001 | 814~832 | CCAGCACATACCGGGA | 24 | AGGUCCCGGUAUGUGC | 25 |
| | | | CCT | | UGG | |
| | #091 | 1509~1527 | GGTGAGGTTACCAACC | 26 | ACUGGUUGGUAACCUC | 27 |
| | | | AGT | | ACC | |
| | #131 | 1687~1705 | CCCGAAAACGAATTGG | 28 | AAGCCAAUUCGUUUUC | 29 |
| | | | CTT | | GGG | |
| | #135 | 1700~1718 | TGGCTTTGCTGTCAGCG | 30 | AGCGCUGACAGCAAAG | 31 |
| | | | CT | | CCA | |
| | #177 | 2198~2216 | TACTCTTGGTCACCTCA | 32 | UUUGAGGUGACCAAGA | 33 |
| | | | AA | | GUA | |
| | #175 | 2196~2214 | AATACTCTTGGTCACCT | 34 | UGAGGUGACCAAGAGU | 35 |
| | | | CA | | AUU | |
| | #093 | 1519~1537 | CCAACCAGTCCTTCCG | 36 | AUGCGGAAGGACUGGU | 37 |
| | | | CAT | | UGG | |
| | #056 | 1221~1239 | ATCCGGCGGGAGTGGT | 38 | AAUACCACUCCCGCCG | 39 |
| | | | ATT | | GAU | |
| | #009 | 830~848 | CCTCCGGAAGGGTGTG | 40 | AUACACACCCUUCCGG | 41 |
| | | | TAT | | AGG | |
| | #002 | 815~833 | CAGCACATACCGGGAC | 42 | GAGGUCCCGGUAUGUG | 43 |
| | | | CTC | | CUG | |
| | #072 | 1258~1276 | GGGTGGAGATCAATGG | 44 | UGUCCAUUGAUCUCCA | 45 |
| | | | ACA | | CCC | |
| hu-MAPT | #059 | 1704~1722 | GGGCTGATGGTAAAAC | 46 | UUCGUUUUACCAUCAG | 47 |
| (NM_016835.4) | | | GAA | | CCC | |
| | #326 | 4832~4850 | CTGGTCTGGCTTGCGGC | 48 | GCGCCGCAAGCCAGAC | 49 |
| | | | GC | | CAG | |
| | #024 | 355~373 | GGAAGATCACGCTGGG | 50 | CGUCCCAGCGUGAUCU | 51 |
| | | | ACG | | UCC | |
| | #310 | 4708~4726 | AGCTAAGGAGGCCGTT | 52 | CUGAACGGCCUCCUUA | 53 |
| | | | CAG | | GCU | |
| | #319 | 4750~4768 | GGATTAGGACTGAAGC | 54 | AUCGCUUCAGUCCUAA | 55 |
| | | | GAT | | UCC | |
| | #102 | 1960~1978 | CCGTACTCCACCCAAG | 56 | CGACUUGGGUGGAGUA | 57 |
| | | | TCG | | CGG | |
| | #103 | 1961~1979 | CGTACTCCACCCAAGT | 58 | GCGACUUGGGUGGAGU | 59 |
| | | | CGC | | ACG | |
| | #327 | 4837~4855 | CTGGCTTGCGGCGCGA | 60 | UCCUCGCGCCGCAAGC | 61 |
| | | | GGA | | CAG | |
| | #286 | 4569~4587 | TTCTAACCCACCCTCAC | 62 | UCGUGAGGGUGGGUUA | 63 |
| | | | GA | | GAA | |
| | #118 | 2042~2060 | AAGTCCAAGATCGGCT | 64 | UGGAGCCGAUCUUGGA | 65 |
| | | | CCA | | CUU | |
| | #111 | 1976~1994 | TCGCCGTCTTCCGCCAA | 66 | UCUUGGCGGAAGACGG | 67 |
| | | | GA | | CGA | |
| | #091 | 1849~1867 | GGATCGCAGCGGCTAC | 68 | GCUGUAGCCGCUGCGA | 69 |
| | | | AGC | | UCC | |
| ms-Bace1 | #035 | 940~958 | AACGTCACTGTGCGTG | 70 | UGGCACGCACAGUGAC | 71 |
| (NM_011792.6) | | | CCA | | GUU | |
| | #024 | 616~634 | CGGGAGACCGACGAGG | 72 | AUUCCUCGUCGGUCUC | 73 |
| | | | AAT | | CCG | |
| | #032 | 874~892 | GGAGGAGCCTGGCCGG | 74 | UCUCCGGCCAGGCUCC | 75 |
| | | | AGA | | UCC | |
| | #034 | 923~941 | TCCGGCTGCCCCTTCGC | 76 | CUGCGAAGGGGCAGCC | 77 |
| | | | AG | | GGA | |
| | #006 | 124~142 | GGAGCCCGGAGTCCCT | 78 | GACAGGGACUCCGGGC | 79 |
| | | | GTC | | UCC | |
| | #003 | 63~81 | TGCCTAGGTGCTGGGA | 80 | AGCUCCCAGCACCUAG | 81 |
| | | | GCT | | GCA | |
| | #028 | 764~782 | CGGGCAGTAGTAACTT | 82 | GCAAAGUUACUACUGC | 83 |
| | | | TGC | | CCG | |
| | #030 | 831~849 | GCTGTCCAGCACATAT | 84 | UCGAUAUGUGCUGGAC | 85 |
| | | | CGA | | AGC | |
| | #029 | 800~818 | ACCCTTTCCTGCATCGC | 86 | UAGCGAUGCAGGAAAG | 87 |
| | | | TA | | GGU | |
| | #022 | 542~560 | AGGGAACCCATCTCGG | 88 | AUGCCGAGAUGGGUUC | 89 |
| | | | CAT | | CCU | |
| | #023 | 560~578 | GCATCCCTCATGGCCC | 90 | UUGGGGCCAUGAGGGA | 91 |
| | | | CAA | | UGC | |
| | #002 | 39~57 | ACAAGCCTTTCCGCCTC | 92 | GGGAGGCGGAAAGGCU | 93 |
| | | | CC | | UGU | |
| | #004 | 87~105 | CTGGATTATGGTGGCCT | 94 | UCAGGCCACCAUAAUC | 95 |
| | | | GA | | CAG | |
| | #017 | 375~393 | CAGTCCTGATGCCGCC | 96 | CUCGGCGGCAUCAGGA | 97 |
| | | | GAG | | CUG | |
| | #015 | 332~350 | CGCCGCCTGGACCGGG | 98 | GUUCCCGGUCCAGGCG | 99 |
| | | | AAC | | GCG | |
| | #033 | 906~924 | GGGCACCGACCTGGTG | 100 | GCUCACCAGGUCGGUG | 101 |
| | | | AGC | | CCC | |
| | #027 | 709~727 | TATGTGGAGATGACCG | 102 | CUACGGUCAUCUCCAC | 103 |
| | | | TAG | | AUA | |
| | #026 | 684~702 | GAGGGGAAAGTCCGGC | 104 | CUGGCCGGACUUUCCC | 105 |
| | | | CAG | | CUC | |
| | #025 | 636~654 | CCCTACACCCAGGGCA | 106 | ACUUGCCCUGGGUGUA | 107 |
| | | | AGT | | GGG | |
| ms-Mapt | #038 | 2459~2477 | TGGGACTTTAGGGCTA | 108 | GGUUAGCCCUAAAGUC | 109 |
| (NM_ | | | ACC | | CCA | |
| 001038609.2) | #089 | 4311~4329 | TGGGGTGATCAGCCCA | 110 | UUUUGGGCUGAUCACC | 111 |
| | | | AAA | | CCA | |
| | #001 | 2~20 | CGCCGGCCTCCAGAAC | 112 | CGCGUUCUGGAGGCCG | 113 |
| | | | GCG | | GCG | |
| | #101 | 4576~4594 | ATTGCTTGCGTTGTGGG | 114 | CCCCCACAACGCAAGC | 115 |
| | | | GG | | AAU | |
| | #096 | 4462~4480 | GGTGGGCTAGATAGAT | 116 | CCUAUCUAUCUAGCCC | 117 |
| | | | AGG | | ACC | |
| | #102 | 4599~4617 | GGAGGAGGAACACATT | 118 | CUUAAUGUGUUCCUCC | 119 |
| | | | AAG | | UCC | |
| | #121 | 5189~5207 | GGGCATGATCTCCAGT | 120 | AGCACUGGAGAUCAUG | 121 |
| | | | GCT | | CCC | |
| | #103 | 4633~4651 | AAAGGGAGGTCTTGGA | 122 | CACUCCAAGACCUCCC | 123 |
| | | | GTG | | UUU | |
| | #011 | 585~603 | GGCAAAACCGGGGCGA | 124 | UCUUCGCCCCGGUUUU | 125 |
| | | | AGA | | GCC | |
| | #097 | 4484~4502 | TACAATTGGCCTGGTTC | 126 | AGGAACCAGGCCAAUU | 127 |
| | | | CT | | GUA | |
| | #008 | 525~543 | GCTCGTGTGGCCAGCA | 128 | CUUUGCUGGCCACACG | 129 |
| | | | AAG | | AGC | |
| | #095 | 4447~4465 | GAGCCAATGTCTTTGG | 130 | CACCCAAAGACAUUGG | 131 |
| | | | GTG | | CUC | |

TABLE 2D
Selected species from the results of the primary screening where sense strand is RNA
and antisense strand is DNA.
Selected species from the results of the primary screening (RNA sense-DNA antisense)

| | | | | SEQ ID | | SEQ ID |
|---|---|---|---|---|---|---|
| Target | Pair # | position | Sense (RNA) | NO: | Antisense (DNA) | NO: |
| hu- | #081 | 1460~1478 | AGGCACCACCCCUUGGA | 136 | GTTCCAAGGGGTG | 137 |
| BACE1 | | | AC | | GTGCCT | |
| (NM_ | #001 | 814~832 | CCAGCACAUACCGGGAC | 138 | AGGTCCCGGTATG | 139 |
| 012104.4 | | | CU | | TGCTGG | |
| | #091 | 1509~1527 | GGUGAGGUUACCAACC | 140 | ACTGGTTGGTAAC | 141 |
| | | | AGU | | CTCACC | |
| | #131 | 1687~1705 | CCCGAAAACGAAUUGG | 142 | AAGCCAATTCGTT | 143 |
| | | | CUU | | TTCGGG | |
| | #135 | 1700~1718 | UGGCUUUGCUGUCAGC | 144 | AGCGCTGACAGCA | 145 |
| | | | GCU | | AAGCCA | |
| | #177 | 2198~2216 | UACUCUUGGUCACCUCA | 146 | TTTGAGGTGACCA | 147 |
| | | | AA | | AGAGTA | |
| | #175 | 2196~2214 | AAUACUCUUGGUCACC | 148 | TGAGGTGACCAAG | 149 |
| | | | UCA | | AGTATT | |
| | #093 | 1519~1537 | CCAACCAGUCCUUCCGC | 150 | ATGCGGAAGGACT | 151 |
| | | | AU | | GGTTGG | |
| | #056 | 1221~1239 | AUCCGGCGGGAGUGGU | 152 | AATACCACTCCCG | 153 |
| | | | AUU | | CCGGAT | |
| | #009 | 830~848 | CCUCCGGAAGGGUGUG | 154 | ATACACACCCTTC | 155 |
| | | | UAU | | CGGAGG | |
| | #002 | 815~833 | CAGCACAUACCGGGACC | 156 | GAGGTCCCGGTAT | 157 |
| | | | UC | | GTGCTG | |
| | #072 | 1258~1276 | GGGUGGAGAUCAAUGG | 158 | TGTCCATTGATCTC | 159 |
| | | | ACA | | CACCC | |
| hu- | #059 | 1704~1722 | GGGCUGAUGGUAAAAC | 160 | TTCGTTTTACCATC | 161 |
| MAPT | | | GAA | | AGCCC | |
| (NM_ | #326 | 4832~4850 | CUGGUCUGGCUUGCGG | 162 | GCGCCGCAAGCCA | 163 |
| 016835.4) | | | CGC | | GACCAG | |
| | #024 | 355~373 | GGAAGAUCACGCUGGG | 164 | CGTCCCAGCGTGA | 165 |
| | | | ACG | | TCTTCC | |
| | #310 | 4708~4726 | AGCUAAGGAGGCCGUU | 166 | CTGAACGGCCTCC | 167 |
| | | | CAG | | TTAGCT | |
| | #319 | 4750~4768 | GGAUUAGGACUGAAGC | 168 | ATCGCTTCAGTCC | 169 |
| | | | GAU | | TAATCC | |
| | #102 | 1960~1978 | CCGUACUCCACCCAAGU | 170 | CGACTTGGGTGGA | 171 |
| | | | CG | | GTACGG | |
| | #103 | 1961~1979 | CGUACUCCACCCAAGUC | 172 | GCGACTTGGGTGG | 173 |
| | | | GC | | AGTACG | |
| | #327 | 4837~4855 | CUGGCUUGCGGCGCGA | 174 | TCCTCGCGCCGCA | 175 |
| | | | GGA | | AGCCAG | |
| | #286 | 4569~4587 | UUCUAACCCACCCUCAC | 176 | TCGTGAGGGTGGG | 177 |
| | | | GA | | TTAGAA | |
| | #118 | 2042~2060 | AAGUCCAAGAUCGGCU | 178 | TGGAGCCGATCTT | 179 |
| | | | CCA | | GGACTT | |
| | #111 | 1976~1994 | UCGCCGUCUUCCGCCAA | 180 | TCTTGGCGGAAGA | 181 |
| | | | GA | | CGGCGA | |
| | #091 | 1849~1867 | GGAUCGCAGCGGCUAC | 182 | GCTGTAGCCGCTG | 183 |
| | | | AGC | | CGATCC | |
| ms- | #035 | 940~958 | AACGUCACUGUGCGUG | 184 | TGGCACGCACAGT | 185 |
| Bace1 | | | CCA | | GACGTT | |
| (NM_ | #024 | 616~634 | CGGGAGACCGACGAGG | 186 | ATTCCTCGTCGGT | 187 |
| 011792.6) | | | AAU | | CTCCCG | |
| | #032 | 874~892 | GGAGGAGCCUGGCCGG | 188 | TCTCCGGCCAGGC | 189 |
| | | | AGA | | TCCTCC | |
| | #034 | 923~941 | UCCGGCUGCCCCUUCGC | 190 | CTGCGAAGGGGCA | 191 |
| | | | AG | | GCCGGA | |
| | #006 | 124~142 | GGAGCCCGGAGUCCCUG | 192 | GACAGGGACTCCG | 193 |
| | | | UC | | GGCTCC | |
| | #003 | 63~81 | UGCCUAGGUGCUGGGA | 194 | AGCTCCCAGCACC | 195 |
| | | | GCU | | TAGGCA | |
| | #028 | 764~782 | CGGGCAGUAGUAACUU | 196 | GCAAAGTTACTAC | 197 |
| | | | UGC | | TGCCCG | |
| | #030 | 831~849 | GCUGUCCAGCACAUAUC | 198 | TCGATATGTGCTG | 199 |
| | | | GA | | GACAGC | |
| | #029 | 800~818 | ACCCUUUCCUGCAUCGC | 200 | TAGCGATGCAGGA | 201 |
| | | | UA | | AAGGGT | |
| | #022 | 542~560 | AGGGAACCCAUCUCGGC | 202 | ATGCCGAGATGGG | 203 |
| | | | AU | | TTCCCT | |
| | #023 | 560~578 | GCAUCCCUCAUGGCCCC | 204 | TTGGGGCCATGAG | 205 |
| | | | AA | | GGATGC | |
| | #002 | 39~57 | ACAAGCCUUUCCGCCUC | 206 | GGGAGGCGGAAA | 207 |
| | | | CC | | GGCTTGT | |
| | #004 | 87~105 | CUGGAUUAUGGUGGCC | 208 | TCAGGCCACCATA | 209 |
| | | | UGA | | ATCCAG | |
| | #017 | 375~393 | CAGUCCUGAUGCCGCCG | 210 | CTCGGCGGCATCA | 211 |
| | | | AG | | GGACTG | |
| | #015 | 332~350 | CGCCGCCUGGACCGGGA | 212 | GTTCCCGGTCCAG | 213 |
| | | | AC | | GCGGCG | |
| | #033 | 906~924 | GGGCACCGACCUGGUG | 214 | GCTCACCAGGTCG | 215 |
| | | | AGC | | GTGCCC | |
| | #027 | 709~727 | UAUGUGGAGAUGACCG | 216 | CTACGGTCATCTC | 217 |
| | | | UAG | | CACATA | |
| | #026 | 684~702 | GAGGGGAAAGUCCGGC | 218 | CTGGCCGGACTTT | 219 |
| | | | CAG | | CCCCTC | |
| | #025 | 636~654 | CCCUACACCCAGGGCAA | 220 | ACTTGCCCTGGGT | 221 |
| | | | GU | | GTAGGG | |
| ms- | #038 | 2459~2477 | UGGGACUUUAGGGCUA | 222 | GGTTAGCCCTAAA | 223 |
| Mapt | | | ACC | | GTCCCA | |
| (NM_ | #089 | 4311~4329 | UGGGGUGAUCAGCCCA | 224 | TTTTGGGCTGATC | 225 |
| 001038609.2) | | | AAA | | ACCCCA | |
| | #001 | 2~20 | CGCCGGCCUCCAGAACG | 226 | CGCGTTCTGGAGG | 227 |
| | | | CG | | CCGGCG | |
| | #101 | 4576~4594 | AUUGCUUGCGUUGUGG | 228 | CCCCCACAACGCA | 229 |
| | | | GGG | | AGCAAT | |
| | #096 | 4462~4480 | GGUGGGCUAGAUAGAU | 230 | CCTATCTATCTAG | 231 |
| | | | AGG | | CCCACC | |
| | #102 | 4599~4617 | GGAGGAGGAACACAUU | 232 | CTTAATGTGTTCCT | 233 |
| | | | AAG | | CCTCC | |
| | #121 | 5189~5207 | GGGCAUGAUCUCCAGU | 234 | AGCACTGGAGATC | 235 |
| | | | GCU | | ATGCCC | |
| | #103 | 4633~4651 | AAAGGGAGGUCUUGGA | 236 | CACTCCAAGACCT | 237 |
| | | | GUG | | CCCTTT | |
| | #011 | 585~603 | GGCAAAACCGGGGCGA | 238 | TCTTCGCCCCGGTT | 239 |
| | | | AGA | | TTGCC | |
| | #097 | 4484~4502 | UACAAUUGGCCUGGUU | 240 | AGGAACCAGGCCA | 241 |
| | | | CCU | | ATTGTA | |
| | #008 | 525~543 | GCUCGUGUGGCCAGCA | 242 | CTTTGCTGGCCAC | 243 |
| | | | AAG | | ACGAGC | |
| | #095 | 4447~4465 | GAGCCAAUGUCUUUGG | 244 | CACCCAAAGACAT | 245 |
| | | | GUG | | TGGCTC | |

TABLE 2E
Selected species from the results of the primary screening where sense strand is RNA
and antisense strand is RNA.
Selected species from the results of the primary screening (RNA sense-RNA antisense)

| | | | | SEQ | | SEQ |
|---|---|---|---|---|---|---|
| | | | | ID | | ID |
| Target | Pair # | position | sense (RNA) | NO: | antisense (RNA) | NO: |
| hu- | #081 | 1460~1478 | AGGCACCACCCCUUGG | 136 | GUUCCAAGGGGUGGU | 23 |
| BACE1 | | | AAC | | GCCU | |
| (NM_ | #001 | 814~832 | CCAGCACAUACCGGGA | 138 | AGGUCCCGGUAUGUG | 25 |
| 012104.4) | | | CCU | | CUGG | |
| | #091 | 1509~1527 | GGUGAGGUUACCAACC | 140 | ACUGGUUGGUAACCU | 27 |
| | | | AGU | | CACC | |
| | #131 | 1687~1705 | CCCGAAAACGAAUUGG | 142 | AAGCCAAUUCGUUUU | 29 |
| | | | CUU | | CGGG | |
| | #135 | 1700~1718 | UGGCUUUGCUGUCAGC | 144 | AGCGCUGACAGCAAA | 31 |
| | | | GCU | | GCCA | |
| | #177 | 2198~2216 | UACUCUUGGUCACCUC | 146 | UUUGAGGUGACCAAG | 33 |
| | | | AAA | | AGUA | |
| | #175 | 2196~2214 | AAUACUCUUGGUCACC | 148 | UGAGGUGACCAAGAG | 35 |
| | | | UCA | | UAUU | |
| | #093 | 1519~1537 | CCAACCAGUCCUUCCG | 150 | AUGCGGAAGGACUGG | 37 |
| | | | CAU | | UUGG | |
| | #056 | 1221~1239 | AUCCGGCGGGAGUGGU | 152 | AAUACCACUCCOGCC | 39 |
| | | | AUU | | GGAU | |
| | #009 | 830~848 | CCUCCGGAAGGGUGUG | 154 | AUACACACCCUUCCG | 41 |
| | | | UAU | | GAGG | |
| | #002 | 815~833 | CAGCACAUACCGGGAC | 156 | GAGGUCCCGGUAUGU | 43 |
| | | | CUC | | GCUG | |
| | #072 | 1258~1276 | GGGUGGAGAUCAAUGG | 158 | UGUCCAUUGAUCUCC | 45 |
| | | | ACA | | ACCC | |
| hu- | #059 | 1704~1722 | GGGCUGAUGGUAAAAC | 160 | UUCGUUUUACCAUCA | 47 |
| MAPT | | | GAA | | GCCC | |
| (NM_ | #326 | 4832~4850 | CUGGUCUGGCUUGCGG | 162 | GCGCCGCAAGCCAGA | 49 |
| 016835.4) | | | CGC | | CCAG | |
| | #024 | 355~373 | GGAAGAUCACGCUGGG | 164 | CGUCCCAGCGUGAUC | 51 |
| | | | ACG | | UUCC | |
| | #310 | 4708~4726 | AGCUAAGGAGGCCGUU | 166 | CUGAACGGCCUCCUU | 53 |
| | | | CAG | | AGCU | |
| | #319 | 4750~4768 | GGAUUAGGACUGAAGC | 168 | AUCGCUUCAGUCCUA | 55 |
| | | | GAU | | AUCC | |
| | #102 | 1960~1978 | CCGUACUCCACCCAAG | 170 | CGACUUGGGUGGAGU | 57 |
| | | | UCG | | ACGG | |
| | #103 | 1961~1979 | CGUACUCCACCCAAGU | 172 | GCGACUUGGGUGGAG | 59 |
| | | | CGC | | UACG | |
| | #327 | 4837~4855 | CUGGCUUGCGGCGCGA | 174 | UCCUCGCGCCGCAAG | 61 |
| | | | GGA | | CCAG | |
| | #286 | 4569~4587 | UUCUAACCCACCCUCA | 176 | UCGUGAGGGUGGGUU | 63 |
| | | | CGA | | AGAA | |
| | #118 | 2042~2060 | AAGUCCAAGAUCGGCU | 178 | UGGAGCCGAUCUUGG | 65 |
| | | | CCA | | ACUU | |
| | #111 | 1976~1994 | UCGCCGUCUUCCGCCA | 180 | UCUUGGCGGAAGACG | 67 |
| | | | AGA | | GCGA | |
| | #091 | 1849~1867 | GGAUCGCAGCGGCUAC | 182 | GCUGUAGCCGCUGCG | 69 |
| | | | AGC | | AUCC | |
| ms- | #035 | 940~958 | AACGUCACUGUGCGUG | 184 | UGGCACGCACAGUGA | 71 |
| Bace1 | | | CCA | | CGUU | |
| (NM_ | #024 | 616~634 | CGGGAGACCGACGAGG | 186 | AUUCCUCGUCGGUCU | 73 |
| 011792.6) | | | AAU | | CCCG | |
| | #032 | 874~892 | GGAGGAGCCUGGCCGG | 188 | UCUCCGGCCAGGCUC | 75 |
| | | | AGA | | CUCC | |
| | #034 | 923~941 | UCCGGCUGCCCCUUCG | 190 | CUGCGAAGGGGCAGC | 77 |
| | | | CAG | | CGGA | |
| | #006 | 124~142 | GGAGCCCGGAGUCCCU | 192 | GACAGGGACUCCGGG | 79 |
| | | | GUC | | CUCC | |
| | #003 | 63~81 | UGCCUAGGUGCUGGGA | 194 | AGCUCCCAGCACCUA | 81 |
| | | | GCU | | GGCA | |
| | #028 | 764~782 | CGGGCAGUAGUAACUU | 196 | GCAAAGUUACUACUG | 83 |
| | | | UGC | | CCCG | |
| | #030 | 831~849 | GCUGUCCAGCACAUAU | 198 | UCGAUAUGUGCUGGA | 85 |
| | | | CGA | | CAGC | |
| | #029 | 800~818 | ACCCUUUCCUGCAUCG | 200 | UAGCGAUGCAGGAAA | 87 |
| | | | CUA | | GGGU | |
| | #022 | 542~560 | AGGGAACCCAUCUCGG | 202 | AUGCCGAGAUGGGUU | 89 |
| | | | CAU | | CCCU | |
| | #023 | 560~578 | GCAUCCCUCAUGGCCC | 204 | UUGGGGCCAUGAGGG | 91 |
| | | | CAA | | AUGC | |
| | #002 | 39~57 | ACAAGCCUUUCCGCCU | 206 | GGGAGGCGGAAAGGC | 93 |
| | | | CCC | | UUGU | |
| | #004 | 87~105 | CUGGAUUAUGGUGGCC | 208 | UCAGGCCACCAUAAU | 95 |
| | | | UGA | | CCAG | |
| | #017 | 375~393 | CAGUCCUGAUGCCGCC | 210 | CUCGGCGGCAUCAGG | 97 |
| | | | GAG | | ACUG | |
| | #015 | 332~350 | CGCCGCCUGGACCGGG | 212 | GUUCCCGGUCCAGGC | 99 |
| | | | AAC | | GGCG | |
| | #033 | 906~924 | GGGCACCGACCUGGUG | 214 | GCUCACCAGGUCGGU | 101 |
| | | | AGC | | GCCC | |
| | #027 | 709~727 | UAUGUGGAGAUGACCG | 216 | CUACGGUCAUCUCCA | 103 |
| | | | UAG | | CAUA | |
| | #026 | 684~702 | GAGGGGAAAGUCCGGC | 218 | CUGGCCGGACUUUCC | 105 |
| | | | CAG | | CCUC | |
| | #025 | 636~654 | CCCUACACCCAGGGCA | 220 | ACUUGCCCUGGGUGU | 107 |
| | | | AGU | | AGGG | |
| ms- | #038 | 2459~2477 | UGGGACUUUAGGGCUA | 222 | GGUUAGCCCUAAAGU | 109 |
| Mapt | | | ACC | | CCCA | |
| (NM_ | #089 | 4311~4329 | UGGGGUGAUCAGCCCA | 224 | UUUUGGGCUGAUCAC | 111 |
| 001038609.2) | | | AAA | | CCCA | |
| | #001 | 2~20 | CGCCGGCCUCCAGAAC | 226 | CGCGUUCUGGAGGCC | 113 |
| | | | GCG | | GGCG | |
| | #101 | 4576~4594 | AUUGCUUGCGUUGUGG | 228 | CCCCCACAACGCAAGC | 115 |
| | | | GGG | | AAU | |
| | #096 | 4462~4480 | GGUGGGCUAGAUAGAU | 230 | CCUAUCUAUCUAGCC | 117 |
| | | | AGG | | CACC | |
| | #102 | 4599~4617 | GGAGGAGGAACACAUU | 232 | CUUAAUGUGUUCCUC | 119 |
| | | | AAG | | CUCC | |
| | #121 | 5189~5207 | GGGCAUGAUCUCCAGU | 234 | AGCACUGGAGAUCAU | 121 |
| | | | GCU | | GCCC | |
| | #103 | 4633~4651 | AAAGGGAGGUCUUGGA | 236 | CACUCCAAGACCUCCC | 123 |
| | | | GUG | | UUU | |
| | #011 | 585~603 | GGCAAAACCGGGGCGA | 238 | UCUUCGCCCCGGUUU | 125 |
| | | | AGA | | UGCC | |
| | #097 | 4484~4502 | UACAAUUGGCCUGGUU | 240 | AGGAACCAGGCCAAU | 127 |
| | | | CCU | | UGUA | |
| | #008 | 525~543 | GCUCGUGUGGCCAGCA | 242 | CUUUGCUGGCCACAC | 129 |
| | | | AAG | | GAGC | |
| | #095 | 4447~4465 | GAGCCAAUGUCUUUGG | 244 | CACCCAAAGACAUUG | 131 |
| | | | GUG | | GCUC | |

TABLE 2F
Selected species from the results of the primary screening where sense strand is DNA
and antisense strand is DNA.
Selected species from the results of the primary screening (DNA sense-DNA
antisense)

| | | | | SEQ | | SEQ |
|---|---|---|---|---|---|---|
| | | | | ID | | ID |
| Target | Pair # | position | Sense (DNA) | NO: | Antisense (DNA) | NO: |
| hu- | #081 | 1460~1478 | AGGCACCACCCCTTGGA | 22 | GTTCCAAGGGGTGGTGCC | 137 |
| BACE1 | | | AC | | T | |
| (NM_ | #001 | 814~832 | CCAGCACATACCGGGAC | 24 | AGGTCCCGGTATGTGCTG | 139 |
| 012104.4) | | | CT | | G | |
| | #091 | 1509~1527 | GGTGAGGTTACCAACCA | 26 | ACTGGTTGGTAACCTCAC | 141 |
| | | | GT | | C | |
| | #131 | 1687~1705 | CCCGAAAACGAATTGGC | 28 | AAGCCAATTCGTTTTCGG | 143 |
| | | | TT | | G | |
| | #135 | 1700~1718 | TGGCTTTGCTGTCAGCG | 30 | AGCGCTGACAGCAAAGC | 145 |
| | | | CT | | CA | |
| | #177 | 2198~2216 | TACTCTTGGTCACCTCA | 32 | TTTGAGGTGACCAAGAGT | 147 |
| | | | AA | | A | |
| | #175 | 2196~2214 | AATACTCTTGGTCACCT | 34 | TGAGGTGACCAAGAGTA | 149 |
| | | | CA | | TT | |
| | #093 | 1519~1537 | CCAACCAGTCCTTCCGC | 36 | ATGCGGAAGGACTGGTT | 151 |
| | | | AT | | GG | |
| | #056 | 1221~1239 | ATCCGGCGGGAGTGGTA | 38 | AATACCACTCCCGCCGGA | 153 |
| | | | TT | | T | |
| | #009 | 830~848 | CCTCCGGAAGGGTGTGT | 40 | ATACACACCCTTCCGGAG | 155 |
| | | | AT | | G | |
| | #002 | 815~833 | CAGCACATACCGGGACC | 42 | GAGGTCCCGGTATGTGCT | 157 |
| | | | TC | | G | |
| | #072 | 1258~1276 | GGGTGGAGATCAATGGA | 44 | TGTCCATTGATCTCCACC | 159 |
| | | | CA | | C | |
| hu- | #059 | 1704~1722 | GGGCTGATGGTAAAACG | 46 | TTCGTTTTACCATCAGCC | 161 |
| MAPT | | | AA | | C | |
| (NM_ | #326 | 4832~4850 | CTGGTCTGGCTTGCGGC | 48 | GCGCCGCAAGCCAGACC | 163 |
| 016835.4) | | | GC | | AG | |
| | #024 | 355~373 | GGAAGATCACGCTGGGA | 50 | CGTCCCAGCGTGATCTTC | 165 |
| | | | CG | | C | |
| | #310 | 4708~4726 | AGCTAAGGAGGCCGTTC | 52 | CTGAACGGCCTCCTTAGC | 167 |
| | | | AG | | T | |
| | #319 | 4750~4768 | GGATTAGGACTGAAGCG | 54 | ATCGCTTCAGTCCTAATC | 169 |
| | | | AT | | C | |
| | #102 | 1960~1978 | CCGTACTCCACCCAAGT | 56 | CGACTTGGGTGGAGTACG | 171 |
| | | | CG | | G | |
| | #103 | 1961~1979 | CGTACTCCACCCAAGTC | 58 | GCGACTTGGGTGGAGTAC | 173 |
| | | | GC | | G | |
| | #327 | 4837~4855 | CTGGCTTGCGGCGCGAG | 60 | TCCTCGCGCCGCAAGCCA | 175 |
| | | | GA | | G | |
| | #286 | 4569~4587 | TTCTAACCCACCCTCAC | 62 | TCGTGAGGGTGGGTTAGA | 177 |
| | | | GA | | A | |
| | #118 | 2042~2060 | AAGTCCAAGATCGGCTC | 64 | TGGAGCCGATCTTGGACT | 179 |
| | | | CA | | T | |
| | #111 | 1976~1994 | TCGCCGTCTTCCGCCAA | 66 | TCTTGGCGGAAGACGGC | 181 |
| | | | GA | | GA | |
| | #091 | 1849~1867 | GGATCGCAGCGGCTACA | 68 | GCTGTAGCCGCTGCGATC | 183 |
| | | | GC | | C | |
| ms- | #035 | 940~958 | AACGTCACTGTGCGTGC | 70 | TGGCACGCACAGTGACGT | 185 |
| Bace1 | | | CA | | T | |
| (NM_ | #024 | 616~634 | CGGGAGACCGACGAGG | 72 | ATTCCTCGTCGGTCTCCC | 187 |
| 011792.6) | | | AAT | | G | |
| | #032 | 874~892 | GGAGGAGCCTGGCCGGA | 74 | TCTCCGGCCAGGCTCCTC | 189 |
| | | | GA | | C | |
| | #034 | 923~941 | TCCGGCTGCCCCTTCGC | 76 | CTGCGAAGGGGCAGCCG | 191 |
| | | | AG | | GA | |
| | #006 | 124~142 | GGAGCCCGGAGTCCCTG | 78 | GACAGGGACTCCGGGCT | 193 |
| | | | TC | | CC | |
| | #003 | 63~81 | TGCCTAGGTGCTGGGAG | 80 | AGCTCCCAGCACCTAGGC | 195 |
| | | | CT | | A | |
| | #028 | 764~782 | CGGGCAGTAGTAACTTT | 82 | GCAAAGTTACTACTGCCC | 197 |
| | | | GC | | G | |
| | #030 | 831~849 | GCTGTCCAGCACATATC | 84 | TCGATATGTGCTGGACAG | 199 |
| | | | GA | | C | |
| | #029 | 800~818 | ACCCTTTCCTGCATCGCT | 86 | TAGCGATGCAGGAAAGG | 201 |
| | | | A | | GT | |
| | #022 | 542~560 | AGGGAACCCATCTCGGC | 88 | ATGCCGAGATGGGTTCCC | 203 |
| | | | AT | | T | |
| | #023 | 560~578 | GCATCCCTCATGGCCCC | 90 | TTGGGGCCATGAGGGAT | 205 |
| | | | AA | | GC | |
| | #002 | 39~57 | ACAAGCCTTTCCGCCTC | 92 | GGGAGGCGGAAAGGCTT | 207 |
| | | | CC | | GT | |
| | #004 | 87~105 | CTGGATTATGGTGGCCT | 94 | TCAGGCCACCATAATCCA | 209 |
| | | | GA | | G | |
| | #017 | 375~393 | CAGTCCTGATGCCGCCG | 96 | CTCGGCGGCATCAGGACT | 211 |
| | | | AG | | G | |
| | #015 | 332~350 | CGCCGCCTGGACCGGGA | 98 | GTTCCCGGTCCAGGCGGC | 213 |
| | | | AC | | G | |
| | #033 | 906~924 | GGGCACCGACCTGGTGA | 100 | GCTCACCAGGTCGGTGCC | 215 |
| | | | GC | | C | |
| | #027 | 709~727 | TATGTGGAGATGACCGT | 102 | CTACGGTCATCTCCACAT | 217 |
| | | | AG | | A | |
| | #026 | 684~702 | GAGGGGAAAGTCCGGCC | 104 | CTGGCCGGACTTTCCCCT | 219 |
| | | | AG | | C | |
| | #025 | 636~654 | CCCTACACCCAGGGCAA | 106 | ACTTGCCCTGGGTGTAGG | 221 |
| | | | GT | | G | |
| ms- | #038 | 2459~2477 | TGGGACTTTAGGGCTAA | 108 | GGTTAGCCCTAAAGTCCC | 223 |
| Mapt | | | CC | | A | |
| (NM_ | #089 | 4311~4329 | TGGGGTGATCAGCCCAA | 110 | TTTTGGGCTGATCACCCC | 225 |
| 001038609.2) | | | AA | | A | |
| | #001 | 2~20 | CGCCGGCCTCCAGAACG | 112 | CGCGTTCTGGAGGCCGGC | 227 |
| | | | CG | | G | |
| | #101 | 4576~4594 | ATTGCTTGCGTTGTGGG | 114 | CCCCCACAACGCAAGCA | 229 |
| | | | GG | | AT | |
| | #096 | 4462~4480 | GGTGGGCTAGATAGATA | 116 | CCTATCTATCTAGCCCAC | 231 |
| | | | GG | | C | |
| | #102 | 4599~4617 | GGAGGAGGAACACATTA | 118 | CTTAATGTGTTCCTCCTC | 233 |
| | | | AG | | C | |
| | #121 | 5189~5207 | GGGCATGATCTCCAGTG | 120 | AGCACTGGAGATCATGCC | 235 |
| | | | CT | | C | |
| | #103 | 4633~4651 | AAAGGGAGGTCTTGGAG | 122 | CACTCCAAGACCTCCCTT | 237 |
| | | | TG | | T | |
| | #011 | 585~603 | GGCAAAACCGGGGCGA | 124 | TCTTCGCCCCGGTTTTGC | 239 |
| | | | AGA | | C | |
| | #097 | 4484~4502 | TACAATTGGCCTGGTTC | 126 | AGGAACCAGGCCAATTG | 241 |
| | | | CT | | TA | |
| | #008 | 525~543 | GCTCGTGTGGCCAGCAA | 128 | CTTTGCTGGCCACACGAG | 243 |
| | | | AG | | C | |
| | #095 | 4447~4465 | GAGCCAATGTCTTTGGG | 130 | CACCCAAAGACATTGGCT | 245 |
| | | | TG | | C | |

### Example 10-4D: Second Screening

Following the protocol described in Example 1st screening, the selected 55 species of SAMIRNA were individually treated in the respective cell lines according to Example 1-B and 1-C. The expression levels of each target were analyzed in a secondary analysis, and the results of Experiment 2 were obtained. Among the candidate sequences, the SAMiRNA with the highest efficiency was selected based on the relative decrease in target mRNA expression compared to the control group. Specifically, four species of human BACE1, five species of human MAPT, five species of mouse Bace1, and five species of mouse Mapt SAMiRNA, all possessing the respective target-specific sense strand, were selected from the 55 species (Table 2G). Data is shown in FIG. 22E-22H.

TABLE 2G
Selected candidates from the results of the secondary screening

| | NCBI | | | | |
|---|---|---|---|---|---|
| Target | accession# | # | position | sense | antisense |
| hu- | NM_012104.4 | #081 | 1460~1478 | AGGCACCACCCCTTGGA | GUUCCAAGGGGUGGUGC |
| BACE1 | | | | AC (SEQ ID NO: 22) | CU (SEQ ID NO: 23) |
| | | #001 | 814~832 | CCAGCACATACCGGGACC | AGGUCCCGGUAUGUGCU |
| | | | | T (SEQ ID NO: 24) | GG (SEQ ID NO: 25) |
| | | #091 | 1509~1527 | GGTGAGGTTACCAACCA | ACUGGUUGGUAACCUCA |
| | | | | GT (SEQ ID NO: 26) | CC (SEQ ID NO: 27) |
| | | #177 | 2198~2216 | TACTCTTGGTCACCTCAA | UUUGAGGUGACCAAGAG |
| | | | | A (SEQ ID NO: 32) | UA (SEQ ID NO: 33) |
| hu- | NM_016835.4 | #059 | 1704~1722 | GGGCTGATGGTAAAACG | UUCGUUUUACCAUCAGC |
| MAPT | | | | AA (SEQ ID NO: 46) | CC (SEQ ID NO: 47) |
| | | #326 | 4832~4850 | CTGGTCTGGCTTGCGGCG | GCGCCGCAAGCCAGACC |
| | | | | C (SEQ ID NO:48) | AG (SEQ ID NO: 49) |
| | | #024 | 355~373 | GGAAGATCACGCTGGGA | CGUCCCAGCGUGAUCUU |
| | | | | CG (SEQ ID NO: 50) | CC (SEQ ID NO: 51) |
| | | #310 | 4708~4726 | AGCTAAGGAGGCCGTTC | CUGAACGGCCUCCUUAG |
| | | | | AG (SEQ ID NO: 52) | CU (SEQ ID NO: 53) |
| | | #102 | 1960~1978 | CCGTACTCCACCCAAGTC | CGACUUGGGUGGAGUAC |
| | | | | G (SEQ ID NO: 56) | GG (SEQ ID NO: 57) |
| ms- | NM_011792.6 | #035 | 940~958 | AACGTCACTGTGCGTGCC | UGGCACGCACAGUGACG |
| Bace1 | | | | A (SEQ ID NO: 70) | UU (SEQ ID NO: 71) |
| | | #024 | 616~634 | CGGGAGACCGACGAGGA | AUUCCUCGUCGGUCUCC |
| | | | | AT (SEQ ID NO: 72) | CG SEQ ID NO: 73) |
| | | #032 | 874~892 | GGAGGAGCCTGGCCGGA | UCUCCGGCCAGGCUCCUC |
| | | | | GA SEQ ID NO: 74) | C (SEQ ID NO: 75) |
| | | #034 | 923~941 | TCCGGCTGCCCCTTCGCA | CUGCGAAGGGGCAGCCG |
| | | | | G (SEQ ID NO: 76) | GA (SEQ ID NO: 77) |
| | | #006 | 124~142 | GGAGCCCGGAGTCCCTG | GACAGGGACUCCGGGCU |
| | | | | TC (SEQ ID NO: 78) | CC (SEQ ID NO: 79) |
| ms- | NM_ | #038 | 2459~2477 | TGGGACTTTAGGGCTAAC | GGUUAGCCCUAAAGUCC |
| Mapt | 001038609.2 | | | C (SEQ ID NO: 108) | CA (SEQ ID NO: 109) |
| | | #089 | 4311~4329 | TGGGGTGATCAGCCCAA | UUUUGGGCUGAUCACCC |
| | | | | AA (SEQ ID NO: 110) | CA (SEQ ID NO: 111) |
| | | #001 | 2~20 | CGCCGGCCTCCAGAACG | CGCGUUCUGGAGGCCGG |
| | | | | CG (SEQ ID NO: 112) | CG (SEQ ID NO: 113) |
| | | #101 | 4576~4594 | ATTGCTTGCGTTGTGGGG | CCCCCACAACGCAAGCAA |
| | | | | G (SEQ ID NO: 114) | U (SEQ ID NO: 115) |
| | | #096 | 4462~4480 | GGTGGGCTAGATAGATAG | CCUAUCUAUCUAGCCCA |
| | | | | G (SEQ ID NO: 116) | CC (SEQ ID NO: 117) |

### Example 10-4E: In Vitro Knockdown Efficiency Analysis of Glu-SAMiRNA Targeting BACE1 and MAPT Genes Identified Through High-Throughput Screening

This exampled assessed in vitro knockdown efficiency (KD) of glu-SAMiRNAs carrying the sense strand sequences AGGCACCACCCCTTGGAAC (SEQ ID NO: 22) for BACE1 #81 and GGAAGATCACGCTGGGACG for MAPT #24 (SEQ ID NO: 50) (“SRN-008”), which were selected through high-throughput (HT) screening.

For the experiments, human neuronal cell line SH-SY5Y A549 (KCLB #22266, Korean Cell Line Bank) was cultured in Gibco™ RPMI-1640 medium supplemented with 10% Fetal Bovine Serum (Hyclone, US) and 1% Penicillin-Streptomycin (Hyclone, US) under 37° C. and 5% CO2 conditions. SH-SY5Y cells were seeded in 24-well plates (Costar, US) at a density of 1×10\hat{ }5 cells/well using the same medium and incubated for 24 hours at 37° C. and 5% CO2. The cells were then treated with various concentrations of 10, 5, 2.5, 1.25, 0.625, 0.313, 0.156, 0.078, 0.039, 0.020, 0.010, and 0.005 UM of glu-SAMiRNA for 24 hours. Following the treatment, total RNA was extracted from the cells, and cDNA was synthesized. Real-time PCR was performed to comparatively quantify the mRNA expression levels of the target genes, and IC50 values were analyzed (FIG. 22I).

### Example 10-4F: Effects of SAMiRNA-BACE1/Tau on AD Pathogenesis in 3×Tg

Animals and administration. 3×Tg mice purchased at Jackson Laboratory (Bar Harbor, ME, USA) express three familial AD mutations (APPSwedish, MAPTP301L and PSEN1M246V). 8-month-old female 3×Tg mice and 10-week-old female B6129F1/J mice were used in this study and divided into three groups: (1) B6129F1/J+Vehicle (n=9), (2) 3×Tg+Vehicle (n=10), and (3) 3×Tg+glucose-SAMiRNA-BACE1/Tau (n=10). We intravenously administered glucose-SAMiRNA-BACE1/Tau cocktail at 40 mg/kg (sum) once a week for 4 weeks. glucose-SAMiRNA-BACE1/Tau was dissolved in PBS, and the same amount of PBS was administered to the vehicle group. Food was removed for fasting 24 hours before glucose-SAMIRNA-BACE1/Tau treatment. Two hundred microliters of glucose were administered to all groups by intraperitoneal injection 3 hours after glucose-SAMiRNA-BACE1/Tau or vehicle injection. Animals were maintained for the care and use of laboratory animals according to the National Institutes of Health guide.

Preparation of brain tissue. After the behavior test, animals were anesthetized by intraperitoneal injection of Avertin (Tribromoethanol; Sigma-Aldrich, St. Louis, MO, USA) at a dose of 250 μg/kg. Animals were transcardially perfused with PBS and fixed with cold 4% paraformaldehyde in 0.1 M phosphate buffer (PB). Brain tissues were removed and fixed in fixative solution for 20 h at 4° C. before being soaked in a cryoprotectant solution of 30% sucrose in PBS. The tissues were cut into 30-μm-thick coronal plane with a cryostat (Leica Biosystems, Wetzlar, Germany) at −25° C. and stored at 4° C. in a cryoprotectant solution (25% ethylene glycol, 25% glycerol in 0.05 M PB) until immunohistochemistry analysis.

Immunofluorescence labeling. To investigate immunoreactivity, 2 to 3 of CA1 brain samples were obtained at 290-440 μm intervals in the region between −1.58 and −2.46 mm from the bregma. 2 to 3 of subiculum brain sections were obtained 240-360 μm intervals in the region between −2.92 and −3.64 mm from the bregma. The free-floating sections were rinsed at three times for 5 minutes. The brain tissues were permabilized with 0.3% triton X-100 for 30 minutes, washed 3 times for 5 minutes with PBS, and blocked with a 0.5 mg/mL bovine serum albumin (BSA) for 30 minutes. Then, the tissues washed 3 times for 5 minutes with PBS and incubated with one of the following primary antibodies overnight at 4° C.: mouse anti-4G8 antibody (1:2,000; BioLegend, San Diego, CA, USA), mouse anti-phospho-Tau (Ser202, Thr205) monoclonal (AT8) antibody (1:500; Thermo Fisher Scientific, Waltham, Massachusetts, USA), and mouse anti-tau monoclonal (HT7) antibody (1:100; Thermo Fisher Scientific) were prepared at individual dilution ratios in PBS containing 0.5 mg/mL BSA and 0.3% Triton X-100. Before incubation with the anti-4G8 antibody, the brain tissue was incubated with 70% formic acid for 20 min for antigen retrieval. Subsequently, sections were incubated with the following secondary antibodies for 50 min at room temperature: donkey Alexa 488-conjugated anti-mouse IgG (Thermo Fisher Scientific) at 1:300 dilution in PBS containing 0.3% Triton X-100. Immunostained tissues were mounted on slides and cover-slipped with the Fluoroshield mounting medium with 4,6-diamidino-2-phenylindole (DAPI; Sigma-Aldrich).

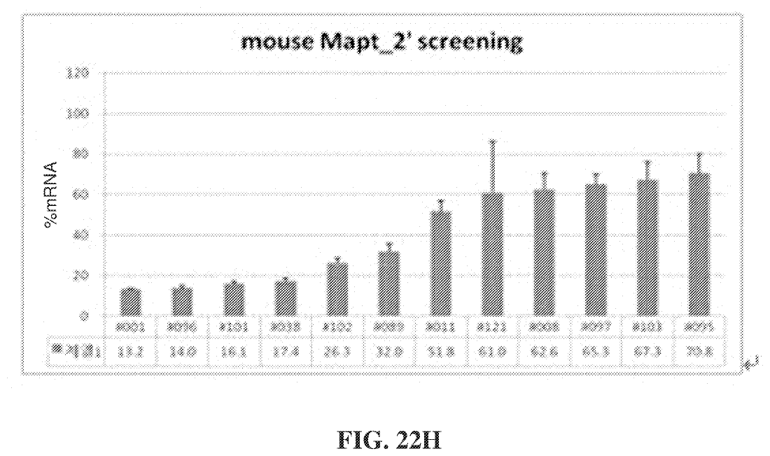

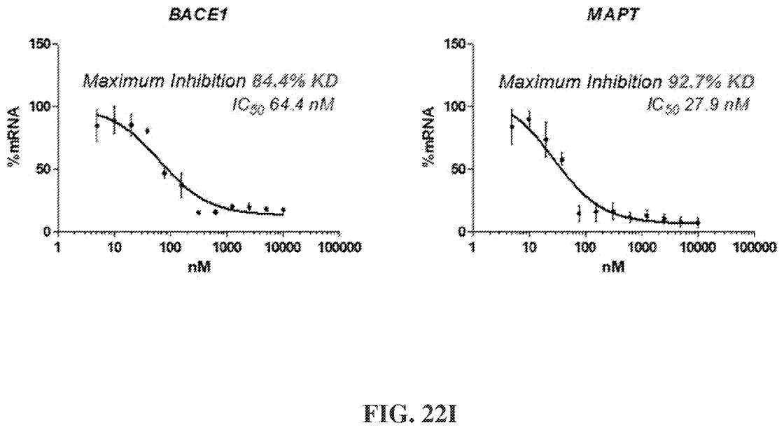

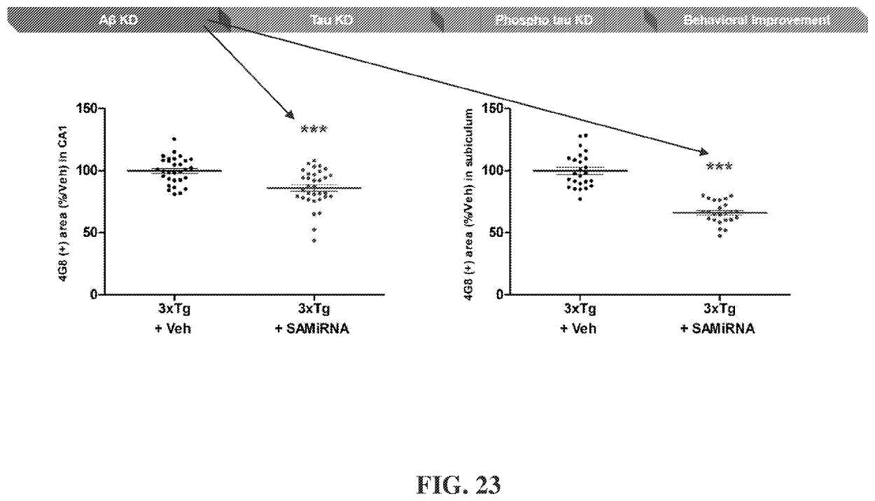

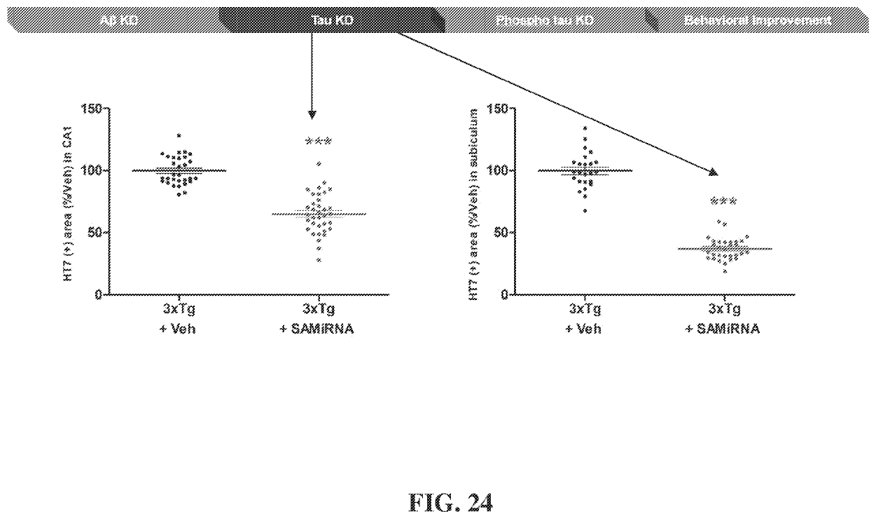

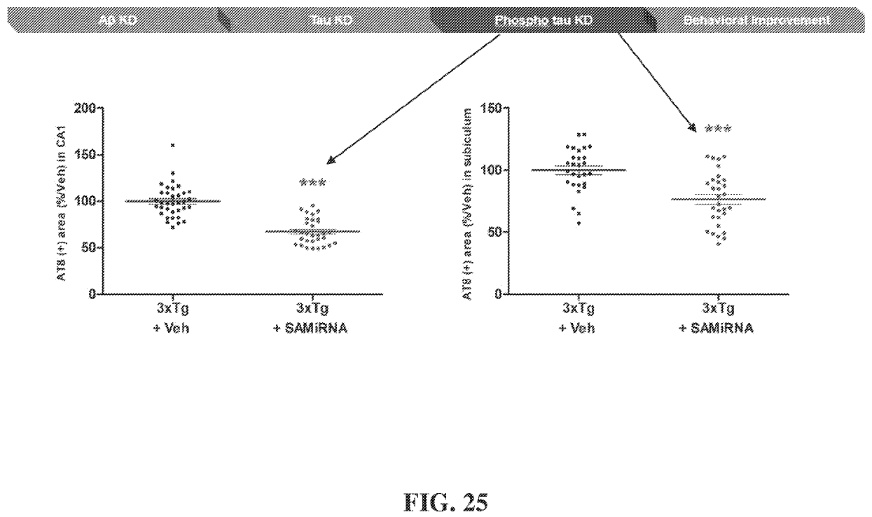

Image acquisition and analysis. To quantify immunoreactivity, images were captured with a Zeiss LSM 700 microscope (Carl Zeiss AG, Oberkochen, Germany) and analyzed using ImageJ software (National Institutes of Health, Bethesda, MD, USA). The analysis was performed blinded to the hippocampal formation of each animal's brain section. To measure the 4G8 (FIG. 23), HT7 (FIG. 24), and AT8 (FIG. 25), the area fractions of the immune-positive signals in the CA1 and subiculum was quantified.

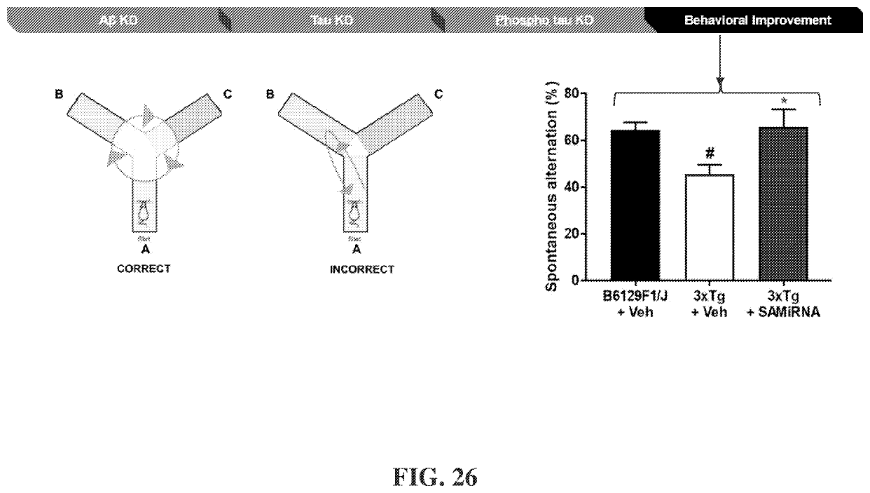

Behavioral memory impairment test. Short-term spatial memory was examined using a Y-maze 24 h after the final administration of glucose-SAMiRNA-BACE1/Tau. The Y-maze is a three-arm maze with equal angles between all arms (30 cm long×3 cm wide×14 cm high). The floor and walls of the maze were constructed using black acrylic. Mice were placed in the center of the Y-maze and allowed to explore each arm freely. Spontaneous alternation and the total number of arm entries were manually counted for each mouse during the 8 min session. The total number of arm entries was used as an indicator of locomotor activity. Spontaneous alternation was defined as successive entries into all arms (i.e., ABC, CBA, or BAC) without repetitions (i.e., ABA or CBC). The percentage of alternations was calculated as [(the number of alterations)/(the total number of arms entered-2)]×100 (FIG. 26)

Statistical analysis. All analyses were randomly conducted in a blinded manner for each group. Statistical analyses were performed using GraphPad Prism 7.0 software (GraphPad Software, Inc., La Jolla, CA, USA). Data are presented as mean±standard error of the mean. The significance of differences between the groups was determined by an independent t-test or one-way analysis of variance, with either Tukey's post hoc test. Differences were considered statistically significant at p<0.05.

### Example 10-4G: Analysis of Blood Biochemical Toxicity Parameters Including Hepatotoxicity, Lipotoxicity, Renal Toxicity, Cardiac Toxicity, and Homeostasis Toxicity in Experimental Animals Following 4-Week Repeated Administration: Measurements in Separated Serum Samples of Example 10

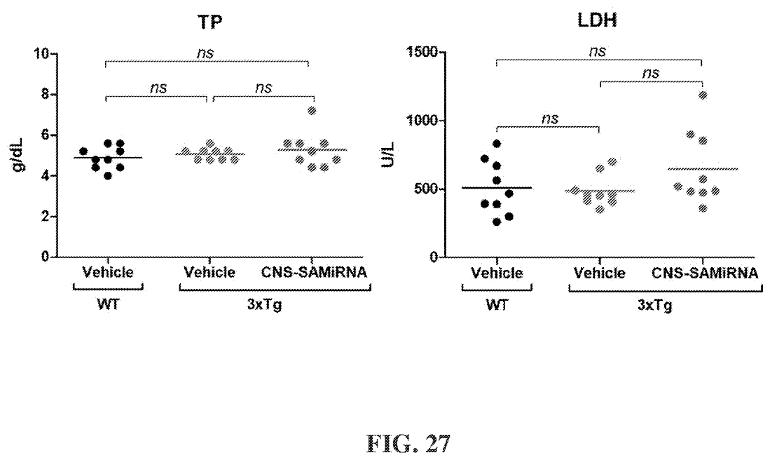

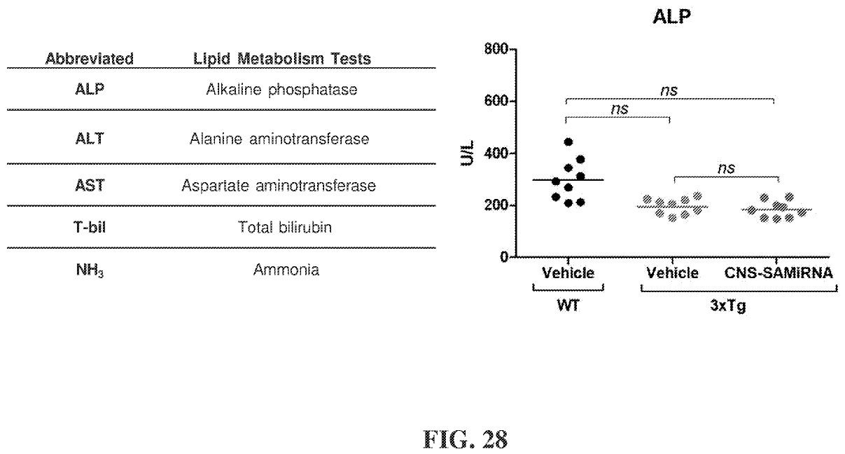

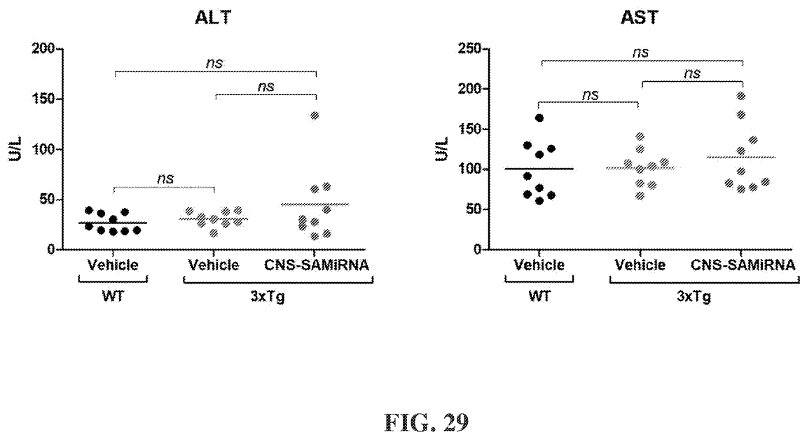

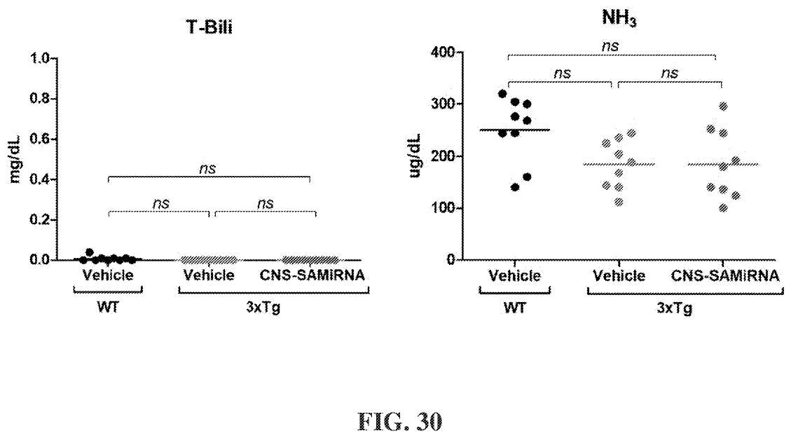

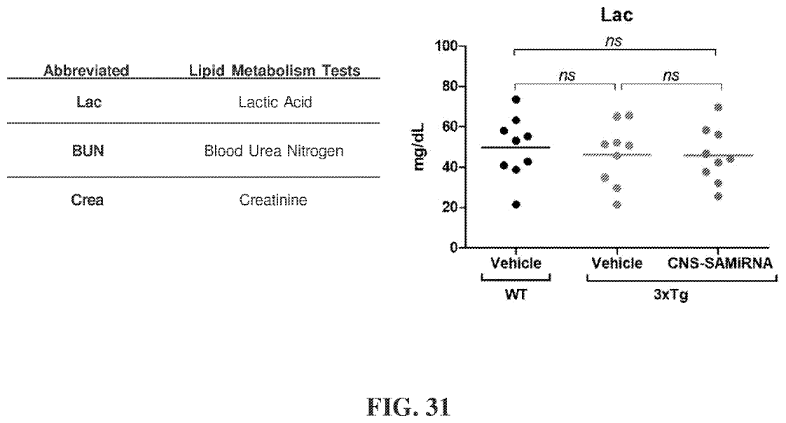

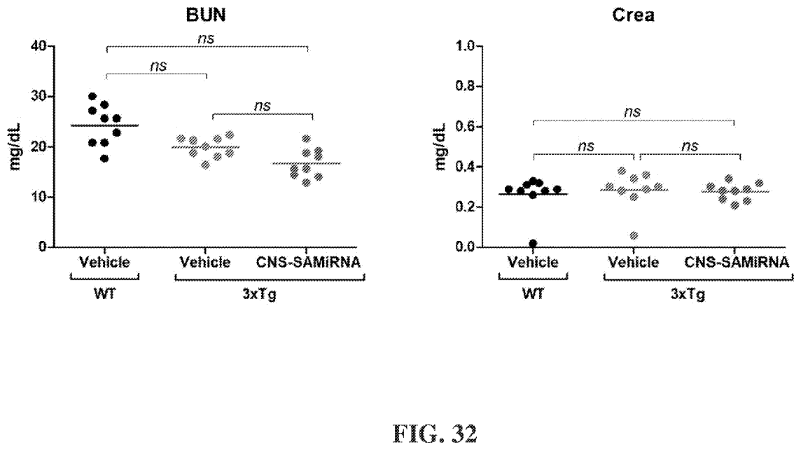

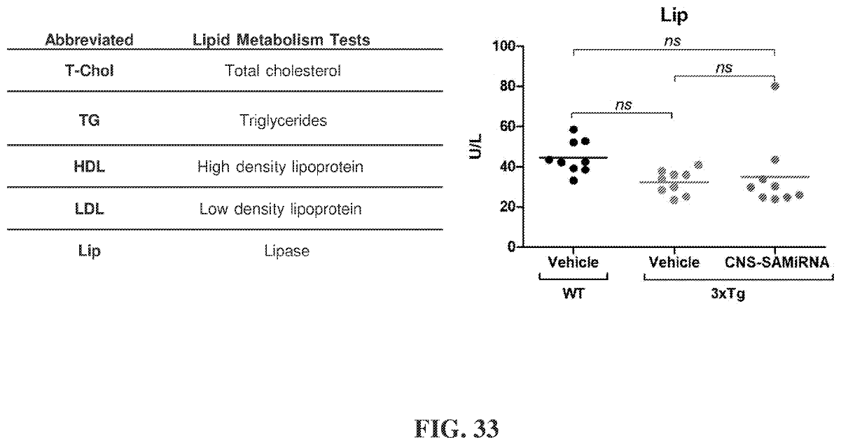

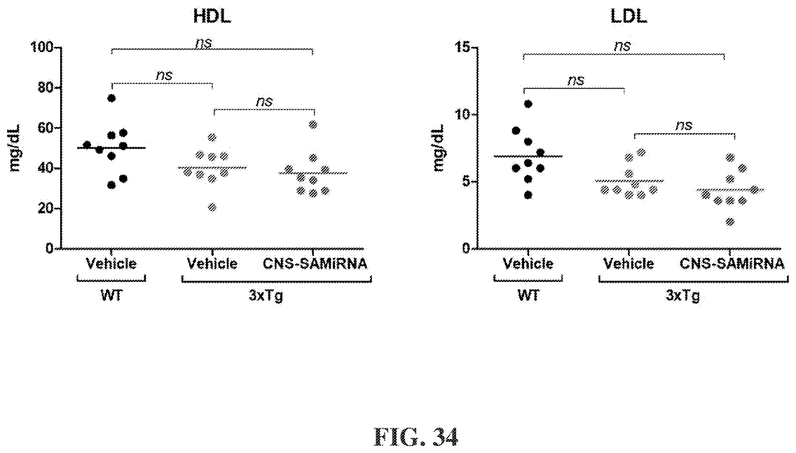

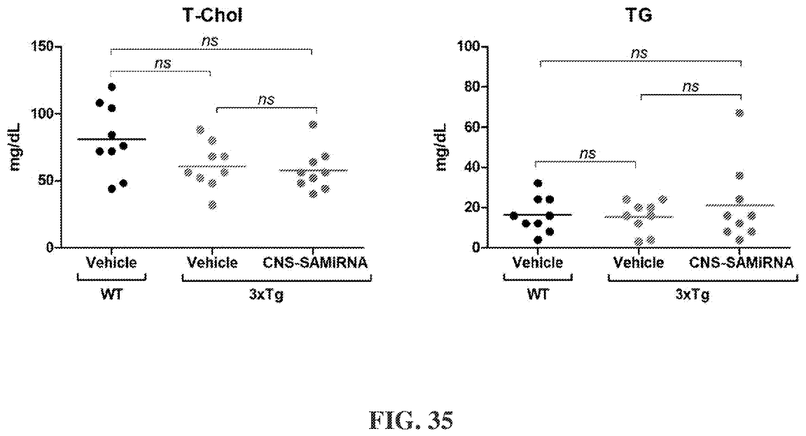

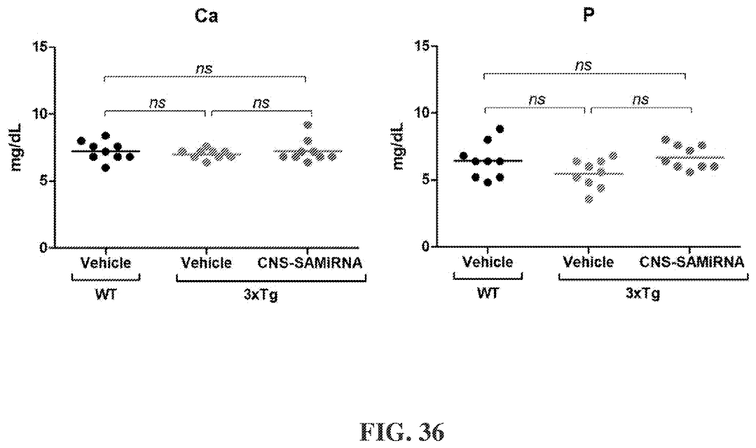

To assess the potential toxicity of the test substances, blood biochemical parameters related to hepatotoxicity (ALT, AST, ALP, LDH, GGT), lipotoxicity (CK, T-bili, T-Chol, TG), renal toxicity (Glu, BUN, Crea), cardiac toxicity (TP, Alb, A/G, Ca, P), and homeostasis toxicity (LDL-C, HDL-C, Lac, NH3, Amy, Lip, Mg, TBA) were measured in separated serum samples obtained from experimental animals (Example 10) following the completion of a 4-week repeated administration regimen. The measurements were performed to analyze the potential effects on various physiological systems (FIG. 27-36).

### Example 10-H: Grouping and Treatment of 6-Week-Old Female Balb/c Mice Purchased from Nara Biotech (Korea) and Analysis of Organ-Specific Distributions

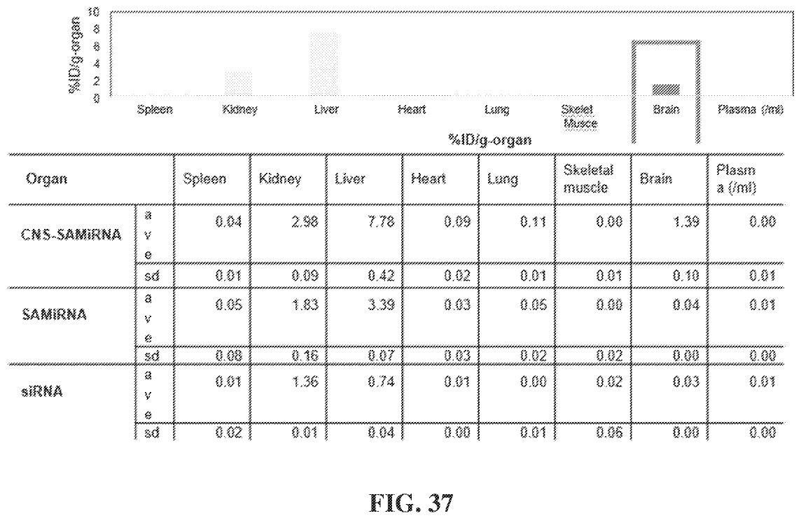

The 6-week-old female Balb/c mice obtained from Nara Biotech (Korea) were divided into four groups as follows: (1) Balb/c+Vehicle (n=2), (2) Balb/c+glucose-SAMiRNA-cy5 40 mpk (n=26), (3) Balb/c+SAMiRNA-cy5 40 mpk (n=2), and (4) Balb/c+siRNA-cy5 40 mpk (n=2). Within Group 3, further subdivision was performed into the following 13 groups: (a) free-fed/no boosting, (b) fasting for 12 hours/no boosting, (c) fasting for 12 hours/boosting at 0.5 hours post-injection, (d) fasting for 12 hours/boosting at 1 hour post-injection, (e) fasting for 12 hours/boosting at 2 hours post-injection, (f) fasting for 12 hours/boosting at 3 hours post-injection, (g) fasting for 12 hours/boosting at 5 hours post-injection, (h) fasting for 24 hours/no boosting, (i) fasting for 24 hours/boosting at 0.5 hours post-injection, (j) fasting for 24 hours/boosting at 1 hour post-injection, (k) fasting for 24 hours/boosting at 2 hours post-injection, (l) fasting for 24 hours/boosting at 3 hours post-injection, and (m) fasting for 24 hours/boosting at 5 hours post-injection. After each fasting condition, glucose-SAMiRNA-cy5, SAMiRNA-cy5, and siRNA-cy5 were intravenously administered at a dose of 40 mpk, followed by intraperitoneal administration of a 200 μl solution of 20% D-glucose at each respective boosting time. After 24 hours, blood was collected via retro-orbital bleeding to obtain plasma samples. Subsequently, the mice were euthanized, and residual substances within the vasculature were removed by perfusion via the heart. Brain tissue, spleen, kidney, liver, heart, lung, and skeletal muscle (femoral) were collected. All experiments were conducted under the supervision of the Animal Care and Use Committee at siRNAgen facilities. The collected organs were homogenized, and the absolute quantification of the administered dose in percentage of weight per organ was determined by generating standard curves using a plate reader (M200PRO, TECAN) at an excitation wavelength of 645 nm and an emission wavelength of 670 nm (FIG. 37).

### Example 10-5. An Exemplary Compound as Provided Herein SRN-008 PoC Study Shows Improved AD-Associated Markers and Cognitive Behavior in 3×Tg AD Mice

When an exemplary compound as provided herein, SRN-008 (a compound comprising BACE1 siRNA (oligonucleotide targeting BACE1) and MAPT siRNA (oligonucleotide targeting MAPT)), the therapeutic candidate for AD, developed using CNS-SAMiRNA™ platform, was administered into 8-month-old 3×Tg AD mice which display human Aβ and neurofibrillary tangles (NFT) of tau protein as well as cognitive symptoms of AD, short-term spatial memory was recovered in Y-maze test to the level of healthy mice after 4 administrations over a month.

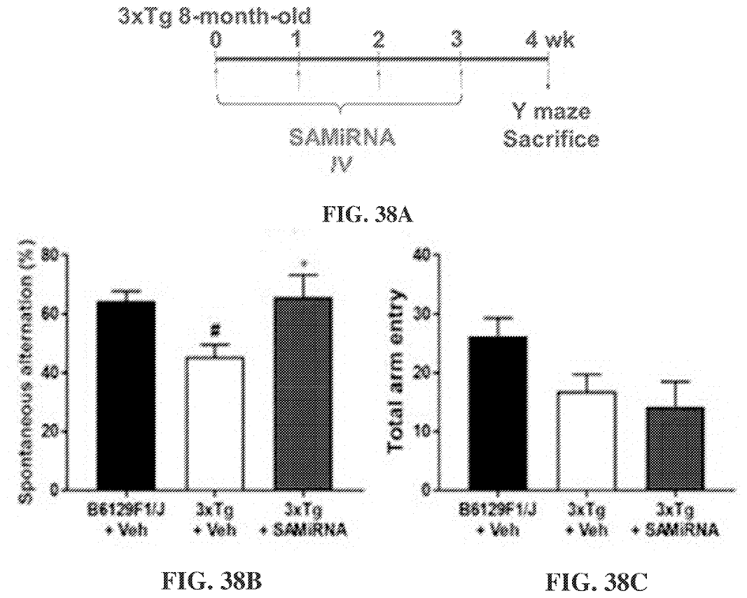

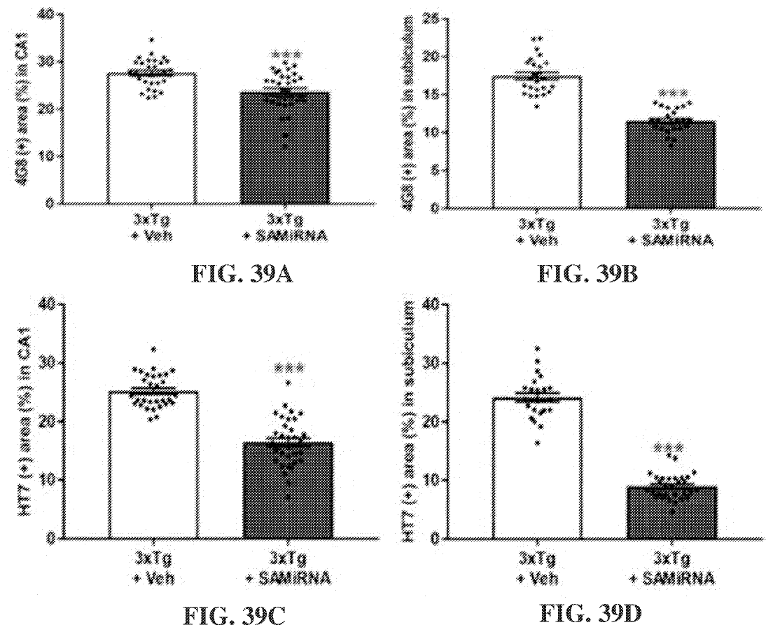

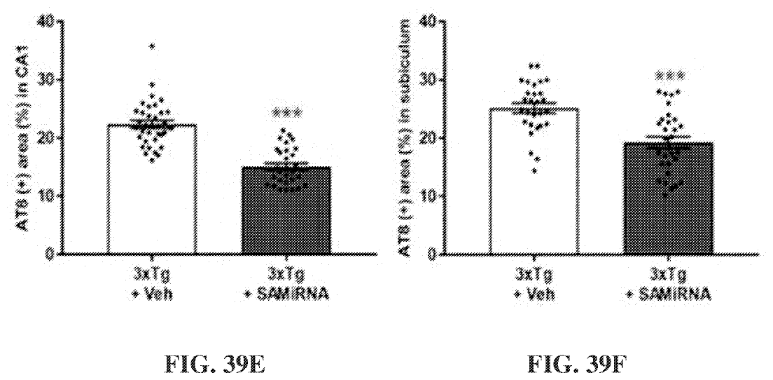

In the AD brains, microtubule-associated protein tau becomes abnormally hyperphosphorylated, leading to the loss of normal physiological function, increased toxicity, and aggregation to form NFTs. Histological analysis verified significant reduction in Aβ deposition, human tau expression, and phosphorylated tau in the CA1 and subiculum regions in the brains of 3×Tg mice treated with SRN-008. These results suggest SRN-008 ameliorates and potentially reverses AD progression (FIGS. 38A-38C, FIGS. 39A-39F).

### Example 10-6. Conclusions

This study showcases the new CNS-SAMiRNA™ as an effective platform for intravenously delivering siRNA to the brain, and the therapeutic potential of an exemplary compound as provided herein SRN-008 for the treatment of AD. The IV-enabling CNS-SAMIRNA™ platform can open myriad doors for developing novel therapies with better patient experience for numerous other neurological diseases with high unmet needs.

A novel siRNA delivery platform was developed that enhances the intravenous delivery of siRNA across BBB by over 20 folds. The IV administration of the therapeutic candidate SRN-008, an exemplary compound as provided herein, for Alzheimer's disease (AD) developed using this platform led to improvements in both AD-associated markers and behavioral scores, demonstrating a significant therapeutic potential for AD, a disease with high unmet needs.

### Example 11. Receptor-Targeting Chemistry is Combined with SAMiRNA™ Technology to Enable CNS Delivery of siRNA and Effective KD

An adult human brain at rest consumes 20-25% of its resting total body glucose consumption rate, showing high activity amounts of glucose transporters.

An exemplary embodiment of CNS platform combines receptor targeting moiety+SAMIRNA™ to deliver across BBB, achieving effective KD without toxicity.

Unlike other brain targeting mechanisms such as transferrin receptors, glucose-receptor mediated delivery can be controlled through blood sugar monitoring.

An exemplary embodiment of Brain Platform delivers to the deep brain via IV, enabling targeted delivery to the regions and cells affected by various CNS diseases.

### Example 12. Disease or Disorder Models

Following are exemplary disease or disorder models to investigate the efficiency of the compounds as provided herein, the compositions as provided herein, nanoparticles as provided herein, or pharmaceutical compositions as provided herein.

TABLE 8
Exemplary diseases models
Table 8. Exemplary diseases models

| Disorder | |
|---|---|
| or disease | Models |
| Alzheimer's | In vivo: SAMP8 mice |
| disease | In vivo: mouse model of Alzheimer disease |
| | In vitro: HFF-1 cells |
| | In vivo: AbPP-PS1 mice |
| | In vivo: APP/PS1 transgenic mice |
| | In vitro: bEnd.3 |
| | In vitro: IMR-32 (human neuroblastoma cells) |
| | In vitro: C17.2 (neural stem cells) |
| | In vivo: TG2576 mice |
| | In vitro: human APP751 (H4APP751) |
| | In vivo: Male SD mice |
| | In vivo: rat model |
| | In vitro: dividing and neural stem cells |
| | In vitro: human neuroblastoma cell line (SH-SY5Y) |
| Parkinson's | In vitro: neuronal cell from P0 newborn |
| disease | C57BL/6J mice |
| | In vitro: human neuroblastoma cells (BE(2)-M17) |
| | In vivo: wild-type C57BL6 female mice |
| | In vivo: Primate Substantia Nigra |
| | In vivo: Thy1-hSNCA mice |
| | In vitro: Drosophila S2 cells and human fibroblasts |
| | In vivo: flies model of PD |
| | In vitro: human neuroblastoma cell line (SH-SY5Y) |
| | In vitro: PC12 cells |
| | In vitro: human neuroblastoma cell line (SH-SY5Y) |
| | In vivo: Thy1-aSyn mice |
| | In vivo: transgenic mouse model of PD |
| Huntington's | In vivo: HD transgenic mouse model, R6/2 |
| disease | In vivo: viral transgenic mouse model of HD |
| | In vitro: COS-7 (African green monkey fibroblasts) |
| | In vitro: SH-SY5Y (human neuroblastoma) |
| | In vitro: Neuro-2A (mouse neuroblastoma) |
| | In vivo: transgenic YAC128 mouse |
| multiple sclerosis | In vivo: EAE mice (mouse model of MS) |
| | In vivo: Mouse model of acute demyelination |
| | In vivo: Male Wistar rats |
| spinal cord | In vitro: C6 glioma cells |
| injury | In vivo: SCI model rat |
| | In vivo: female Wistar rats |
| | In vivo: Female BALB/c mice1 |
| | In vivo: SCI model rat |
| | In vivo: female Sprague-Dawley rats |

Whereas specific parts of the present disclosure have been described in detail in the foregoing, it shall be clear to persons skilled in the art that these detailed descriptions are but preferred embodiments, and that the scope of the present disclosure is not limited hereto. Therefore, the actual scope of the present disclosure is defined by the appended claims and their equivalents.

TABLE 3
Exemplary sugar ligands and their corresponding GLUT receptors
Table 3. Exemplary sugar ligands and their corresponding GLUT receptors

| Sugar ligands | | | | |
|---|---|---|---|---|
| and derivates | Type | UnitProt | Km, mM | Class |
| Fructose | GLUT7 | Q6PXP3 | 0.5 | Class II |
| | GLUT9 | Q9NRM0 | 0.5 | Class II |
| | GLUT11 | Q9BYW1 | 0.5 | Class II |
| | GLUT5 | P22732 | 6 | Class II |
| | GLUT2 | P11168 | 76 | Class I |
| | GLUT8 | Q9NY64 | — | Class III |
| | GLUT12 | Q8TD20 | — | Class III |
| Galactose | GLUT3 | P11169 | 8.5 | Class I |
| | GLUT2 | P11168 | 92 | Class I |
| | GLUT1 | P11166 | — | Class I |
| | GLUT14 | Q8TDB8 | — | Class I |
| | GLUT4 | P14672 | — | Class I |
| Glucosamine | GLUT2 | P11168 | 0.8 | Class I |
| | GLUT1 | P11166 | — | Class I |
| | GLUT4 | P14672 | — | Class I |
| Glucose | GLUT12 | Q8TD20 | 0.3 | Class III |
| | GLUT7 | Q6PXP3 | 0.5 | Class II |
| | GLUT9 | Q9NRM0 | 0.5 | Class II |
| | GLUT11 | Q9BYW1 | 0.5 | Class II |
| | GLUT3 | P11169 | 1.4 | Class I |
| | GLUT10 | O95528 | 2 | Class III |
| | GLUT1 | P11166 | 0.7-3.2 | Class I |
| | GLUT4 | P14672 | 5 | Class I |
| | GLUT6 | Q9UGQ3 | 5 | Class III |
| | GLUT2 | P11168 | 17 | Class I |
| | GLUT8 | Q9NY64 | — | Class III |
| Mannose | GLUT2 | P11168 | 125 | Class I |
| | GLUT1 | P11166 | — | Class I |
| | GLUT3 | P11169 | — | Class I |
| | GLUT4 | P14672 | — | Class I |
| Xylose | GLUT3 | P11169 | — | Class I |
| Dehydroascorbic acid | GLUT4 | P14672 | — | Class I |
| Trehalose | GLUT8 | Q9NY64 | — | Class III |
| Urate | GLUT9 | Q9NRM0 | — | Class II |

TABLE 4
Exemplary organs or cells based on the GLUT expressing type
Table 4. Exemplary organs or cells based on the GLUT expressing type

| | | Tissue | Specific |
|---|---|---|---|
| Class | Type | or organ | expression |
| Class I | GLUT1 | Brain | Blood-brain barrier |
| | | | Astrocyte |
| | | Blood | Erythrocyte |
| | | Cancer | Brain |
| | | | Breast |
| | | | Cervix |
| | | | Colon |
| | | | Kidney |
| | | | Lung |
| | | | Ovary |
| | | | Prostate |
| | | | Skin |
| | | | Thyroid |
| | GLUT2 | Brain | Astrocyte |
| | | Intestine | Gastrointestinal tract |
| | | | Small intestine |
| | | Liver | — |
| | | Kidney | — |
| | | Pancreas | — |
| | GLUT3 | Brain | Neurons |
| | | | Blood-brain barrier |
| | | | Astrocyte |
| | | Embryos | — |
| | | Testis | Sperm |
| | | White blood | — |
| | | cells | |
| | | Cancer | Brain |
| | | | Breast |
| | | | Colon |
| | | | Cervix |
| | | | Kidney |
| | | | Lung |
| | GLUT14 | Testis | — |
| | | Cancer | Brain |
| | GLUT4 | Brain | Neuron |
| | | Muscle | — |
| | | Heart | — |
| | | Fat | Adipocyte |
| Class II | GLUT5 | Brain | Microglia |
| | | | Blood-brain barrier |
| | | Intestine | Small intestine |
| | | Testis | — |
| | | Muscle | — |
| | | Fat | — |
| | | Cancer | Breast |
| | | | Pancreas |
| | | | Ovary |
| | | | Lung |
| | | | Leukemia |
| | GLUT7 | Intestine | Small intestine |
| | | Colon | — |
| | | Prostate | — |
| | | Testis | — |
| | GLUT9 | Liver | — |
| | | Kidney | — |
| | | Intestine | Small intestine |
| | | Colon | — |
| | GLUT11 | Heart | — |
| | | Muscle | — |
| | | Kidney | — |
| | | Pancreas | — |
| | | Placenta | — |
| Class III | GLUT6 | Brain | — |
| | | Spleen | Leukocyte |
| | GLUT8 | Brain | Neurons |
| | | Testis | — |
| | | Liver | — |
| | | Spleen | — |
| | | Fat | Brown adipose |
| | | Blastocysts | — |
| | GLUT10 | Liver | — |
| | | Pancreas | — |
| | | Fat | White adipose |
| | GLUT12 | Brain | Astrocyte |
| | | Muscle | — |
| | | Heart | — |
| | | Fat | — |
| | | Prostate | — |
| | | Cancer | Breast |
| | GLUT13 | Brain | Neurons |

TABLE 5
Exemplary diseases related to the GLUT expressing organ or cell
Table 5. Exemplary diseases related to the GLUT expressing organ or cell

| Class | Type | Associated disease |
|---|---|---|
| Class I | GLUT1 | Brain delivery |
| | | Over expressed in many cancers |
| | | Cancers that express more GLUT1 have been shown to be more aggressive and |
| | | invasive. |
| | | The larger molecular weight GLUT1 is present in the cerebral cortex and |
| | | microvessels, the lower isoform is present in the microvessels-depleted brain |
| | | membranes (neuronal/glial membranes) and synaptosomes, and an intermediate form is |
| | | present in the choroid plexus. It is also present in erythrocytes, granulocytes, |
| | | adipocytes, muscle cells, kidney, colon, and many fetal tissues. It acts as a receptor for |
| | | human T cell leukemia virus type 1. It plays an essential role in CD4+ T cell activation. |
| | | It has affinity for glucose, and also transports galactose, mannose, and glucosamine. |
| | GLUT2 | Plays a significant role in carbohydrate uptake in the intestines |
| | | Type-2 diabetes |
| | | It is expressed in the intestinal absorptive epithelial cells, hepatocytes, pancreatic |
| | | β-cells, and kidney cells. GLUT2 mRNAs are present in the brain nuclei, including the |
| | | nucleus tractus solitatius, the motor nucleus of the vagus, the paraventricular |
| | | hypothalamic nucleus, the lateral hypothalamic area, the arcuate nucleus, and the |
| | | olfactory bulbs. GLUT2 is a low affinity transporter for glucose, galactose, mannose, |
| | | and fructose. It is a high affinity transporter for glucosamine. |
| | GLUT3 | Brain delivery |
| | | Over expressed in many cancers |
| | | Over expressed in glioblastoma |
| | | It is highly expressed in the brain, spermatozoa, placenta, preimplantation embryos, |
| | | fibroblasts human platelets, retinal endothelial cells, and white blood cells. |
| | GLUT14 | Has 96% identity with GLUT3 |
| | | Both isoforms of GLUT14 are specifically expressed in the human testis. |
| | GLUT4 | Over expressed in gastric cancer |
| | | Regulated by insulin as insulin biding receptors translocate GLUT4 to the cell surface |
| | | linked to obesity, T-2 diabetes |
| | | It is expressed in the skeletal muscle cells, cardiomyocytes, adipose tissue, |
| | | hypothalamus, cerebellum, cortex, and hippocampus. GLUT4 is a high affinity |
| | | transporter of glucose. It also transports dehydroascorbic acid and glucosamine. |
| Class II | GLUT5 | Strongly linked to cancer development, progression and metastasis |
| | | Over expressed in colon cancers |
| | | Chron's disease |
| | | It is expressed in the apical membrane of the enterocytes, in the plasma membrane |
| | | of mature spermatozoa, kidney, fat, skeletal muscle, and the brain. |
| | GLUT7 | Expressed in the small intestine and colon |
| | | It is primarily expressed in the small intestine (distal region), colon, testis, and |
| | | the prostate gland. It has a high affinity for both glucose and fructose. It does not |
| | | transport galactose, 2-deoxy-D-glucose and xylose. |
| | GLUT9 | Mainly expressed in the liver and kidney |
| | | GLUT9b is expressed only in the liver and kidney, whereas GLUT9a is present in |
| | | many more tissues and organs, such as liver, kidney, intestine, leukocytes, and |
| | | chondrocytes. It is a glucose, fructose, and urate transporter. GLUT9a and GLUT9b |
| | | transport urate with the same kinetics. |
| | GLUT11 | Various organs |
| | | GLUT11-A is expressed in the heart, skeletal muscle, and kidneys; GLUT11-B is |
| | | expressed in the kidneys, adipose tissue, and placenta; GLUT11-C is expressed in the |
| | | adipose tissue, heart, skeletal muscle, and pancreas. GLUT11 appears to recognize |
| | | fructose with comparable affinity to, possibly with even higher affinity than, glucose |
| | | (Km ~0.2 mM) but not galactose. |
| Class III | GLUT6 | Overexpressed in endometrial cancer |
| | | It is predominantly expressed in the brain, spleen, peripheral leukocytes, and in |
| | | germinal cells of the testis. It exhibits glucose transport activity; however, it appears |
| | | to be a low-affinity facilitator of glucose. |
| | GLUT8 | Suggest that the steatosis pathways that are triggered by high fructose intake are |
| | | reliant on GLUT8 as the primary fructose transporter in hepatic cells. |
| | | Hepatic steatosis |
| | | It is expressed in the testis, cerebellum, adrenal gland, liver, spleen, brown adipose |
| | | tissue, in the spermatocytes and in the human mature spermatozoa. It is a high affinity |
| | | transporter of glucose. |
| | GLUT10 | Arterial tortuosity syndrome |
| | | Overexpressed in leukemia |
| | | It is expressed in the skeletal muscle, heart, and adipose tissue. GLUT10 mRNA is |
| | | expressed in the liver, pancreas, placenta, and kidney. GLUT10 is has a very high |
| | | affinity for both deoxy-D-glucose and D-galactose, but not for fructose. |
| | GLUT12 | Overexpressed in breast cancer |
| | | It is expressed in the heart, skeletal muscle, adipose tissue, prostate gland, kidneys, |
| | | small intestine, chondrocytes, and in the placenta. It exhibits transport activity for |
| | | glucose, galactose, and fructose. |
| | GLUT13 | Brain expression |
| | | It is expressed in the brain, white and brown adipose tissue, and the kidneys. It is a |
| | | H+/myo-inositol cotransporter that exhibits transport activity only for myo-inositol |
| | | with a high affinity. |

TABLE 6A
Exemplary molecular targets for neurological
diseases: Alzheimer's disease
Table 6A. Exemplary molecular targets for neurological
diseases: Alzheimer's disease
siRNA therapeutic applications in Alzheimer's disease

| Target | |
|---|---|
| molecule(s) | Effect(s) |
| BACE1-AS | Improvement of memory and learning behaviors |
| BACE1 | Decreasing amyloid plaque rate |
| | improvement in neuropathological and behavioral signs |
| | Significant suppression of BACE1 expression |
| | Reduction of plaque load in the cerebral cortex and |
| | hippocampus |
| | Increasing the level of synaptophysin |
| | Rescued memory loss |
| presenilin1 | Reducing the level of Aβ 42 |
| (PS1) | |
| ROCK-II | Promoting axonal regeneration |
| I2 PP-2A | Decreasing the level of Aβ and APP and |
| | phosphorylated tau |
| | Improvement of memory and learning ability |
| ACAT-1 | Reducing the enzymatic process of APP |
| | Enhancing the level of free cholesterol |
| Nogo receptor | Promoting the regeneration and repair of cholinergic |
| | neurons |
| mutant | Decreasing the level of amyloid plaque |
| presenilin1 | |
| (L392V PS-1) | |
| BACE1 | |
| APP | Improvement in synaptic activity and mitochondrial |
| Tau | function |
| VDAC1 | |

TABLE 6B
Exemplary molecular targets for neurological
diseases: Parkinson's disease
Table 6B. Exemplary molecular targets for neurological
diseases: Parkinson's disease
siRNA therapeutic applications in Parkinson's disease

| Target | |
|---|---|
| molecule(s) | Effect(s) |
| α-synuclein (SNCA) | Reducing the level of SNCA |
| | the first evidence of successful anti-α- |
| | synuclein intervention in the primate |
| | Decreased hSNCA expression |
| | Rescue of hSNCA-mediated behavioral deficits |
| | Improvement in motor dysfunction |
| | Protect cells from death via apoptosis |
| | Reducing the accumulation of α-syn |
| | Amelioration of inflammatory pathology |

TABLE 6C
Exemplary molecular targets for neurological
diseases: Huntington's disease
Table 6C. Exemplary molecular targets for neurological
diseases: Huntington's disease
siRNA therapeutic applications in Huntington's disease

| Target | |
|---|---|
| molecule(s) | Effect(s) |
| Htt | Inhibition of the Htt expression |
| | reduction of size and number of NIIs |
| | Improvement of some movement problem |
| | Survival of neurons |
| | Decreasing the level of mutant htt protein |

TABLE 6D
Exemplary molecular targets for neurological
diseases: Multiple sclerosis
Table 6D. Exemplary molecular targets for neurological
diseases: Multiple sclerosis
siRNA therapeutic applications in multiple sclerosis

| Target | |
|---|---|
| molecule(s) | Effect(s) |
| T-bet | Specifically regulate IFN |
| | Prevented the onset of disease |
| Notch1 | Promotion of the remyelination |
| | Improve OL differentiation |
| | Increase mature OL |
| LINGO-1 | Better motor function |
| | Repair in histopathological sections |
| NR4A2 | Inhibiting the pathogenic potentials of IFN |
| | and IL-17 |
| TRIF | Alleviating the severity of EAE (experimental |
| | autoimmune encephalomyelitis) via the inhibition |
| | of interleukin and cytokine release |
| caspase-2 | Significant inhibition of nerve cell loss |
| | Decreasing in RNFL thickness |
| | Increased survival of RGC after ON |
| CaMKII | Reduced mechanical and thermal hypersensitivity |
| | Essential role of CaMKII in inducing and maintaining |
| | the evoked and non-evoked pain in EAE. |

TABLE 6E
Exemplary molecular targets for neurological
diseases: Spinal cord injury
Table 6E. Exemplary molecular targets for neurological
diseases: Spinal cord injury
siRNA therapeutic applications in spinal cord injury

| Target | |
|---|---|
| molecule(s) | Effect(s) |
| GFAP | Improvement of urinary function |
| Vimentin | |
| EphB3 | Improvement in axonal regeneration and the motor function |
| iNOS | Improvement of the secondary damage following SCI |
| Nischarin | Improvement of motor function |
| RhoA | Improvement in walking |
| | declining of allodynia |

TABLE 7
Exemplary linkersTable 7. Exemplary linkers
—NH(CH₂)nCONH—CH₂—CH(OH)—CH₂—
hexaethylene glycol

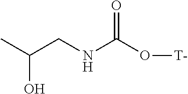

Compound 4
T refers to 1 to 15 repetitive Compound 1 represented below, and G is selected from the group
consisting of CH2, O, S and NH.

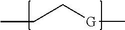

Compound 1
ether linkage

### Example 13. Evaluation of Cancer Cell Uptake and Gene Inhibition Efficiency of Glucose-SAMiRNA Nanoparticles

### Example 13-1. Comparative Evaluation of Delivery Efficiency in GLUTs Overexpressing Cancer Cell Lines

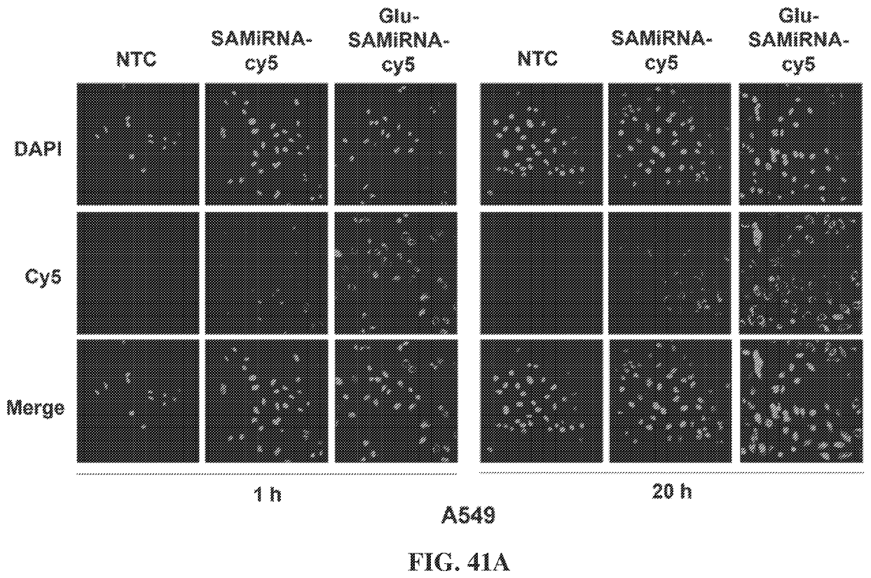

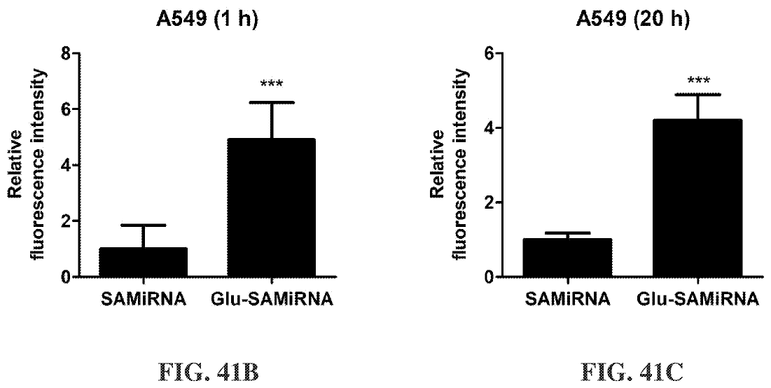

A549, a lung cancer cell line, was cultured in RPMI-1640 medium with normal glucose concentration (Hyclone #SH30027.01) containing 10% FBS on coverslips coated with 0.01% poly-L-lysine in a 12-well plate under conditions of 37° C. and 5% CO2. After 24 hours, the cells were washed with RPMI-1640 medium without glucose (Gibco #11879-020) containing 10% FBS and treated with SAMiRNA-cy5 or glu-SAMiRNA-cy5 at a concentration of 2 μM for 1 hour or 20 hours. Subsequently, the cells were fixed with 4% PFA, mounted with a mounting solution containing DAPI, and subjected to fluorescence imaging (FIG. 41A, blue: DAPI, red: Cy5) using a DragonFly confocal microscope (Andor) at a magnification of 40× and a size bar of 40 μm. The acquired fluorescence signals were quantified using ImageJ software, and the fluorescence intensity ratio of glu-SAMiRNA-cy5 normalized to SAMiRNA-cy5 was plotted (FIGS. 41B-41C).

As shown in FIGS. 41A-41C, the results demonstrated improved cellular delivery efficiency of glu-SAMiRNA-cy5 compared to SAMiRNA-cy5 in both the 1-hour and 20-hour treatment groups. The delivery to GLUT overexpressing cancer cells was enhanced by 4.9±1.3-fold and 4.2±0.7-fold (Mean±SD) after 1 hour and 20 hours, respectively (FIGS. 41B-41C). To compare the inhibitory efficacy resulting from improved delivery, an additional in vitro experiment was conducted in Example 13-2.

### Example 13-2. Comparative Evaluation of KD Efficacy in GLUTs Overexpressing Cancer Cell Lines

A549, a lung cancer cell line, was cultured in a 12-well plate at a density of 6×10⁴ cells/well in RPMI-1640 medium without glucose (Gibco #11879-020) containing 10% FBS under conditions of 37° C. and 5% CO2. After 24 hours, the cells were treated with SAMiRNA or glu-SAMiRNA at concentrations of 10, 2, 0.4, 0.08, 0.016, and 0.032 μM. After 20 hours, the cells were harvested for RT-qPCR analysis. Total RNA was extracted using the AccuPrep® Universal RNA Extraction Kit (BIONEER) and 100 units of DNase I, RNase-free (Enzynomics). The extracted total RNA was quantified using the Infinite M200 Pro (TECAN), and cDNA was synthesized using the AccuPower® RocketScript™ Cycle RT PreMix (BIONEER) on Mygenie 96 (BIONEER) with cycles of 37° C. 30 seconds, 48° C. 4 minutes, 55° C. 30 seconds for 12 cycles, and 95° C. 5 min. qPCR was performed using the AccuPower® 2× GreenStar™ qPCR Master Mix (BIONEER), DEPC-DW (BIONEER), and 500 nM primers (AREG_F: ACACCTACTCTGGGAAGCGT (SEQ ID NO: 132), AREG_R: GCCAGGTATTTGTGGTTCGT (SEQ ID NO: 133), RPL13A_F: GTGTTTGACGGCATCCCACC (SEQ ID NO: 134), RPL13A_R: TAGGCTTCAGACGCACGACC (SEQ ID NO: 135) on an Armadillo PCR Plate, 384-well, clear, white wells (Thermo Scientific) and Exicycler™ 384 (BIONEER) using a method of 95° C. for 10 min, followed by 40 cycles of 95° C. for 5 sec, 58° C. for 25 sec, and 72° C. for 30 sec. The relative quantification of RPL13A was performed. The KD values according to the treatment concentrations were determined using GraphPad Prism through nonlinear fitting to calculate the IC50 values (Fit R² value: SAMIRNA, 0.9916; glu-SAMiRNA, 0.9885).

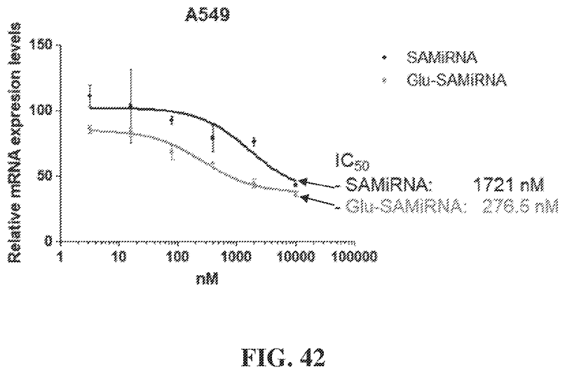

As shown in FIG. 42, the IC50 values for SAMiRNA and glu-SAMiRNA were 1721.0 nM and 276.5 nM, respectively, indicating that glu-SAMiRNA exhibited a gene inhibition efficacy over six times higher than SAMiRNA.

Clinical studies have revealed a correlation between glucose uptake in cancer patients and the diagnosis as well as prognosis of tumors. Numerous reports have demonstrated the overexpression of GLUTs (glucose transporters) in various cancer cells (Yamamoto T., et al. 1990; Nishioka T., et al. 1992; Brown R. S., et al. 2018; Cantuaria G., et al. 2001). This indicates that regardless of cancer type, an excessive amount of glucose is being absorbed through the overexpressed GLUTs, and this is related to the progression of the disease, showing a positive correlation. Additionally, extensive research has identified different types of GLUTs predominantly expressed in each cancer type, and the affinity between sugars such as glucose, fructose, and galactose to each specific GLUT varies.

In our study, aiming to develop various solid tumor therapeutics using the SAMiRNA platform, a lung cancer cell line overexpressing GLUT1 was utilized to evaluate the target delivery efficiency and inhibitory efficacy of the therapeutics. The results demonstrated that glucose-SAMiRNA exhibited over 4-fold improved delivery efficiency and over 6-fold higher inhibitory rate. These enhanced values solely resulted from the effects of glucose conjugation on SAMIRNA, indicating the potential of glucose-SAMiRNA as a targeted nanoparticle therapeutic through active delivery, deviating from the conventional passive delivery approach.

As described earlier, by utilizing various GLUTs with different expression patterns in different cancer types and their respective binding affinities to various sugars, it is possible to develop drugs with optimal efficacy specific to each cancer type. For instance, Dehydrose specifically binds to GLUT4, which is known to be overexpressed in gastric cancer and has a significant association with obesity. Similarly, Urate exhibits specificity to GLUT9, which is predominantly overexpressed in the liver and kidney. Therefore, by using cancer-specific or organ/tissue-specific GLUTs along with high-affinity sugars as targeting ligands, SAMiRNA can enable highly specific delivery tailored to specific cancer types, organs, or tissues, offering great potential for the development of efficient and low-side-effect therapeutics.

Thus, the optimal combination of various sugars and GLUTs pairing as well as glucose-SAMIRNA as described in this disclosure has sufficient evidence and potential to be developed as highly efficient anticancer agent specific to solid tumors.

### OTHER EXEMPLARY EMBODIMENTS

The following enumerated clauses are illustrative examples of aspects and combination of aspects of the foregoing embodiments and examples.
Embodiment 1. A double strand oligonucleotide structure comprising the structure of Formula 1 below:

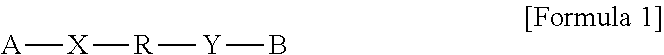

where, in Formula 1, A is a hydrophilic substance, B is a hydrophobic substance, X and Y each independently represent a simple covalent bond or linked-mediated covalent bond, and R refers to a double strand oligonucleotide comprising a sense strand and an antisense strand comprising a sequence complementary thereto;
and a monosaccharide is bonded to the A or B terminal.
Embodiment 2. The double strand oligonucleotide structure of Embodiment 1, wherein the monosaccharide is glucose, fructose, or galactose.
Embodiment 3. The double strand oligonucleotide structure of Embodiment 1, wherein a glucose is bonded to the terminal of the hydrophilic substance A, and which comprises the structure of Formula 14:

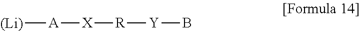

where, in Formula 14, A, B, X and Y are as defined in Formula 1 above; L is a monosaccharide; and i is an integer of 1 through 10.
Embodiment 4. The double strand oligonucleotide structure of Embodiment 3, wherein the A and Lᵢ are joined by a linker.
Embodiment 5. The double strand oligonucleotide structure of Embodiment 4, wherein the linker has the Formula of NH(CH₂)ₙCONH—CH₂—CH(OH)—CH₂—.
Embodiment 6. The double strand oligonucleotide structure of Embodiment 1, wherein the sense strand or anti-sense strand is comprised of 19 to 31 nucleotides.
Embodiment 7. The double strand oligonucleotide structure of Embodiment 1, wherein the oligonucleotide is siRNA, shRNA or miRNA.
Embodiment 8. The double strand oligonucleotide structure of Embodiment 1, wherein the sense or anti-sense strand is independently DNA or RNA.
Embodiment 9. The double strand oligonucleotide structure of Embodiment 1, wherein the sense strand or anti-sense strand of the double strand oligonucleotide comprises a chemical modification.
Embodiment 10. The double strand oligonucleotide structure of Embodiment 9, wherein the chemical modification is at least one selected from the group comprised of:
modifications wherein a hydroxy group (—OH) at the 2′ carbon position of a sugar structure in the nucleotide is substituted by any one selected from the group comprised of a methyl group (—CH₃), a methoxy group (—OCH₃), an amine group (—NH₂), a fluorine (—F), an O-2-methoxyethyl group, an O-propyl group, an O-2-methylthioethyl group, an O-3-aminopropyl group, an O-3-dimethylaminopropyl group, an O—N-methylacetamino group and an O-dimethylamidooxyethyl group;
modifications wherein the oxygen of a sugar structure in the nucleotide is substituted by sulfur;
modifications wherein the nucleotide bond becomes any one selected from the group comprised of a phosphorothioate bond, a boranophosphate bond and a methyl phosphonate bond; and,
modifications to a PNA (peptide nucleic acid), LNA (locked nucleic acid) or UNA (unlocked nucleic acid) form.
Embodiment 11. The double strand oligonucleotide structure of Embodiment 1, wherein at least one phosphate group is bonded to the 5′ terminal of the anti-sense strand.
Embodiment 12. The double strand oligonucleotide structure of Embodiment 1, comprising the structure of Formula 2 below:

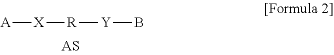

where, in Formula 2, S and AS refer to the sense strand and anti-sense strand of the double strand oligonucleotide of Embodiment 9, and the definitions of A, B, X and Y are identical to those of Embodiment 1.
Embodiment 13. The double strand oligonucleotide structure of Embodiment 12, comprising the structure of Formula 3 or Formula 3′ below:

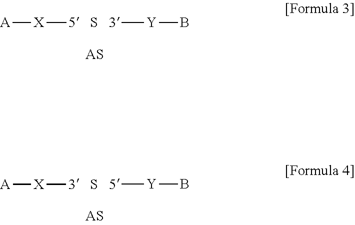

where, in Formula 3 and Formula 3′, the definitions of A, B, X, Y, S and AS are identical to those of Embodiment 6, and 5′ and 3′ refer to the 5′ and 3′ terminals of the sense strand.
Embodiment 14. The double strand oligonucleotide structure of Embodiment 1, wherein the hydrophilic substance is selected from the group comprised of polyethylene glycol (PEG), polyvinyl pyrrolidone and polyoxazoline.
Embodiment 15. The double strand oligonucleotide structure of Embodiment 1, wherein the hydrophilic substance has the structure of Formula 4 or Formula 5 below:

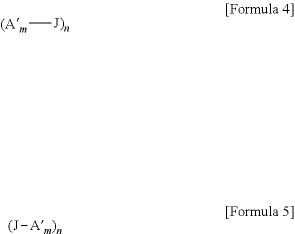

where, in Formula 4 and Formula 5 above, A′ refers to a hydrophilic substance monomer, J refers to a linker connecting a number m of hydrophilic monomers to each other or a number m of hydrophilic monomer to siRNAs, m is an integer of 1 to 15, and n is an integer of 1 to 10;
where the hydrophilic monomer A′ is any one compound selected from among Compound 1 through Compound 3 below, and the linker (J) is selected from the group comprised of PO₃⁻, SO₃⁻ and CO₂⁻;

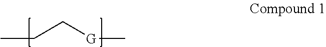

in Compound 1 above, G is selected from the group comprised of O, S and NH;

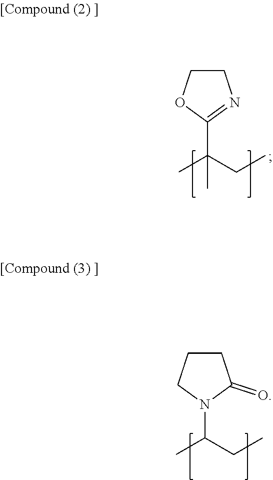

Embodiment 16. The double strand oligonucleotide structure of Embodiment 15, having the structure of Formula 6 or Formula 7 below:

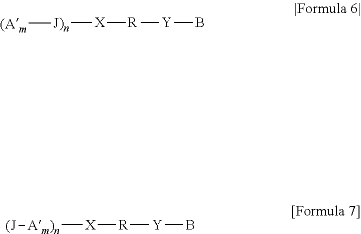

Embodiment 17. The double strand oligonucleotide structure of Embodiment 1, wherein the molecular weight of the hydrophilic substance is 200 to 10,000.
Embodiment 18. The double strand oligonucleotide structure of Embodiment 1, wherein the molecular weight of the hydrophobic substance is 250 to 1,000.
Embodiment 19. The double strand oligonucleotide structure of Embodiment 18, wherein the hydrophobic substance is any one selected from the group comprised of steroid derivatives, glyceride derivatives, glycerol ether, poplypropylene glycol, unsaturated or saturated hydrocarbon of C₁₂ through C₅₀, diacylphosphatidylcholine, fatty acid, phospholipid, lipopolyamine, lipid, tocopherol and tocotrienol.
Embodiment 20. The double strand oligonucleotide structure of Embodiment 19, wherein the steroid derivative is one selected from the group comprised of cholesterol, cholestanol, cholic acid, cholesteryl formate, cotestanyl formate, and colistanylamine.
Embodiment 21. The double strand oligonucleotide structure of Embodiment 19, wherein the glyceride derivative is one selected from the group comprised of mono-glyceride, diglyceride and tri-glyceride.
Embodiment 22. The double strand oligonucleotide structure of Embodiment 1, wherein the covalent bonds represented by X and Y are non-degradable bonds or degradable bonds.
Embodiment 23. The double strand oligonucleotide structure of Embodiment 22, wherein the non-degradable bond is an amide bond or a phosphorylated bond.
Embodiment 24. The double strand oligonucleotide structure of Embodiment 22, wherein the degradable bond is one selected from the group comprised of a disulfide bond, an acid-degradable bond, an ester bond, an anhydride bond, a biodegradable bond, and an enzyme degradable bond.
Embodiment 25. A nanoparticle comprising the double strand oligonucleotide structure according to any one of Embodiment 1 through Embodiment 24.
Embodiment 26. A composition for blood brain barrier permeation, the composition comprising the double strand oligonucleotide structure according to any one of Embodiment 1 through Embodiment 24.
Embodiment 27. A pharmaceutical composition for preventing or treating a disease, the composition comprising the double strand oligonucleotide structure according to any one of Embodiment 1 through Embodiment 24.
Embodiment 28. The pharmaceutical composition of Embodiment 27, wherein the disease is a neurological disorder selected from Alzheimer's Disease (AD), stroke, dementia, muscular dystrophy (MD), multiple sclerosis (MS), amyotrophic lateral sclerosis (ALS), cystic fibrosis, Angelman syndrome, Liddle syndrome, Parkinson's Disease, Pick's Disease, Pagets Disease, cancer, and traumatic brain damage.
Embodiment 29. The pharmaceutical composition of Embodiment 27, wherein the disease is a cancer.

1-215. (canceled)

216. A double strand oligonucleotide structure comprising the structure of Formula 1: 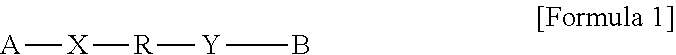
wherein:
R is a double strand oligonucleotide comprising a sense strand and an antisense strand comprising a sequence complementary thereto;
X and Y are independently a covalent bond or linker;
A is a hydrophilic moiety; and
B is a hydrophobic moiety;
wherein a ligand molecule for a glucose transporter (GLUT) or a functional analog of the ligand is covalently linked to a terminus of A and/or B via a second linker.

217. The double strand oligonucleotide structure of claim 216, wherein the structure of Formula 1 is represented by Formula 14 or Formula 15: 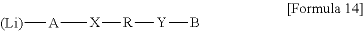
wherein:
L is a ligand molecule for a glucose transporter (GLUT) or a functional analog of the ligand; and
i is an integer from 1 to 10;
or 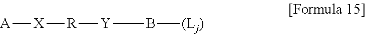
wherein:
L is a ligand molecule for a glucose transporter (GLUT) or a functional analog of the ligand; and
j is an integer from 1 to 10.

218. The double strand oligonucleotide structure of claim 217, wherein L is a sugar.

219. The double strand oligonucleotide structure of claim 218, wherein L is a monosaccharide.

220. The double strand oligonucleotide structure of claim 218, wherein the sugar is selected from fructose, galactose, glucosamine, glucose, mannose, xylose, dehydroascorbic acid, and trehalose.

221. The double strand oligonucleotide structure of claim 216, wherein the glucose transporter (GLUT) is selected from GLUT1, GLUT2, GLUT3, GLUT4, GLUT5, GLUT6, GLUT7, GLUT8, GLUT9, GLUT10, GLUT11, GLUT12, GLUT13, GLUT14.

222. The double strand oligonucleotide structure of claim 216, wherein A is a hydrophilic moiety selected from polyethylene glycol (PEG), hexaethylene glycol (HEG), polyvinyl pyrrolidone, and polyoxazoline.

223. The double strand oligonucleotide structure of claim 216, wherein B comprises a hydrophobic moiety of a molecular weight from 250 to 1,000.

224. The double strand oligonucleotide structure of claim 216, wherein B comprises a hydrophobic moiety selected from steroid derivatives, glyceride derivatives, glycerol ether, polypropylene glycol, unsaturated or saturated C₁₂-C₅₀ hydrocarbon chain, diacylphosphatidylcholine, fatty acid, phospholipid, lipopolyamine, lipid, tocopherol, and tocotrienol.

225. The double strand oligonucleotide structure of claim 224, wherein B comprises a hydrophobic moiety selected from glyceride derivative, glycerol ether, unsaturated or saturated C₁₂-C₅₀ hydrocarbon chain, and lipopolyamine, optionally wherein B is C₆—S—S—C₁₈.

226. The double strand oligonucleotide structure of claim 216, wherein A is represented by 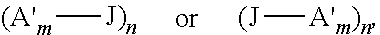
wherein
A′ is a hydrophilic moiety selected from Compounds 1, 2, or 3: 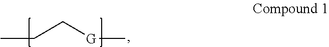
wherein G is selected from O, S, and NH; 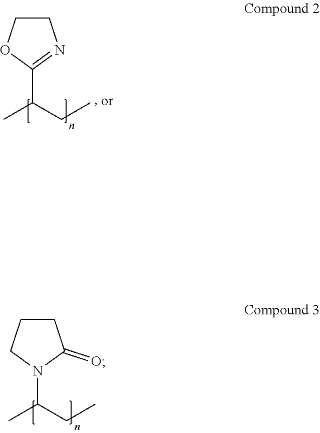
and
J is selected from PO₃⁻, SO₃⁻, or ester;
m is an integer from 1 to 15; and
n is an integer from 1 to 10.

227. The double strand oligonucleotide structure of claim 226, wherein G is O and J is PO₃⁻.

228. The double strand oligonucleotide structure of claim 227, wherein m is 5 to 10 and n is 2 to 7.

229. The double strand oligonucleotide structure of claim 216, wherein the linker is an ether linkage; hexaethylene glycol; —NH(CH₂)ₙCONH—CH₂—CH(OH)—CH₂—, —N(COCH₃) (CH₂)ₙCONH—CH₂—CH(OH)—CH₂—, wherein n is an integer from 1 to 10; or 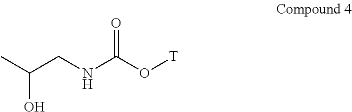
wherein T is 1-15 repetitive Compound 1: 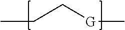
wherein G is selected from CH₂, O, S, and NH.

230. The double strand oligonucleotide structure of claim 229,
wherein n is 5 and G is O.

231. The double strand oligonucleotide structure of claim 216, wherein the double strand oligonucleotide comprises a sequence targeting hu-BACE1 or hu-MAPT.

232. The double strand oligonucleotide structure of claim 231, wherein the double strand oligonucleotide comprises a sequence targeting hu-BACE1, wherein the sequence is selected from SEQ ID NO: 22-45.

233. The double strand oligonucleotide structure of claim 231, wherein the double strand oligonucleotide comprises a sequence targeting hu-MAPT, optionally wherein the sequence is selected from SEQ ID NO: 46-68.

234. The double strand oligonucleotide structure of claim 216, wherein the double strand oligonucleotide comprises a sequence selected from SEQ ID Nos: 22-131.

235. A nanoparticle comprising the double strand oligonucleotide structure of claim 216.

236. A pharmaceutical composition comprising the double strand oligonucleotide structure of claim 216.

237. A method of treating a neurological disease, comprising administration of the double strand oligonucleotide structure of claim 216.

238. The method of claim 237, wherein the neurological disease is Alzheimer's disease (AD), stroke, dementia, muscular dystrophy (MD), multiple sclerosis (MS), amyotrophic lateral sclerosis (ALS), cystic fibrosis, Angel-man syndrome, Liddle syndrome, Parkinson's disease, Pick's disease, Paget's disease, cancer, or traumatic brain damage.


SEQUENCE LISTING (248 sequences)

SEQ ID NO: 1 - DNA, 50 bp, synthetic construct
  source 1..50: mol_type = other DNA
gtcgtatccagtgcagggtccgaggtattcgcactggatacgacacggga

SEQ ID NO: 2 - DNA, 18 bp, synthetic construct
  source 1..18: mol_type = other DNA
gcgatggcatgcacagtc

SEQ ID NO: 3 - DNA, 16 bp, synthetic construct
  source 1..16: mol_type = other DNA
gtgcagggtccgaggt

SEQ ID NO: 4 - DNA, 18 bp, synthetic construct
  source 1..18: mol_type = other DNA
tgaagtgctggcctctgt

SEQ ID NO: 5 - DNA, 22 bp, synthetic construct
  source 1..22: mol_type = other DNA
gatcctgtccattgatctccac

SEQ ID NO: 6 - DNA, 22 bp, synthetic construct
  source 1..22: mol_type = other DNA
  modified_base 1: mod_base = OTHER; note = Tetramethylrhodamine (TAMRA) azide modified nucleotide
  modified_base 22: mod_base = OTHER; note = Excellent Bioneer Quencher (EBQ) modified nucleotide
acacccatccggcgggagtggt

SEQ ID NO: 7 - DNA, 18 bp, synthetic construct
  source 1..18: mol_type = other DNA
accacagccaccttctcc

SEQ ID NO: 8 - DNA, 21 bp, synthetic construct
  source 1..21: mol_type = other DNA
agccatcctggttcaaagttc

SEQ ID NO: 9 - DNA, 23 bp, synthetic construct
  source 1..23: mol_type = other DNA
  modified_base 1: mod_base = OTHER; note = Tetramethylrhodamine (TAMRA) azide modified nucleotide
  modified_base 23: mod_base = OTHER; note = Excellent Bioneer Quencher (EBQ) modified nucleotide
cgctgtcctctcccgtcctcgcc

SEQ ID NO: 10 - DNA, 20 bp, synthetic construct
  source 1..20: mol_type = other DNA
tgcagactttgctttccttg

SEQ ID NO: 11 - DNA, 22 bp, synthetic construct
  source 1..22: mol_type = other DNA
ctggcttatatccaacacttcg

SEQ ID NO: 12 - DNA, 23 bp, synthetic construct
  source 1..23: mol_type = other DNA
  modified_base 1: mod_base = OTHER; note = Texas Red fluorophore modified nucleotide
  modified_base 23: mod_base = OTHER; note = Excellent Bioneer Quencher (EBQ) modified nucleotide
ccaaagatggtcaaggtcgcaag

SEQ ID NO: 13 - DNA, 21 bp, synthetic construct
  source 1..21: mol_type = other DNA
gcaaaaaccctttggctttga

SEQ ID NO: 14 - DNA, 20 bp, synthetic construct
  source 1..20: mol_type = other DNA
ctgctcaggccaccataatc

SEQ ID NO: 15 - DNA, 20 bp, synthetic construct
  source 1..20: mol_type = other DNA
  modified_base 1: mod_base = OTHER; note = Fluorescein modified nucleotide
  modified_base 20: mod_base = OTHER; note = Excellent Bioneer Quencher (EBQ) modified nucleotide
cgcctccccagcctgcctag

SEQ ID NO: 16 - DNA, 21 bp, synthetic construct
  source 1..21: mol_type = other DNA
cgccttctgtcgattatcagg

SEQ ID NO: 17 - DNA, 20 bp, synthetic construct
  source 1..20: mol_type = other DNA
ctccagcatggtcttccatt

SEQ ID NO: 18 - DNA, 21 bp, synthetic construct
  source 1..21: mol_type = other DNA
  modified_base 1: mod_base = OTHER; note = Fluorescein modified nucleotide
  modified_base 21: mod_base = OTHER; note = Excellent Bioneer Quencher (EBQ) modified nucleotide
tggctgaccctcgccaggagt

SEQ ID NO: 19 - DNA, 22 bp, synthetic construct
  source 1..22: mol_type = other DNA
aaacttgcccaagtattctcag

SEQ ID NO: 20 - DNA, 21 bp, synthetic construct
  source 1..21: mol_type = other DNA
atgaaaggtatccctccaacc

SEQ ID NO: 21 - DNA, 23 bp, synthetic construct
  source 1..23: mol_type = other DNA
  modified_base 1: mod_base = OTHER; note = Tetramethylrhodamine (TAMRA) azide modified nucleotide
  modified_base 23: mod_base = OTHER; note = Excellent Bioneer Quencher (EBQ) modified nucleotide
tgccagctggactgcaggcgact

SEQ ID NO: 22 - DNA, 19 bp, synthetic construct
  source 1..19: mol_type = other DNA
aggcaccaccccttggaac

SEQ ID NO: 23 - RNA, 19 nt, synthetic construct
  source 1..19: mol_type = other RNA
gttccaaggggtggtgcct

SEQ ID NO: 24 - DNA, 19 bp, synthetic construct
  source 1..19: mol_type = other DNA
ccagcacataccgggacct

SEQ ID NO: 25 - RNA, 19 nt, synthetic construct
  source 1..19: mol_type = other RNA
aggtcccggtatgtgctgg

SEQ ID NO: 26 - DNA, 19 bp, synthetic construct
  source 1..19: mol_type = other DNA
ggtgaggttaccaaccagt

SEQ ID NO: 27 - RNA, 19 nt, synthetic construct
  source 1..19: mol_type = other RNA
actggttggtaacctcacc

SEQ ID NO: 28 - DNA, 19 bp, synthetic construct
  source 1..19: mol_type = other DNA
cccgaaaacgaattggctt

SEQ ID NO: 29 - RNA, 19 nt, synthetic construct
  source 1..19: mol_type = other RNA
aagccaattcgttttcggg

SEQ ID NO: 30 - DNA, 19 bp, synthetic construct
  source 1..19: mol_type = other DNA
tggctttgctgtcagcgct

SEQ ID NO: 31 - RNA, 19 nt, synthetic construct
  source 1..19: mol_type = other RNA
agcgctgacagcaaagcca

SEQ ID NO: 32 - DNA, 19 bp, synthetic construct
  source 1..19: mol_type = other DNA
tactcttggtcacctcaaa

SEQ ID NO: 33 - RNA, 19 nt, synthetic construct
  source 1..19: mol_type = other RNA
tttgaggtgaccaagagta

SEQ ID NO: 34 - DNA, 19 bp, synthetic construct
  source 1..19: mol_type = other DNA
aatactcttggtcacctca

SEQ ID NO: 35 - RNA, 19 nt, synthetic construct
  source 1..19: mol_type = other RNA
tgaggtgaccaagagtatt

SEQ ID NO: 36 - DNA, 19 bp, synthetic construct
  source 1..19: mol_type = other DNA
ccaaccagtccttccgcat

SEQ ID NO: 37 - RNA, 19 nt, synthetic construct
  source 1..19: mol_type = other RNA
atgcggaaggactggttgg

SEQ ID NO: 38 - DNA, 19 bp, synthetic construct
  source 1..19: mol_type = other DNA
atccggcgggagtggtatt

SEQ ID NO: 39 - RNA, 19 nt, synthetic construct
  source 1..19: mol_type = other RNA
aataccactcccgccggat

SEQ ID NO: 40 - DNA, 19 bp, synthetic construct
  source 1..19: mol_type = other DNA
cctccggaagggtgtgtat

SEQ ID NO: 41 - RNA, 19 nt, synthetic construct
  source 1..19: mol_type = other RNA
atacacacccttccggagg

SEQ ID NO: 42 - DNA, 19 bp, synthetic construct
  source 1..19: mol_type = other DNA
cagcacataccgggacctc

SEQ ID NO: 43 - RNA, 19 nt, synthetic construct
  source 1..19: mol_type = other RNA
gaggtcccggtatgtgctg

SEQ ID NO: 44 - DNA, 19 bp, synthetic construct
  source 1..19: mol_type = other DNA
gggtggagatcaatggaca

SEQ ID NO: 45 - RNA, 19 nt, synthetic construct
  source 1..19: mol_type = other RNA
tgtccattgatctccaccc

SEQ ID NO: 46 - DNA, 19 bp, synthetic construct
  source 1..19: mol_type = other DNA
gggctgatggtaaaacgaa

SEQ ID NO: 47 - RNA, 19 nt, synthetic construct
  source 1..19: mol_type = other RNA
ttcgttttaccatcagccc

SEQ ID NO: 48 - DNA, 19 bp, synthetic construct
  source 1..19: mol_type = other DNA
ctggtctggcttgcggcgc

SEQ ID NO: 49 - RNA, 19 nt, synthetic construct
  source 1..19: mol_type = other RNA
gcgccgcaagccagaccag

SEQ ID NO: 50 - DNA, 19 bp, synthetic construct
  source 1..19: mol_type = other DNA
ggaagatcacgctgggacg

SEQ ID NO: 51 - RNA, 19 nt, synthetic construct
  source 1..19: mol_type = other RNA
cgtcccagcgtgatcttcc

SEQ ID NO: 52 - DNA, 19 bp, synthetic construct
  source 1..19: mol_type = other DNA
agctaaggaggccgttcag

SEQ ID NO: 53 - RNA, 19 nt, synthetic construct
  source 1..19: mol_type = other RNA
ctgaacggcctccttagct

SEQ ID NO: 54 - DNA, 19 bp, synthetic construct
  source 1..19: mol_type = other DNA
ggattaggactgaagcgat

SEQ ID NO: 55 - RNA, 19 nt, synthetic construct
  source 1..19: mol_type = other RNA
atcgcttcagtcctaatcc

SEQ ID NO: 56 - DNA, 19 bp, synthetic construct
  source 1..19: mol_type = other DNA
ccgtactccacccaagtcg

SEQ ID NO: 57 - RNA, 19 nt, synthetic construct
  source 1..19: mol_type = other RNA
cgacttgggtggagtacgg

SEQ ID NO: 58 - DNA, 19 bp, synthetic construct
  source 1..19: mol_type = other DNA
cgtactccacccaagtcgc

SEQ ID NO: 59 - RNA, 19 nt, synthetic construct
  source 1..19: mol_type = other RNA
gcgacttgggtggagtacg

SEQ ID NO: 60 - DNA, 19 bp, synthetic construct
  source 1..19: mol_type = other DNA
ctggcttgcggcgcgagga

SEQ ID NO: 61 - RNA, 19 nt, synthetic construct
  source 1..19: mol_type = other RNA
tcctcgcgccgcaagccag

SEQ ID NO: 62 - DNA, 19 bp, synthetic construct
  source 1..19: mol_type = other DNA
ttctaacccaccctcacga

SEQ ID NO: 63 - RNA, 19 nt, synthetic construct
  source 1..19: mol_type = other RNA
tcgtgagggtgggttagaa

SEQ ID NO: 64 - DNA, 19 bp, synthetic construct
  source 1..19: mol_type = other DNA
aagtccaagatcggctcca

SEQ ID NO: 65 - RNA, 19 nt, synthetic construct
  source 1..19: mol_type = other RNA
tggagccgatcttggactt

SEQ ID NO: 66 - DNA, 19 bp, synthetic construct
  source 1..19: mol_type = other DNA
tcgccgtcttccgccaaga

SEQ ID NO: 67 - RNA, 19 nt, synthetic construct
  source 1..19: mol_type = other RNA
tcttggcggaagacggcga

SEQ ID NO: 68 - DNA, 19 bp, synthetic construct
  source 1..19: mol_type = other DNA
ggatcgcagcggctacagc

SEQ ID NO: 69 - RNA, 19 nt, synthetic construct
  source 1..19: mol_type = other RNA
gctgtagccgctgcgatcc

SEQ ID NO: 70 - DNA, 19 bp, synthetic construct
  source 1..19: mol_type = other DNA
aacgtcactgtgcgtgcca

SEQ ID NO: 71 - RNA, 19 nt, synthetic construct
  source 1..19: mol_type = other RNA
tggcacgcacagtgacgtt

SEQ ID NO: 72 - DNA, 19 bp, synthetic construct
  source 1..19: mol_type = other DNA
cgggagaccgacgaggaat

SEQ ID NO: 73 - RNA, 19 nt, synthetic construct
  source 1..19: mol_type = other RNA
attcctcgtcggtctcccg

SEQ ID NO: 74 - DNA, 19 bp, synthetic construct
  source 1..19: mol_type = other DNA
ggaggagcctggccggaga

SEQ ID NO: 75 - RNA, 19 nt, synthetic construct
  source 1..19: mol_type = other RNA
tctccggccaggctcctcc

SEQ ID NO: 76 - DNA, 19 bp, synthetic construct
  source 1..19: mol_type = other DNA
tccggctgccccttcgcag

SEQ ID NO: 77 - RNA, 19 nt, synthetic construct
  source 1..19: mol_type = other RNA
ctgcgaaggggcagccgga

SEQ ID NO: 78 - DNA, 19 bp, synthetic construct
  source 1..19: mol_type = other DNA
ggagcccggagtccctgtc

SEQ ID NO: 79 - RNA, 19 nt, synthetic construct
  source 1..19: mol_type = other RNA
gacagggactccgggctcc

SEQ ID NO: 80 - DNA, 19 bp, synthetic construct
  source 1..19: mol_type = other DNA
tgcctaggtgctgggagct

SEQ ID NO: 81 - RNA, 19 nt, synthetic construct
  source 1..19: mol_type = other RNA
agctcccagcacctaggca

SEQ ID NO: 82 - DNA, 19 bp, synthetic construct
  source 1..19: mol_type = other DNA
cgggcagtagtaactttgc

SEQ ID NO: 83 - RNA, 19 nt, synthetic construct
  source 1..19: mol_type = other RNA
gcaaagttactactgcccg

SEQ ID NO: 84 - DNA, 19 bp, synthetic construct
  source 1..19: mol_type = other DNA
gctgtccagcacatatcga

SEQ ID NO: 85 - RNA, 19 nt, synthetic construct
  source 1..19: mol_type = other RNA
tcgatatgtgctggacagc

SEQ ID NO: 86 - DNA, 19 bp, synthetic construct
  source 1..19: mol_type = other DNA
accctttcctgcatcgcta

SEQ ID NO: 87 - RNA, 19 nt, synthetic construct
  source 1..19: mol_type = other RNA
tagcgatgcaggaaagggt

SEQ ID NO: 88 - DNA, 19 bp, synthetic construct
  source 1..19: mol_type = other DNA
agggaacccatctcggcat

SEQ ID NO: 89 - RNA, 19 nt, synthetic construct
  source 1..19: mol_type = other RNA
atgccgagatgggttccct

SEQ ID NO: 90 - DNA, 19 bp, synthetic construct
  source 1..19: mol_type = other DNA
gcatccctcatggccccaa

SEQ ID NO: 91 - RNA, 19 nt, synthetic construct
  source 1..19: mol_type = other RNA
ttggggccatgagggatgc

SEQ ID NO: 92 - DNA, 19 bp, synthetic construct
  source 1..19: mol_type = other DNA
acaagcctttccgcctccc

SEQ ID NO: 93 - RNA, 19 nt, synthetic construct
  source 1..19: mol_type = other RNA
gggaggcggaaaggcttgt

SEQ ID NO: 94 - DNA, 19 bp, synthetic construct
  source 1..19: mol_type = other DNA
ctggattatggtggcctga

SEQ ID NO: 95 - RNA, 19 nt, synthetic construct
  source 1..19: mol_type = other RNA
tcaggccaccataatccag

SEQ ID NO: 96 - DNA, 19 bp, synthetic construct
  source 1..19: mol_type = other DNA
cagtcctgatgccgccgag

SEQ ID NO: 97 - RNA, 19 nt, synthetic construct
  source 1..19: mol_type = other RNA
ctcggcggcatcaggactg

SEQ ID NO: 98 - DNA, 19 bp, synthetic construct
  source 1..19: mol_type = other DNA
cgccgcctggaccgggaac

SEQ ID NO: 99 - RNA, 19 nt, synthetic construct
  source 1..19: mol_type = other RNA
gttcccggtccaggcggcg

SEQ ID NO: 100 - DNA, 19 bp, synthetic construct
  source 1..19: mol_type = other DNA
gggcaccgacctggtgagc

SEQ ID NO: 101 - RNA, 19 nt, synthetic construct
  source 1..19: mol_type = other RNA
gctcaccaggtcggtgccc

SEQ ID NO: 102 - DNA, 19 bp, synthetic construct
  source 1..19: mol_type = other DNA
tatgtggagatgaccgtag

SEQ ID NO: 103 - RNA, 19 nt, synthetic construct
  source 1..19: mol_type = other RNA
ctacggtcatctccacata

SEQ ID NO: 104 - DNA, 19 bp, synthetic construct
  source 1..19: mol_type = other DNA
gaggggaaagtccggccag

SEQ ID NO: 105 - RNA, 19 nt, synthetic construct
  source 1..19: mol_type = other RNA
ctggccggactttcccctc

SEQ ID NO: 106 - DNA, 19 bp, synthetic construct
  source 1..19: mol_type = other DNA
ccctacacccagggcaagt

SEQ ID NO: 107 - RNA, 19 nt, synthetic construct
  source 1..19: mol_type = other RNA
acttgccctgggtgtaggg

SEQ ID NO: 108 - DNA, 19 bp, synthetic construct
  source 1..19: mol_type = other DNA
tgggactttagggctaacc

SEQ ID NO: 109 - RNA, 19 nt, synthetic construct
  source 1..19: mol_type = other RNA
ggttagccctaaagtccca

SEQ ID NO: 110 - DNA, 19 bp, synthetic construct
  source 1..19: mol_type = other DNA
tggggtgatcagcccaaaa

SEQ ID NO: 111 - RNA, 19 nt, synthetic construct
  source 1..19: mol_type = other RNA
ttttgggctgatcacccca

SEQ ID NO: 112 - DNA, 19 bp, synthetic construct
  source 1..19: mol_type = other DNA
cgccggcctccagaacgcg

SEQ ID NO: 113 - RNA, 19 nt, synthetic construct
  source 1..19: mol_type = other RNA
cgcgttctggaggccggcg

SEQ ID NO: 114 - DNA, 19 bp, synthetic construct
  source 1..19: mol_type = other DNA
attgcttgcgttgtggggg

SEQ ID NO: 115 - RNA, 19 nt, synthetic construct
  source 1..19: mol_type = other RNA
cccccacaacgcaagcaat

SEQ ID NO: 116 - DNA, 19 bp, synthetic construct
  source 1..19: mol_type = other DNA
ggtgggctagatagatagg

SEQ ID NO: 117 - RNA, 19 nt, synthetic construct
  source 1..19: mol_type = other RNA
cctatctatctagcccacc

SEQ ID NO: 118 - DNA, 19 bp, synthetic construct
  source 1..19: mol_type = other DNA
ggaggaggaacacattaag

SEQ ID NO: 119 - RNA, 19 nt, synthetic construct
  source 1..19: mol_type = other RNA
cttaatgtgttcctcctcc

SEQ ID NO: 120 - DNA, 19 bp, synthetic construct
  source 1..19: mol_type = other DNA
gggcatgatctccagtgct

SEQ ID NO: 121 - RNA, 19 nt, synthetic construct
  source 1..19: mol_type = other RNA
agcactggagatcatgccc

SEQ ID NO: 122 - DNA, 19 bp, synthetic construct
  source 1..19: mol_type = other DNA
aaagggaggtcttggagtg

SEQ ID NO: 123 - RNA, 19 nt, synthetic construct
  source 1..19: mol_type = other RNA
cactccaagacctcccttt

SEQ ID NO: 124 - DNA, 19 bp, synthetic construct
  source 1..19: mol_type = other DNA
ggcaaaaccggggcgaaga

SEQ ID NO: 125 - RNA, 19 nt, synthetic construct
  source 1..19: mol_type = other RNA
tcttcgccccggttttgcc

SEQ ID NO: 126 - DNA, 19 bp, synthetic construct
  source 1..19: mol_type = other DNA
tacaattggcctggttcct

SEQ ID NO: 127 - RNA, 19 nt, synthetic construct
  source 1..19: mol_type = other RNA
aggaaccaggccaattgta

SEQ ID NO: 128 - DNA, 19 bp, synthetic construct
  source 1..19: mol_type = other DNA
gctcgtgtggccagcaaag

SEQ ID NO: 129 - RNA, 19 nt, synthetic construct
  source 1..19: mol_type = other RNA
ctttgctggccacacgagc

SEQ ID NO: 130 - DNA, 19 bp, synthetic construct
  source 1..19: mol_type = other DNA
gagccaatgtctttgggtg

SEQ ID NO: 131 - RNA, 19 nt, synthetic construct
  source 1..19: mol_type = other RNA
cacccaaagacattggctc

SEQ ID NO: 132 - DNA, 20 bp, synthetic construct
  source 1..20: mol_type = other DNA
acacctactctgggaagcgt

SEQ ID NO: 133 - DNA, 20 bp, synthetic construct
  source 1..20: mol_type = other DNA
gccaggtatttgtggttcgt

SEQ ID NO: 134 - DNA, 20 bp, synthetic construct
  source 1..20: mol_type = other DNA
gtgtttgacggcatcccacc

SEQ ID NO: 135 - DNA, 20 bp, synthetic construct
  source 1..20: mol_type = other DNA
taggcttcagacgcacgacc

SEQ ID NO: 136 - RNA, 19 nt, synthetic construct
  source 1..19: mol_type = other RNA
aggcaccaccccttggaac

SEQ ID NO: 137 - DNA, 19 bp, synthetic construct
  source 1..19: mol_type = other DNA
gttccaaggggtggtgcct

SEQ ID NO: 138 - RNA, 19 nt, synthetic construct
  source 1..19: mol_type = other RNA
ccagcacataccgggacct

SEQ ID NO: 139 - DNA, 19 bp, synthetic construct
  source 1..19: mol_type = other DNA
aggtcccggtatgtgctgg

SEQ ID NO: 140 - RNA, 19 nt, synthetic construct
  source 1..19: mol_type = other RNA
ggtgaggttaccaaccagt

SEQ ID NO: 141 - DNA, 19 bp, synthetic construct
  source 1..19: mol_type = other DNA
actggttggtaacctcacc

SEQ ID NO: 142 - RNA, 19 nt, synthetic construct
  source 1..19: mol_type = other RNA
cccgaaaacgaattggctt

SEQ ID NO: 143 - DNA, 19 bp, synthetic construct
  source 1..19: mol_type = other DNA
aagccaattcgttttcggg

SEQ ID NO: 144 - RNA, 19 nt, synthetic construct
  source 1..19: mol_type = other RNA
tggctttgctgtcagcgct

SEQ ID NO: 145 - DNA, 19 bp, synthetic construct
  source 1..19: mol_type = other DNA
agcgctgacagcaaagcca

SEQ ID NO: 146 - RNA, 19 nt, synthetic construct
  source 1..19: mol_type = other RNA
tactcttggtcacctcaaa

SEQ ID NO: 147 - DNA, 19 bp, synthetic construct
  source 1..19: mol_type = other DNA
tttgaggtgaccaagagta

SEQ ID NO: 148 - RNA, 19 nt, synthetic construct
  source 1..19: mol_type = other RNA
aatactcttggtcacctca

SEQ ID NO: 149 - DNA, 19 bp, synthetic construct
  source 1..19: mol_type = other DNA
tgaggtgaccaagagtatt

SEQ ID NO: 150 - RNA, 19 nt, synthetic construct
  source 1..19: mol_type = other RNA
ccaaccagtccttccgcat

SEQ ID NO: 151 - DNA, 19 bp, synthetic construct
  source 1..19: mol_type = other DNA
atgcggaaggactggttgg

SEQ ID NO: 152 - RNA, 19 nt, synthetic construct
  source 1..19: mol_type = other RNA
atccggcgggagtggtatt

SEQ ID NO: 153 - DNA, 19 bp, synthetic construct
  source 1..19: mol_type = other DNA
aataccactcccgccggat

SEQ ID NO: 154 - RNA, 19 nt, synthetic construct
  source 1..19: mol_type = other RNA
cctccggaagggtgtgtat

SEQ ID NO: 155 - DNA, 19 bp, synthetic construct
  source 1..19: mol_type = other DNA
atacacacccttccggagg

SEQ ID NO: 156 - RNA, 19 nt, synthetic construct
  source 1..19: mol_type = other RNA
cagcacataccgggacctc

SEQ ID NO: 157 - DNA, 19 bp, synthetic construct
  source 1..19: mol_type = other DNA
gaggtcccggtatgtgctg

SEQ ID NO: 158 - RNA, 19 nt, synthetic construct
  source 1..19: mol_type = other RNA
gggtggagatcaatggaca

SEQ ID NO: 159 - DNA, 19 bp, synthetic construct
  source 1..19: mol_type = other DNA
tgtccattgatctccaccc

SEQ ID NO: 160 - RNA, 19 nt, synthetic construct
  source 1..19: mol_type = other RNA
gggctgatggtaaaacgaa

SEQ ID NO: 161 - DNA, 19 bp, synthetic construct
  source 1..19: mol_type = other DNA
ttcgttttaccatcagccc

SEQ ID NO: 162 - RNA, 19 nt, synthetic construct
  source 1..19: mol_type = other RNA
ctggtctggcttgcggcgc

SEQ ID NO: 163 - DNA, 19 bp, synthetic construct
  source 1..19: mol_type = other DNA
gcgccgcaagccagaccag

SEQ ID NO: 164 - RNA, 19 nt, synthetic construct
  source 1..19: mol_type = other RNA
ggaagatcacgctgggacg

SEQ ID NO: 165 - DNA, 19 bp, synthetic construct
  source 1..19: mol_type = other DNA
cgtcccagcgtgatcttcc

SEQ ID NO: 166 - RNA, 19 nt, synthetic construct
  source 1..19: mol_type = other RNA
agctaaggaggccgttcag

SEQ ID NO: 167 - DNA, 19 bp, synthetic construct
  source 1..19: mol_type = other DNA
ctgaacggcctccttagct

SEQ ID NO: 168 - RNA, 19 nt, synthetic construct
  source 1..19: mol_type = other RNA
ggattaggactgaagcgat

SEQ ID NO: 169 - DNA, 19 bp, synthetic construct
  source 1..19: mol_type = other DNA
atcgcttcagtcctaatcc

SEQ ID NO: 170 - RNA, 19 nt, synthetic construct
  source 1..19: mol_type = other RNA
ccgtactccacccaagtcg

SEQ ID NO: 171 - DNA, 19 bp, synthetic construct
  source 1..19: mol_type = other DNA
cgacttgggtggagtacgg

SEQ ID NO: 172 - RNA, 19 nt, synthetic construct
  source 1..19: mol_type = other RNA
cgtactccacccaagtcgc

SEQ ID NO: 173 - DNA, 19 bp, synthetic construct
  source 1..19: mol_type = other DNA
gcgacttgggtggagtacg

SEQ ID NO: 174 - RNA, 19 nt, synthetic construct
  source 1..19: mol_type = other RNA
ctggcttgcggcgcgagga

SEQ ID NO: 175 - DNA, 19 bp, synthetic construct
  source 1..19: mol_type = other DNA
tcctcgcgccgcaagccag

SEQ ID NO: 176 - RNA, 19 nt, synthetic construct
  source 1..19: mol_type = other RNA
ttctaacccaccctcacga

SEQ ID NO: 177 - DNA, 19 bp, synthetic construct
  source 1..19: mol_type = other DNA
tcgtgagggtgggttagaa

SEQ ID NO: 178 - RNA, 19 nt, synthetic construct
  source 1..19: mol_type = other RNA
aagtccaagatcggctcca

SEQ ID NO: 179 - DNA, 19 bp, synthetic construct
  source 1..19: mol_type = other DNA
tggagccgatcttggactt

SEQ ID NO: 180 - RNA, 19 nt, synthetic construct
  source 1..19: mol_type = other RNA
tcgccgtcttccgccaaga

SEQ ID NO: 181 - DNA, 19 bp, synthetic construct
  source 1..19: mol_type = other DNA
tcttggcggaagacggcga

SEQ ID NO: 182 - RNA, 19 nt, synthetic construct
  source 1..19: mol_type = other RNA
ggatcgcagcggctacagc

SEQ ID NO: 183 - DNA, 19 bp, synthetic construct
  source 1..19: mol_type = other DNA
gctgtagccgctgcgatcc

SEQ ID NO: 184 - RNA, 19 nt, synthetic construct
  source 1..19: mol_type = other RNA
aacgtcactgtgcgtgcca

SEQ ID NO: 185 - DNA, 19 bp, synthetic construct
  source 1..19: mol_type = other DNA
tggcacgcacagtgacgtt

SEQ ID NO: 186 - RNA, 19 nt, synthetic construct
  source 1..19: mol_type = other RNA
cgggagaccgacgaggaat

SEQ ID NO: 187 - DNA, 19 bp, synthetic construct
  source 1..19: mol_type = other DNA
attcctcgtcggtctcccg

SEQ ID NO: 188 - RNA, 19 nt, synthetic construct
  source 1..19: mol_type = other RNA
ggaggagcctggccggaga

SEQ ID NO: 189 - DNA, 19 bp, synthetic construct
  source 1..19: mol_type = other DNA
tctccggccaggctcctcc

SEQ ID NO: 190 - RNA, 19 nt, synthetic construct
  source 1..19: mol_type = other RNA
tccggctgccccttcgcag

SEQ ID NO: 191 - DNA, 19 bp, synthetic construct
  source 1..19: mol_type = other DNA
ctgcgaaggggcagccgga

SEQ ID NO: 192 - RNA, 19 nt, synthetic construct
  source 1..19: mol_type = other RNA
ggagcccggagtccctgtc

SEQ ID NO: 193 - DNA, 19 bp, synthetic construct
  source 1..19: mol_type = other DNA
gacagggactccgggctcc

SEQ ID NO: 194 - RNA, 19 nt, synthetic construct
  source 1..19: mol_type = other RNA
tgcctaggtgctgggagct

SEQ ID NO: 195 - DNA, 19 bp, synthetic construct
  source 1..19: mol_type = other DNA
agctcccagcacctaggca

SEQ ID NO: 196 - RNA, 19 nt, synthetic construct
  source 1..19: mol_type = other RNA
cgggcagtagtaactttgc

SEQ ID NO: 197 - DNA, 19 bp, synthetic construct
  source 1..19: mol_type = other DNA
gcaaagttactactgcccg

SEQ ID NO: 198 - RNA, 19 nt, synthetic construct
  source 1..19: mol_type = other RNA
gctgtccagcacatatcga

SEQ ID NO: 199 - DNA, 19 bp, synthetic construct
  source 1..19: mol_type = other DNA
tcgatatgtgctggacagc

SEQ ID NO: 200 - RNA, 19 nt, synthetic construct
  source 1..19: mol_type = other RNA
accctttcctgcatcgcta

SEQ ID NO: 201 - DNA, 19 bp, synthetic construct
  source 1..19: mol_type = other DNA
tagcgatgcaggaaagggt

SEQ ID NO: 202 - RNA, 19 nt, synthetic construct
  source 1..19: mol_type = other RNA
agggaacccatctcggcat

SEQ ID NO: 203 - DNA, 19 bp, synthetic construct
  source 1..19: mol_type = other DNA
atgccgagatgggttccct

SEQ ID NO: 204 - RNA, 19 nt, synthetic construct
  source 1..19: mol_type = other RNA
gcatccctcatggccccaa

SEQ ID NO: 205 - DNA, 19 bp, synthetic construct
  source 1..19: mol_type = other DNA
ttggggccatgagggatgc

SEQ ID NO: 206 - RNA, 19 nt, synthetic construct
  source 1..19: mol_type = other RNA
acaagcctttccgcctccc

SEQ ID NO: 207 - DNA, 19 bp, synthetic construct
  source 1..19: mol_type = other DNA
gggaggcggaaaggcttgt

SEQ ID NO: 208 - RNA, 19 nt, synthetic construct
  source 1..19: mol_type = other RNA
ctggattatggtggcctga

SEQ ID NO: 209 - DNA, 19 bp, synthetic construct
  source 1..19: mol_type = other DNA
tcaggccaccataatccag

SEQ ID NO: 210 - RNA, 19 nt, synthetic construct
  source 1..19: mol_type = other RNA
cagtcctgatgccgccgag

SEQ ID NO: 211 - DNA, 19 bp, synthetic construct
  source 1..19: mol_type = other DNA
ctcggcggcatcaggactg

SEQ ID NO: 212 - RNA, 19 nt, synthetic construct
  source 1..19: mol_type = other RNA
cgccgcctggaccgggaac

SEQ ID NO: 213 - DNA, 19 bp, synthetic construct
  source 1..19: mol_type = other DNA
gttcccggtccaggcggcg

SEQ ID NO: 214 - RNA, 19 nt, synthetic construct
  source 1..19: mol_type = other RNA
gggcaccgacctggtgagc

SEQ ID NO: 215 - DNA, 19 bp, synthetic construct
  source 1..19: mol_type = other DNA
gctcaccaggtcggtgccc

SEQ ID NO: 216 - RNA, 19 nt, synthetic construct
  source 1..19: mol_type = other RNA
tatgtggagatgaccgtag

SEQ ID NO: 217 - DNA, 19 bp, synthetic construct
  source 1..19: mol_type = other DNA
ctacggtcatctccacata

SEQ ID NO: 218 - RNA, 19 nt, synthetic construct
  source 1..19: mol_type = other RNA
gaggggaaagtccggccag

SEQ ID NO: 219 - DNA, 19 bp, synthetic construct
  source 1..19: mol_type = other DNA
ctggccggactttcccctc

SEQ ID NO: 220 - RNA, 19 nt, synthetic construct
  source 1..19: mol_type = other RNA
ccctacacccagggcaagt

SEQ ID NO: 221 - DNA, 19 bp, synthetic construct
  source 1..19: mol_type = other DNA
acttgccctgggtgtaggg

SEQ ID NO: 222 - RNA, 19 nt, synthetic construct
  source 1..19: mol_type = other RNA
tgggactttagggctaacc

SEQ ID NO: 223 - DNA, 19 bp, synthetic construct
  source 1..19: mol_type = other DNA
ggttagccctaaagtccca

SEQ ID NO: 224 - RNA, 19 nt, synthetic construct
  source 1..19: mol_type = other RNA
tggggtgatcagcccaaaa

SEQ ID NO: 225 - DNA, 19 bp, synthetic construct
  source 1..19: mol_type = other DNA
ttttgggctgatcacccca

SEQ ID NO: 226 - RNA, 19 nt, synthetic construct
  source 1..19: mol_type = other RNA
cgccggcctccagaacgcg

SEQ ID NO: 227 - DNA, 19 bp, synthetic construct
  source 1..19: mol_type = other DNA
cgcgttctggaggccggcg

SEQ ID NO: 228 - RNA, 19 nt, synthetic construct
  source 1..19: mol_type = other RNA
attgcttgcgttgtggggg

SEQ ID NO: 229 - DNA, 19 bp, synthetic construct
  source 1..19: mol_type = other DNA
cccccacaacgcaagcaat

SEQ ID NO: 230 - RNA, 19 nt, synthetic construct
  source 1..19: mol_type = other RNA
ggtgggctagatagatagg

SEQ ID NO: 231 - DNA, 19 bp, synthetic construct
  source 1..19: mol_type = other DNA
cctatctatctagcccacc

SEQ ID NO: 232 - RNA, 19 nt, synthetic construct
  source 1..19: mol_type = other RNA
ggaggaggaacacattaag

SEQ ID NO: 233 - DNA, 19 bp, synthetic construct
  source 1..19: mol_type = other DNA
cttaatgtgttcctcctcc

SEQ ID NO: 234 - RNA, 19 nt, synthetic construct
  source 1..19: mol_type = other RNA
gggcatgatctccagtgct

SEQ ID NO: 235 - DNA, 19 bp, synthetic construct
  source 1..19: mol_type = other DNA
agcactggagatcatgccc

SEQ ID NO: 236 - RNA, 19 nt, synthetic construct
  source 1..19: mol_type = other RNA
aaagggaggtcttggagtg

SEQ ID NO: 237 - DNA, 19 bp, synthetic construct
  source 1..19: mol_type = other DNA
cactccaagacctcccttt

SEQ ID NO: 238 - RNA, 19 nt, synthetic construct
  source 1..19: mol_type = other RNA
ggcaaaaccggggcgaaga

SEQ ID NO: 239 - DNA, 19 bp, synthetic construct
  source 1..19: mol_type = other DNA
tcttcgccccggttttgcc

SEQ ID NO: 240 - RNA, 19 nt, synthetic construct
  source 1..19: mol_type = other RNA
tacaattggcctggttcct

SEQ ID NO: 241 - DNA, 19 bp, synthetic construct
  source 1..19: mol_type = other DNA
aggaaccaggccaattgta

SEQ ID NO: 242 - RNA, 19 nt, synthetic construct
  source 1..19: mol_type = other RNA
gctcgtgtggccagcaaag

SEQ ID NO: 243 - DNA, 19 bp, synthetic construct
  source 1..19: mol_type = other DNA
ctttgctggccacacgagc

SEQ ID NO: 244 - RNA, 19 nt, synthetic construct
  source 1..19: mol_type = other RNA
gagccaatgtctttgggtg

SEQ ID NO: 245 - DNA, 19 bp, synthetic construct
  source 1..19: mol_type = other DNA
cacccaaagacattggctc

SEQ ID NO: 246 - protein, 10 aa, synthetic construct
  VARIANT 1..10: note = This sequence may encompass 3-10 residues
HHHHHHHHHH

SEQ ID NO: 247 - protein, 8 aa, synthetic construct
  VARIANT 1..8: note = This sequence may encompass 5-8 residues
HHHHHHHH

SEQ ID NO: 248 - protein, 6 aa, synthetic construct
HHHHHH